(19) United States
(12) Patent Application Publication
(10) Pub. No.: US 2025/0313575 A1
(43) Pub. Date: Oct. 9, 2025

(54) MACROCYCLES AS LRRK2 INHIBITORS, PHARMACEUTICAL COMPOSITIONS, AND USES THEREOF

(71) Applicant: Merck Sharp & Dohme LLC, Rahway, NJ (US)

(72) Inventors: Elsie Yu, Brighton, MA (US); Peter Fuller, Ashland, MA (US); Hua Zhou, Acton, MA (US); Kaitlyn M. Logan, Jamaica Plain, MA (US); Solomon D. Kattar, Wakefield, MA (US); Anmol Gulati, Watertown, MA (US); Mitchell H. Keylor, Mansfield, MA (US); Kelsey E. Poremba, Natick, MA (US); Michael Joseph Ardolino, Arlington, MA (US); Xin Yan, Newton Highlands, MA (US); Rachel L. Palte, Melrose, MA (US); Dong Xiao, Warren, NJ (US)

(73) Assignee: Merck Sharp & Dohme LLC, Rahway, NJ (US)

(21) Appl. No.: 18/866,775

(22) PCT Filed: May 15, 2023
(86) PCT No.: PCT/US2023/022204

§ 371 Date: Nov. 18, 2024

Related U.S. Application Data
(60) Provisional application No. 63/344,331, filed on May 20, 2022.

(51) Int. Cl. C07D 498/22; A61K 31/529; A61K 31/5386; C07D 487/18; C07D 498/18; C07D 498/20

(52) U.S. Cl. CPC C07D 498/22; A61K 31/529; A61K 31/5386; C07D 487/18; C07D 498/18; C07D 498/20

(57) ABSTRACT

The present invention is directed to certain 2-aminoquinzaoline derivatives of Formula (I) and (Ia); and pharmaceutically acceptable salts thereof, which are potent inhibitors of LRRK2 kinase and may be useful in the treatment or prevention of diseases in which the LRRK2 kinase is involved, such as Parkinson's Disease and other diseases and disorders described herein. The invention is also directed to pharmaceutical compositions comprising these compounds and the use of these compounds and compositions in the prevention or treatment of such diseases in which LRRK-2 kinase is involved.

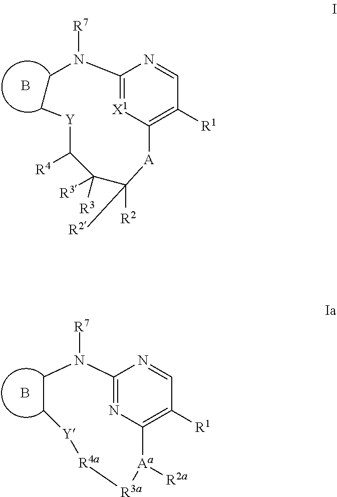

### BACKGROUND OF THE INVENTION

Parkinson's disease (PD) is a common neurodegenerative disease caused by progressive loss of mid-brain dopaminergic neurons leading to abnormal motor symptoms such as bradykinesia, rigidity and resting tremor. Many PD patients also experience a variety of non-motor symptoms including cognitive dysfunction, autonomic dysfunction, emotional changes and sleep disruption. The combined motor and non-motor symptoms of Parkinson's disease severely impact patient quality of life.

While the majority of PD cases are idiopathic, there are several genetic determinants such as mutations in SNCA, Parkin, PINK1, DJ-1 and LRRK2. Linkage analysis studies have demonstrated that multiple missense mutations in the Leucine-Rich Repeat Kinase 2 (LRRK2) gene lead to an autosomal late onset form of PD. LRRK2 is a 286 kDa cytoplasmic protein containing kinase and GTPase domains as well as multiple protein-protein interaction domains. See for example, Aasly et al., Annals of Neurology, Vol. 57 (5), May 2005, pp. 762-765; Adams et al., Brain, Vol. 128, 2005, pp. 2777-85; Gilks et al., Lancet, Vol. 365, Jan. 29, 2005, pp. 415-416, Nichols et al., Lancet, Vol. 365, Jan. 29, 2005, pp. 410-412, and U. Kumari and E. Tan, FEBS journal 276 (2009) pp. 6455-6463.

In vitro biochemical studies have demonstrated that LRRK2 proteins harboring the PD associated proteins generally confer increased kinase activity and decreased GTP hydrolysis compared to the wild type protein (Guo et al., Experimental Cell Research, Vol, 313, 2007, pp. 3658-3670) thereby suggesting that small molecule LRRK2 kinase inhibitors may be able to block aberrant LRRK2-dependent signaling in PD. In support of this notion, it has been reported that inhibitors of LRRK2 are protective in models of PD (Lee et al., Nature Medicine, Vol 16, 2010, pp. 998-1000).

LRRK2 expression is highest in the same brain regions that are affected by PD. LRRK2 is found in Lewy bodies, a pathological hallmark of PD as well as other neurodegenerative diseases such as Lewy body dementia (Zhu et al., Molecular Neurodegeneration, Vol 30, 2006, pp. 1-17). Further, LRRK2 mRNA levels are increased in the striatum of MPTP-treated marmosets, an experimental model of Parkinson's disease, and the level of increased mRNA correlates with the level of L-Dopa induced dyskinesia suggesting that inhibition of LRRK2 kinase activity may have utility in ameliorating L-Dopa induced dyskinesias. These and other recent studies indicate that a potent, selective and brain penetrant LRRK2 kinase inhibitor could be a therapeutic treatment for PD. (Lee et al., Nat. Med. 2010 September; 16 (9): 998-1000; Zhu, et al., Mol. Neurodegeneration 2006 Nov. 30; 1:17; Daher, et al., J Biol Chem. 2015 Aug. 7; 290 (32): 19433-44; Volpicelli-Daley et al., J Neurosci. 2016 Jul. 13; 36 (28): 7415-27).

LRRK2 mutations have been associated with Alzheimer's-like pathology (Zimprach et al., Neuron. 2004 Nov. 18; 44 (4): 601-7) and the LRRK2 R1628P variant has been associated with an increased risk of developing AD (Zhao et al., Neurobiol Aging. 2011 November; 32 (11): 1990-3). Mutations in LRRK2 have also been identified that are clinically associated with the transition from mild cognitive impairment to Alzheimer's disease (see WO2007149798). Together these data suggest that LRRK2 inhibitors may be useful in the treatment of Alzheimer's disease and other dementias and related neurodegenerative disorders.

LRRK2 has been reported to phosphorylate tubulin-associated tau and this phosphorylation is enhanced by the kinase activating LRRK2 mutation G2019S (Kawakami et al., PLOS One. 2012; 7(1):e30834; Bailey et al., Acta Neuropathol. 2013 December; 126(6):809-27.). Additionally, over expression of LRRK2 in a tau transgenic mouse model resulted in the aggregation of insoluble tau and its phosphorylation at multiple epitopes (Bailey et al., 2013). Hyperphosphorylation of tau has also been observed in LRRK2 R1441G overexpressing transgenic mice (Li et al., Nat Neurosci. 2009 July; 12(7):826-8.). Inhibition of LRRK2 kinase activity may therefore be useful in the treatment of tauopathy disorders characterized by hyperphosphorylated of tau such as argyrophilic grain disease, Picks disease, corticobasal degeneration, progressive supranuclear palsy, inherited frontotemporal dementia and parkinson's linked to chromosome 17 (Goedert and Jakes Biochim Biophys Acta. 2005 Jan. 3.).

A growing body of evidence suggests a role for LRRK2 in immune cell function in the brain with LRRK2 inhibitors demonstrated to attenuate microglial inflammatory responses (Moehle et al., J Neurosci. 2012 Feb. 1; 32(5):1602-11.). As neuroinflammation is a hallmark of a number of neurodegenerative diseases such PD, AD, MS, HIV-induced dementia, ALS, ischemic stroke, MS, traumatic brain injury and spinal cord injury, LRRK2 kinases inhibitors may have utility in the treatment of neuroinflammation in these disorders. Significantly elevated levels of LRRK2 mRNA have been observed in muscle biopsy samples taken from patients with ALS (Shtilbans et al., Amyotroph Lateral Scler. 2011 July; 12(4):250-6.). LRRK2 inhibitors have been disclosed in the art, e.g., WO2016036586.

LRRK2 is also expressed in cells of the immune system and recent reports suggest that LRRK2 may play a role in the regulation of the immune system and modulation of inflammatory responses. LRRK2 kinase inhibitors may therefore be of utility in a number of diseases of the immune system such as lymphomas, leukemias, multiple sclerosis rheumatoid arthritis, systemic lupus erythematosus autoimmune hemolytic anemia, pure red cell aplasia, idiopathic thrombocytopenia pupura (ITP), Evans Syndrome, vasculitis, bullous skin disorder, type I diabetes mellitus, Sjorgen's syndrome, Delvic's disease, inflammatory myopathies (Engel at al., Pharmacol Rev. 2011 March; 63(1):127-56; Homam et al., Homam et al., Clin Neuromuscluar disease, 2010) and ankylosing spondylitis (Danoy et al., PLOS Genet. 2010 Dec. 2; 6(12).). Increased incidence of certain types of non-skin cancers such as renal, breast, lung, prostate, and acute myelogenous leukemia (AML) have been reported in patients with the LRRK2 G2019S mutation (Agalliu et al., JAMA Neurol. 2015 January; 72(1); Saunders-Pullman et al., Mov Disord. 2010 Nov. 15; 25(15):2536-41.). LRRK2 has amplification and overexpression has been reported in papillary renal and thyroid carcinomas. Inhibiting LRRK2 kinase activity may therefore be useful in the treatment of cancer (Looyenga et al., Proc Natl Acad Sci USA. 2011 Jan. 25; 108(4):1439-44).

Genome-wide association studies also highlight LRRK2 in the modification of susceptibility to the chronic autoimmune Crohn's disease and leprosy (Zhang et al., The New England Jopuranl of Medicine, Vol 361, 2009, pp. 2609-2618; Umeno et al., Inflammatory Bowel Disease Vol 17, 2011, pp. 2407-2415).

### SUMMARY OF THE INVENTION

The present invention is directed to certain macrocycles and pharmaceutically acceptable salts thereof, which are collectively or individually referred to herein as “compound(s) of the invention” or “compounds of Formula (I)”, as described herein. Applicant has found, surprisingly and advantageously, that the macrocycles of Formula I and Formula Ia, each of which possess a phenyl or five or six membered heterocycle moiety and six membered nitrogen containing heteroaryl, both of which are linked together through an amino substituent and a macrocyclic chain that is optionally substituted by an oxygen, nitrogen or carbon atom, exhibit excellent LRRK2 inhibitory activity. In some embodiments, the compounds of the invention exhibit unexpectedly superior potency as inhibitors of LRRK2 kinase, as evidenced by the data reported herein. The compounds of the invention may be useful in the treatment or prevention of diseases (or one or more symptoms associated with such diseases) in which the LRRK2 kinase is involved, including Parkinson's disease and other indications, diseases and disorders as described herein. The invention is also directed to pharmaceutical compositions comprising a compound of the invention and to methods for the use of such compounds and compositions for the treatments described herein.

### DETAILED DESCRIPTION OF THE INVENTION

For each of the following embodiments, any variable not explicitly defined in the embodiment is as defined in Formula I and Formula Ia. In each of the embodiments described herein, each variable is selected independently of the other unless otherwise noted.

In one aspect of the invention are provided, compounds of structural Formula I and Formula Ia:

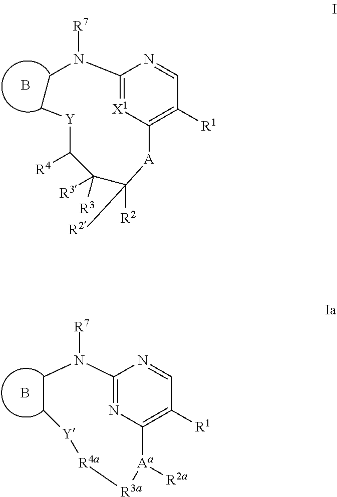

or a pharmaceutically acceptable salt thereof, wherein,
B is a C₅₋₆ heteroaryl or C₆₋₁₀ aryl, said heteroaryl and aryl optionally substituted with 1 to 3 groups of R^{x};
Y is O, OCH₂ or CH₂;
Y¹ is O, OCH₂, or CH₂;
X¹ is N or CH;
R¹ is selected from C₁₋₆alkyl, C₃₋₆cycloalkyl, halogen, C₁₋₃haloalkyl,
R² is selected from hydrogen, and C₁₋₆alkyl, said alkyl optionally substituted from 1 to 3 groups selected from C₁₋₆ alkyl, CF₃, and CN,
R^{2a} is hydrogen
R³ is selected from hydrogen, and C₁₋₆alkyl, said alkyl optionally substituted from 1 to 3 groups selected from C₁₋₆ alkyl, CF₃, and CN,
R^{3a} is CHR^{4b},
R^{4a} is (CH₂)ₙ; or
R^{3a} and R^{4a} combine to form a C₃₋₁₀ cycloalkyl, or C₃₋₁₀ heterocyclyl, said cycloalkyl and heterocyclyl optionally substituted with 1 to 3 groups of R^{b},
R^{4b} is selected from hydrogen and C₁₋₃alkyl,
R^{2′} and R^{3′} are independently hydrogen, or
R^{2′} and R² together form a spiro-C₃₋₆cycloalkyl, or a spiro-C₃₋₁₀heterocyclyl; said cycloalkyl and heterocyclyl optionally substituted with 1 to 3 groups of C₁₋₆alkyl, C₃₋₁₀heterocyclyl, and halogen, or
R^{3′} and R³ together form a spiro-C₃₋₆cycloalkyl, or a spiro-C₃₋₁₀heterocyclyl; said cycloalkyl and heterocyclyl optionally substituted with 1 to 3 groups of C₁₋₆alkyl, C₃₋₁₀heterocyclyl, and halogen, or
R² and R³ can combine with the atoms to which they are attached to form a cyclic group selected from C₃₋₆cycloalkyl and C₃₋₁₀heterocyclyl, said cycloalkyl and heterocyclyl optionally substituted with 1 to 3 groups of C₁₋₆alkyl, C₃₋₁₀heterocyclyl, and halogen, said heterocyclyl optionally substituted with 1 to 3 halogen substituents;
R⁴ is hydrogen; or
R³ and R⁴ can combine with the atoms to which they are attached to form a cyclic group selected from C₃₋₆cycloalkyl and C₃₋₁₀heterocyclyl, said cycloalkyl and heterocyclyl optionally substituted with 1 to 3 groups of C₁₋₆alkyl, C₃₋₁₀heterocyclyl, and halogen, said heterocyclyl optionally substituted with 1 to 3 groups of halogen;
R^{x} is selected from C₁₋₆alkyl, C₂₋₆alkenyl, C(O)OC₁₋₆alkyl, C₃₋₆cycloalkyl, C₁₋₃haloalkyl, and C₃₋₁₀heterocyclyl, said alkyl, alkenyl, cycloalkyl and heterocyclyl optionally substituted with 1 to 3 R^{a} substituents each independently selected from hydrogen, C₁₋₆ alkyl, —OC₁₋₆alkyl, CN, SO₂, OH, C(O)OC₁₋₆alkyl, C₁₋₃haloalkyl, C₃₋₁₀ heteroaryl, C₃₋₁₀ heterocyclyl, and halogen, said heteroaryl and heterocyclyl of R^{a} optionally substituted with 1 to 3 R^{a′} substituents wherein each R^{a′} is independently selected from C₁₋₆ alkyl, CF₃, and CN;
A is O, NH or CH₂;
A^{a} is N or CH; or
A^{a}, R^{2a} and R^{3a} combine with the atoms to which they are attached to form a C₃₋₁₀ heterocyclyl, said heterocyclyl optionally substituted with 1 to 3 groups of R^{b};
R^{b} is selected from C₁₋₆ alkyl, OC₁₋₆ alkyl, CN, SO₂, OH, C(O)OC₁₋₆alkyl, C₁₋₃haloalkyl, C₃₋₁₀ cycloalkyl, and halogen;
R⁷ is hydrogen or C₁₋₆alkyl, and
n is 0, 1, 2, 4, or 4.

An embodiment of the invention is realized by Formula I.

An embodiment of the invention is realized by Formula Ia.

An embodiment of the invention of Formula I and Ia is realized when B is an optionally substituted C₅₋₆ heteroaryl. A subembodiment of this aspect of Formula I and Ia is realized when B is selected from optionally substituted pyrazolyl and pyridyl. Another subembodiment of this aspect of Formula I and Ia is realized when B is optionally substituted pyrazolyl. Another subembodiment of this aspect of Formula I and Ia is realized when B is optionally substituted pyridyl.

An embodiment of the invention of Formula I and Ia is realized when B is an optionally substituted C₆₋₁₀ aryl. A subembodiment of this aspect Formula I and Ia is realized when B is optionally substituted phenyl.

An embodiment of the invention of Formula I is realized when R^{2′} and R^{3′} are hydrogen.

Another embodiment of the invention of Formula I is realized when A is NH. Another embodiment of the invention of Formula I is realized when A is CH₂.

Another embodiment of the invention of Formula I is realized when Y is O. Another embodiment of the invention of Formula I is realized when Y is CH₂. Another embodiment of the invention of Formula I is realized when Y is OCH₂.

Another embodiment of the invention of Formula Ia is realized when Y′ is O. Another embodiment of the invention of Formula Ia is realized when Y′ is CH₂. Still another embodiment of the invention of Formula Ia is realized when Y′ is OCH₂.

Another embodiment of the invention of Formula I and Ia is realized when R¹ is C₁₋₆alkyl. A subembodiment of this aspect of the invention is realized when R¹ is C₁₋₆alkyl is selected from methyl, ethyl, propyl, butyl, pentyl and hexyl. Another embodiment of the invention of Formula I and Ia is realized when R¹ is C₃₋₆cycloalkyl. A subembodiment of this aspect of the invention is realized when R¹ is C₃₋₆cycloalkyl selected from cyclopropyl and cyclobutyl. Another embodiment of the invention of Formula I and Ia is realized when R¹ is halogen. A subembodiment of this aspect of Formula I and Ia is realized when R¹ is halogen selected from chlorine and fluorine. Another embodiment of the invention of Formula I and Ia is realized when R¹ is C₁₋₃haloalkyl. A subembodiment of this aspect of Formula I and Ia is realized when R¹ is C₁₋₃haloalkyl selected from CF₃, CHF₂, and CH₂F. An aspect of this embodiment is realized when R¹ is CF₃.

Another embodiment of the invention of Formula I is realized when R² and R³ are each hydrogen. Another embodiment of the invention of Formula I is realized when R⁴ is hydrogen and R² and R³ are independently C₁₋₆alkyl. A subembodiment of this aspect of Formula I is realized when R⁴ is hydrogen and R² and R³ are independently selected from methyl, ethyl, propyl, butyl, and hexyl. An aspect of this embodiment is realized when R² and R³ are each methyl. Another embodiment of the invention of Formula I is realized when R⁴ is hydrogen and R² and R³ are independently C₃₋₆cycloalkyl. A subembodiment of this aspect of the invention is realized when R⁴ is hydrogen and R² and R³ are independently selected from cyclopropyl and cyclobutyl. Another embodiment of the invention of Formula I is realized when R² and R³ are independently C₃₋₁₀heterocyclyl. A subembodiment of this aspect of the invention is realized when R² and R³ are independently oxaspirooctanyl.

An embodiment of the invention of Formula Ia is realized when A^{a}, R^{2a} and R^{3a} combine with the atoms to which they are attached to form a substituted C₃₋₁₀ heterocyclyl, said heterocyclyl optionally substituted with 1 to 3 groups of R^{b} and R^{4a} is (CH₂)ₙ. A subembodiment of this aspect of Formula Ia is realized when the heterocyclyl is selected from optionally substituted morpholinyl and tetrahydrofuranyl. Another subembodiment of this aspect of Formula Ia is realized when the heterocyclyl is optionally substituted morpholinyl. Another subembodiment of this aspect of Formula Ia is realized when the heterocyclyl is optionally substituted tetrahydrofuranyl.

An embodiment of the invention of Formula Ia is realized when R^{3a} and R^{4a} combine to form an optionally substituted C₃₋₁₀ cycloalkyl, or C₃₋₁₀ heterocyclyl and R^{2a} is hydrogen.

Another embodiment of the invention of Formula I is realized when R⁴ is hydrogen and R² and R³ combine with the atoms to which they are attached to form a cyclic group selected from optionally substituted C₃₋₆cycloalkyl and C₃₋₁₀heterocyclyl.

Another embodiment of the invention of Formula I is realized when R⁴ is hydrogen and R² and R³ combine with the atoms to which they are attached to form optionally substituted C₃₋₆cycloalkyl. A subembodiment of Formula I is realized when R⁴ is hydrogen and R² and R³ combine to form a group selected from optionally substituted cyclopropyl, cyclobutyl, cyclopentyl and cyclohexyl. Another further aspect of this subembodiment is realized when R² and R³ combine to form optionally substituted cyclopropyl. Another further aspect of this subembodiment is realized when R⁴ is hydrogen and R² and R³ combine to form optionally substituted cyclobutyl. Another further aspect of this subembodiment is realized when R⁴ is hydrogen and R² and R³ combine to form optionally substituted cyclopentyl. Another further aspect of this subembodiment is realized when R² and R³ combine to form optionally substituted cyclohexyl.

Another embodiment of the invention of Formula I is realized when R⁴ is hydrogen and R² and R³ combine with the atoms to which they are attached to form optionally substituted C₃₋₁₀heterocyclyl. A subembodiment of this aspect of Formula I is realized when R⁴ is hydrogen and R² and R³ combine to form a group selected from optionally substituted tetrahydrofuranyl, tetrahydropyranyl, oxetanyl, azetidinyl, piperizinyl, pyrrolidinyl and pyrrolidinyl. A subembodiment of this aspect of the invention is realized when R⁴ is hydrogen and R² and R³ combine to form optionally substituted tetrahydrofuranyl. A subembodiment of this aspect of the invention is realized when R⁴ is hydrogen and R² and R³ combine to form optionally substituted teterahydropyranyl. A subembodiment of this aspect of the invention is realized when R⁴ is hydrogen and R² and R³ combine to form optionally substituted pyrrolidinyl. A subembodiment of this aspect of the invention is realized when R⁴ is hydrogen and R² and R³ combine to form optionally substituted oxetanyl. A subembodiment of this aspect of the invention is realized when R⁴ is hydrogen and R² and R³ combine to form optionally substituted azetidinyl. A subembodiment of this aspect of the invention is realized when R⁴ is hydrogen and R² and R³ combine to form optionally substituted piperazinyl. A subembodiment of this aspect of the invention is realized when R⁴ is hydrogen and R² and R³ combine to form optionally substituted piperidinyl. A subembodiment of this aspect of the invention is realized when R⁴ is hydrogen and R² and R³ combine to form optionally substituted pyrrolidinyl.

Another embodiment of this invention of Formula I is realized when R⁴ is hydrogen.

Another embodiment of the invention of Formula I is realized when R^{3′} is hydrogen and R³ and R⁴ combine with the atoms to which they are attached to form a group selected from optionally substituted cyclopropyl, cyclobutyl, cyclopentyl, cyclohexyl, tetrahydrofuranyl, tetrahydropyranyl, oxetanyl, azetidinyl, piperazinyl, piperidinyl, and pyrrolidinyl.

Another embodiment of the invention of Formula I and Ia is realized when R⁷ is hydrogen. Another embodiment of the invention of Formula I and Ia when R⁷ is C₁₋₆alkyl. A subembodiment of this aspect of Formula I and Ia is realized when R⁷ is CH₃.

In one embodiment, the compounds of the invention of Formula I have structural Formula I″:

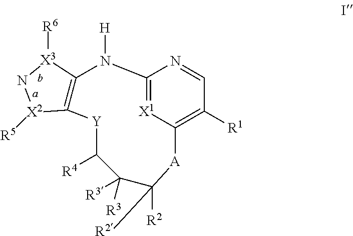

or a pharmaceutically acceptable salt thereof, wherein:
A, Y, X¹, R¹, R², R^{2′}, R³, R^{3′} and R⁴ are as described herein,
bond “a” is a double bond when X² is C
bond “b” is a double bond when X³ is C; provided that only one of X² or X³ is carbon at the same time;
R⁵ is selected from hydrogen, C₁₋₆alkyl, C(O)OC₁₋₆alkyl, C₃₋₆cycloalkyl, C₁₋₃haloalkyl, and C₃₋₁₀heterocyclyl, said alkyl, cycloalkyl and heterocyclyl optionally substituted with 1 to 3 R^{a} substituents each independently selected from hydrogen, C₁₋₆ alkyl, CN, SO₂, OH, C(O)OC₁₋₆alkyl, C₁₋₃haloalkyl, C₃₋₁₀ heteroaryl, C₃₋₁₀ heterocyclyl, and halogen, said heteroaryl and heterocyclyl of R^{a} optionally substituted with 1 to 3 R^{a′} substituents wherein each R^{a′} is independently selected from C₁₋₆ alkyl, CF₃, and CN; and
R⁶ is selected from C₁₋₆alkyl, C₂₋₆alkenyl, C₃₋₆cycloalkyl, C₁₋₃haloalkyl, and C₃₋₁₀heterocyclyl, said alkyl, alkenyl, cycloalkyl and heterocyclyl optionally substituted with 1 to 3 groups of R^{b} selected from hydrogen, C₁₋₆ alkyl, OH, —OC₁₋₆alkyl, and halogen.

Another embodiment of the invention of Formula I″ is realized when X¹ is N. Another embodiment of the invention of Formula I″ is realized when X¹ is CH.

Another embodiment of the invention of Formula I″ is realized when X² is C. Another embodiment of the invention of Formula I″ is realized when X² is N.

Another embodiment of the invention of Formula I″ is realized when X³ is C. Another embodiment of the invention of Formula I″ is realized when X³ is N.

In one embodiment of the present invention of Formula I″ X² and X³ are not the same.

In yet another embodiment of the present invention of Formula I″ one of X² and X³ is C and the other of X² and X³ is N.

Another embodiment of the invention of Formula I″ is realized when X¹, X² and X³ are N, C and N, respectively.

Another embodiment of the invention of Formula I″ is realized when X¹, X² and X³ are N, N and C, respectively.

Another embodiment of the invention of Formula I″ is realized when A, X¹, X² and X³ are NH, N, C and N, respectively.

Another embodiment of the invention of Formula I″ is realized when A, X¹, X² and X³ are NH, N, N and C, respectively.

Another embodiment of the invention of Formula I″ is realized when Y, A, X¹, X² and X³ are O, NH, N, C and N, respectively.

Another embodiment of the invention of Formula I″ is realized when Y, A, X¹, X² and X³ are O, NH, N, N and C, respectively.

Another embodiment of the invention of Formula I″ is realized when Y, A, X¹, X² and X³ are OCH₂, NH, N, C and N, respectively.

Another embodiment of the invention of Formula I″ is realized when Y, A, X¹, X² and X³ are OCH₂, NH, N, N and C, respectively.

Another embodiment of the invention of Formula I″ is realized when Y, A, X¹, X² and X³ are CH₂, NH, N, C and N, respectively.

Another embodiment of the invention of Formula I″ is realized when Y, A, X¹, X² and X³ are CH₂, NH, N, N and C, respectively.

An embodiment of the invention of Formula I″ is realized when R^{2′} and R^{3′} are hydrogen.

An embodiment of the invention of Formula I″ is realized when R¹, is selected from CH₃, CF₃, Cl, Fl, and cyclopropyl and A is NH. An embodiment of the invention of Formula I″ is realized when R⁵ and R⁶ are independently selected from —CH₂R^{a}, —CH₂(CH₃)₂R^{a}, —C(CH₃)₂R^{a}, —C(CH₃)₂CH₂R^{a}, —CH₂R^{a}, —CH(CH₃)₂, —CH₂(CH₃)₂R^{b}, —CH₃, —CH₂CH₃, —C(CH₃)₂R^{b}, —C(CH₃)₂CH₂R^{b}, CF₃, CHF₂, CH₂F, cyclopropyl, and cyclobutyl. A subembodiment of this aspect of the invention of Formula I″ is realized when X² and X³ are C and N, respectively. A subembodiment of this aspect of the invention is realized when X² and X³ are N and C, respectively.

Another embodiment of the invention of Formula I″ is realized when R⁴ is hydrogen and R² and R³ combine with the atoms to which they are attached to form a cyclic group selected from optionally substituted C₃₋₆cycloalkyl and C₃₋₁₀heterocyclyl. A subembodiment of this aspect of the invention of Formula I″ is realized when R⁴ is hydrogen and R² and R³ combine with the atoms to which they are attached to form optionally substituted cyclopropyl, cyclobutyl, cyclopentyl, cyclohexyl, tetrahydrofuranyl, tetrahydropyranyl, oxetanyl, azetidinyl, piperizinyl, piperidinyl, and pyrrolidinyl. A further subembodiment of this aspect of the invention of Formula I″ is realized when R⁴ is hydrogen and R² and R³ combine with the atoms to which they are attached to form optionally substituted cyclopropyl, cyclobutyl, and tetrahydrofuranyl. A further subembodiment of this aspect of the invention of Formula I″ is realized when R⁴ is hydrogen and R² and R³ combine with the atoms to which they are attached to form optionally substituted cyclopropyl. A further subembodiment of this aspect of the invention of Formula I″ is realized when R⁴ is hydrogen and R² and R³ combine with the atoms to which they are attached to form optionally substituted cyclobutyl. A further subembodiment of this aspect of the invention of Formula I″ is realized when R² and R³ combine with the atoms to which they are attached to form optionally substituted tetrahydrofuranyl.

Another embodiment of the invention of Formula I″ is realized when R⁴ is hydrogen and R² and R³ combine with the atoms to which they are attached to form a group selected from optionally substituted cyclopropyl, cyclobutyl, cyclopentyl, cyclohexyl, tetrahydrofuranyl, tetrahydropyranyl, oxetanyl, azetidinyl, piperazinyl, piperidinyl, and pyrrolidinyl and X¹, X² and X³ are N, C and N, respectively.

Another embodiment of the invention of Formula I″ is realized when R⁴ is hydrogen and R² and R³ combine with the atoms to which they are attached to form a group selected from optionally substituted cyclopropyl, cyclobutyl, cyclopentyl, cyclohexyl, tetrahydrofuranyl, tetrahydropyranyl, oxetanyl, azetidinyl, piperazinyl, piperidinyl, and pyrrolidinyl and X¹, X² and X³ are N, N and C, respectively.

Another embodiment of the invention of Formula I″ is realized when R⁴ is hydrogen and R² and R³ combine with the atoms to which they are attached to form a group selected from optionally substituted cyclopropyl, cyclobutyl, cyclopentyl, cyclohexyl, tetrahydrofuranyl, tetrahydropyranyl, oxetanyl, azetidinyl piperazinyl, piperidinyl, and pyrrolidinyl and A, X¹, X² and X³ are NH, N, C and N, respectively.

Another embodiment of the invention of Formula I″ is realized when R⁴ is hydrogen and R² and R³ combine with the atoms to which they are attached to form a group selected from optionally substituted cyclopropyl, cyclobutyl, cyclopentyl, cyclohexyl, tetrahydrofuranyl, tetrahydropyranyl, oxetanyl, azetidinyl, piperazinyl, piperadinyl, and pyrrolidinyl and A, X¹, X² and X³ are NH, N, N and C, respectively.

Another embodiment of the invention of Formula I″ is realized when R⁴ is hydrogen and R² and R³ combine with the atoms to which they are attached to form a group selected from optionally substituted cyclopropyl, cyclobutyl, cyclopentyl, cyclohexyl, tetrahydrofuranyl, tetrahydropyranyl, oxetanyl, azetidinyl, piperazinyl, piperidinyl, and pyrrolidinyl and Y, A, X¹, X² and X³ are O, NH, N, C and N, respectively.

Another embodiment of the invention of Formula I″ is realized when R⁴ is hydrogen and R² and R³ combine with the atoms to which they are attached to form a group selected from optionally substituted cyclopropyl, cyclobutyl, cyclopentyl, cyclohexyl, tetrahydrofuranyl, tetrahydropyranyl, oxetanyl, azetidinyl, piperazinyl, piperidinyl, and pyrrolidinyl and Y, A, X¹, X² and X³ are O, NH, N, N and C, respectively.

Another embodiment of the invention of Formula I″ is realized when R⁵ is optionally substituted C₁₋₆alkyl or optionally substituted C₁₋₃haloalkyl. A subembodiment of this aspect of the invention is realized when R⁵ is selected from —CH₂R^{a}, —CH₂(CH₃)₂R^{a}, —CH₃, —CH₂CH₃, —C(CH₃)₂R^{a}, —C(CH₃)₂CH₂R^{a}, CF₃, CHF₂, CH₂F. A further aspect of this subembodiment is realized when R⁵ is selected from optionally substituted-CH₃, —CH₂CH₃, —C(CH₃)₂R^{a}, and —C(CH₃)₂CH₂R^{a}.

Another embodiment of the invention of Formula I″ is realized when R⁵ is optionally substituted C₃₋₆cycloalkyl. A subembodiment of this aspect of the invention is realized when R⁵ is selected from optionally substituted cyclopropyl and cyclobutyl.

Another embodiment of the invention of Formula I″ is realized when R⁵ is optionally substituted C₃₋₁₀heterocyclyl. A subembodiment of this aspect of the invention is realized when R⁵ is selected from optionally substituted tetrahydrofuranyl, azetidinly, piperazinyl, piperidinyl, oxetanyl, and thietanyl sulfoxide.

Another embodiment of the invention of Formula I″ is realized when R⁶ is optionally substituted C₁₋₆alkyl or optionally substituted C₁₋₃haloalkyl. A subembodiment of this aspect of the invention is realized when R⁶ is selected from —CH₂R^{b}, —CH(CH₃)₂, —CH₂(CH₃)₂R^{b}, —CH₃, —CH₂CH₃, —C(CH₃)₂R^{b}, —C(CH₃)₂CH₂R^{b}, CF₃, CHF₂, CH₂F. A further aspect of this subembodiment is realized when R⁶ is selected from —CH₃, —CH(CH₃)₂, —CH₂CH₃, —C(CH₃)₂R^{b}, and —C(CH₃)₂CH₂R^{b}.

Another embodiment of the invention of Formula I″ is realized when R⁶ is optionally substituted C₃₋₆cycloalkyl. A subembodiment of this aspect of the invention is realized when R⁶ is selected from optionally substituted cyclopropyl and cyclobutyl.

An embodiment of the invention of Formula I is realized by structural formulas II and III:

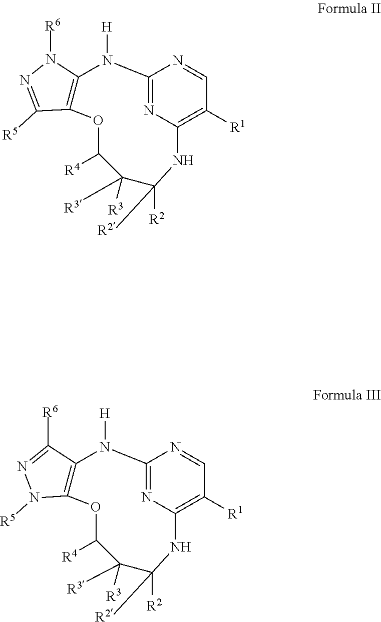

wherein R¹, R², R^{2′}, R³, R^{3′}, R⁴, R⁵, and R⁶ are as described herein. An embodiment of the invention of Formulas II and II is realized when R^{2′} and R^{3′} are hydrogen. An embodiment of the invention of Formulas II and III is realized when R¹ is independently selected from CH₃, CF₃, Cl, Fl, and cyclopropyl. A subembodiment of this aspect of the invention is realized when R¹ is CF₃. An embodiment of the invention of Formulas II and III is realized when R⁵ and R⁶ are independently selected from optionally substituted-CH₂R^{a}, —CH₂(CH₃)₂R^{a}, —C(CH₃)₂R^{a}, —C(CH₃)₂CH₂R^{a}, —CH₂R^{a}, —CH(CH₃)₂, —CH₂(CH₃)₂R^{b}, —CH₃, —CH₂CH₃, —C(CH₃)₂R^{b}, —C(CH₃)₂CH₂R^{b}, CF₃, CHF₂, CH₂F, cyclopropyl, and cyclobutyl.

Another embodiment of the invention of Formula II and III is realized when R⁴ is hydrogen and R² and R³ combine with the atoms to which they are attached to form a cyclic group selected from optionally substituted C₃₋₆cycloalkyl and C₃₋₁₀heterocyclyl. A subembodiment of this aspect of the invention of Formula II and III is realized when R⁴ is hydrogen and R² and R³ combine with the atoms to which they are attached to form optionally substituted cyclopropyl, cyclobutyl, cyclopentyl, cyclohexyl, tetrahydrofuranyl, tetrahydropyranyl, oxetanyl, azetidinyl, piperizinyl, piperidinyl, and pyrrolidinyl. A further subembodiment of this aspect of the invention of Formula II and III is realized when R⁴ is hydrogen and R² and R³ combine with the atoms to which they are attached to form optionally substituted cyclopropyl, cyclobutyl, and tetrahydrofuranyl. A further subembodiment of this aspect of the invention of Formula II and III is realized when R⁴ is hydrogen and R² and R³ combine with the atoms to which they are attached to form optionally substituted cyclopropyl. A further subembodiment of this aspect of the invention of Formula II and III is realized when when R⁴ is hydrogen and R² and R³ combine with the atoms to which they are attached to form optionally substituted cyclobutyl. A further subembodiment of this aspect of the invention of Formula II and III is realized when R⁴ is hydrogen and R² and R³ combine with the atoms to which they are attached to form optionally substituted tetrahydrofuranyl.

Another embodiment of the invention Formula Ia is represented by Formula Ia″

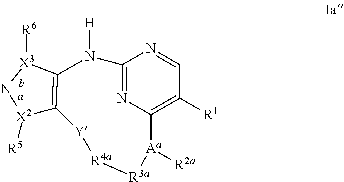

or a pharmaceutically acceptable salt thereof, wherein a, b, X², X³, Y′, A^{a}, R¹, R^{2a}, R^{3a}, R^{4a}, R⁵, and R⁶ are as described herein.

An embodiment of the invention of Formula Ia″ is realized when A^{a}, R^{2a} and R^{3a} combine with the atoms to which they are attached to form a substituted C₃₋₁₀ heterocyclyl, said heterocyclyl optionally substituted with 1 to 3 groups of R^{b} and R^{4a} is (CH₂)ₙ. A subembodiment of this aspect of Formula Ia″ is realized when the heterocyclyl is selected from optionally substituted morpholinyl and tetrahydrofuranyl. Another subembodiment of this aspect of Formula Ia is realized when the heterocyclyl is optionally substituted morpholinyl. Another subembodiment of this aspect of Formula Ia″ is realized when the heterocyclyl is optionally substituted tetrahydrofuranyl.

An embodiment of the invention of Formula Ia″ is realized when R^{3a} and R^{4a} combine to form an optionally substituted C₃₋₁₀ cycloalkyl, or C₃₋₁₀ heterocyclyl, A^{a} is N or CH₂ and R^{2a} is hydrogen.

Another embodiment of the invention of Formula Ia″ is realized when R⁵ is optionally substituted C₁₋₆alkyl or optionally substituted C₁₋₃haloalkyl. A subembodiment of this aspect of the invention is realized when R⁵ is selected from —CH₂R^{a}, —CH₂(CH₃)₂R^{a}, —CH₃, —CH₂CH₃, —C(CH₃)₂R^{a}, —C(CH₃)₂CH₂R^{a}, CF₃, CHF₂, CH₂F. A further aspect of this subembodiment is realized when R⁵ is selected from optionally substituted —CH₃, —CH₂CH₃, —C(CH₃)₂R^{a}, and —C(CH₃)₂CH₂R^{a}.

Another embodiment of the invention of Formula Ia″ is realized when R⁵ is optionally substituted C₃₋₆cycloalkyl. A subembodiment of this aspect of the invention is realized when R⁵ is selected from optionally substituted cyclopropyl and cyclobutyl.

Another embodiment of the invention of Formula Ia″ is realized when R⁵ is optionally substituted C₃₋₁₀heterocyclyl. A subembodiment of this aspect of the invention is realized when R⁵ is selected from optionally substituted tetrahydrofuranyl, azetidinly, piperazinyl, piperidinyl, oxetanyl, and thietanyl sulfoxide.

Another embodiment of the invention of Formula Ia″ is realized when R⁶ is optionally substituted C₁₋₆alkyl or optionally substituted C₁₋₃haloalkyl. A subembodiment of this aspect of the invention is realized when R⁶ is selected from —CH₂R^{b}, —CH(CH₃)₂, —CH₂(CH₃)₂R^{b}, —CH₃, —CH₂CH₃, —C(CH₃)₂R^{b}, —C(CH₃)₂CH₂R^{b}, CF₃, CHF₂, CH₂F. A further aspect of this subembodiment is realized when R⁶ is selected from —CH₃, —CH(CH₃)₂, —CH₂CH₃, —C(CH₃)₂R^{b}, and —C(CH₃)₂CH₂R^{b}.

Another embodiment of the invention of Formula Ia″ is realized when R⁶ is optionally substituted C₃₋₆cycloalkyl. A subembodiment of this aspect of the invention is realized when R⁶ is selected from optionally substituted cyclopropyl and cyclobutyl.

An embodiment of the invention of Formula Ia″ is realized when R¹, is selected from CH₃, CF₃, Cl, Fl, and cyclopropyl and A is NH. An embodiment of the invention of Formula Ia″ is realized when R⁵ and R⁶ are independently selected from —CH₂R^{a}, —CH₂(CH₃)₂R^{a}, —C(CH₃)₂R^{a}, —C(CH₃)₂CH₂R^{a}, —CH₂R^{a}, —CH(CH₃)₂, —CH₂(CH₃)₂R^{b}, —CH₃, —CH₂CH₃, —C(CH₃)₂R^{b}, —C(CH₃)₂CH₂R^{b}, CF₃, CHF₂, CH₂F, cyclopropyl, and cyclobutyl. A subembodiment of this aspect of the invention of Formula Ia″ is realized when X² and X³ are C and N, respectively. A subembodiment of this aspect of the invention is realized when X² and X³ are N and C, respectively.

In another embodiment, the compounds of the invention include those identified herein as Examples in the tables below, and pharmaceutically acceptable salts thereof.

In another embodiment, the present invention provides pharmaceutical compositions comprising a pharmaceutically acceptable carrier and a compound of the invention or a pharmaceutically acceptable salt thereof.

In another embodiment, the present invention provides a method of treating a disease or disorder in which the LRRK2 kinase is involved, or one or more symptoms or conditions associated with said diseases or disorders, said method comprising administering to a subject (e.g., mammal, person, or patient) in need of such treatment an effective amount of a compound of the invention, or a pharmaceutically acceptable salt thereof, or pharmaceutically acceptable composition thereof. Non-limiting examples of such diseases or disorders, and symptoms associated with such diseases or disorders, each of which comprise additional independent embodiments of the invention, are described below.

Another embodiment provides the use of a compound of the invention, or a pharmaceutically acceptable salt thereof, and a pharmaceutically acceptable carrier, for the manufacture of a medicament for the treatment of Parkinson's Disease. The invention may also encompass the use of a compound of the invention, or a pharmaceutically acceptable salt thereof, and a pharmaceutically acceptable carrier, in therapy.

Another embodiment provides for medicaments or pharmaceutical compositions which may be useful for treating diseases or disorders in which LRRK2 is involved, such as Parkinson's Disease, which comprise a compound of the invention, or a pharmaceutically acceptable salt thereof, and a pharmaceutically acceptable carrier.

Another embodiment provides for the use of a compound of the invention which may be useful for treating diseases or disorders in which LRRK2 is involved, such as Parkinson's Disease.

Another embodiment provides a method for the manufacture of a medicament or a composition which may be useful for treating diseases or disorders in which LRRK2 is involved, such as Parkinson's Disease, comprising combining a compound of the invention with one or more pharmaceutically acceptable carriers.

### DETAILED DESCRIPTION OF THE INVENTION

The compounds of the invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Unless a specific stereochemistry is indicated, the present invention is meant to encompass all such isomeric forms of these compounds.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

In the compounds of Formulae I, I″ II, and III the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formulae I, I″, II, and III. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formulae I, I″, II, and III can be prepared without undue experimentation by conventional techniques well known to those skilled in the art or by processes analogous to those described in the Schemes and Examples herein using appropriate isotopically-enriched reagents and/or intermediates.

When a compound of the invention is capable of forming tautomers, all such tautomeric forms are also included within the scope of the present invention. For example, compounds including carbonyl —CH₂C(O)— groups (keto forms) may undergo tautomerism to form hydroxyl —CH═C(OH)— groups (enol forms). Both keto and enol forms, where present, are included within the scope of the present invention.

When any variable (e.g. R⁵, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be understood as meaning that the group in question is either unsubstituted or may be substituted with one or more substituents.

Absolute stereochemistry is illustrated by the use of hashed and solid wedge bonds. As shown in Illus-I and Illus-2. Accordingly, the methyl group of Illus-I is emerging from the page of the paper and the ethyl group in Illus-2 is descending into the page, where the cyclohexene ring resides within the plane of the paper. It is assumed that the hydrogen on the same carbon as the methyl group of Illus-I descends into the page and the hydrogen on the same carbon as the ethyl group of Illus-2 emerges from the page. The convention is the same where both a hashed and solid rectangle are appended to the same carbon as in Illus-3, the methyl group is emerging from the plane of the paper and the ethyl group is descending into the plane of the paper with the cyclohexene ring in the plane of the paper.

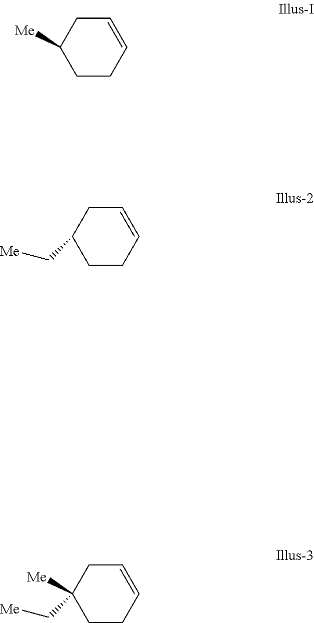 [SMILES: C[C@H]1CC=CCC1 ; CC[C@@H]1CC=CCC1 ; CC[C@]1(C)CC=CCC1]

As is conventional, unless otherwise noted in accompanying text, ordinary “stick” bonds or “wavy” bonds indicate that all possible stereochemistry is represented, including, pure compounds, mixtures of isomers, and racemic mixtures.

As used herein, unless otherwise specified, the following terms have the following meanings:

The phrase “at least one” used in reference to the number of components comprising a composition, for example, “at least one pharmaceutical excipient” means that one member of the specified group is present in the composition, and more than one may additionally be present. Components of a composition are typically aliquots of isolated pure material added to the composition, where the purity level of the isolated material added into the composition is the normally accepted purity level for a reagent of the type.
“at least one” used in reference to substituents appended to a compound, for example, a halogen or a moiety appended to a portion of a structure replacing a hydrogen, means that one substituent of the group of substituents specified is present, and more than one of said substituents may be bonded to any of the defined or chemically accessible bonding points of the structure.

Whether used in reference to a substituent on a compound or a component of a pharmaceutical composition the phrase “one or more”, means the same as “at least one”;
“concurrently” and “contemporaneously” both include in their meaning (1) simultaneously in time (e.g., at the same time); and (2) at different times but within the course of a common treatment schedule;
“optionally interrupted” means that the carbon atom can be replaced by a heteroatom selected oxygen and/or nitrogen.
“consecutively” means one following the other;
“sequentially” refers to a series administration of therapeutic agents that awaits a period of efficacy to transpire between administering each additional agent; this is to say that after administration of one component, the next component is administered after an effective time period after the first component; the effective time period is the amount of time given for realization of a benefit from the administration of the first component;
“effective amount” or “therapeutically effective amount” is meant to describe the provision of an amount of at least one compound of the invention or of a composition comprising at least one compound of the invention which is effective in treating or inhibiting a disease or condition described herein, and thus produce the desired therapeutic, ameliorative, inhibitory or preventative effect. For example, in treating central nervous system diseases or disorders with one or more of the compounds described herein “effective amount” (or “therapeutically effective amount”) means, for example, providing the amount of at least one compound of I, I″, II, or III that results in a therapeutic response in a patient afflicted with a central nervous system disease or disorder (“condition”), including a response suitable to manage, alleviate, ameliorate, or treat the condition or alleviate, ameliorate, reduce, or eradicate one or more symptoms attributed to the condition and/or long-term stabilization of the condition, for example, as may be determined by the analysis of pharmacodynamic markers or clinical evaluation of patients afflicted with the condition;
“patient” and “subject” means an animal, such as a mammal (e.g., a human being) and is preferably a human being;
“prodrug” means compounds that are rapidly transformed, for example, by hydrolysis in blood, in vivo to the parent compound, a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this invention includes prodrugs of the novel compounds of this invention;

The term “substituted,” as used herein, means that one or more hydrogens on the designated atom is replaced with a selection from the indicated group, provided that the designated atom's normal valency under the existing circumstances is not exceeded, and that the substitution results in a stable compound. Unless expressly stated to the contrary, substitution by a named substituent is permitted on any atom provided such substitution is chemically allowed, and results in a stable compound. Combinations of substituents and/or variables are permissible only if such combinations result in stable compounds.

Where optional substitution of a moiety is described (e.g. “optionally substituted”) the term means that if substituents are present, one or more of the enumerated substituents for the specified moiety can be present on the moeity in a bonding position normally occupied by the default substituent normally occupying that position. For example, a default substituent on the carbon atoms of an alkyl moiety is a hydrogen atom, an optional substituent can replace the default substituent.

As used herein, unless otherwise specified, the following terms used to describe moieties, whether comprising the entire definition of a variable portion of a structural representation of a compound of the invention or a substituent appended to a variable portion of a structural representation of a group of compounds of the invention have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl moieties, and the like); moieties are equivalently described herein by structure, typographical representation or chemical terminology without intending any differentiation in meaning, for example, an “acyl” substituent may be equivalently described herein by the term “acyl”, by typographical representations “R′—(C═O)—” or “R′—C(O)—”, or by a structural representation:

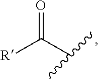

equally, with no differentiation implied using any or all of these representations;

The term “alkyl” (including the alkyl portions of other moieties, such as trifluoromethyl-alkyl- and alkoxy-) means a straight or branched aliphatic hydrocarbon moiety comprising up to about 20 carbon atoms (for example, a designation of “C₁₋₂₀-alkyl” indicates an aliphatic hydrocarbon moiety of from 1 to 20 carbon atoms). In some embodiments, alkyls preferably comprise up to about 10 carbon atoms, unless the term is modified by an indication that a shorter chain is contemplated, for example, an alkyl moiety of from 1 up to 8 carbon atoms is designated herein “C₁₋₈-alkyl”. Where the term “alkyl” is indicated with two hyphens (i.e., “-alkyl-” it indicates that the alkyl moiety is bonded in a manner that the alkyl moiety connects the substituents on either side of it, for example, “-alkyl-OH” indicates the alkyl moiety functions as a linker between the hydroxyl group and the rest of the molecule.

The term “cycloalkyl” means a moiety having a main hydrocarbon chain forming a mono- or bicyclo-cyclic aliphatic moiety comprising at least 3 carbon atoms (the minimum number necessary to provide a monocyclic moiety) up to the maximum number of specified carbon atoms, generally 8 for a monocyclic moiety and 10 for a bicyclic moiety. Examples of cycloalkyl moieties include, but are not limited to, cyclopropyl, cyclobutyl, cyclopentyl or cyclohexyl. The term “cycloalkyl” also includes non-aromatic, fused multicyclic ring system comprising up to 20 carbon atoms which may optionally be substituted as defined herein for “alkyl” generally. Suitable multicyclic cycloalkyls are, for example, but are not limited to: 1-decalin; norbornyl; adamantly; and the like;

As used herein, when the term “alkyl” is modified by “substituted” or “optionally substituted”, it means that one or more of the hydrogen atoms on the alkyl moiety is replaced with a selection from the indicated group of “substituents.” “Optionally substituted” means “unsubstituted or substituted.”

Where a structural formula represents bonding between a moiety and a the rest of the molecule using a bonding line that terminates in the middle of a ring, for example the following representations:

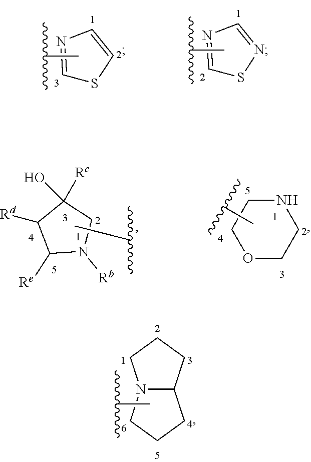

whether or not numbered the structure indicates that unless otherwise defined the moiety may be bonded to the molecule through any of available ring atom, for example, the numbered atoms of the example moieties;

The term “heterocyclyl” (or heterocycloalkyl) means a non-aromatic saturated monocyclic or multicyclic ring system comprising 3 to 10 ring atoms, preferably 5 to 10 ring atoms, in which one or more of the atoms in the ring system is an element other than carbon, for example nitrogen (e.g. piperidyl- or pyrrolidinyl), oxygen (e.g. furanyl and tetrahydropyranyl) or sulfur (e.g. tetrahydrothiophenyl and tetrahydrothiopyranyl); and wherein the heteroatoms can be alone or in combination provided that the moiety does not contain adjacent oxygen and/or sulfur atoms present in the ring system; preferred heterocyclyl moieties contain 5 to 6 ring atoms; the prefix aza, oxa or thia before the heterocyclyl root name means that at least one nitrogen, oxygen or sulfur atom, respectively, is present as a ring atom; the heterocyclyl can be optionally substituted by one or more independently selected substituents;

The nitrogen or sulfur atom of the heterocyclyl can be optionally oxidized to the corresponding N-oxide, S-oxide or S,S-dioxide (SO₂); non-limiting examples of suitable monocyclic heterocyclyl rings include piperidyl, pyrrolidinyl, piperazinyl, morpholinyl-

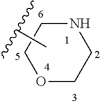

(where unless otherwise noted the moiety is bonded through any of ring carbon atoms C₂, C₃, C₅, or C₆), thiomorpholinyl, thiazolidinyl, 1,3-dioxolanyl, 1,4-dioxanyl, tetrahydrofuranyl, tetrahydrothiophenyl, tetrahydrothiopyranyl, and the like. The term heterocyclyl includes fused and bridged polycyclicheterocyclyl rings, for example, moieties of the structure:

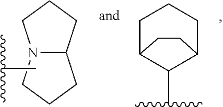

and the like.

The term “halogen” means fluorine, chlorine, bromine, or iodine; preferred halogens, unless specified otherwise where the term is used, are fluorine, chlorine and bromin. A substituent which is a halogen atom means —F, —Cl, —Br, or —I, and “halo” means fluoro, chloro, bromo, or iodo substituents bonded to the moiety defined, for example, “haloalkyl” means an alkyl, as defined above, wherein one or more of the bonding positions on the alkyl moiety typically occupied by hydrogen atoms are instead occupied by a halo group.

The term “hydroxyl” and “hydroxy” means an HO— group, “hydroxyalkyl” means a substituent of the formula: Non-limiting examples of suitable hydroxyalkyl groups include hydroxymethyl and 2-hydroxyethyl; and (are there others?)

The bonding sequence is indicated by hyphens where moieties are represented in text, for example -alkyl, indicates a single bond between the alkyl moiety and the atom to which it is bonded, -alkyl-X, indicates that the alkyl group is further bonded to an “X” substituent, and in structural representation, bonding sequence is indicated by a wavy line terminating a bond representation, for example:

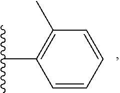 [SMILES: *c1ccccc1C]

indicates that the methylphenyl moiety is bonded through a carbon atom ortho to the methyl substituent, while a bond representation terminated with a wavy line and drawn into a structure without any particular indication of an atom to which it is bonded indicates that the moiety may be bonded via any of the atoms in the moiety which are available for bonding as described in the examples above.

The line -, as a bond generally indicates a mixture of, or either of, the possible isomers, e.g., containing (R)- and (S)- stereochemical configuration. For example:

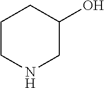

encompasses

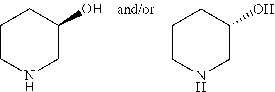 [SMILES: O[C@@H]1CCCNC1 ; O[C@H]1CCCNC1]

Furthermore, unwedged-bolded or unwedged-hashed lines are used in structures containing multiple stereocenters in order to depict relative configuration where it is known. For example:

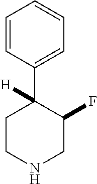 [SMILES: F[C@H]1CNCC[C@@H]1c1ccccc1]

means that the fluorine and hydrogen atoms are on the same face of the piperidine ring, but represents a mixture of, or one of, the possible isomers at right

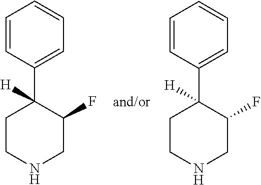 [SMILES: F[C@H]1CNCC[C@@H]1c1ccccc1 ; F[C@@H]1CNCC[C@H]1c1ccccc1]

whereas:

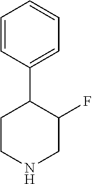 [SMILES: FC1CNCCC1c1ccccc1]

represents a mixture of, or one of, the possible isomers at right

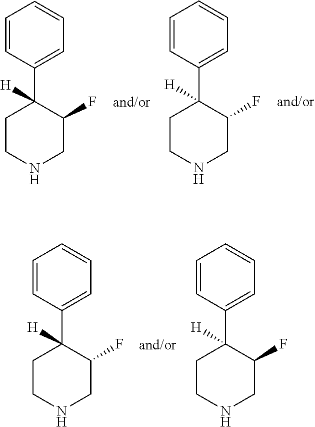 [SMILES: F[C@H]1CNCC[C@@H]1c1ccccc1 ; F[C@@H]1CNCC[C@H]1c1ccccc1 ; F[C@@H]1CNCC[C@@H]1c1ccccc1 ; F[C@H]1CNCC[C@H]1c1ccccc1]

In all cases, compound name(s) accompany the structure drawn and are intended to capture each of the stereochemical permutations that are possible for a given structural isomer based on the synthetic operations employed in its preparation. Lists of discrete stereoisomers that are conjoined using or indicate that the presented compound (e.g. ‘Example number’) was isolated as a single stereoisomer, and that the identity of that stereoisomer corresponds to one of the possible configurations listed. Lists of discrete stereoisomers that are conjoined using and indicate that the presented compound was isolated as a racemic mixture or diastereomeric mixture.

A specific absolute configuration is indicated by use of a wedged-bolded or wedged-hashed line. Unless a specific absolute configuration is indicated, the a structure of the present invention is meant to encompass all such stereoisomeric forms of these compounds.

In this specification, where there are multiple oxygen and/or sulfur atoms in a ring system, there cannot be any adjacent oxygen and/or sulfur present in said ring system.

As well known in the art, a bond drawn from a particular atom wherein no moiety is depicted at the terminal end of the bond indicates a methyl group bound through that bond to the atom, unless stated otherwise. For example:

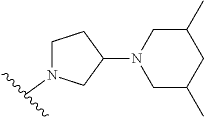 [SMILES: *N1CCC(N2CC(C)CC(C)C2)C1]

represents

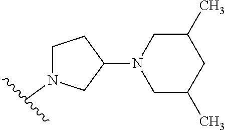 [SMILES: *N1CCC(N2CC(C)CC(C)C2)C1]

Unsatisfied valences in the text, schemes, examples, structural formulae, and any Tables herein is assumed to have a hydrogen atom or atoms of sufficient number to satisfy the valences.

One or more compounds of the invention may also exist as, or optionally be converted to, a solvate. Preparation of solvates is generally known. Thus, for example, M. Caira et al, J. Pharmaceutical Sci., 93 (3), 601-611 (2004) describe the preparation of the solvates of the antifungal fluconazole in ethyl acetate as well as from water. Similar preparations of solvates, and hemisolvate, including hydrates (where the solvent is water or aqueous-based) and the like are described by E. C. van Tonder et al, AAPS PharmSciTech., 5 (1), article 12 (2004); and A. L. Bingham et al, Chem. Commun., 603-604 (2001). A typical, non-limiting, process involves dissolving the inventive compound in desired amounts of the desired solvent (for example, an organic solvent, an aqueous solvent, water or mixtures of two or more thereof) at a higher than ambient temperature, and cooling the solution, with or without an antisolvent present, at a rate sufficient to form crystals which are then isolated by standard methods. Analytical techniques such as, for example I.R. spectroscopy, show the presence of the solvent (including water) in the crystals as a solvate (or hydrate in the case where water is incorporated into the crystalline form).

This invention also includes the compounds of this invention in isolated and purified form obtained by routine techniques. Polymorphic forms of the compounds of Formulae I, I″, II, and III and of the salts, solvates and prodrugs of the compounds of Formulae I, I″, II, and III are intended to be included in the present invention. Certain compounds of the invention may exist in different isomeric forms (e.g., enantiomers, diastereoisomers, atropisomers). The inventive compounds include all isomeric forms thereof, both in pure form and admixtures of two or more, including racemic mixtures.

In the same manner, unless indicated otherwise, presenting a structural representation of any tautomeric form of a compound which exhibits tautomerism is meant to include all such tautomeric forms of the compound. Accordingly, where compounds of the invention, their salts, and solvates and prodrugs thereof, may exist in different tautomeric forms or in equilibrium among such forms, all such forms of the compound are embraced by, and included within the scope of the invention. Examples of such tautomers include, but are not limited to, ketone/enol tautomeric forms, imine-enamine tautomeric forms, and for example heteroaromatic forms such as the following moieties:

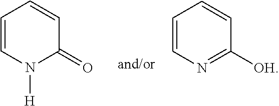 [SMILES: O=c1cccc[nH]1 ; Oc1ccccn1]

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Salts in the solid form may exist in more than one crystal structure and may also be in the form of hydrates. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Salts derived from inorganic bases include aluminum, ammonium, calcium, copper, ferric, ferrous, lithium, magnesium, manganic salts, manganous, potassium, sodium, zinc, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one aspect of the invention the salts are citric, hydrobromic, hydrochloric, maleic, phosphoric, sulfuric, fumaric, and tartaric acids. Similarly, the salts of the acidic compounds are formed by reactions with the appropriate inorganic or organic base.

The terms “treating” or “treatment” (of, e.g., a disease, disorder, or conditions or associated symptoms, which together or individually may be referred to as “indications”) as used herein include: inhibiting the disease, disorder or condition, i.e., arresting or reducing the development of the disease or its biological processes or progression or clinical symptoms thereof; or relieving the disease, i.e., causing regression of the disease or its biological processes or progression and/or clinical symptoms thereof. “Treatment” as used herein also refers to control, amelioration, or reduction of risks to the subject afflicted with a disease, disorder or condition in which LRRK2 is involved. The terms “preventing” or “prevention” or “prophylaxis” of a disease, disorder or condition as used herein includes: impeding the development or progression of clinical symptoms of the disease, disorder, or condition in a mammal that may be exposed to or predisposed to the disease, disorder or condition but does not yet experience or display symptoms of the disease, and the like.

As would be evident to those skilled in the art, subjects treated by the methods described herein are generally mammals, including humans and non-human animals (e.g., laboratory animals and companion animals), in whom the inhibition of LRRK2 kinase activity is indicated or desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising a compound of the invention or a pharmaceutically acceptable salt thereof, together with one or more additional specified ingredients in the specified amounts, as well as any product which results, directly or indirectly, from combination of the specified ingredients in the specified amounts. Such term in relation to a pharmaceutical composition, is intended to encompass a product comprising the active ingredient(s), which include a compound of the invention or a pharmaceutically acceptable salt thereof, optionally together with one or more additional active ingredients, and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention, or a pharmaceutically acceptable salt thereof, and a pharmaceutically acceptable carrier. By “pharmaceutically acceptable” it is meant the carrier, diluent or excipient must be compatible with the other ingredients of the formulation and not deleterious to the recipient thereof.

As noted above, additional embodiments of the present invention are each directed to a method for the treatment a disease, disorder, or condition, or one or more symptoms thereof (“indications”) in which the LRRK2 kinase is involved and for which the inhibition of LRRK2 kinase is desired, which method comprises administering to a subject in need of such treatment a therapeutically effective amount of a compound of the invention, or a pharmaceutically acceptable salt thereof, or a pharmaceutical composition comprising said compound or salt thereof.

In another embodiment, the present invention is directed to a method for the manufacture of a medicament for inhibition of LRRK2 receptor activity in a subject comprising combining a compound of the present invention, or a pharmaceutically acceptable salt thereof, with a pharmaceutical carrier or diluent.

One such embodiment provides a method of treating Parkinson's disease in a subject in need thereof, said method comprising administering to a subject in need of such treatment a therapeutically effective amount of a compound of the invention, or a pharmaceutically acceptable salt thereof, or a pharmaceutical composition comprising said compound or salt thereof. In one such embodiment, the subject is a human.

Another embodiment provides a method for the treatment or prophylaxis of neurologic damage associated with Parkinson's disease in a subject in need thereof. Another embodiment provides a method of treating or improving dopaminergic tone to provide symptomatic relief in a subject in need thereof, for example, in treating, alleviating, ameliorating, or managing motor and non-motor symptoms of Parkinson's disease.

Another embodiment provides a method for the treatment or prophylaxis of abnormal motor symptoms associated with Parkinson's disease (including but not limited to bradykinesia, rigidity and resting tremor). Another embodiment provides a method for the treatment or prophylaxis of abnormal non-motor symptoms associated with Parkinson's disease (including but not limited to cognitive dysfunction, autonomic dysfunction, emotional changes and sleep disruption); Lewy body dementia; and L-Dopa induced dyskinesias. Each said method independently comprises administering to a patient in need of such treatment an effective amount of a compound of the invention, or a pharmaceutically acceptable salt thereof, or pharmaceutically acceptable composition thereof.

Non-limiting examples of additional indications in which LRRK2 is involved and in which the treatment or prophylaxis of said indications in a subject in need thereof are contemplated include the following, each of which, alone or in combination, comprise additional embodiments of the invention: Alzheimer's disease, mild cognitive impairment, the transition from mild cognitive impairment to Alzheimer's disease, tauopathy disorders characterized by hyperphosphorylation of tau such as argyrophilic grain disease, Pick's disease, corticobasal degeneration, progressive supranuclear palsy, inherited frontotemporal dementia, and Parkinson's disease linked to chromosome 17.

Additional indications include neuroinflammation, including neuroinflammation associated with of microglial inflammatory responses associated with multiple sclerosis, HIV-induced dementia, ALS, ischemic stroke, traumatic brain injury and spinal cord injury.

Additional indications include diseases of the immune system including lymphomas, leukemias, multiple sclerosis, rheumatoid arthritis, systemic lupus erythematosus, autoimmune hemolytic anemia, pure red cell aplasia, idiopathic thrombocytopenia pupura (ITP), Evans Syndrome, vasculitis, bullous skin disorder, type I diabetes mellitus, Sjorgen's syndrome, Delvic's disease, inflammatory myopathies, and ankylosing spondylitis.

Additional indications include renal cancer, breast cancer, lung cancer, prostate cancer, and acute myelogenous leukemia (AML) in subjects expressing the LRRK2 G2019S mutation.

Additional indications include papillary renal and thyroid carcinomas in a subject in whom LRRK2 is amplified or overexpressed.

Additional indications include chronic autoimmune diseases including Crohn's disease and leprosy.

The present invention includes within its scope prodrugs of the compounds of this invention. In general, such prodrugs will be functional derivatives of the compounds of this invention which are readily convertible in vivo into the required compound. Thus, in the methods of treatment of the present invention, the terms “administration of” or “administering a” compound shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

The compounds of the present invention may be used in combination with one or more other drugs in the treatment, prevention, control, amelioration, or reduction of risk of diseases or conditions for which compounds of Formula I, Formula I″, Formula II, Formula III, and Formula IV, or the other drugs may have utility, where the combination of the drugs together are safer or more effective than either drug alone. Such other drug(s) may be administered, by a route and in an amount commonly used therefore, contemporaneously or sequentially with a compound of Formula I. When a compound of Formula I, I″, II, and III is used contemporaneously with one or more other drugs, a pharmaceutical composition in unit dosage form containing such other drugs and the compound of Formulae I, I″, II, or III is preferred. However, the combination therapy may also include therapies in which the compound of Formula I, I,″ II, and III, and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I, I″, II, and III.

For example, the present compounds may be used in conjunction with one or more additional therapeutic agents, for example: L-DOPA; dopaminergic agonists such as quinpirole, ropinirole, pramipexole, pergolide and bromocriptine; MAO-B inhibitors such as rasagiline, deprenyl and selegiline; DOPA decarboxylase inhibitors such as carbidopa and benserazide; and COMT inhibitors such as tolcapone and entacapone; or potential therapies such as an adenosine A2a antagonists, metabotropic glutamate receptor 4 modulators, or growth factors such as brain derived neurotrophic factor (BDNF), and a pharmaceutically acceptable carrier.

The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefore, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the other active ingredient(s) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s), and via the same or different routes of administration.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, buccal or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, solutions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated, or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or acetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions and the like, containing the compounds of the present invention are employed. Similarly, transdermal patches may also be used for topical administration.

The pharmaceutical composition and method of the present invention may further comprise other therapeutically active compounds as noted herein which are usually applied in the treatment of the above-mentioned pathological conditions.

In the treatment, prevention, control, amelioration, or reduction of risk of conditions which require inhibition of LRRK2 kinase activity an appropriate dosage level will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day or may be administered once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Methods for preparing the compounds of this invention are illustrated in the following Schemes and Examples. Starting materials are made according to procedures known in the art or as illustrated herein.

### PREPARATIVE EXAMPLES

The compounds of the present invention can be prepared according to the following schemes and specific examples, or modifications thereof, using readily available starting materials, reagents and conventional synthesis procedures. It is also possible to make use of variants which are themselves known to those of ordinary skill in this art but are not mentioned in detail. The general procedures for making the compounds claimed in this invention can be readily understood by one skilled in the art from viewing the following schemes and descriptions. Abbreviations used in the experimentals may include, but are not limited to the following:

| | |
|---|---|
| AcOH | Acetic acid |
| AcONa | Sodium acetate |
| aq. | Aqueous |
| B(pin) | 4,4,5,5-tetramethyl-1,3,2-dioxaborolanyl |
| B₂(pin)₂ | Bis(pinacolato)diboron |
| Bn | Benzyl |
| Boc₂O | Di-tert-butyl dicarbonate |
| BrettPhos Pd G4 | 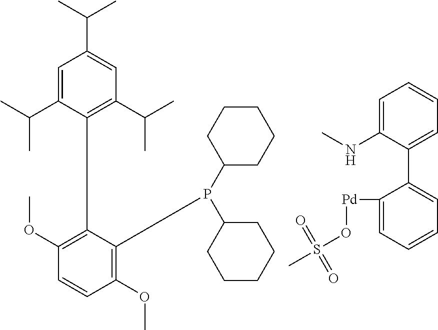 |
| | Methanesulfonato(2-dicyclohexylphosphino-3,6- |
| | dimethoxy-2′,4′,6′-tri-i-propyl-1,1′-biphenyl)(2′- |
| | methylamino-1,1′-biphenyl-2-yl)palladium(II) |
| C₂Cl₆ | Hexachloroethane |
| Catacxium A-Pd G2 | Chloro[(di(1-adamanty1)-N-butylphosphine)-2-(2- |
| | aminobiphenyl)]palladium(II) |
| | 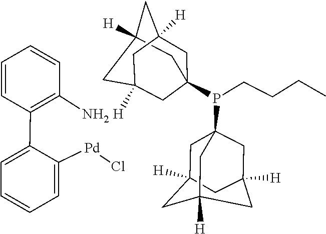 |
| CbzCl | Benzyl chloroformate |
| Celite | Trademark for diatomaceous earth |
| CMBP | Cyanomethylene)tributylphosphorane |
| Co(acac)₂ | Cobalt(II) acetylacetonate |
| CPME | Cyclopentyl methyl ether |
| Cu(OAc)₂ | Copper(II) acetate |
| DAST | Diethylaminosulfur trifluoride |
| DCE | Dichloroethane |
| DCM | Dichloromethane |
| DEA | N,N-Diisopropylethylamine |
| DIAD | Diisopropyl azodicarboxylate |
| DIPEA | N,N-Diisopropylethylamine |
| DMA | Dimethylacetamide |
| DMAP | 4-Dimethylaminopyridine |
| DME | Dimethoxyethane |
| DMF | Dimethylformamide |
| DMF.DMA | N,N-dimethylformamide dimethyl acetal |
| DMP | Dess-Martin periodinane |
| DMSO | Dimethyl sulfoxide |
| DPPA | Diphenylphosphoryl azide |
| Et₂AlCN | Diethylaluminum cyanide |
| Et₃N | Triethylamine |
| EtOAc | Ethyl acetate |
| EtOH | Ethanol |
| ESI | Electrospray ionization |
| h | Hours |
| ¹H NMR | Proton nuclear magnetic resonance |
| HPLC | High performance liquid chromatography |
| iPr | Isopropyl |
| i-PrOB(pin) | 2-Isopropoxy-4,4,5,5-tetramethyl-1,3,2-dioxaborolane |
| IPA | Isopropyl alcohol |
| KOAc | Potassium acetate |
| LAH | Lithium aluminum hydride |
| LCMS | Liquid chromatography-mass spectrometry |
| LiHMDS | Lithium bis(trimethylsilyl)amide |
| m-CPBA | meta-Chloroperoxybenzoic acid |
| m/z | Mass to charge ratio |
| MeCN | Acetonitrile |
| MeMgBr | Methyl magnesium bromide |
| MeOH | Methanol |
| MS | Mass spectrometry |
| MSA | Methanesulfonic acid |
| MsCl | Methanesulfonyl chloride |
| MW | Microwave |
| n-BuLi | n-Butyl lithium |
| NaBH₃CN | Sodium cyanoborohydride |
| NaOMe | Sodium methoxide |
| NBS | N-bromosuccinimide |
| NIS | N-iodosuccinimide |
| Pd/C | Palladium on carbon |
| Pd(dppf)Cl₂ | [1,1′- |
| | Bis(diphenylphosphino)ferrocene]dichloropalladium(II) |
| Pd(OH)₂/C | Palladium hydroxide on carbon |
| PE | Petroleum ether |
| PPh₃ | Triphenylphosphine |
| psi | Pounds per square inch |
| PTSA | p-Toluenesulfonic acid |
| RT | Room temperature |
| sat. | saturated |
| SEM/SEMCl | 2-(Trimethylsilyl)ethoxymethyl/2- |
| | (Trimethylsilyl)ethoxymethyl chloride |
| SFC | Supercritical fluid chromatography |
| t-BuOH | tert-Butanol |
| TEA | Triethylamine |
| TFA | Trifluoroacetic acid |
| TfOH | Triflic acid |
| THF | Tetrahydrofuran |
| TLC | Thin Layer Chromatography |
| TMS | Trimethylsilyl |
| t_{R} | Retention time |
| TBAF | Tetra-n-butylammonium fluoride |
| TBAI | |
| TBME | Tert-butyl methyl ether |
| Tf2O | Trifluoromethanesulfonic anhydride |
| TMSCF₃ | Trifluoromethyltrimethylsilane |
| XantPhos Pd G4 | 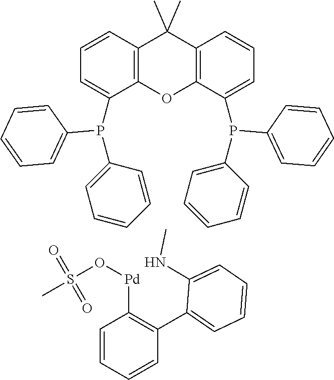 |
| | [(4,5-Bis(diphenylphosphino)-9,9-dimethylxanthene)-2-(2′- |
| | methylamino-1,1′-biphenyl)]palladium(II) |
| | methanesulfonate |

### General Experimental Information

Unless otherwise noted, all reactions are magnetically allowed to stir, diethyl ether used in the experiments described below is Fisher ACS certified material stabilized with BHT, and “concentrated” and/or “solvent removed under reduced pressure” means evaporating the solvent from a solution or mixture using a rotary evaporator or vacuum pump. Unless otherwise noted, flash chromatography is carried out on a Teledyne Isco (Lincoln, NE), Analogix (Burlington, WI), or Biotage (Stockholm, SWE) automated chromatography system using a commercially available cartridge as the column. Columns may be purchased from Teledyne Isco, Analogix, Biotage, Varian (Palo Alto, CA), or Supelco (Bellefonte, PA) and are usually filled with silica gel as the stationary phase. Reverse phase prep-HPLC conditions, where used, can be found at the end of each experimental section. Aqueous solutions were concentrated on a Genevac (Ipswich, ENG) or by freeze-drying/lyophilization. Unless otherwise noted, all LRRK2 pIC₅₀ data presented in tables refers to the LRRK2 G2019S Kₘ ATP LanthaScreen™ assay (Life Technologies Corp., Carlsbad, CA) that is described in the Biological Assay section.

### Synthesis of Common Intermediates

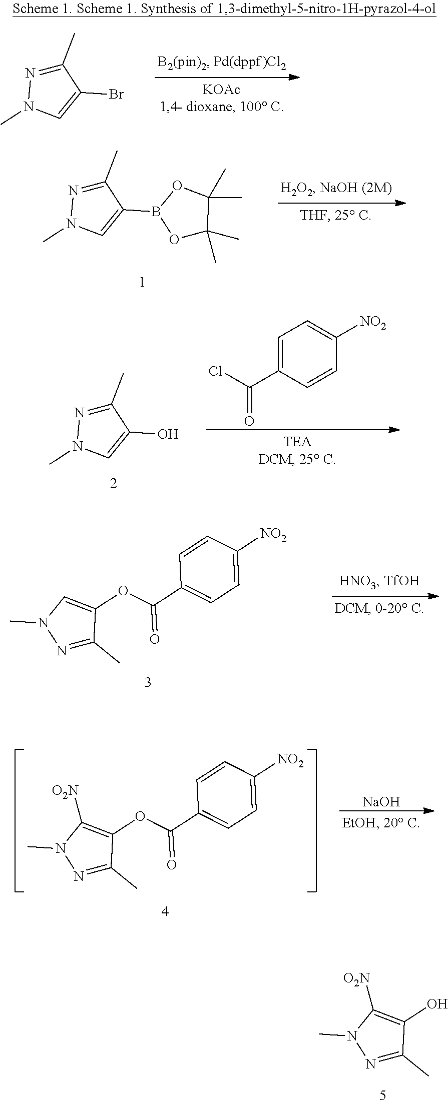

### 1,3-Dimethyl-4-(4,4,5,5-tetramethyl-1,3,2-dioxaborolan-2-yl)-1H-pyrazole (1)

To a 5-L round-bottom flask was added 4-bromo-1,3-dimethyl-1H-pyrazole (200 g, 1.14 mol), B₂(pin)₂ (348 g, 1.37 mol) and KOAc (224 g, 2.29 mol) and 1,4-dioxane (3.0 L) to a 5.0 L round-bottom flask. Pd(dppf)Cl₂—CH₂Cl₂ (46.7 g, 57.1 mmol) was added at 25° C. The reaction mixture was allowed to stir at 100° C. for 12 h under N₂. The reaction mixture was concentrated under reduced pressure. The crude residue was diluted with water (10 L) and extracted with EtOAc (3×10 L). The combined organic layers were dried over Na₂SO₄. The solution was filtered and concentrated under reduced pressure. The resulting residue was purified by flash chromatography on silica gel (gradient elution of 10-100% EtOAc/PE) to afford the title compound.

### 1,3-Dimethyl-1H-pyrazol-4-ol (2)

To a 5-L round-bottom flask was added 1,3-dimethyl-4-(4,4,5,5-tetramethyl-1,3,2-dioxaborolan-2-yl)-1H-pyrazole (200 g, 900 mmol) and THF (1.0 L). To the mixture was slowly added aqueous NaOH (2 M, 1.35 L) at 25° C. To the mixture was slowly added H₂O₂ (30% w/w in H₂O, 175 mL, 1.82 mol) at 25° C. The reaction mixture was allowed to stir at 25° C. for 12 h. The reaction was quenched by the addition of saturated aqueous Na₂SO₃ (1.2 L) and acidified with aqueous 2 M HCl (aq) until pH 4. The reaction was extracted with EtOAc (4×2.0 L). The combined organic layers were dried with anhydrous Na₂SO₄. The mixture was filtered and concentrated under reduced pressure. The crude residue was re-crystallized from EtOAc/PE (10%, 500 mL) to afford the title compound.

### 1,3-Dimethyl-1H-pyrazol-4-yl 4-nitrobenzoate (3)

To a 5-L round-bottom flask was added 1,3-dimethyl-1H-pyrazol-4-ol (140 g, 1.25 mol), 4-nitrobenzoyl chloride (255 g, 1.37 mol) and DCM (3.0 L). To the mixture was added dropwise TEA (348 mL, 2.50 mol). The reaction mixture was allowed to stir at 25° C. for 1 h. The reaction mixture was diluted with H₂O (1 L). The aqueous layer was extracted with DCM (2×1.0 L). The combined organic layers were washed with saturated aqueous NaHCO₃ (1.0 L) and dried over Na₂SO₄. The solution was filtered and concentrated under reduced pressure. The residue was triturated with TBME (1.0 L) at 25° C. for 1 h. The mixture was filtered to afford the title compound. The product was used in the next step directly without further purification.

### 1,3-Dimethyl-5-nitro-1H-pyrazol-4-ol (4)

To a flask containing a solution of TfOH (216 mL, 2.45 mol) in dry DCM (400 mL) was carefully added HNO₃ (56.3 mL, 1.25 mol) at 0° C. The reaction mixture was allowed to stir at 0° C. for 30 min. To the mixture was added a solution of 1,3-dimethyl-1H- pyrazol-4-yl 4-nitrobenzoate (80.0 g, 306 mmol) in dry DCM (800 mL) at 0° C. and allowed to stir for 1 h before warming to 25° C. The reaction was allowed to stir for an additional 12 h to afford 1,3-dimethyl-5-nitro-1H-pyrazol-4-yl 4-nitrobenzoate. To the mixture was added dropwise 4 M NaOH (995 mL) at 10-20° C. To the mixture was added EtOH (400 mL) and allowed to stir at 25° C. for 2 h. The reaction mixture was extracted with DCM (2×1 L). The aqueous layer was acidified with 6 M HCl until pH ˜3-4. The mixture was filtered and washed with H₂O (2×500 mL). The filtrate was extracted with TBME (5×1 L). The combined organic layers were concentrated under reduced pressure. The crude residue was purified by reversed-phase HPLC (Column: DAC-150, Agela C18; Mobile phase A: Water w/0.1% HCl; Mobile phase B: MeOH; 25-40% 30 min; 40% hold for 30 min) to afford the title compound. MS (ESI): m/z calc'd for C₅H₇N₃O₃ [M+H]⁺: 158, found 158; ¹H NMR: (400 MHz, DMSO-d₆) δ: 10.02 (br s, 1H), 3.77-4.15 (m, 3H), 1.94-2.27 (m, 3H).

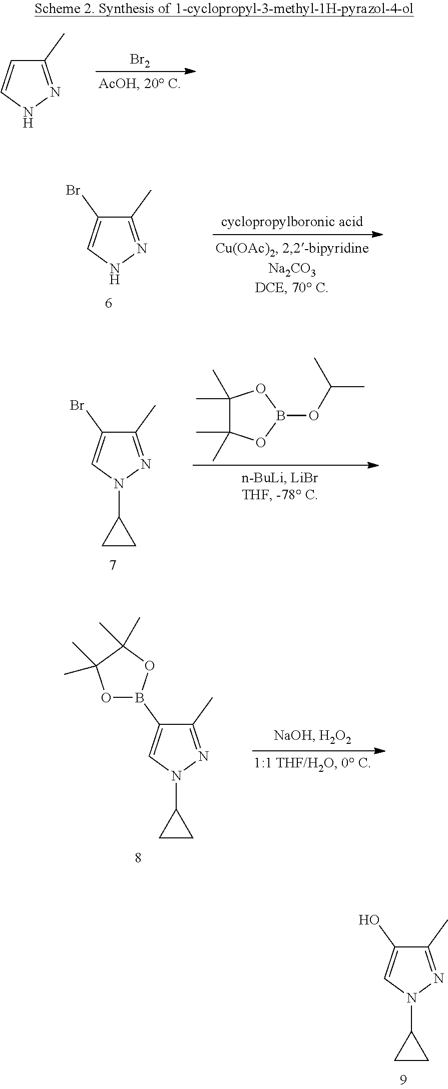

### 4-Bromo-3-methyl-1H-pyrazole (5)

To a 3 L flask was added 3-methyl-1H-pyrazole (300 g, 3.64 mol) and AcOH (1.50 L). The mixture was added Br₂ (613 g, 3.84 mol). The mixture was allowed to stir at 20° C. for 1 h. The mixture was filtered and washed with ethyl acetate (2×500 mL). The mixture was diluted with ethyl acetate (800 mL) and basified with 2 M NaOH to pH 10. The layers were separated. The organic layer was washed with brine and dried with anhydrous Na₂SO₄. The solution was filtered and concentrated under reduced pressure to afford the title compound 6.

### 4-Bromo-1-cyclopropyl-3-methyl-1H-pyrazole (6)

To a 5-L round-bottom flask was added 2,2′-bipyridine (243 g, 2.02 mol), Na₂CO₃ (329 g, 3.11 mol) Cu(OAc)₂ (282 g, 1.55 mol) and DCE (3.20 L). The mixture was allowed to stir at 20° C. for 30 min and then heated to 70° C., stirring for 1 h. To the mixture was added 4-bromo-3-methyl-1H-pyrazole 6 (250 g, 1.55 mol) at 70° C. The reaction mixture was allowed to stir at 70° C. for 1 h. To the mixture was added cyclopropylboronic acid (173 g, 2.02 mol) at 70° C. The reaction mixture was allowed to stir at 70° C. for 3 h. The reaction mixture was diluted with NH₄OH (750 mL), H₂O (750 mL) and extracted with DCM (3×750 mL). The combined organic layers were washed with H₂O (750 mL), 4 N HCl (375 mL) and brine. The organic layer was dried over Na₂SO₄, filtered and concentrated under reduced pressure. The crude residue was purified by flash chromatography on silica gel (gradient elution of 0.5-2.5% EtOAc/PE) to afford the title compound 7.

### 1-Cyclopropyl-3-methyl-4-(4,4,5,5-tetramethyl-1,3,2-dioxaborolan-2-yl)-1H-pyrazole (7)

To a 2-L round-bottom flask was added 4-bromo-1-cyclopropyl-3-methyl-1H-pyrazole (100 g, 0.50 mol) and THF (700 mL). To the mixture was added 2-isopropoxy-4,4,5,5-tetramethyl-1,3,2-dioxaborolane (111 g, 0.60 mol) and LiBr (47.5 g, 0.55 mol). To the mixture was added n-BuLi (2.5 M in hexanes, 299 mL, 0.71 mol) −78° C. under N₂. The mixture was allowed to stir at −78° C. for 1 h. The reaction mixture was diluted with AcOH/THF (300 mL) and extracted with ethyl acetate (3×500 mL), dried over Na₂SO₄. The mixture was filtered and concentrated under reduced pressure to afford the title compound 8. The product was used in the next step directly without further purification.

### 1-Cyclopropyl-3-methyl-1H-pyrazol-4-ol (8)

To a 3-L round-bottom flask was added 1-cyclopropyl-3-methyl-4-(4,4,5,5-tetramethyl-1,3,2-dioxaborolan-2-yl)-1H-pyrazole (123 g, 0.50 mol) and THF (615 mL). To the mixture was added H₂O₂ (30% w/w in H₂O, 112 g, 0.99 mol), NaOH (59.5 g, 1.49 mol) and H₂O (615 mL) at 0° C. under N₂. The reaction mixture was allowed to stir at 0° C. for 4 h, then was diluted with Na₂SO₃ (100 mL) and extracted with ethyl acetate (20×500 mL). The combined organic layers were dried over Na₂SO₄. The mixture was filtered and concentrated under reduced pressure. The crude residue was purified by preparative HPLC (Column & dimensions: Welch Xtimate C18 (250×70 mm, 10 μm); Mobile phase A: 10 mM NH₄HCO₃ in H₂O; Mobile phase B: MeCN; 2-25%, 20 min) to afford the title compound 9. MS (ESI): m/z calc'd for C₇H₁₀N₂O [M+H]⁺: 139, found 139; ¹H NMR (400 MHz, CDCl₃) δ 7.98 (s, 1H), 6.98 (s, 1H), 3.38-3.32 (m, 1H), 2.11 (s, 3H), 0.96-0.87 (m, 4H).

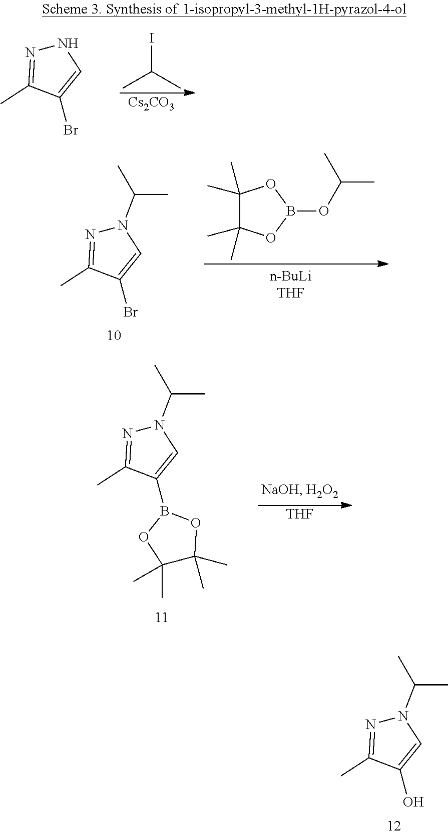

### 4-Bromo-1-isopropyl-3-methyl-1H-pyrazole (10)

To a solution of 4-bromo-3-methyl-1H-pyrazole (260 g, 1.61 mol, 1 eq.) in acetonitrile (10V, 2.6 L) were added 2-iodopropane (824 g, 4.84 mol) and Cs₂CO₃ (792 g, 2.42 mol, 1.5 eq.) in portions at 25° C. under a nitrogen atmosphere. The reaction mixture was allowed to stir for 1 h at 25° C. under nitrogen atmosphere and was filtered. The filter cake was washed with acetonitrile (3×260 mL). The filtrate was concentrated under reduced pressure. The residue was dissolved in ethyl acetate (1.3 L). The aqueous layer was extracted with EtOAc (2×1.3 L). The combined organic layers were washed with brine (1×1.3 L), dried over anhydrous Na₂SO₄. After filtration, the filtrate was concentrated under reduced pressure to afford the title compound 10. The crude product was used in the next step directly without further purification.

### 1-Isopropyl-3-methyl-4-(4,4,5,5-tetramethyl-1,3,2-dioxaborolan-2-yl)-1H-pyrazole (11)

To a solution of 4-bromo-1-isopropyl-3-methylpyrazole 10 (311 g, 1.53 mol) in THF (10 V, 3.11 L) was added n-butyllithium (2.5 M, 137 g, 2.14 mol) dropwise at −78° C. under a nitrogen atmosphere. The reaction mixture was allowed to stir for 30 min at −78° C. To the mixture was added a solution of 2-isopropoxy-4,4,5,5-tetramethyl-1,3,2-dioxaborolane (627 g, 3.37 mol, 2.2 eq.) in THF (1.5 V, 390 mL), and the mixture was warmed 20° C. over 30 min. The reaction was quenched with sat. NH₄Cl (aq.) at room temperature. The aqueous layer was extracted with EtOAc (3×3 L). The combined organic layers were washed with brine (1×3 L), dried over anhydrous Na₂SO₄. After filtration, the filtrate was concentrated under reduced pressure to afford the title compound 11. The crude product was used in the next step directly without further purification.

### 1-Isopropyl-3-methyl-1H-pyrazol-4-ol (12)

To a allowed to stir mixture of 1-isopropyl-3-methyl-4-(4,4,5,5-tetramethyl-1,3,2-dioxaborolan-2-yl) pyrazole 11 (490 g, 1.96 mol) in THF (4.9 L, 10 V) was added NaOH (2.94 L) dropwise at 10° C. under nitrogen atmosphere. To the above mixture was added H₂O₂ (30% w/w, 444.3 g, 3.92 mol) dropwise over 1 h at 0° C. The reaction mixture was allowed to stir for additional overnight at room temperature. The reaction was quenched by the addition of sat. Na₂SO₃ (aq.) (2.45 L) at 0° C. The aqueous layer was extracted with EtOAc (2×2.45 L). The aqueous layer was acidified to pH 4 with HCl (2 N). The aqueous layer was extracted with CH₂Cl₂ (6×2.45 L). The combined organic layers were dried over anhydrous Na₂SO₄. After filtration, the filtrate was concentrated under reduced pressure. The residue was purified by silica gel column chromatography, eluted with PE/EA (10:1-3:1) to afford crude product. The crude product was purified by Prep-SFC with the following conditions (Column: CHIRALPAK IC, 3×25 cm, 5 μm; Mobile Phase A: CO₂; Mobile Phase B: IPA (0.5% 2M NH3-MeOH); 210 nm) to afford the title compound 12. MS (ESI): m/z calc'd for C₇H₁₂N₂O [M+H]⁺: 141, found 141; ¹H NMR (400 MHz, DMSO-d₆): δ 7.92 (s, 1H), 7.09 (s, 1H), 4.20 (hept, J=6.5 Hz, 1H), 2.00 (s, 3H), 1.30 (dd, J=6.8, 1.1 Hz, 6H).

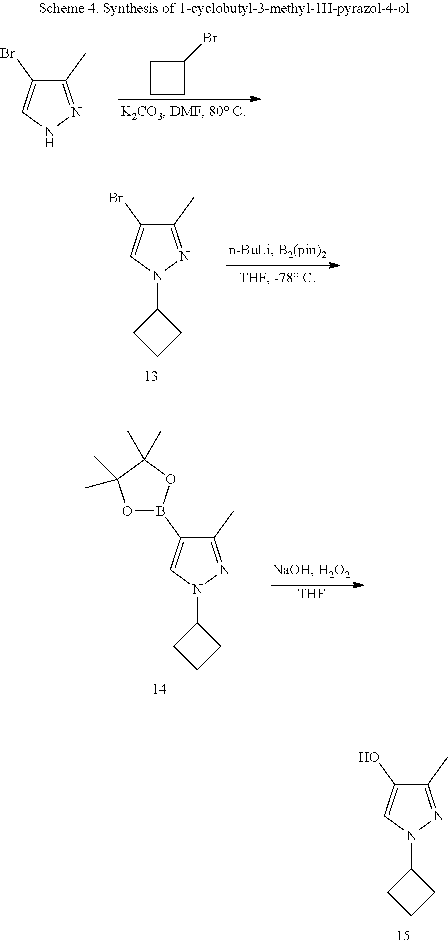

### 4-Bromo-1-cyclobutyl-3-methyl-1H-pyrazole (139)

To a solution of 4-bromo-3-methyl-1H-pyrazole 6 (20.0 g, 124 mmol) and bromocyclobutane (20.1 g, 149 mmol) in anhydrous DMF (100 mL) was added K₂CO₃ (25.8 g, 186 mmol). The mixture was allowed to stir at 80° C. for 16 h, then concentrated under reduced pressure. The crude residue purified by flash chromatography on silica gel (gradient elution of 0-25% EtOAc/PE) to afford the title compound 13.

### 1-Cyclobutyl-3-methyl-4-(4,4,5,5-tetramethyl-1,3,2-dioxaborolan-2-yl)-1H-pyrazole (14)

The reaction mixture was allowed to stir at −78° C. for 30 min. To the mixture was added 2-isopropoxy-4,4,5,5-tetramethyl-1,3,2-dioxaborolane (13.0 g, 69.7 mmol). The reaction mixture was allowed to warm to 20° C. and allowed to stir for 2 h. The reaction was quenched by the addition of saturated aqueous NH₄Cl solution (100 mL) and extracted with EtOAc (3×130 mL). The combined organic layers were washed with brine and dried over MgSO₄. The solution was filtered and concentrated under reduced pressure. The residue was purified by flash chromatography on silica gel (gradient elution of 0-25% EtOAc/PE) to afford the title compound 14.

### 1-Cyclobutyl-3-methyl-1H-pyrazol-4-ol (15)

To a solution of 1-cyclobutyl-3-methyl-4-(4,4,5,5-tetramethyl-1,3,2-dioxaborolan-2-yl)-1H-pyrazole 14 (11.0 g, 21.0 mmol) in THF (40 mL) was added NaOH (2.52 g, 62.9 mmol) and H₂O₂ (4.29 ml, 42.0 mmol). The solution was allowed to stir at 20° C. for 2 h. Excess H₂O₂ was quenched by the addition of saturated aqueous Na₂SO₃ (40 mL). The mixture was extracted with EtOAc (3×50 mL). The aqueous phase was concentrated under reduced pressure. The residue was purified by reversed-phase HPLC (Column Phenomenex Genimi NX C18 5 μm, 150×40 mm; Mobile phase A: 10 mM NH₄HCO₃ in H₂O; Mobile phase B: MeCN, 5-45% 10 min; 100% 2 min). The isomers were separated by preparative HPLC (Column: Phenomenex Titank C18 Bulk (250×100 mm, 10 μm); Mobile phase A: 10 mM NH₄HCO₃; Mobile Phase B: MeCN]; 5-35% 20 min). The solution was concentrated. The residue was extracted with EtOAc (5.00 L, 4.00 L, 4.00 L, 3.00 L). The combined organic layers were washed with brine (800 mL) and dried over Na₂SO₄. The solution was filtered and concentrate under reduced pressure to afford the title compound 15. MS (ESI): m/z calc'd for C₈H₁₂N₂O [M+H]⁺: 153, found 153; ¹H NMR: (400 MHz, CDCl₃) δ: 7.20 (s, 1H), 7.04 (s, 1H), 4.53 (m, 1H), 2.26-2.45 (m, 4H), 2.13 (s, 3H), 1.67-1.84 (m, 2H).

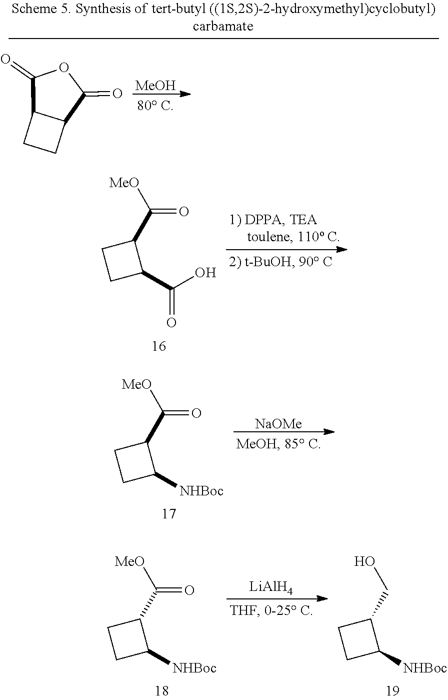

### (1R,2S)- and (1S,2R)-2-(Methoxycarbonyl)cyclobutane-1-carboxylic acid (16)

To a flask was added (1R,5S)- and (1S,5R)-3-oxabicyclo[3.2.0]heptane-2,4-dione (200 g, 1.59 mol) and MeOH (1.40 L). The mixture was heated to 80° C. and allowed to stir for 12 h. The reaction mixture was concentrated under reduced pressure to afford the title compound 16. The product was used in the next step directly without further purification. ¹H NMR (400 MHz, CDCl₃) δ:11.53 (s, 1H), 3.69 (s, 3H), 3.41-3.44 (m, 2H), 2.35-2.42 (m, 2H), 2.21-2.24 (m, 2H).

### Methyl (1S,2R)- and (1R,2S)-2-((tert-butoxycarbonyl)amino)cyclobutane-1-carboxylate (17)

To a flask containing a solution of (1R,2S)- and (1S,2R)-2-(methoxycarbonyl)cyclobutane-1-carboxylic acid 16 (255 g, 1.61 mol) in toluene (1.65 L) was added DPPA (384 mL, 1.77 mol) and TEA (247 mL, 1.77 mol). The reaction mixture was allowed to stir at 25° C. for 3 h. The reaction mixture was then heated to 110° C. and allowed to stir for 2 h. The reaction mixture was cooled to 20° C. and concentrated under reduced pressure. The crude residue was dissolved in t-BuOH (1.65 L). The reaction mixture was heated to 90° C. and allowed to stir for 12 h. The reaction mixture was concentrated under reduced pressure. The residue was dissolved in H₂O (1.50 L) and extracted with MTBE (1.00 L, 800 mL). The combined organic layers were washed with brine and dried over Na₂SO₄. The solution was filtered and concentrated under reduced pressure. The resulting residue was purified by flash chromatography on silica gel (gradient elution of 5-100% EtOAc/PE) to afford the title compound 17. ¹H NMR (400 MHz, CDCl₃) δ: 5.34 (s, 1H), 4.44-4.48 (m, 1H), 3.71 (s, 3H), 3.38 (m, 1H), 2.34-2.36 (m, 1H), 2.19-2.24 (m, 1H), 1.95-1.99 (m, 2H), 1.42 (s, 9H).

### Methyl (1S,2S)- and (1R,2R)-2-((tert-butoxycarbonyl)amino)cyclobutane-1-carboxylate (17)

To a solution of methyl (1S,2R)- and methyl (1R,2S)-2-((tert-butoxy carbonyl)amino)cyclobutane-1-carboxylate 17 (40.0 g, 174 mmol) in MeOH (4.00 L) was added NaOMe (5.4 M, 64.6 mL). The reaction mixture was heated to 85° C. and allowed to stir for 2 h. The reaction mixture was cooled to 20° C. and acidified with 1 N HCl to pH 5. The organic solvent was concentrated under reduced pressure. The reaction mixture was extracted with EtOAc (2.00 L, 1.50 L, 1.00 L). The combined organic layers were washed with brine (800 mL) and dried with anhydrous Na₂SO₄. The solution was filtered and concentrated under reduced pressure. The resulting residue was purified by flash chromatography on silica gel (gradient elution of 50-80% EtOAc/PE). The crude product was triturated with PE (200 mL) at 0° C. for 30 min to afford the title compound 18. ¹H NMR (400 MHz, CDCl₃) δ: 5.09 (s, 1H), 4.18-4.21 (m, 1H), 3.73-3.78 (m, 1H), 3.56-3.61 (m, 1H), 2.71 (s, 2H), 2.35-2.46 (m, 2H), 1.90-1.92 (m, 1H), 1.78-1.89 (m, 1H), 1.60-1.64 (m, 1H), 1.44 (s, 1H).

### tert-Butyl ((1S,2S)-2-(hydroxymethyl)cyclobutyl)carbamate (19)

To a solution of methyl (1S,2S)- and (1R,2R)-2-((tert-butoxycarbonyl)amino)cyclobutane-1-carboxylate 18 (100 g, 436 mmol) in THF (1.00 L) was added portion-wise LiAlH₄ (49.6 g, 1.31 mol) at 0° C. The reaction mixture was warmed to 25° C. and allowed to stir for 1 h. The reaction mixture was carefully quenched by the addition of H₂O (49.6 mL), 15% aqueous NaOH (49.6 mL) and H₂O (149 mL). The reaction mixture was allowed to stir at 25° C. for 30 min. The reaction mixture was filtered. The solution was concentrated under reduced pressure. The resulting residue was purified by flash chromatography on silica gel (gradient elution of 5-100% EtOAc/PE). Chiral resolution of the two aza-indane diastereomers was achieved by chiral preparative SFC (column: DAICEL CHIRALPAK AD (250 mm×50 mm, 10 μm); Mobile phase A: CO₂; Mobile phase B: 0.1% NH₄OH in MeOH]; 11% hold for 3.5 min) to afford the title compound 19 (t_{R}=1.3 min). MS (ESI): m/z calc'd for C₁₀H₁₉NO₃ [M-C₄H₈]⁺: 146, found 146; ¹H NMR: (400 MHz, CDCl₃) δ 4.86 (s, 1H), 4.06 (br s, 1H), 3.48-3.63 (m, 3H), 2.25-2.33 (m, 1H), 2.20-2.23 (m, 1H), 1.70-1.83 (m, 2H), 1.44 (s, 9H), 1.37-1.39 (m, 1H).

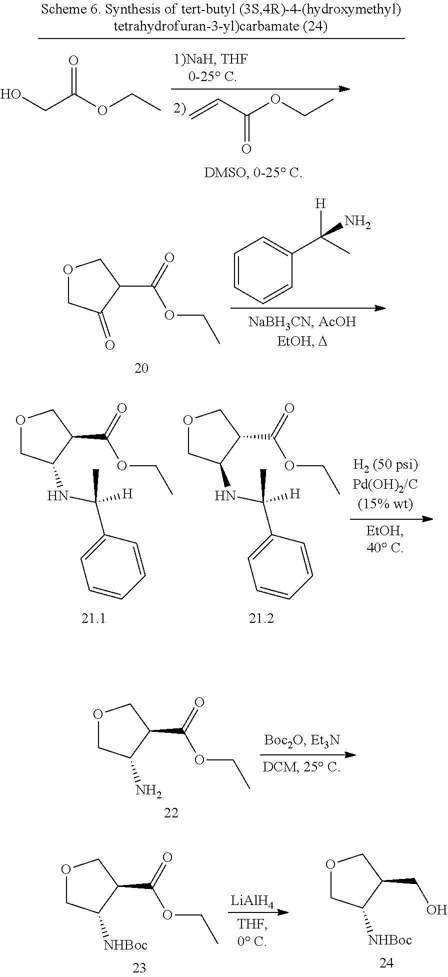

### Ethyl (R)- and (S)-4-oxotetrahydrofuran-3-carboxylate (20)

To a 20-L four-neck flask was added THF (2.8 L) and NaH (131 g, 5.44 mol) under N₂ atmosphere. The mixture was cooled to 0° C. and added ethyl 2-hydroxyacetate (567 g, 5.44 mol) dropwise at 0-5° C. The mixture was allowed to stir at 0° C. for 1 h and then warmed to 25° C. to stir for an additional 1 h. The mixture cooled to 0° C., and DMSO (5.7 L) was added dropwise slowly. To the mixture was then added dropwise ethyl acrylate (654 g, 6.53 mol). The reaction mixture was allowed to stir overnight at 25° C. The reaction mixture was diluted with 1 N HCl (10 L) and ice water (15 L) and extracted with EtOAc (2×10 L). The combined organic layers were washed with H₂O (10 L×1), brine (10 L), and dried over MgSO₄. The solution was filtered and concentrated under reduced pressure. The residue was purified by flash chromatography on silica gel (15% EtOAc/PE) to afford the title compound 20.

### Ethyl (3S,4S)- and (3R,4R)-4-(((S)-1-phenylethyl)amino)tetrahydrofuran-3-carboxylate (21.1 and 21.2)

To a solution of ethyl (R)- and (S)-4-oxotetrahydrofuran-3-carboxylate 20 (771 g, 4.88 mol) and (S)-α-phenylethylamine (709 g, 5.85 mol) in EtOH (8 L) was added AcOH (293 g, 4.88 mol) under a N₂ atmosphere. The resulting solution was allowed to stir at 25° C. for 2 h. To the mixture was then added portion-wise NaBH₃CN (460 g, 7.31 mol). The reaction mixture was refluxed overnight, then cooled to room temp. The solution was filtered, concentrated under reduced pressure and diluted with H₂O (10 L) and ethyl acetate (5 L), then extracted with EtOAc (2×5 L). The combined organic layers were washed with H₂O (10 L) and brine (10 L) and dried over Na₂SO₄. The solution was filtered and concentrated under reduced pressure. The residue was purified by flash chromatography on silica gel (25% EtOAc/PE) to afford a racemic mixture of 20. Separation of the isomers were achieved by chiral preparative SFC (Column, Chiralpak SC; Mobile phase A: CO₂; Mobile phase B: 0.5% NH₃ in IPA; Detector, 220 nm; Gradient: 30% B) to afford the title compounds 21.1 (t_{R}=5.09 min) and 21.2 (t_{R}=4.21 min).

### Ethyl (3S,4S)-4-aminotetrahydrofuran-3-carboxylate (22)

To a solution of ethyl (3S,4S)-4-(((S)-1-phenylethyl)amino)tetrahydrofuran-3-carboxylate 21.2 (279 g, 1.06 mol) in EtOH (3 L) was added Pd(OH)₂/C (83.7 g, 15% wt, 50% H₂O) in a pressure tank. The mixture was hydrogenated at 40° C. under 50 psi of H₂ overnight. The reaction was then cooled to RT. The reaction mixture was filtered through a Celite pad and washed with EtOH. The combined filtrate was concentrated under reduced pressure to afford the title compound 22. The product was used in the next step directly without further purification.

### Ethyl (3S,4S)-4-((tert-butoxycarbonyl)amino)tetrahydrofuran-3-carboxylate (23)

To a solution of ethyl (3S,4S)-4-aminotetrahydrofuran-3-carboxylate 22 (165 g, 1.03 mol) and Et₃N (210 g, 2.08 mol) in DCM (3 L) was added di-tert-butyl dicarbonate (340 g, 1.56 mol) at RT under N₂ atmosphere. The reaction mixture was allowed to stir at RT overnight and then diluted with H₂O (5 L). The mixture was extracted with DCM (2×1 L). The combined organic layers were dried over Na₂SO₄. The solution was filtered and concentrated under reduced pressure. The residue was purified by flash chromatography on silica gel (30% EtOAc/PE) to afford the title compound 23.

### tert-Butyl ((3S,4R)-4-(hydroxymethyl)tetrahydrofuran-3-yl)carbamate (24)

To a solution of ethyl (3S,4S)-4-((tert-butoxy carbonyl)amino)tetrahydrofuran-3-carboxylate 23 (12.4 g, 47.8 mmol) in THF (250 mL) was added dropwise LiAlH₄ (2.5M in THF, 19.2 mL, 47.8 mmol) at 0° C. under N₂ atmosphere. The reaction mixture was allowed to stir at RT for 30 min. The reaction mixture was quenched by the addition of Na₂SO₄·10H₂O until the evolution of gas ceased. The reaction mixture was filtered and washed with DCM. The combined filtrate was concentrated under reduced pressure. The residue was purified by flash chromatography on silica gel (50% EtOAc/PE) to afford the title compound 24. MS (ESI): m/z calc'd for C₁₀H₁₉NO₄ [M+Na]⁺: 240, found 240; ¹H NMR (400 MHz, DMSO-d₆): δ 7.10 (d, J=6.8 Hz, 1H), 4.69 (t, J=5.2 Hz, 1H), 3.79 (ddd, J=12.2, 8.6, 6.8 Hz, 2H), 3.70 (dd, J=7.8, 4.2 Hz, 1H), 3.53-3.34 (m, 3H), 3.29 (ddd, J=10.7, 7.8, 5.5 Hz, 1H), 2.15 (ddt, J=12.9, 7.6, 5.5 Hz, 1H), 1.38 (s, 9H). When formatting the section above, space between the title and the description of each step can be reduced. These can be compacted to take up less space.

### General Synthetic Schemes and Preparative Examples

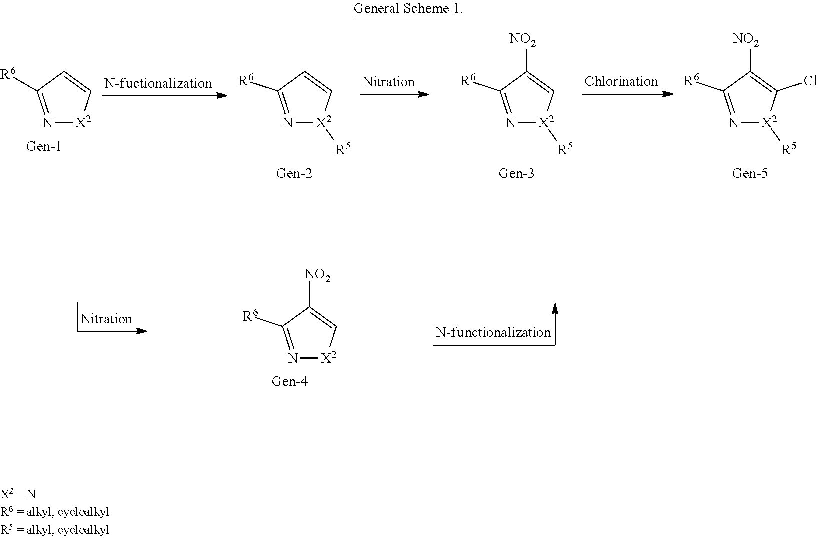

In General Scheme 1, commercially available or synthetically prepared 3-substituted pyrazoles Gen-1 could be alkylated using a number of synthetic transformations commonly known to those skilled in the art, including, but not limited to, a base-mediated alkylation or a Mitsunobu reaction to afford N-alkyl 3-substituted pyrazoles Gen-2. These transformations typically afforded a mixture of N1- and N2-alkyl substituted pyrazoles and could be separated by achiral or chiral purification methods. A number of intermediates of the form Gen-2 are commercially available, including alkyl groups of the depicted substitution pattern. Likewise, alkyl groups in this substitution pattern can be accessed synthetically by known methods. N-alkylated Gen-2 could be functionalized at the 4-position by treatment with nitric acid to form Gen-3. Alternatively, Gen-1 could be nitrated to Gen-4 and subsequent base-mediated alkylation or Mitsunobu to synthetically prepare Gen-3. The nitrated pyrazole Gen-3 could be functionalized at the 5-position through metal-mediated deprotonation and chlorination sequence to afford elaborated compounds of the form Gen-5. Representative preparative examples from each sequence are described in more detail below.

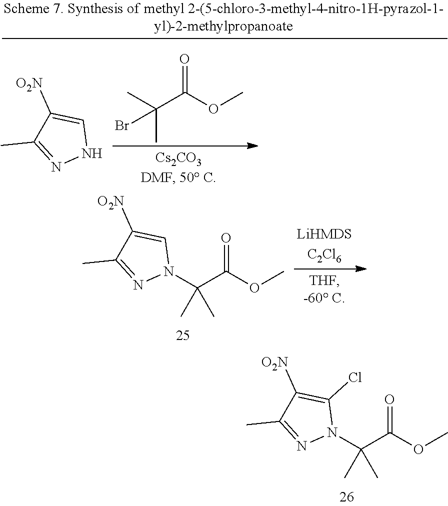

### Methyl 2-methyl-2-(3-methyl-4-nitro-1H-pyrazol-1-yl)propanoate (25)

To a flask was containing 3-methyl-4-nitro-1H-pyrazole (2.00 g, 15.7 mmol) was added cesium carbonate (7.7 g, 23.6 mmol) and DMF (100 mL). To the reaction mixture was added methyl 2-bromo-2-methylpropanoate (3.00 mL, 23.2 mmol). The reaction mixture was heated to 50° C. and allowed to stir for 105 min. The mixture was filtered and the filtrate was concentrated. The crude residue was purified by flash chromatography on silica gel (gradient elution of 0-50% EtOAc/hexanes) to afford the title compound 25. MS (ESI): m/z calc'd for C₉H₁₃N₃O₄ [M+H]⁺: 228, found 228.

### Methyl 2-(5-chloro-3-methyl-4-nitro-1H-pyrazol-1-yl)-2-methylpropanoate (26)

To a solution of methyl 2-methyl-2-(3-methyl-4-nitro-1H-pyrazol-1-yl)propanoate 25 (1.05 g, 4.62 mmol) in THF (12 mL), was added LiHMDS (1.5 M in THF, 4.00 mL, 6.00 mmol) at −60 C under N₂ atmosphere. The reaction was allowed to stir at −60° C. for 1.5 h. To the reaction mixture was added dropwise a solution of hexachloroethane (3.28 g, 13.9 mmol) in THF (6 mL) −50° C. The reaction was allowed to warm to −30° C. and stir for 1.5 h. The reaction was quenched by the addition of NH₄Cl (sat.) and extracted with EtOAc (3×). The combined organic layers were washed with brine and dried over MgSO₄. The solution was filtered and concentrated under reduced pressure. The crude residue was purified by flash chromatography on silica gel (gradient elution of 0-30% EtOAc/hexanes) to afford the title compound 26. MS (ESI): m/z calc'd for C₉H₁₂ClN₃O₄ [M+H]⁺: 262, found 262.

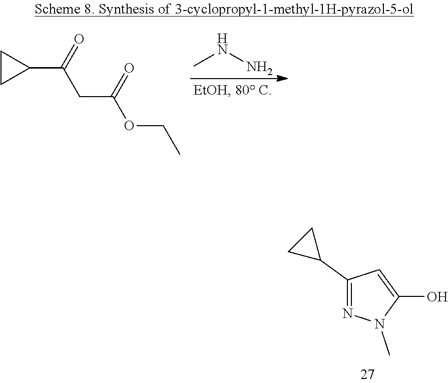

### 3-Cyclopropyl-1-methyl-1H-pyrazol-5-ol (27)

The crude residue was recrystallized from EtOAc (30 mL) to afford the title compound 27. ¹H NMR (400 MHz, CDCl₃) δ: 3.25 (s, 3H), 3.03 (s, 2H), 1.82-1.71 (m, 1H), 0.99-0.92 (m, 2H), 0.80-0.74 (m, 2H).

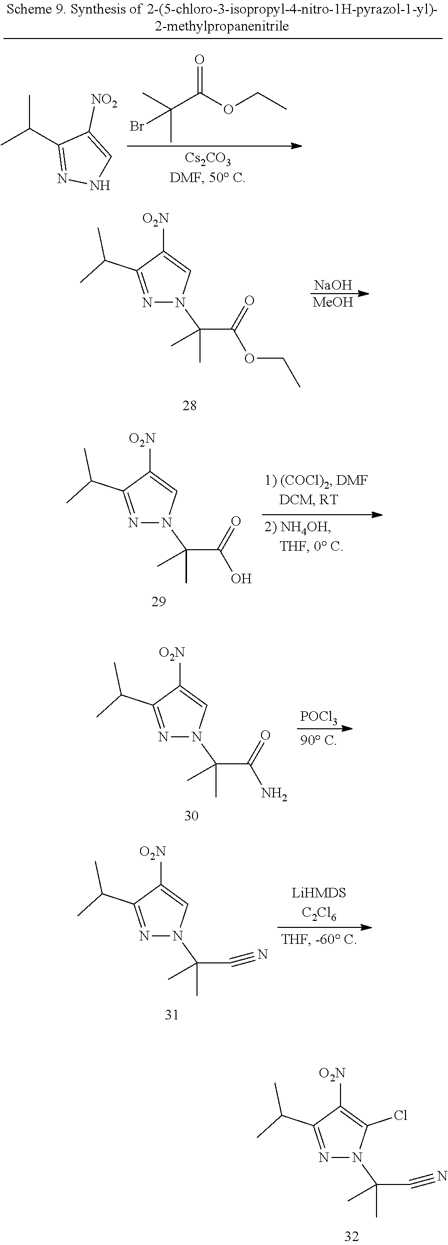

### Ethyl 2-(3-isopropyl-4-nitro-1H-pyrazol-1-yl)-2-methylpropanoate (28)

To a flask were added 3-isopropyl-4-nitro-1H-pyrazole (2.5 g, 16.11 mmol), cesium carbonate (7.87 g, 24.17 mmol) and DMF (100 mL). To this mixture was added ethyl 2-bromo-2-methylpropanoate (2.97 mL, 20.25 mmol) at RT. The mixture was allowed to stir at 50° C. for 4 h, then was filtered. The filtrate was diluted with water and extracted with EtOAc (3×). The combined organic layers were washed with brine and dried over MgSO₄. The solution was filtered and concentrated under reduced pressure. The crude residue was purified by flash chromatography on silica gel (gradient elution of 0-50% EtOAc/PE) to afford the title compound 28. MS (ESI): m/z calc'd for C₁₂H₁₉N₃O₄ [M+H]⁺: 270, found 270.

### 2-(3-Isopropyl-4-nitro-1H-pyrazol-1-yl)-2-methylpropanoic acid (29)

To a flask containing ethyl 2-(3-isopropyl-4-nitro-1H-pyrazol-1-yl)-2-methylpropanoate 28 (1.8 g, 6.68 mmol) was added THF (30 mL), NaOH (1.0 N, 15 mL, 15.00 mmol) and MeOH (10 mL). The mixture was allowed to stir at RT for overnight. The organic volatiles were removed under reduced pressure. The solution was acidified to pH ˜4. The mixture was filtered to afford the title compound. The supernatant was extracted with CHCl₃/IPA (3×). The organic layers were combined, washed with brine and dried over MgSO₄. The solution was filtered and concentrated under reduced pressure to afford additional quantity of the title compound. The two batches were combined to afford the title compound 29. MS (ESI): m/z calc'd for C₁₀H₁₅N₃O₄ [M+H]⁺: 242, found 242.

### 2-(3-Isopropyl-4-nitro-1H-pyrazol-1-yl)-2-methylpropanamide (30)

To a solution of 2-(3-isopropyl-4-nitro-1H-pyrazol-1-yl)-2-methylpropanoic acid 29 (977 mg, 4.05 mmol) in DCM (20 mL) was added dropwise of oxalyl chloride (900 μL, 10.3 mmol) and 2 drops of DMF. The mixture was allowed to stir at RT for 2 h. The reaction mixture was concentrated under reduced pressure. The residue was dissolved in THF (10 mL) and to the mixture was added dropwise NH₄OH (28% in H₂O, 6.00 mL, 43.1 mmol) at 0° C. The mixture was allowed to stir at RT for 18 h. The solution was concentrated under reduced pressure and dissolved in EtOAc and water. The layers were separated. The aqueous phase was extracted with EtOAc. The combined organic layers were washed with saturated aqueous NH₄Cl and dried over anhydrous MgSO₄. The solution was filtered and concentrated to afford the title compound 30. The product was used in the next step directly without further purification. MS (ESI): m/z calc'd for C₁₀H₁₆N₄O₃ [M+H]⁺: 241, found 241.

### 2-(3-Isopropyl-4-nitro-1H-pyrazol-1-yl)-2-methylpropanenitrile (31)

To a vial containing 2-(3-isopropyl-4-nitro-1H-pyrazol-1-yl)-2-methylpropanamide 30 (705 mg, 2.93 mmol) was added POCl₃ (2.50 mL, 26.8 mmol). The reaction mixture was heated to 90° C. allowed to stir for 40 min. The reaction mixture was concentrated under reduced pressure. The residue was diluted with ice water. The solution was neutralized with saturated aqueous NaHCO₃ to pH ˜6 and extracted with EtOAc. The organic layer was washed with brine and dried over MgSO₄. The solution was filtered and concentrated under reduced pressure to afford the title compound 31. ¹H NMR (600 MHz, CDCl₃) δ 8.41 (s, 1H), 3.69-3.54 (m, 1H), 2.04 (s, 6H), 1.34 (d, J=6.9 Hz, 6H).

### 2-(5-Chloro-3-isopropyl-4-nitro-1H-pyrazol-1-yl)-2-methylpropanenitrile (32)

To a solution of 2-(3-isopropyl-4-nitro-1H-pyrazol-1-yl)-2-methylpropanenitrile 31 (623 mg, 2.80 mmol) in THF (10 mL) was added LiHMDS (1.5 M in hexanes, 2.50 mL, 3.75 mmol) at −65° C. under N₂. The reaction was allowed to stir at −60° C. for 50 min. To the solution was then

### 2-(5-Chloro-3-isopropyl-4-nitro-1H-pyrazol-1-yl)-2-methylpropanenitrile (32)

To a solution of 2-(3-isopropyl-4-nitro-1H-pyrazol-1-yl)-2-methylpropanenitrile 31 (623 mg, 2.80 mmol) in THF (10 mL) was added LiHMDS (1.5 M in hexanes, 2.50 mL, 3.75 mmol) at −65° C. under N₂. The reaction was allowed to stir at −60° C. for 50 min. To the solution was then added hexachloroethane (995 mg, 4.20 mmol) in THF (3 mL) dropwise at −60° C. The reaction mixture was quenched by the addition of saturated aqueous NH₄Cl and extracted with EtOAc. The combined organic layers were washed with brine and dried over MgSO₄. The solution was filtered and concentrated under reduced pressure. The crude residue was purified by flash chromatography on silica gel (gradient elution of 0-20% EtOAc/hexanes) to afford the title compound 32. MS (ESI): m/z calc'd for C₁₀H₁₃ClN₄O₂ [M+H]⁺: 257, found 257.

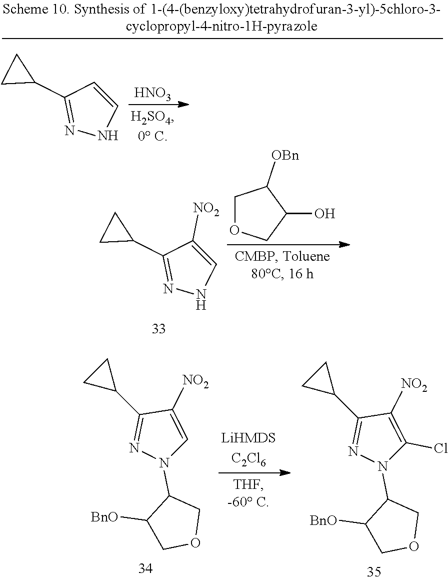

### 3-Cyclopropyl-4-nitro-1H-pyrazole (33)

To a solution of 3-cyclopropyl-1H-pyrazole (500 mg, 4.62 mmol) in H₂SO₄ (5 mL) was added nitric acid (5 mL, 131 mmol) at 0° C. The mixture was allowed to stir at 0° C. for 1 h, and then poured into a flask containing ice water (40 mL) and extracted with EtOAc (50 mL×3). The combined organic layers were washed with saturated NaHCO₃ (100 mL), dried over Na₂SO₄ The solution was filtered and concentrated under reduced pressure to afford the title compound 33. The product was used in the next step directly without further purification. MS (ESI): m/z calc'd for C₆H₇N₃O₂ [M+H]⁺: 154, found 154.

### 1-(4-(Benzyloxy)tetrahydrofuran-3-yl)-3-cyclopropyl-4-nitro-1H-pyrazole (34)

To a solution of 4-(benzyloxy)tetrahydrofuran-3-ol (2.54 g, 13.1 mmol) and 3-cyclopropyl-4-nitro-1H-pyrazole 33 (2.00 g, 13.1 mmol) in anhydrous toluene (20 mL) was added (tributylphosphoranylidene) acetonitrile (3.47 g, 14.4 mmol), and the reaction mixture was allowed to stir at 80° C. under N₂ for 16 h. The mixture was concentrated under reduced pressure. The residue was purified by flash chromatography on silica gel (gradient elution of 0-30% EtOAc/hexanes) to afford the title compound 34. MS (ESI): m/z calc'd for C₁₇H₁₉N₃O₄ [M+H]⁺: 330, found 330.

### 1-(4-(Benzyloxy)tetrahydrofuran-3-yl)-5-chloro-3-cyclopropyl-4-nitro-1H-pyrazole (35)

To a solution of 1-(4-(benzyloxy)tetrahydrofuran-3-yl)-3-cyclopropyl-4-nitro-1H-pyrazole 34 (1000 mg, 3.04 mmol) in THF (10 mL), was added LiHMDS (1.0 M in hexanes, 4.55 mL, 4.55 mmol) at −78° C. under N₂. The reaction was allowed to stir at −78° C. for 1.5 h. To the reaction mixture was added a solution of hexachloroethane (1290 mg, 5.47 mmol) in THF (5 mL) dropwise at −78° C. After addition, the mixture was quenched by the addition of saturated aqueous NH₄Cl (20 mL) and extracted with EtOAc (3×30 mL). The combined organic layers were washed with brine (20 mL) and dried over MgSO₄. The solution was filtered and concentrated under reduced pressure. The crude residue was purified by flash chromatography on silica gel (gradient elution of 0-30% EtOAc/hexanes) to afford the title compound 35. MS (ESI): m/z calc'd for C₁₇H₁₈ClN₃O₄ [M+H]⁺: 364, found 364.

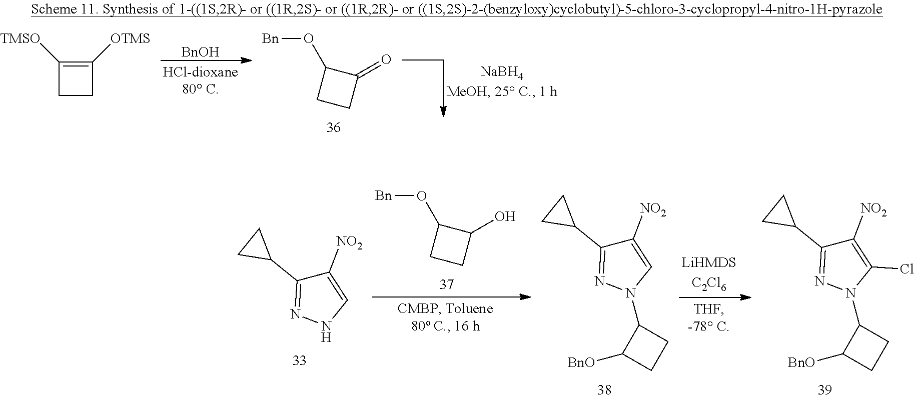

### 2-(Benzyloxy)cyclobutan-1-one (36)

To a solution of phenylmethanol (24 mL, 232 mmol) and HCl (4.0 M in dioxane, 5 mL, 20.00 mmol) was added 1,2-bis((trimethylsilyl)oxy)cyclobut-1-ene (5 g, 12.15 mmol) dropwise at 0° C. The reaction mixture was heated at 80° C. and allowed to stir for 6 h. The mixture was concentrated under reduced pressure. The residue was purified by flash chromatography on silica gel (gradient elution of 0-5% EtOAc/PE) to afford the title compound 36. ¹H NMR (400 MHz, CDCl₃) δ: 7.38-7.32 (m, 5H), 4.81-4.75 (m, 1H), 4.67-4.60 (m, 1H), 2.86-2.70 (m, 2H), 2.39-2.26 (m, 1H), 2.03-1.90 (m, 1H).

### (1S,2R)- and ((1R,2S)- and (1R,2R)- and (1S,2S)-2-(Benzyloxy)cyclobutan-1-ol (37)

To a solution of 2-(benzyloxy)cyclobutan-1-one 36 (2.00 g, 6.81 mmol) in MeOH (10 mL) was added NaBH₄ (890 mg, 14.5 mmol) at 0° C. The reaction mixture was allowed to stir at 25° C. for 1 h. The mixture was poured into a flask containing ice water and extracted with EtOAc (3×50 mL). The combined organic layers were dried over Na₂SO₄. The solution was filtered and concentrated under reduced pressure. The residue was purified by flash chromatography on silica gel (gradient elution of 0-20% EtOAc/PE) to afford the title compound 37.

### 1-((1S,2R)- and ((1R,2S)- or ((1R,2R)- or ((1S,2S)-2-(Benzyloxy)cyclobutyl)-5-chloro-3-cyclopropyl-4-nitro-1H-pyrazole (39)

Intermediate 39 was prepared in accordance with the synthetic protocol described in Scheme 10 and the accompanying text by substituting 4-(benzyloxy)tetrahydrofuran-3-ol for intermediate 37. MS (ESI): m/z calc'd for C₁₇H₁₈ClN₃O₃ [M+H]⁺: 348, found 348.

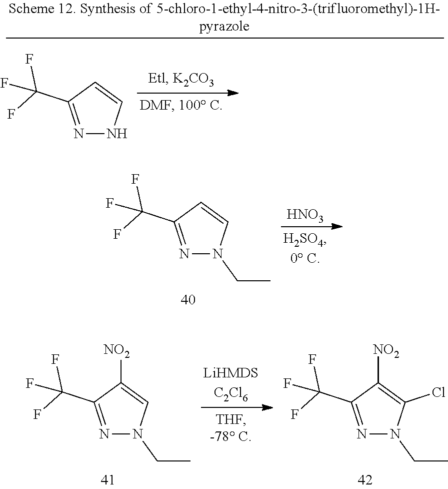

### 1-Ethyl-3-(trifluoromethyl)-1H-pyrazole (40)

To a solution of 3-(trifluoromethyl)-1H-pyrazole (1.00 g, 7.35 mmol), potassium carbonate (1.52 g, 11.0 mmol) in DMF (10 mL) was added iodoethane (1.38 g, 8.82 mmol). The reaction mixture was allowed to stir at 100° C. for 4 h. The reaction mixture was poured into a flask containing ice water (30 mL) and extracted with EtOAc (3×30 mL). The combined organic layers were washed with brine (20 mL) and dried over Na₂SO₄. The solution was filtered and concentrated under reduced pressure to afford the title compound 40. The product was used in the next step directly without further purification. MS (ESI): m/z calc'd for C₆H₇F₃N₂ [M+H]⁺: 165, found 165.

### 1-Ethyl-4-nitro-3-(trifluoromethyl)-1H-pyrazole (41)

To a solution of 1-ethyl-3-(trifluoromethyl)-1H-pyrazole 40 (540 mg, 3.29 mmol) in H₂SO₄ (6 mL) was added nitric acid (1040 mg, 16.4 mmol) at 0° C. and the reaction mixture was allowed to stir at 20° C. for 12 h. The reaction mixture was poured into ice water (30 mL) and extracted with EtOAc (3×30 mL). The combined organic layers were dried over Na₂SO₄. The solution was filtered and concentrated under reduced pressure. The crude residue was purified by flash chromatography on silica gel (gradient elution of 0-30% EtOAc/PE) to afford the title compound 41. ¹H NMR (400 MHz, CDCl₃) δ: 8.26 (s, 1H), 4.25-4.32 (m, 2H), 1.60 (t, J=7.4 Hz, 3H), 0.00-0.00 ppm (m, 1H).

### 5-Chloro-1-ethyl-4-nitro-3-(trifluoromethyl)-1H-pyrazole (42)

To a solution of 1-ethyl-4-nitro-3-(trifluoromethyl)-1H-pyrazole 41 (440 mg, 2.10 mmol) in THF (4.00 mL) was added LiHMDS (1.0 M in THF. 3.16 mL, 3.16 mmol) at −78° C. The mixture was allowed to stir at −78° C. for 1.5 h under N₂ atmosphere. To the mixture was added hexachloroethane (897 mg, 3.79 mmol) in THF (2 mL) at −78° C. The reaction mixture was allowed to stir at −78° C. for 1 h. The mixture was poured into a flask containing ice water (30 mL) and extracted with EtOAc (3×30 mL). The combined organic layers were dried over MgSO₄. The solution was filtered and concentrated under reduced pressure. The residue was purified by flash chromatography on silica gel (gradient elution of 0-15% EtOAc/PE) to afford the title compound 42. ¹H NMR (400 MHz, CDCl₃) δ: 4.32 (q, 2H), 1.54 (t, 3H).

Each of the substituted pyrazoles presented in Table 1 below were prepared in accordance with the synthetic routes in General Scheme 1, using procedures analogous to those described above.

TABLE 1
Intermediates

| Intermediate | Structure |
|---|---|
| 43 | 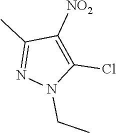 |
| 44 | 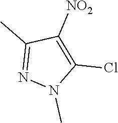 |
| 46 | 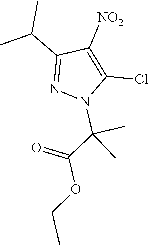 [SMILES: CCOC(=O)C(C)(C)n1nc(C(C)C)c([N+](=O)[O-])c1Cl] |
| 47 | 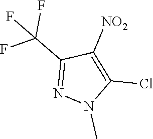 |
| 48 | 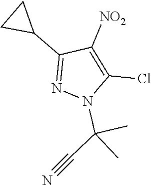 [SMILES: CC(C)(C#N)n1nc(C2CC2)c([N+](=O)[O-])c1Cl] |
| 49 | 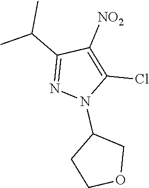 [SMILES: CC(C)c1nn(C2CCOC2)c(Cl)c1[N+](=O)[O-]] |
| 50 | 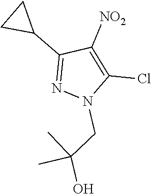 [SMILES: CC(C)(O)Cn1nc(C2CC2)c([N+](=O)[O-])c1Cl] |
| 51 | 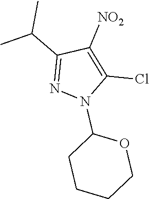 [SMILES: CC(C)c1nn(C2CCCCO2)c(Cl)c1[N+](=O)[O-]] |

### General Synthetic Schemes and Preparative Examples

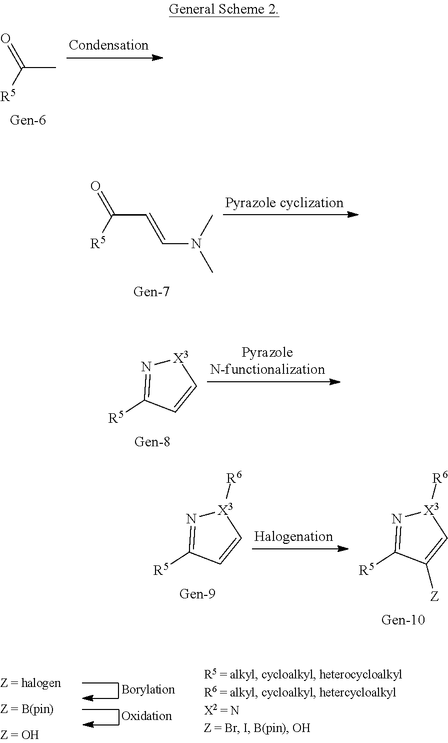

In General Scheme 2, commercially available or synthetically prepared ketones Gen-6 could be

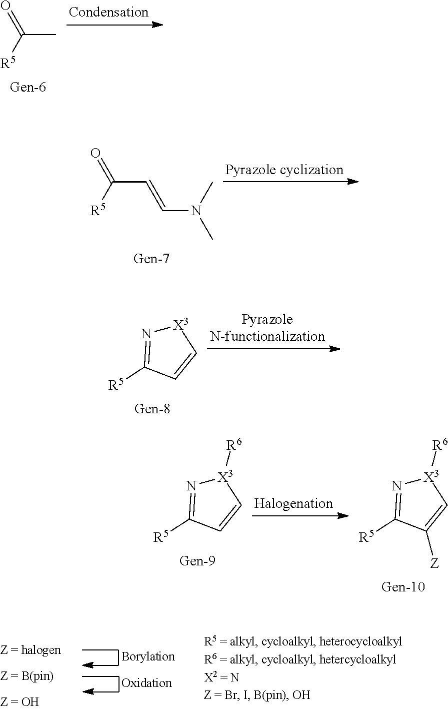

In General Scheme 2, commercially available or synthetically prepared ketones Gen-6 could be condensed with acylating reagents to access vinylogous amides Gen-7. Condensation of Gen-7 with hydrazine could provide pyrazoles of the form Gen-8. Commercially available or synthetically prepared pyrazoles of the form Gen-8 could be alkylated using a number of synthetic transformations commonly known to those skilled in the art, including, but not limited to, a base-mediated alkylation or a Mitsunobu reaction to afford N-alkyl pyrazoles 3-substituted Gen-9. These transformations typically afforded a mixture of 3- and 5-substituted pyrazoles and could be separated by achiral or chiral purification methods. A number of intermediates of the form Gen-9 are commercially available, including alkyl groups of the depicted substitution pattern. N-substituted pyrazoles of the form Gen-9 could be halogenated at the 4-position through common halogenating reagents. In cases of Gen-10 where Z=Br or I, the halogen could optionally be transformed to the boronic acid pinacol ester Gen-10 where Z=B(pin) through known metal-catalyzed or mediated borylation methods. In cases of Gen-10 where Z=B(pin), the boronic acid pinacol ester could be transformed through known oxidation methods to the alcohol Gen-10 where Z=OH. Representative preparative examples from each sequence are described in more detail below.

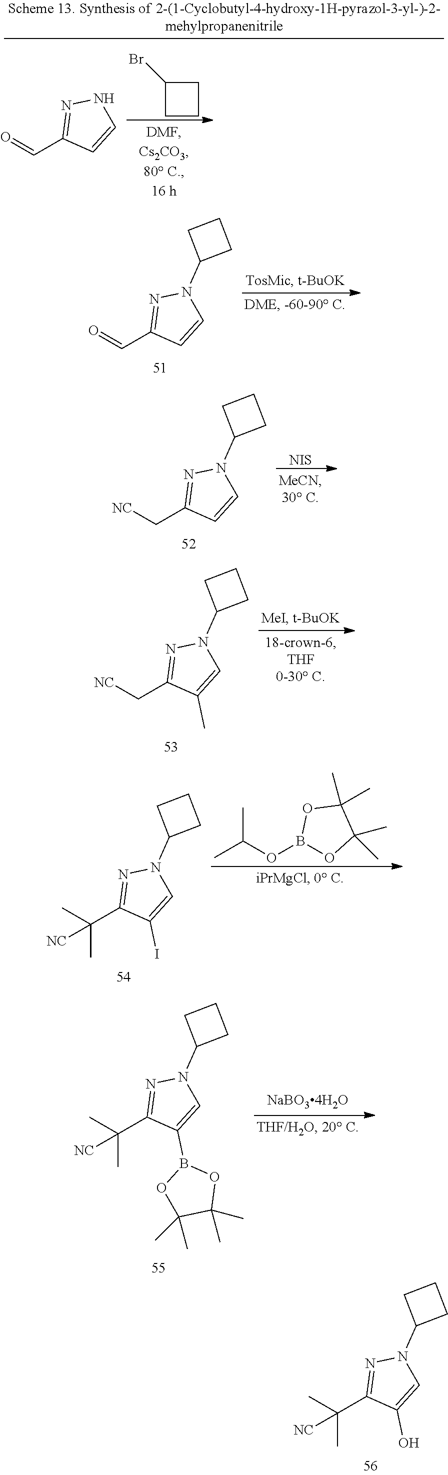

### 1-Cyclobutyl-1H-pyrazole-3-carbaldehyde (51)

To a solution of 1H-pyrazole-3-carbaldehyde (10 g, 104 mmol) in anhydrous DMF (500 mL) was added Cs₂CO₃ (67.8 g, 208 mmol) and bromocyclobutane (28.1 g, 208 mmol). The reaction mixture was heated to 80° C. and allowed to stir for 16 h. The reaction mixture was poured into water (500 mL) and extracted with EtOAc (3×500 mL). The organic layer was washed with water (2×300 mL) and dried over Na₂SO₄. The solution was filtered and concentrated under reduced pressure. The crude residue was purified by flash chromatography on silica gel (gradient elution of 0-10% EtOAc/PE) to afford title compound 51.

### 2-(1-Cyclobutyl-1H-pyrazol-3-yl) acetonitrile (52)

To a mixture of potassium 2-methylpropan-2-olate (7.47 g, 66.6 mmol) in DME (100 mL) was added dropwise a solution of 1-((isocyanomethyl) sulfonyl)-4-methylbenzene (7.15 g, 36.6 mmol) in DME (50 mL) at −60° C. To the reaction mixture was added dropwise a solution of 1-cyclobutyl-1H-pyrazole-3-carbaldehyde 51 (5.00 g, 33.3 mmol) in DME (40 mL) at −60° C. The reaction mixture was allowed to stir at −60° C. for 1 h. To the reaction mixture was added MeOH (40 mL), and the reaction mixture was warmed to 15° C. and allowed to stir for 1 h. The reaction was then warmed to 90° C. and allowed to stir for an additional 1 h. After cooling to RT, the volatiles were removed under reduced pressure. The residue was diluted with brine (100 mL) and extracted with EtOAc (3×50 mL). The combined organic layers were dried over Na₂SO₄. The solution was filtered and concentrated under reduced pressure. The residue was purified by flash chromatography on silica gel (gradient elution of 0-16% EtOAc/PE) to afford the title compound 52.

and dried over Na₂SO₄. The solution was filtered and concentrated under reduced pressure. The crude product was purified by flash chromatography on silica gel (gradient elution of 0-10% EtOAc/PE) to afford the title compound 54.

### 2-(1-Cyclobutyl-4-(4,4,5,5-tetramethyl-1,3,2-dioxaborolan-2-yl)-1H-pyrazol-3-yl)-2-methylpropanenitrile (55)

To a solution of 2-(1-cyclobutyl-4-iodo-1H-pyrazol-3-yl)-2-methylpropanenitrile 54 (2.60 g, 8.25 mmol) in THF (60 mL), cooled to 0° C., was added dropwise isopropylmagnesium chloride (6.20 mL, 12.4 mmol). The reaction mixture was allowed to stir for 30 min. To the mixture was added 2-isopropoxy-4,4,5,5-tetramethyl-1,3,2-dioxaborolane (3.38 g, 18.15 mmol). The mixture was allowed to stir at 0° C. for 2 h, then quenched by the addition of saturated NH₄Cl (50 mL), extracted with EtOAc (3×50 mL) and dried over Na₂SO₄. The solution was filtered and concentrated under reduced pressure. The crude residue was purified by flash chromatography on silica gel (gradient elution of 0-10% EtOAc/PE) to afford the title compound 55.

### 2-(1-Cyclobutyl-4-hydroxy-1H-pyrazol-3-yl)-2-methylpropanenitrile (56)

To a solution of 2-(1-cyclobutyl-4-(4,4,5,5-tetramethyl-1,3,2-dioxaborolan-2-yl)-1H-pyrazol-3-yl)-2-methylpropanenitrile 55 (2.30 g, 7.30 mmol) in THF (30 mL) were added sodium hydroxide (11.0 mL, 22.0 mmol) and hydrogen peroxide (1.66 g, 14.6 mmol). The solution was allowed to stir at 20° C. for 1 h. Excess of H₂O₂ was quenched by saturated aqueous Na₂SO₃. The mixture was acidified with 2 M HCl to pH 4. The mixture was filtered. The filtrate was concentrated under reduced pressure. The crude residue was purified by reversed phase HPLC (Column: Phenomenex Synergi C18 (250×21.2 mm, 4 μm); Mobile phase A: Water w/0.2% Formic acid; Mobile phase B: MeCN) to afford the title compound 56. MS (ESI): m/z calc'd for C₁₁H₁₅N₃O [M+H]⁺: 206, found 206.

### Scheme 14. Synthesis of (1S,2S)- and (1R,2R)-2-(1-cyclobutyl-4-hydroxy-1H-pyrazol-3-yl)cyclopropane-1-carbonitrile

hydroxide (11.0 mL, 22.0 mmol) and hydrogen peroxide (1.66 g, 14.6 mmol). The solution was allowed to stir at 20° C. for 1 h. Excess of H₂O₂ was quenched by saturated aqueous Na₂SO₃. The mixture was acidified with 2 M HC to pH 4. The mixture was filtered. The filtrate was concentrated under reduced pressure. The crude residue was purified by reversed phase HPLC (Column: Phenomenex Synergi C18 (250×21.2 mm, 4 μm); Mobile phase A: Water w/0.2% Formic acid; Mobile phase B: MeCN) to afford the title compound 56. MS (ESI): m/z calc'd for C₁₁H₁₅N₃O [M+H]⁺: 206, found 206.

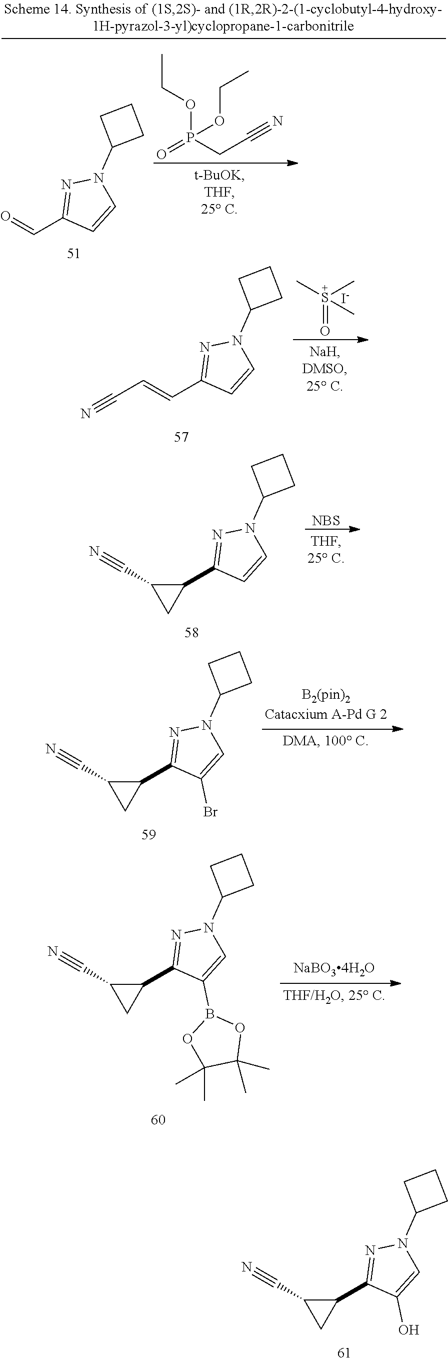

### (E)-3-(1-Cyclobutyl-1H-pyrazol-3-yl)acrylonitrile (57)

To a suspension of diethyl (cyanomethyl)phosphonate (24.8 g, 140 mmol) in THF (140 mL) was added potassium tert-butoxide (140 mL, 140 mmol) at 0° C. under N₂ atmosphere. The reaction mixture was allowed to stir for 30 min, then a solution of 1-cyclobutyl-1H-pyrazole-3-carbaldehyde 51 (14 g, 93 mmol) in THF (140 mL) was added dropwise. The reaction was allowed to stir at 25° C. for 16 h, then was poured into water (500 mL) and extracted with EtOAc (3×200 mL). The combined organic layers were washed with brine (200 mL) and dried over Na₂SO₄. The solution was filtered and concentrated under reduced pressure. The crude product was purified by flash chromatography on silica gel (gradient elution of 0-15% EtOAc/PE) to afford the title compound 57.

### (1S,2S)- and (1R,2R)-2-(1-cyclobutyl-1H-pyrazol-3-yl)cyclopropane-1-carbonitrile (58)

### (1S,2S)- and (1R,2R)-2-(4-bromo-1-cyclobutyl-1H-pyrazol-3-yl)cyclopropane-1-carbonitrile (59)

To a solution of 2-(1-cyclobutyl-1H-pyrazol-3-yl)cyclopropane-1-carbonitrile 58 (4.20 g, 22.4 mmol) in MeCN (40 mL) was added N-bromosuccinimide (4.79 g, 26.9 mmol). The reaction mixture was allowed to stir at 25° C. for 1 h, then was quenched by the addition of water and extracted with EtOAc (3×20 mL). The organic layer was washed with brine (30 mL), dried over Na₂SO₄. The solution was filtered and concentrated under reduced pressure. The crude product was purified by flash chromatography on silica gel (gradient elution of 0-1.5% EtOAc/PE) to afford the title compound 59.

### (1S,2S)- and (1R,2R)-2-(1-cyclobutyl-4-hydroxy-1H-pyrazol-3-yl)cyclopropane-1-carbonitrile (61)

Intermediate 61 was prepared in accordance with the synthetic protocol described in Scheme 13 and the accompanying text by substituting iodo intermediate 54 for bromo intermediate 59. MS (ESI): m/z calc'd for C₁₁H₁₃N₃O [M+H]⁺: 204, found 204.

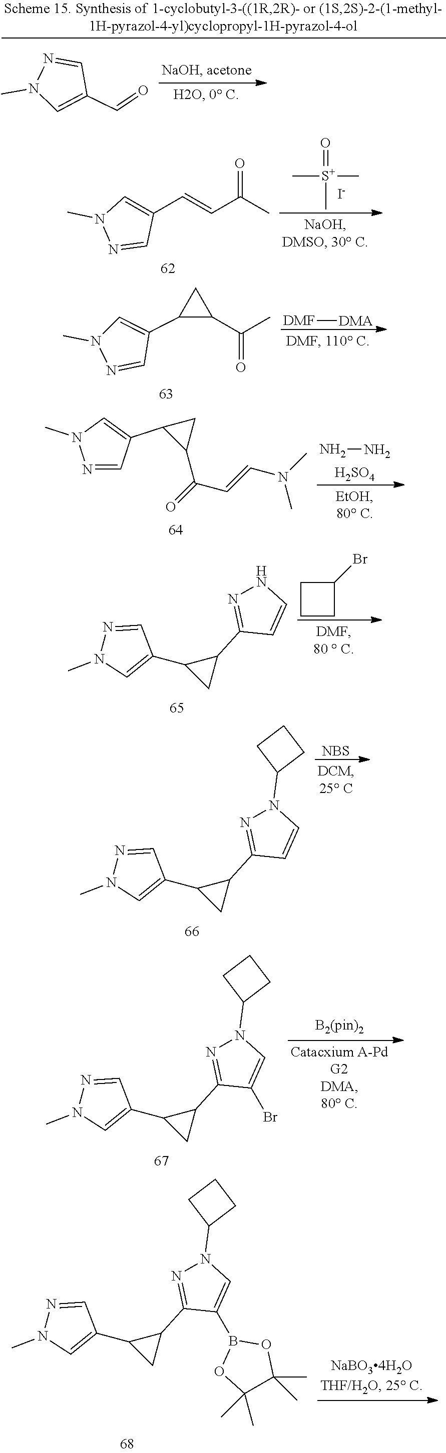

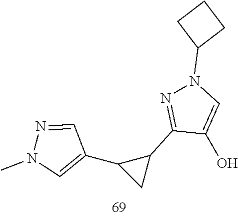 [SMILES: Cn1cc(C2CC2c2nn(C3CCC3)cc2O)cn1]

### (E)-4-(1-Methyl-1H-pyrazol-4-yl)but-3-en-2-one (62)

To a solution of 1-methyl-1H-pyrazole-4-carbaldehyde (15 g, 91 mmol) in acetone (126 mL) and water (60 mL) under N₂ was added dropwise sodium hydroxide (5.446 g, 91 mmol) in water (60 mL) at 0° C. The mixture was allowed to stir at 0° C. for 0.5 h under N₂. The organic volatiles were concentrated under reduced pressure. The solution was acidified with 2 N HCl (30 mL) pH 3. The mixture was extracted with EtOAc (3×50 mL). The combined organic layers were dried over Na₂SO₄. The solution was filtered and concentrated under reduced pressure to give residue. The residue was purified by flash chromatography on silica gel (gradient elution of 0-50% EtOAc/hexanes) to afford the title compound 62.

### 1-((1R,2R)- and (1S,2S)-2-(1-Methyl-1H-pyrazol-4-yl)cyclopropyl)ethan-1-one (63)

To a mixture of NaOH (4.98 g, 83 mmol) in DMSO (100 mL) was added trimethylsulfoxonium iodide (31 g, 95 mmol). The reaction mixture was allowed to stir at 30° C. for 1.5 h under N₂ atmosphere, then a solution of (E)-4-(1-methyl-1H-pyrazol-4-yl)but-3-en-2-one 62 (17.8 g, 79 mmol) in DMSO (170 mL) was added dropwise. The reaction mixture was allowed to stir at 30° C. for 1.5 h. The mixture was added to a flask containing ice water (50 mL) and extracted with EtOAc (3×100 mL). The combined organic layers were extracted with brine (3×50 mL), dried over Na₂SO₄. The solution was filtered and concentrated under reduced pressure. The crude residue was purified by flash chromatography on silica gel (gradient elution of 0-50% EtOAc/hexanes) to afford the title compound 63.

### (E)-3-(Dimethylamino)-1-((1R,2R)- and (1S,2S)-2-(1-methyl-1H-pyrazol-4-yl)cyclopropyl)prop-2-en-1-one (64)

To a solution of 1-(2-(1-methyl-1H-pyrazol-4-yl)cyclopropyl)ethan-1-one (9.2 g, 56 mmol) 63 in DMF (80 mL) was added N,N-dimethylformamide dimethyl acetal (40 g, 340 mmol). The mixture was allowed to stir at 110° C. for 15 h, then was added to a flask containing ice water (40 mL) and extracted with EtOAc (3×40 mL). The combined organic layers were extracted with brine (3×40 mL), dried over Na₂SO₄. The solution was filtered and concentrated under reduced pressure to give the residue. The residue was purified by flash chromatography on silica gel (gradient elution of 0-100% EtOAc/PE) to afford the title compound 64.

### 4-((1R,2R)- and (1S,2S)-2-(1H-pyrazol-3-yl)cyclopropyl)-1-methyl-1H-pyrazole (65)

To a solution of (E)-3-(dimethylamino)-1-(2-(1-methyl-1H-pyrazol-4-yl)cyclopropyl)prop-2-en-1-one 64 (8.4 g, 38 mmol) in EtOH (80 mL) was added hydrazine sulfate (7.5 g, 58 mmol). The reaction mixture was heated to 80° C. and allowed to stir for 2 h, then concentrated under reduced pressure. The solution was basified with saturated aqueous NaHCO₃ (40 mL) pH 9. The mixture was extracted with EtOAc (3×50 mL). The combined organic layers were dried over Na₂SO₄. The solution was filtered and concentrated under reduced pressure to afford the title compound 65. The product was used in the next step directly without further purification.

### 1-Cyclobutyl-3-((1R,2R)- or (1S,2S)-2-(1-methyl-1H-pyrazol-4-yl)cyclopropyl)-1H-pyrazole (66)

To a solution of 4-(2-(1H-pyrazol-3-yl)cyclopropyl)-1-methyl-1H-pyrazole 65 (7.4 g, 39.3 mmol) in anhydrous DMF (80 mL) was added bromocyclobutane (7.96 g, 59.0 mmol) and cesium carbonate (25.6 g, 79 mmol), and the reaction mixture was allowed to stir at 80° C. for 24 h. The reaction mixture was poured into a flask containing water (100 mL) and extracted with EtOAc (3×100 mL). The combined organic layers were washed with brine (3×100 mL), dried over Na₂SO₄. The solution was filtered and concentrated under reduced pressure. The crude product was purified by preparative HPLC (0.1% TFA modifier with MeCN/water) to afford the title compound 66.

### 1-Cyclobutyl-3-((1R,2R)- and (1S,2S)-2-(1-methyl-1H-pyrazol-4-yl)cyclopropyl)-1H-pyrazol-4-ol (69)

Intermediate 69 was prepared in accordance with the synthetic protocol described in Scheme 14 and the accompanying text by substituting intermediate 58 for intermediate 65. MS (ESI): m/z calc'd for C₁₄H₁₈N₄O [M+H]⁺: 259, found 259.

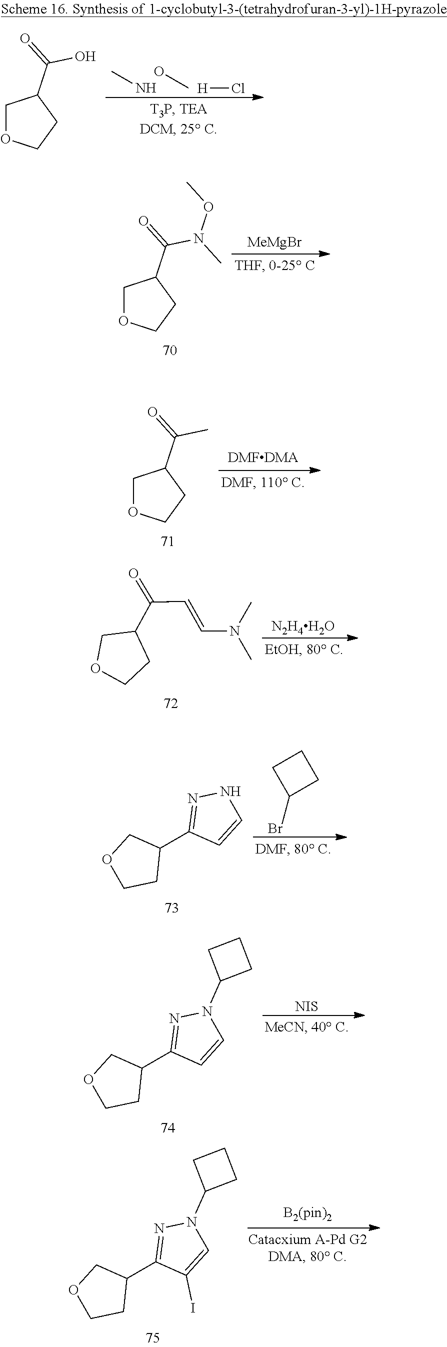

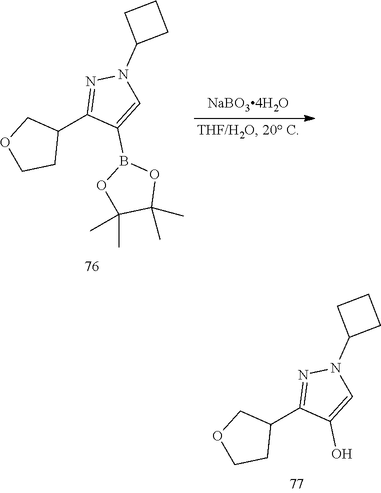 [SMILES: CC1(C)OB(c2cn(C3CCC3)nc2C2CCOC2)OC1(C)C ; Oc1cn(C2CCC2)nc1C1CCOC1]

### (S)- and (R)-N-methoxy-N-methyltetrahydrofuran-3-carboxamide (70)

To a solution of tetrahydrofuran-3-carboxylic acid (10 g, 86 mmol), N,O-dimethylhydroxylamine hydrochloride (10.92 g, 112 mmol) and triethylamine (23.94 mL, 172 mmol) in DCM (100 mL) was added 1-propanephosphonic anhydride solution (66.3 mL, 129 mmol) at 0° C. The mixture was allowed to stir at 25° C. for 2 h. The reaction mixture was diluted with water (200 mL) and saturated aqueous NaHCO₃ (100 mL). The mixture was extracted with DCM (3×150 mL) and dried over Na₂SO₄. The solution was filtered and concentrated under reduced pressure to afford the title compound 70. The product was used in the next step directly without further purification.

### (S)- and (R)-1-(Tetrahydrofuran-3-yl)ethan-1-one (71)

To a solution of N-methoxy-N-methyltetrahydrofuran-3-carboxamide 70 (10.0 g, 62.8 mmol) in THF (100 mL) was added methylmagnesium bromide (41.9 mL, 126 mmol) at 0° C. under N₂ atmosphere. The mixture was allowed to stir at 0° C. for 1 h, then was warmed to 25° C. and allowed to stir for 1 h. The mixture was poured into flask with ice water (180 mL), acidified with 2 N HCl (200 mL) to pH 3, and extracted with EtOAc (3×120 mL). The combined organic layers were dried over Na₂SO₄. The solution was filtered concentrated under reduced pressure to afford the title compound 71. The product was used in the next step directly without further purification.

### (S,E)- and (R,E)-3-(Dimethylamino)-1-(tetrahydrofuran-3-yl)prop-2-en-1-one (72)

To a solution of 1-(tetrahydrofuran-3-yl)ethan-1-one 71 (5 g, 43.8 mmol) in DMF (40 mL) was added DMF-DMA (31.3 g, 263 mmol). The mixture was allowed to stir at 110° C. for 16 h, then was concentrated under reduced pressure to afford the title compound 72. The product was used in the next step directly without further purification.

### (S)- and (R)-3-(Tetrahydrofuran-3-yl)-1H-pyrazole (73)

To a solution of hydrazine hydrate (2.07 g, 41.4 mmol) in anhydrous EtOH (70 mL) was added (E)-3-(dimethylamino)-1-(tetrahydrofuran-3-yl)prop-2-en-1-one 72 (7.00 g, 41.4 mmol), and the reaction mixture was allowed to stir at 80° C. for 2 h. The reaction mixture was filtered and the filtrate was concentrated under reduced pressure to afford the title compound 73. The product was used in the next step directly without further purification.

### (S)- and (R)-1-Cyclobutyl-3-(tetrahydrofuran-3-yl)-1H-pyrazole (74)

To a solution of 3-(tetrahydrofuran-3-yl)-1H-pyrazole 73 (3.5 g, 25.3 mmol) in anhydrous DMF (50 mL) was added bromocyclobutane (5.13 g, 38.0 mmol) and cesium carbonate (16.51 g, 50.7 mmol), and the reaction mixture was allowed to stir at 80° C. for 16 h. After LCMS showed the reaction was completed, the reaction mixture was poured into water (200 mL) and extracted with EtOAc (100 mL×3). The organic layer was washed with brine (3×80 mL), dried over Na₂SO₄. After filtration, the solvent was concentrated to afford the title compound 74. The product was used in the next step directly without further purification.

### (S)- and (R)-1-Cyclobutyl-3-(tetrahydrofuran-3-yl)-1H-pyrazol-4-ol (77)

Intermediate 77 was prepared in accordance with the synthetic protocol described in Scheme 14 and the accompanying text by substituting intermediate 58 for intermediate 74. MS (ESI): m/z calc'd for C₁₁H₁₆N₂O₂ [M+H]⁺: 209, found 209.

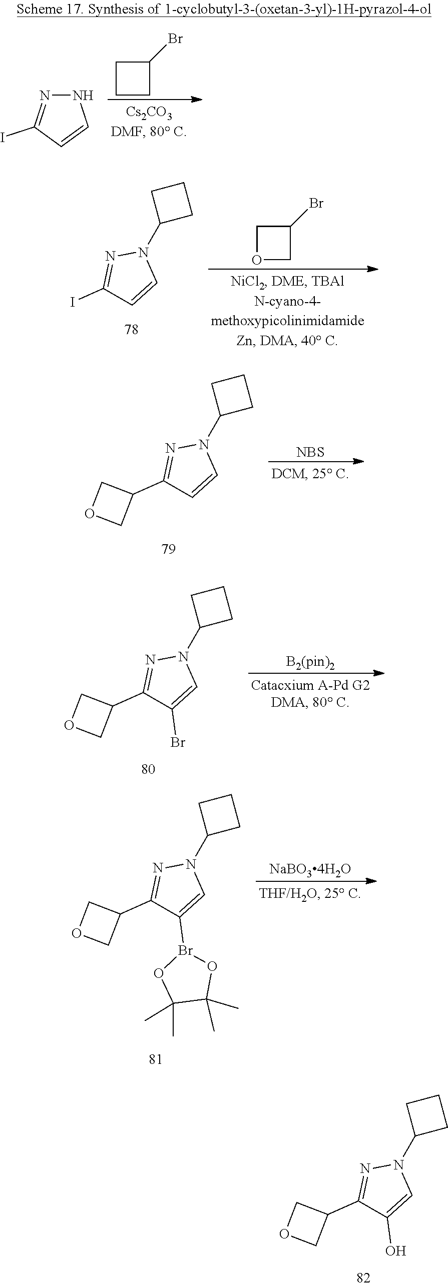

### 1-Cyclobutyl-3-iodo-1H-pyrazole (78)

To a solution of bromocyclobutane (12.5 g, 93 mmol) in DMF (150 mL) was added cesium carbonate (25 g, 77 mmol) and 3-iodo-1H-pyrazole (15 g, 77 mmol). The reaction mixture was allowed to stir at 80° C. for 16 h. The reaction mixture was poured into a flask with water (200 mL) and extracted with EtOAc (3×200 mL). The combined organic layers were washed with brine (3×100 mL), dried over Na₂SO₄. After filtration and concentration, the crude product was purified by flash chromatography on silica gel (gradient elution of 0-5% EtOAc/PE) to afford the title compound 78.

### 1-Cyclobutyl-3-(oxetan-3-yl)-1H-pyrazole (79)

To a solution of 3-bromooxetane (1657 mg, 12.09 mmol) and zinc (1054 mg, 16.12 mmol) in DMA (12 mL) was added tetrabutylammonium iodide (1489 mg, 4.03 mmol), nickel (II) chloride ethylene glycol dimethyl ether complex (177 mg, 0.806 mmol), N-cyano-4-methoxypicolinimidamide (284 mg, 1.612 mmol) and 1-cyclobutyl-3-iodo-1H-pyrazole 78 (1000 mg, 4.03 mmol). The mixture was allowed to stir for 3 h at 40° C. EtOAc (500 mL) was added to the solution, and the mixture was filtered. The filtrate was diluted with brine. The layers were separated, and the aqueous phase was extracted with EtOAc (3×200 mL). The combined organic layers were washed with brine and dried over MgSO₄. The solution was filtered and concentrated under reduced pressure to afford the title compound 79. The product was used in the next step directly without further purification.

### 1-Cyclobutyl-3-(oxetan-3-yl)-1H-pyrazol-4-ol (82)

Intermediate 82 was prepared in accordance with the synthetic protocol described in Scheme 14 and the accompanying text by substituting intermediate 58 for intermediate 79. MS (ESI): m/z calc'd for C₁₀H₁₄N₂O₂ [M+H]⁺: 195, found 195.

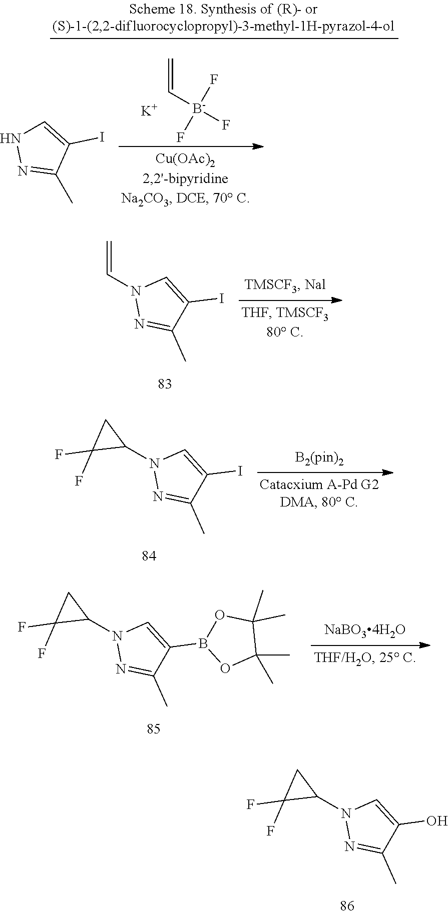

### 4-Iodo-3-methyl-1-vinyl-1H-pyrazole (83)

A solution of copper (II) acetate (4.37 g, 24.04 mmol) and 2,2′-bipyridine (7.51 g, 48.1 mmol) in DCE (30 mL) was heated to 70° C. for 15 min. Then this mixture was transferred to a suspension of 4-iodo-3-methyl-1H-pyrazole (5 g, 24.04 mmol), potassium vinyltrifluoroborate (6.44 g, 48.1 mmol) and Na₂CO₃ (5.10 g, 48.1 mmol) in DCE (20 mL). The mixture was allowed to stir at 70° C. for 12 h. TLC showed the reaction was complete. The mixture was poured into NH₄OH (37% in H₂O, 100 mL), and extracted with DCM (50 mL×2). The combined organic layers were washed with 2 M HCl (100 mL×3), brine (200 mL), dried over Na₂SO₄ and concentrated under reduced pressure. The crude residue was purified by flash chromatography on silica gel (gradient elution of 0-1% EtOAc/hexanes) to afford the title compound 83.

### (R)- and (S)-1-(2,2-Difluorocyclopropyl)-4-iodo-3-methyl-1H-pyrazole (84)

To a microwave tube was added sodium iodide (160 mg, 1.07 mmol), 4-iodo-3-methyl-1-vinyl-1H-pyrazole 83 (500 mg, 2.14 mmol) and dry THF (2.50 mL). The reaction mixture was heated to 80° C. and allowed to stir for 5 min under N₂ atmosphere. To the suspension was added dropwise (trifluoromethyl)trimethylsilane (1.52 g, 10.7 mmol). The reaction was allowed to stir at 80° C. for 16 h. The mixture was concentrated under reduced pressure. The crude residue was purified by purified by flash chromatography on silica gel (gradient elution of 0-1% EtOAc/hexanes) to afford the title compound 84.

### (R)- and (S)-1-(2,2-difluorocyclopropyl)-3-methyl-1H-pyrazol-4-ol (86)

Intermediate 86 was prepared in accordance with the synthetic protocol described in Scheme 14 and the accompanying text by substituting intermediate 59 for intermediate 84. MS (ESI): m/z calc'd for C₇H₈F₂N₂O [M+H]⁺: 175, found 175.

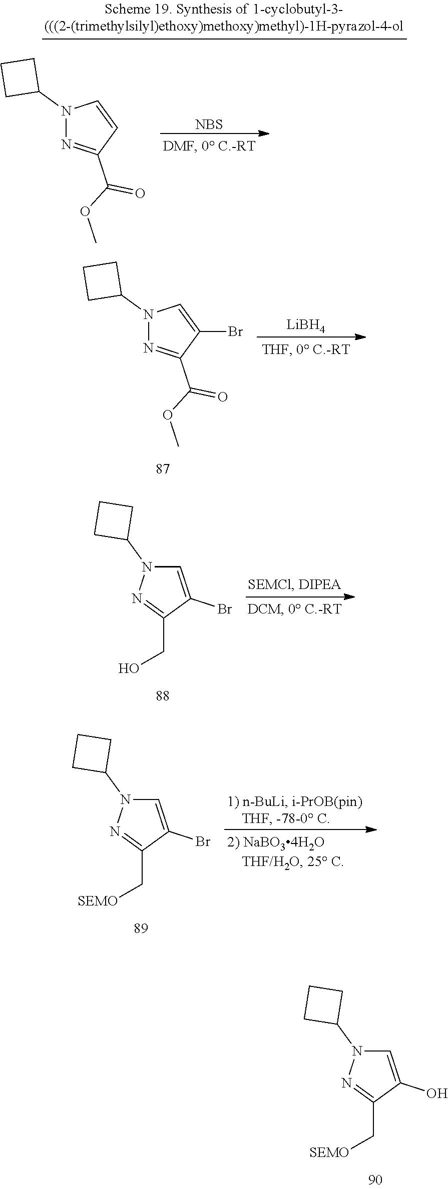

### Methyl 4-bromo-1-cyclobutyl-1H-pyrazole-3-carboxylate (87)

To a 100 mL round-bottom flask was added N-bromosuccinimide (1.09 g, 6.10 mmol) and DMF (25 mL), and the reaction mixture was cooled to 0° C. To the reaction mixture was added slowly methyl 1-cyclobutyl-1H-pyrazole-3-carboxylate (1.0 g, 5.6 mmol). The reaction was allowed to warm to RT and stir overnight, then diluted with saturated aqueous NaHCO₃ and extracted with EtOAc. The combined organic extracts were washed with water, brine, and dried over Na₂SO₄. The solution was filtered and concentrated under reduced pressure. The crude residue was purified by flash chromatography on silica gel (gradient elution of 0-100% EtOAc/hexanes) to afford the title compound 87.

### (4-Bromo-1-cyclobutyl-1H-pyrazol-3-yl)methanol (88)

To a 100 mL round-bottom flask was added methyl 4-bromo-1-cyclobutyl-1H-pyrazole-3-carboxylate 87 (1.3 g, 5.02 mmol) and THF (25 mL) under N₂ atmosphere. The reaction mixture was cooled to 0° C., and lithium borohydride (2 M in THF, 3.76 mL, 7.53 mmol) was added dropwise. The reaction was allowed to warm to RT and stir overnigh, followed by dropwise addition of lithium borohydride (2 M in THF, 1.00 mL, 2.00 mmol). The reaction mixture was allowed to stir at RT overnight, then was quenched by the slow addition of H₂O and extracted with EtOAc. The combined organic layers were dried over Na₂SO₄. The solution was filtered and concentrated under reduced pressure to afford the title compound 88. The product was used in the next step directly without further purification.

### 4-Bromo-1-cyclobutyl-3-(((2-(trimethylsilyl)ethoxy)methoxy)methyl)-1H-pyrazole (89)

To a 100 mL round-bottom flask containing a solution of (4-bromo-1-cyclobutyl-1H-pyrazol-3-yl)methanol 88 (550 mg, 2.38 mmol) in DCM (12 mL) was added N,N-diisopropylethylamine (0.65 mL, 3.7 mmol). The reaction mixture was cooled to 0° C., and then 2-(trimethylsilyl)ethoxymethyl chloride (0.85 mL, 4.79 mmol) was added dropwise. The reaction was allowed to warm to RT and stir overnight, then was quenched by the addition of water and saturated aqueous NaHCO₃ and extracted DCM. The combined organic layers were dried over Na₂SO₄. The solution was filtered and concentrated under reduced pressure. The crude residue was purified by flash chromatography on silica gel (gradient elution of 0-100% EtOAc/hexanes) to afford the title compound 89.

### 1-Cyclobutyl-3-(((2-(trimethylsilyl)ethoxy)methoxy)methyl)-1H-pyrazol-4-ol (90)

To a 100 mL round-bottom flask containing a solution of 4-bromo-1-cyclobutyl-3-(((2-(trimethylsilyl)ethoxy)methoxy)methyl)-1H-pyrazole 89 (847 mg, 2.34 mmol) in THF (15 mL) under a N₂ atmosphere, stirring at −78° C., was added dropwise n-butyllithium (2.5 M in hexanes, 1.80 mL, 4.50 mmol). The reaction mixture was allowed to stir at −78° C. for 30 min, then 2-isopropoxy-4,4,5,5-tetramethyl-1,3,2-dioxaborolane (1.00 mL, 4.90 mmol) was added dropwise. The reaction was allowed to warm to RT, allowed to stir for 1 h, diluted with water (8 mL), and sodium perborate tetrahydrate (1440 mg, 9.38 mmol) was added. The reaction mixture was allowed to stir at RT for 18 h, then was diluted with water and extracted with EtOAc. The combined organic layers were dried over Na₂SO₄. The solution was filtered and concentrated under reduced pressure. The crude residue was purified by flash chromatography on silica gel (gradient elution of 0-100% EtOAc/hexanes) to afford the title compound 90. MS (ESI): m/z calc'd for C₁₄H₂₇N₂O₃Si [M+H]⁺: 299, found 299.

Each of the substituted pyrazoles presented in Table 2 below were prepared in accordance with the synthetic routes in General Scheme 2, using procedures analogous to those described above.

TABLE 2
Intermediates

| Intermediate | Structure |
|---|---|
| 91 | 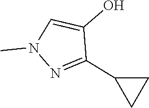 |
| 92 | 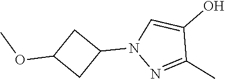 |
| 93 | 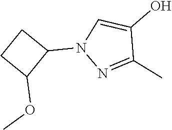 |
| 94 | 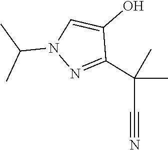 |
| 95 | 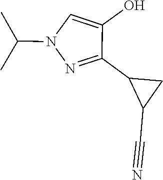 |
| 96 | 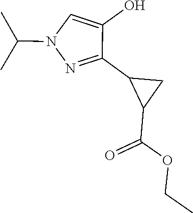 [SMILES: CCOC(=O)C1CC1c1nn(C(C)C)cc1O] |
| 97 | 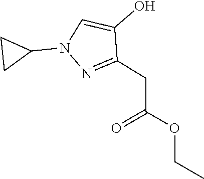 |
| 98 | 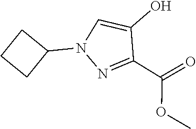 |
| 99 | 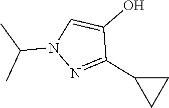 |
| 100 | 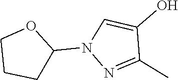 |
| 101 | 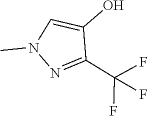 |
| 102 | 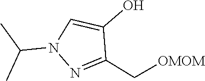 |

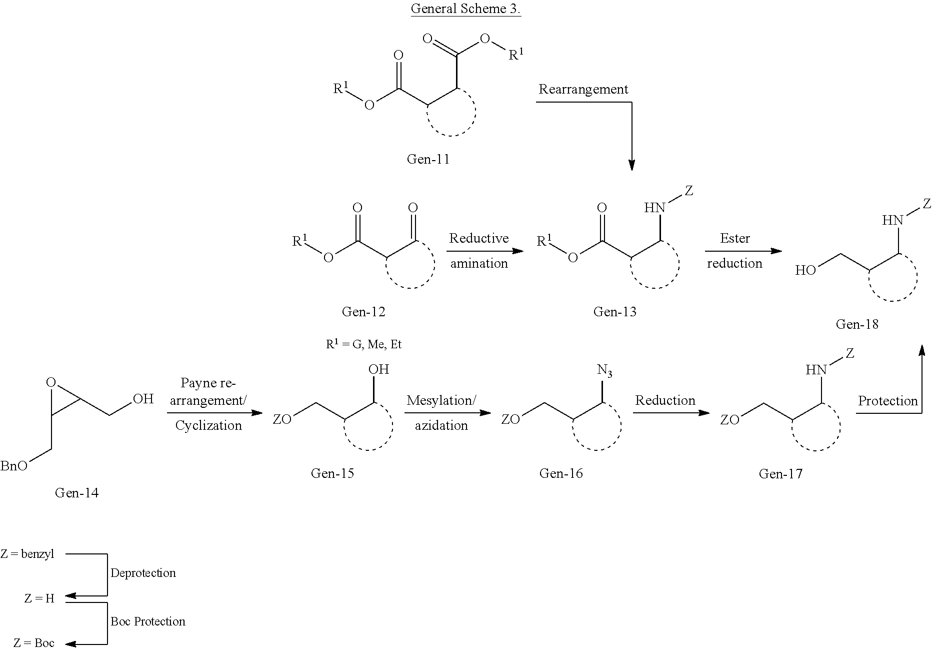

In General Scheme 3, commercially available or synthetically prepared 4-alkoxy-4-oxoaliphatic acids Gen-11 could be converted to the amine through a Schmidt transformation with reagents commonly known to those skilled in the art to afford β-amino esters Gen-13. Alternatively, 1,3-ketoesters Gen-12 could be converted to amine Gen-13 through reductive aminations commonly known to those skilled in the art. The amine of Gen-13 could optionally be cleaved of substituted or unsubstituted benzyl groups to the primary amine through a number of deprotection transformations commonly known to those skilled in the art including, but not limited to, hydrogenation, and then optionally substituted with protecting groups commonly known to those skilled in the art including, but not limited to, tert-butyloxycarbonyl. The ester of Gen-13 could be functionalized to the alcohol through ester reduction with reagents commonly known to those skilled in the art. In an alternate route, commercially available or synthetically prepared epoxy alcohols Gen-14 could be transformed to the 1,3-diol Gen-15 through a rearrangement and cyclization sequence. The alcohol Gen-15 could be transformed to the amine Gen-17 through leaving group formation and azide displacement to afford Gen-16, which could be reduced to the amine Gen-17. The representative compounds are described in more detail below.

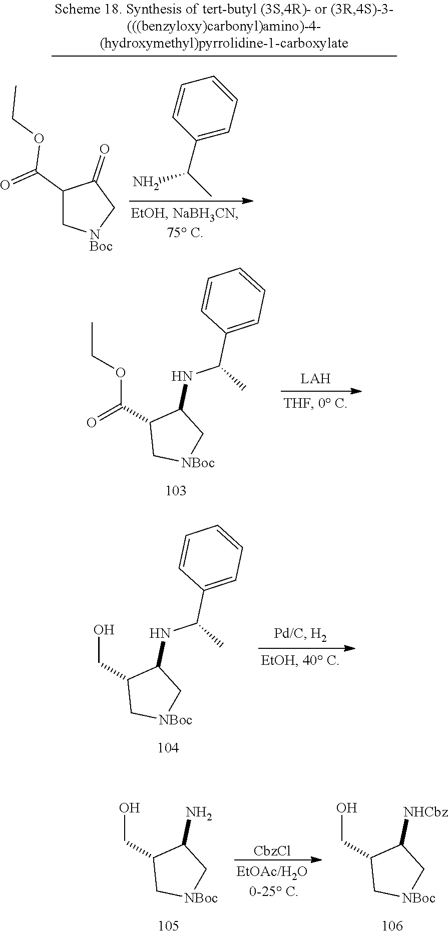

### 1-(tert-Butyl) 3-ethyl (3S,4R)- and (3R,4S)-4-(((S)-1-phenylethyl)amino) pyrrolidine-1,3-dicarboxylate (103)

To a solution of 1-(tert-butyl) 3-ethyl 4-oxopyrrolidine-1,3-dicarboxylate (35.0 g, 136 mmol) and (S)-1-phenylethan-1-amine (30.0 g, 248 mmol) in anhydrous EtOH (400 mL) was added NaBH₃CN (20 g, 318 mmol), followed by acetic acid (9.00 mL, 136 mmol). The reaction mixture was allowed to stir at 25° C. under N₂ for 4 h, then heated to 75° C. and allowed to stir for 16 h. The mixture was cooled, filtered and the organic volatiles were concentrated under reduced pressure. The reaction mixture was poured into water (150 mL) and extracted with EtOAc (3×150 mL). The organic layer was washed with water (2×100 mL) and dried over Na₂SO₄. The solution was filtered and concentrated under reduced pressure. The crude product was purified by flash chromatography on silica gel (gradient elution of 0-50% EtOAc/PE) to afford the title compound 103.

### tert-Butyl (3S,4R)- and (3R,4S)-3-(hydroxymethyl)-4-(((S)-1-phenylethyl)amino) pyrrolidine-1-carboxylate (104)

mixture was quenched by the addition of water (10 mL). The mixture was filtered and concentrated under reduced pressure to afford title compound 104. The product was used in the next step directly without further purification.

### tert-Butyl (3S,4R)- and (3R,4S)-3-amino-4-(hydroxymethyl) pyrrolidine-1-carboxylate (105)

To a solution of tert-butyl (3S,4R)- or (3R,4S)-3-(hydroxymethyl)-4-((1-phenylethyl)amino) pyrrolidine-1-carboxylate 104 (14.0 g, 43.7 mmol) in anhydrous EtOH (300 mL) was added Pd/C (4.65 g, 4.37 mmol), and the reaction mixture was allowed to stir at 45° C. under H₂ (45 psi) for 16 h. The mixture was filtered and concentrated under reduced pressure to afford title compound 105. The product was used in the next step directly without further purification.

### tert-Butyl (3S,4R)- and (3R,4S)-3-(((benzyloxy) carbonyl)amino)-4-(hydroxymethyl) pyrrolidine-1-carboxylate (106)

To a solution of tert-butyl (3S,4R)- or (3R,4S)-3-amino-4-(hydroxymethyl) pyrrolidine-1-carboxylate 105 (5.00 g, 23.1 mmol) in ethyl acetate (120 mL) and water (20 mL) was added sodium bicarbonate (2.33 g, 27.7 mmol) and CbzCl (3.96 mL, 27.7 mmol) at 0° C. The reaction was allowed to stir for 16 h at 20° C., then poured into water (10 mL) and extracted with EtOAc (3×15 mL×3). The combined organic layers were dried over Na₂SO₄. The solution was filtered and concentrated under reduced pressure. The crude product was purified by flash chromatography on silica gel (gradient elution of 0-60% EtOAc/PE) to afford the title compound 106. MS (ESI): m/z calc'd for C₁₈H₂₆N₂O₅ [M+H]⁺: 351, found 351.

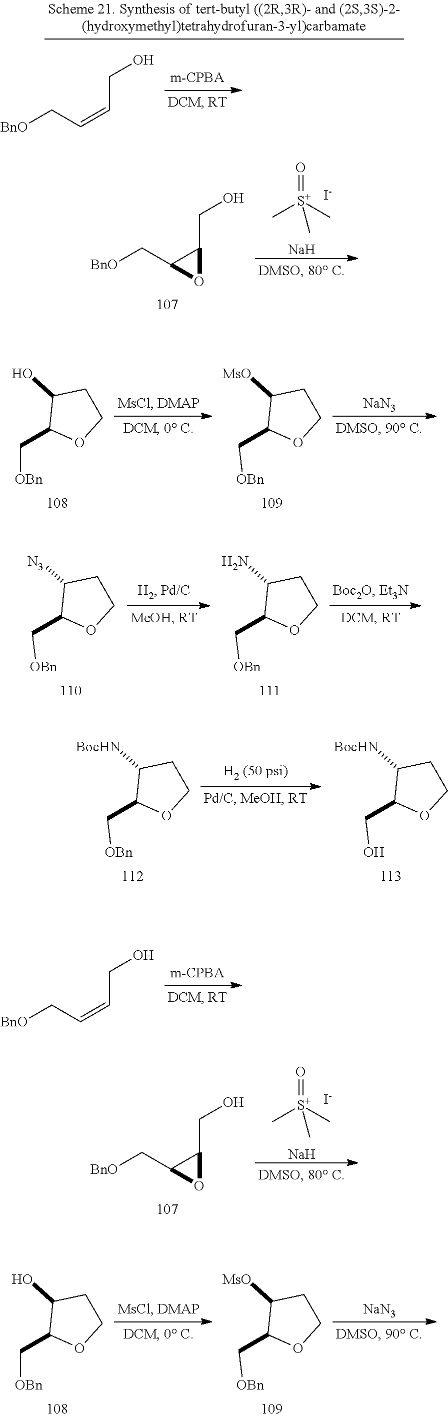

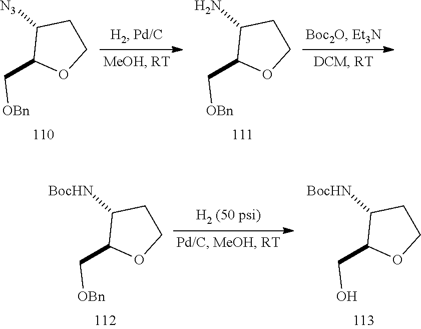 [SMILES: [N-]=[N+]=N[C@@H]1CCO[C@H]1COCc1ccccc1 ; N[C@@H]1CCO[C@H]1COCc1ccccc1 ; CC(C)(C)OC(=O)N[C@@H]1CCO[C@H]1COCc1ccccc1 ; CC(C)(C)OC(=O)N[C@@H]1CCO[C@H]1CO]

### ((2R,3S)- and (2S,3R)-3-((Benzyloxy)methyl)oxiran-2-yl)methanol (107)

To a flask containing cis-4-benzyloxy-2-buten-1-ol (943 μL, 5.61 mmol) in CH₂Cl₂ (22.4 mL) was added 3-chloroperoxybenzoic acid (1890 mg, 8.42 mmol). The reaction was allowed to react at RT overnight, then cooled to 0° C. and filtered through a pad of Celite. The precipitate was washed with cold DCM. The filtrate was washed with saturated aqueous Na₂SO₃. The aqueous layer was extracted with DCM. The combined organic layers were washed with saturated aqueous NaHCO₃, brine, and dried over Na₂SO₄. The solution was filtered and concentrated under reduced pressure to afford the title compound 107.

### (2R,3R)- and (2S,3S)-2-((Benzyloxy)methyl)tetrahydrofuran-3-ol (108)

To a flask containing a solution of sodium hydride (673 mg, 16.8 mmol) in DMSO (56.1 mL) was added trimethylsulfoxonium iodide (3700 mg, 16.8 mmol). The reaction mixture was allowed to stir for 30 min at RT, then a solution of ((2R,3S)- or (2S,3R)-3-((Benzyloxy)methyl)oxiran-2-yl)methanol 107 (1090 mg, 5.61 mmol) in DMSO (14 mL) was added. The reaction mixture was allowed to stir for 14 h, then diluted with 50 ml of water and 1 mL of saturated aqueous NH₄Cl, and extracted with ethyl acetate. The combined organics were washed with brine and dried over Na₂SO₄. The solution was filtered and concentrated under reduced pressure. The crude residue was purified by flash chromatography on silica gel (gradient elution of 0-100% EtOAc/hexanes) to afford the title compound 108.

### (2R,3R)- and (2S,3S)-2-((Benzyloxy)methyl)tetrahydrofuran-3-yl methanesulfonate (109)

To a solution of (2R,3R)- or (2S,3S)-2-((benzyloxy)methyl)tetrahydrofuran-3-ol 108 (723 mg, 3.47 mmol) stirring in DCM (6.95 mL) at 0° C., was added 4-dimethylaminopyridine (85.0 mg, To a flask containing (2R,3R)- or (2S,3S)-2-((benzyloxy)methyl)tetrahydrofuran-3-yl methanesulfonate 109 (994 mg, 3.47 mmol) stirring in DMSO (2310 μL), was added sodium azide (1540 mg, 23.6 mmol). The reaction mixture was heated to 90° C. and allowed to stir for 2 h, then diluted with water and ethyl acetate. The layers were separated. The aqueous layer was extracted with ethyl acetate. The combined organic layers were washed with brine and dried over Na₂SO₄. The solution was filtered and concentrated under reduced pressure. The crude residue was purified by flash chromatography on silica gel (gradient elution of 0-100% EtOAc/hexanes) to afford the title compound 110.

### (2R,3R)- and (2S,3S)-2-((Benzyloxy)methyl)tetrahydrofuran-3-amine (111)

To a flask containing (2R,3R)- or (2S,3S)-3-azido-2-((benzyloxy)methyl)tetrahydrofuran 110 (63 mg, 2.72 mmol) stirring in MeOH (27.2 mL), was added Pd/C (289 mg, 0.272 mmol). The reaction mixture was degassed with a H₂ balloon before allowing to stir at 22° C. overnight. The reaction was carefully filtered and concentrated under reduced pressure to afford the title compound 111. The product was used in the next step directly without further purification.

### tert-Butyl ((2R,3R)- and (2S,3S)-2-((benzyloxy)methyl)tetrahydrofuran-3-yl)carbamate (112)

To a flask containing (2R,3R)- or (2S,3S)-2-((benzyloxy)methyl)tetrahydrofuran-3-amine 111 stirring in DCM (18.2 mL), was added di-tert-butyl dicarbonate (597 μL, 2.74 mmol) and then triethylamine (508 μL, 3.65 mmol). The reaction mixture was allowed to stir overnight at RT. The reaction mixture was concentrated under reduced pressure and purified by flash chromatography on silica gel (gradient elution of 0-50% EtOAc/hexanes) to afford the title compound 112.

### tert-Butyl ((2R,3R)- and (2S,3S)-2-(Hydroxymethyl)tetrahydrofuran-3-yl)carbamate (113)

To a flask containing tert-butyl ((2R,3R)- or (2S,3S)-2-((benzyloxy)methyl)tetrahydrofuran-3-yl)carbamate 112 (406 mg, 1.321 mmol) stirring in MeOH (13.2 mL), was added Pd/C (141 mg, 0.132 mmol). The reaction mixture was degassed with a H₂ balloon before allowing to stir at 22° C. overnight, then was carefully filtered and concentrated under reduced pressure to afford the title compound 113. The product was used in the next step directly without further purification. ¹H NMR (CDCl₃, 500 MHz) δ:4.05-3.96 (1H, m), 3.91 (1H, m), 3.71 (1H, m), 3.67 (1H, m),

### tert-Butyl ((2R,3R)- and (2S,3S)-2-(Hydroxymethyl)tetrahydrofuran-3-yl)carbamate (113)

To a flask containing tert-butyl ((2R,3R)- or (2S,3S)-2-((benzyloxy)methyl)tetrahydrofuran-3-yl)carbamate 112 (406 mg, 1.321 mmol) stirring in MeOH (13.2 mL), was added Pd/C (141 mg, 0.132 mmol). The reaction mixture was degassed with a H₂ balloon before allowing to stir at 22° C. overnight, then was carefully filtered and concentrated under reduced pressure to afford the title compound 113. The product was used in the next step directly without further purification. ¹H NMR (CDCl₃, 500 MHz) δ:4.05-3.96 (1H, m), 3.91 (1H, m), 3.71 (1H, m), 3.67 (1H, m), 3.35 (1H, m), 3.12 (1H, s), 2.29 (1H, m), 1.79 (1H, dq, J=13.2, 6.5 Hz), 1.45 (9H, s).

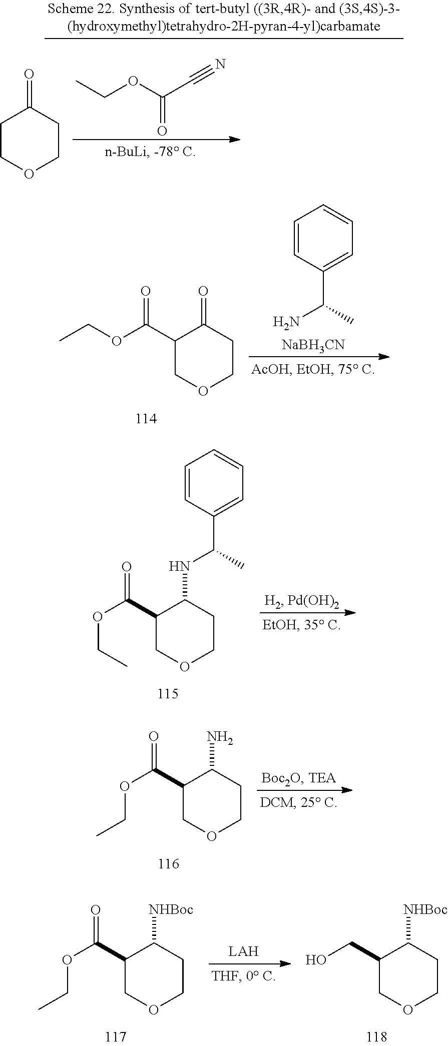

### Ethyl (R)- and (S)-4-oxotetrahydro-2H-pyran-3-carboxylate (114)

A solution of diisopropylamine (22.2 g, 220 mmol) in THF (80 mL) was cooled to −78° C. under N₂ atmosphere and then n-butyl lithium (88.0 mL, 220 mmol) was added dropwise to the solution. The solution was allowed to warm to 0° C. and stir for 1 h. To the reaction mixture was added dropwise a solution of tetrahydro-4H-pyran-4-one (20.0 g, 200 mmol) in THF (1000 mL) at −78° C. The mixture was allowed to warm to 0° C. and stir for 1 h. The reaction solution was cooled to −78° C. To the mixture was added hexamethylphosphoramide (34.9 mL, 200 mmol) and ethyl cyanoformate (21.9 mL, 240 mmol) at the same temperature. The mixture was allowed to warm to 0° C. and stir for 1 h. The reaction was quenched by the addition of saturated aqueous NH₄Cl solution. The mixture was extracted with EtOAc (3×300 mL), washed with brine and dried over MgSO₄. The solution was filtered and concentrated under reduce pressure. The residue was purified by flash chromatography on silica gel (gradient elution of 0-8% Sodium cyanoborohydride (2.41 g, 38.3 mmol) was added to the solution, and this suspension was allowed to stir for 16 h at 75° C. The reaction was concentrated under reduced pressure to give crude residue, which was purified by flash chromatography on silica gel (gradient elution of 0-35% EtOAc/hexanes) to afford the title compound 115.

### Ethyl (3S,4R)- and (3R,4S)-4-aminotetrahydro-2H-pyran-3-carboxylate (116)

To a solution of ethyl (3S,4R)- and (3R,4S)-4-(((S)-1-phenylethyl)amino)tetrahydro-2H-pyran-3-carboxylate 115 (3.20 g, 11.5 mmol) in EtOH (30 mL) was added Pd(OH)₂ (0.810 g, 1.15 mmol) under Ar atmosphere. The reaction was allowed to stir for 18 h at 35° C. under H₂ atmosphere (50 psi). This mixture was filtered, and the filtrate was concentrated under reduced pressure to afford the title compound 116. The product was used in the next step directly without further purification.

### Ethyl (3S,4R)- and (3R,4S)-4-((tert-butoxycarbonyl)amino)tetrahydro-2H-pyran-3-carboxylate (117)

To a solution of ethyl (3S,4R)- and (3R,4S)-4-aminotetrahydro-2H-pyran-3-carboxylate 116 (2.00 g, 11.6 mmol) and TEA (2.41 mL, 17.3 mmol) in DCM (20 mL) was added Boc anhydride (3.49 mL, 15.0 mmol) under N₂ atmosphere. The reaction was allowed to stir for 1 h at 25° C. This mixture was directly purified by flash chromatography on silica gel (gradient elution of 0-15% EtOAc/hexanes) to afford the title compound 117.

### tert-Butyl ((3R,4R)- and (3S,4S)-3-(hydroxymethyl)tetrahydro-2H-pyran-4-yl)carbamate (118)

To a solution of ethyl (3S,4R)- and (3R,4S)-4-((tert-butoxycarbonyl)amino)tetrahydro-2H-pyran-3-carboxylate 117 (500 mg, 1.83 mmol) in anhydrous THF (10 mL) was added LAH (0.104 g, 2.74 mmol) at 0° C. under N₂ atmosphere, and the reaction mixture was allowed to stir at 0° C. for 1 h. The reaction mixture was quenched by the addition of water (0.1 mL), aqueous NaOH (0.3 mL, 15%), and H₂O (0.1 mL), then dried over anhydrous Na₂SO₄. The solution was filtered and concentrated under reduced pressure to afford the title compound 118. The product was used in the next step directly without further purification. MS (ESI): m/z calc'd for C₁₁H₂₁NO₄ [M+H]⁺: 247, found 247.

2.74 mmol) at 0° C. under N₂ atmosphere, and the reaction mixture was allowed to stir at 0° C. for 1 h. The reaction mixture was quenched by the addition of water (0.1 mL), aqueous NaOH (0.3 mL, 15%), and H₂O (0.1 mL), then dried over anhydrous Na₂SO₄. The solution was filtered and concentrated under reduced pressure to afford the title compound 118. The product was used in the next step directly without further purification. MS (ESI): m/z calc'd for C₁₁H₂₁NO₄ [M+H]⁺: 247, found 247.

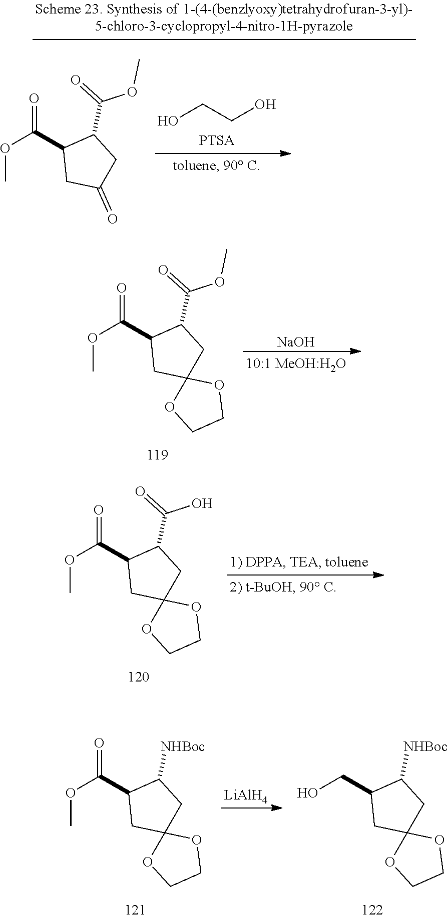

### Dimethyl (7R,8R)- and (7S,8S)-1,4-dioxaspiro[4.4]nonane-7,8-dicarboxylate (119)

To a solution of dimethyl 4-oxocyclopentane-1,2-dicarboxylate (5.00 g, 25.0 mmol) and ethane-1,2-diol (1.94 g, 31.2 mmol) in toluene (80 mL) was added p-toluenesulfonic acid monohydrate (0.119 g, 0.624 mmol). The reaction mixture was allowed to stir at 120° C. for 12 h, then basified with saturated aqueous NaHCO₃ to pH 8-9, extracted with EtOAc (3×100 mL×3) and dried over Na₂SO₄. The solution was filtered and concentrated under reduced pressure. The crude product was purified by flash chromatography on silica gel (gradient elution of 0-45% EtOAc/hexanes) to afford the title compound 119.

### (7R,8R)- and (7S,8S)-8-(Methoxycarbonyl)-1,4-dioxaspiro[4.4]nonane-7-carboxylic acid (120)

To a solution of dimethyl (7R,8R)- and (7S,8S)-1,4-dioxaspiro[4.4]nonane-7,8-dicarboxylate 119 (4.80 g, 19.7 mmol) in MeOH (50 mL) was added sodium hydroxide (0.747 g, 18.7 mmol) in water (5 mL). The solution was allowed to stir for 3 h at 20° C., then was acidified with 1 N HCl to pH 6 and concentrated under reduced pressure. The crude product was purified by flash chromatography on silica gel (gradient elution of 0-45% EtOAc/hexanes) to afford the title compound 120.

### Methyl (7R,8R)- and (7S,8S)-8-((tert-butoxycarbonyl)amino)-1,4-dioxaspiro[4.4]nonane-7-carboxylate (121)

To a solution of (7R,8R)- and (7S,8S)-8-(methoxycarbonyl)-1,4-dioxaspiro[4.4]nonane-7-carboxylic acid 120 (2.50 g, 10.9 mmol) and TEA (2.27 mL, 16.3 mmol) in anhydrous t-BuOH (30 mL) was added diphenylphosphoryl azide (2.81 mL, 13.0 mmol). The reaction mixture was allowed to stir at 90° C. under N₂ for 12 h, then concentrated under reduced pressure. The crude product was purified by flash chromatography on silica gel (gradient elution of 0-45% EtOAc/hexanes) to afford the title compound 121.

### tert-Butyl ((7R,8R)- and (7S,8S)-8-(hydroxymethyl)-1,4-dioxaspiro[4.4]nonan-7-yl)carbamate (122)

To a solution of methyl (7R,8R)- and (7S,8S)-8-((tert-butoxycarbonyl)amino)-1,4-dioxaspiro[4.4]nonane-7-carboxylate 121 (1.3 g, 4.31 mmol) in THF (20 mL) was added LiAlH₄ (0.327 g, 8.63 mmol) at 0° C. The reaction mixture was allowed to stir at 0° C. for 1.5 h, then was quenched by the addition of H₂O (0.4 mL), filtered and concentrated to afford the title compound 122. The product was used in the next step directly without further purification. ¹H NMR (500 MHz, CDCl₃) δ: 4.20-3.95 (m, 1H), 3.95-3.84 (m, 4H), 3.61 (br dd, J=3.1, 11.1 Hz, 1H), 3.56-3.38 (m, 1H), 2.26 (br dd, J=8.5, 13.4 Hz, 2H), 2.04-1.99 (m, 2H), 1.73-1.64 (m, 1H), 1.44 (s, 9H).

Each of the substituted amino alcohols presented in Table 3 below were prepared in accordance with the synthetic routes in General Scheme 3, using procedures analogous to those described above.

TABLE 3
Intermediates

| Intermediate | Structure |
|---|---|
| 123 | 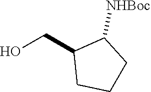 |

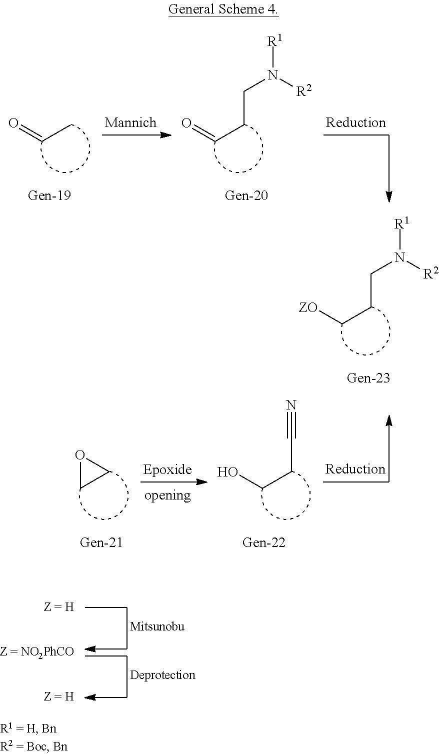

In General Scheme 4, commercially available or synthetically prepared ketones Gen-19 could be aminoalkylated using a number of synthetic transformations commonly known to those skilled in the art including, but not limited to, a Schmidt reaction, to afford β-amino carbonyls Gen-20. The ketones of the form Gen-20 could be reduced using a number of synthetic transformations commonly known to those skilled in the art including, but not limited to, hydride addition, to afford primary alcohols of the form Gen-23. In an alternative route, commercially available or synthetically prepared epoxides Gen-21 could be opened through a number of synthetic transformations commonly known to those skilled in the art including, but not limited to, organometallic addition, to afford nitriles of the form Gen-22. Tandem nickel-catalyzed reduction and of the nitrile Gen-22 could afford the amine Gen-23. The stereochemistry of the alcohol could optionally be transformed through Mitsunobu with benzoic acids commonly known to those skilled in the art. The protected ester could be optionally transformed to the alcohol Gen-23 using a number of synthetic transformations commonly known to those skilled in the art including, but not limited to hydrolysis. The representative compounds are described in more detail below.

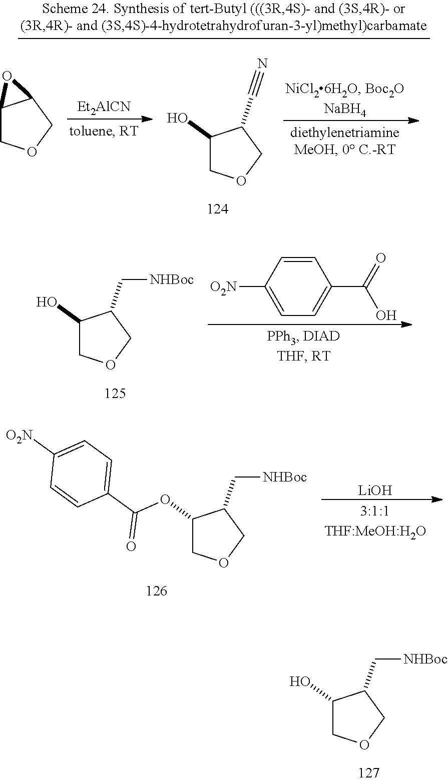

### (3R,4S)- and (3S,4R)-4-Hydroxytetrahydrofuran-3-carbonitrile (124)

To a flask containing a solution of 3,4-epoxytetrahydrofuran (0.5 mL, 7.18 mmol) in toluene (20 mL) was added dropwise diethylaluminum cyanide (1 M in toluene, 14.4 mL, 14.4 mmol). The reaction was allowed to stir at RT overnight. The reaction was cooled to 0° C. and quenched by the dropwise addition of 1 M NaOH. The reaction was diluted with water and allowed to warm to RT. The mixture was filtered and extracted with EtOAc. The combined organic layers were washed with water, with brine and dried over Na₂SO₄. The solution was filtered and concentrated under reduced pressure to afford the title compound 124. The product was used in the next step directly without further purification.

### tert-Butyl (((3R,4S)- and (3S,4R)-4-hydroxytetrahydrofuran-3-yl)methyl)carbamate (125)

To a flask containing nickel (II) chloride hexahydrate (50.8 mg, 0.214 mmol), Boc-anhydride (0.993 mL, 4.28 mmol), and (3R,4S)- and (3S,4R)-4-hydroxytetrahydrofuran-3-carbonitrile 124 (242 mg, 2.139 mmol) was added methanol (16 mL). The reaction mixture was cooled to 0° C. and sodium borohydride (567 mg, 15.0 mmol) was added portion-wise. The reaction was allowed to warm to RT and allowed to stir for 30 min. To the mixture was added diethylenetriamine (0.25 mL, 2.31 mmol). The reaction was allowed to stir for 1 h at RT, then was concentrated under reduced pressure. The residue was dissolved in EtOAc and water. The layers were separated. The organic layer was washed with saturated aqueous NaHCO₃, brine and dried over Na₂SO₄. The solution was filtered and concentrated under reduced pressure to afford the title compound 125. The product was used in the next step directly without further purification. ¹H NMR (CDCl₃, 500 MHz) δ: 4.73 (1H, s), 4.27-4.18 (1H, m), 4.09-4.03 (m, 1H), 3.95 (1H, dd, J=9.6, 5.3 Hz), 3.71-3.66 (1H, m), 3.52 (1H, dd, J=8.8, 5.5 Hz), 3.25-3.18 (1H, m), 3.17-3.11 (1H, m), 2.78-2.60 (1H, m), 2.34-2.24 (1H, m), 1.44 (9H, s).

### (3R,4R)- and (3S,4S)-4-(((tert-Butoxycarbonyl)amino)methyl)tetrahydrofuran-3-yl 4-nitrobenzoate (126)

To a microwave vial containing 4-nitrobenzoic acid (173 mg, 1.04 mmol) was added triphenylphosphine (407 mg, 1.55 mmol) and tert-butyl (((3R,4S)- and (3S,4R)-4-hydroxytetrahydrofuran-3-yl)methyl)carbamate 125 (225 mg, 1.036 mmol). The mixture was dissolved in THF (5 mL), cooled to 0° C., and diisopropyl azodicarboxylate (0.35 ml, 1.78 mmol) was added dropwise. The reaction was allowed to stir at RT for 48 h, then was diluted with saturated aqueous NaHCO₃ and extracted EtOAc. The combined organic layers were dried over Na₂SO₄. The solution was filtered and concentrated under reduced pressure. The crude product was purified by flash chromatography on silica gel (gradient elution of 0-100% 3:1 EtOAc:EtOH/hexanes) to afford the title compound 126.

### tert-Butyl (((3R,4R)- and (3S,4S)-4-hydroxytetrahydrofuran-3-yl)methyl)carbamate (127)

To a flask containing (3R,4R)- and (3S,4S)-4-(((tert-butoxycarbonyl) amino)methyl)tetrahydrofuran-3-yl 4-nitrobenzoate 126 (325 mg, 0.887 mmol) was added lithium hydroxide monohydrate (112 mg, 2.66 mmol). The mixture was dissolved in a mixture of THF (3.00 mL), methanol (1.00 mL) and water (1.000 mL). The reaction was allowed to stir at RT overnight, then concentrated under reduced pressure. The residue was dissolved in water and extracted with EtOAc. The organic layers were washed with saturated aqueous NaHCO₃, with brine and dried over Na₂SO₄. The solution was filtered and concentrated under reduced pressure to afford the title compound 127. ¹H NMR (CDCl₃, 500 MHz) δ: 4.35-4.18 (1H, m), 3.99-3.82 (3H, m), 3.77 (1H, s), 3.59-3.39 (2H, m), 3.05-2.91 (1H, m), 2.28-2.13 (1H, m), 1.45 (9H, s).

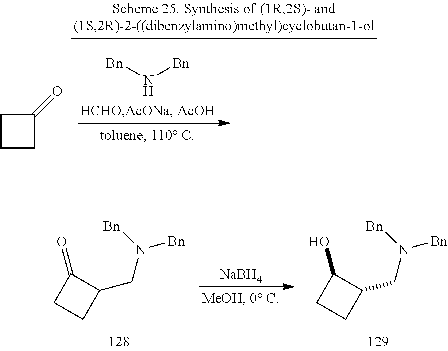 [SMILES: c1ccc(CNCc2ccccc2)cc1 ; O=C1CCC1 ; O=C1CCC1CN(Cc1ccccc1)Cc1ccccc1 ; "NaBH4" ; O[C@@H]1CC[C@H]1CN(Cc1ccccc1)Cc1ccccc1]

### (S)- and (R)-2-((Dibenzylamino)methyl)cyclobutan-1-one (128)

To a suspension of formaldehyde/paraformaldehyde (11.6 g, 143 mmol), cyclobutanone (10.0 g, 143 mmol), dibenzylamine (28.1 g, 143 mmol) and sodium acetate (2.34 g, 28.5 mmol) in toluene (100 mL) was added acetic acid (1.71 g, 28.5 mmol). The reaction was heated to 110° C., allowed to stir for 16 h under N₂, then filtered and concentrated under reduced pressure. The crude residue was purified by flash chromatography on silica gel (gradient elution of 0-10% EtOAc/PE) to afford the title compound 128.

### (1R,2S)- and (1S,2R)-2-((Dibenzylamino)methyl)cyclobutan-1-ol (129)

To a solution of (S)- and (R)-2-((Dibenzylamino)methyl)cyclobutan-1-one 128 (10.0 g, 35.8 mmol) in anhydrous MeOH (50 mL) was added sodium tetrahydroborate (2.71 g, 71.6 mmol) at 0° C. The reaction mixture was allowed to stir at 0° C. for 1 h. The reaction was quenched by the addition of cold water and extracted with EtOAc. The layers were separated. The organic layer was washed with brine and dried over anhydrous Na₂SO₄, filtered and concentrated under reduced pressure to afford the title compound 129. MS (ESI): m/z calc'd for C₁₉H₂₃NO [M+H]⁺: 282, found 282.

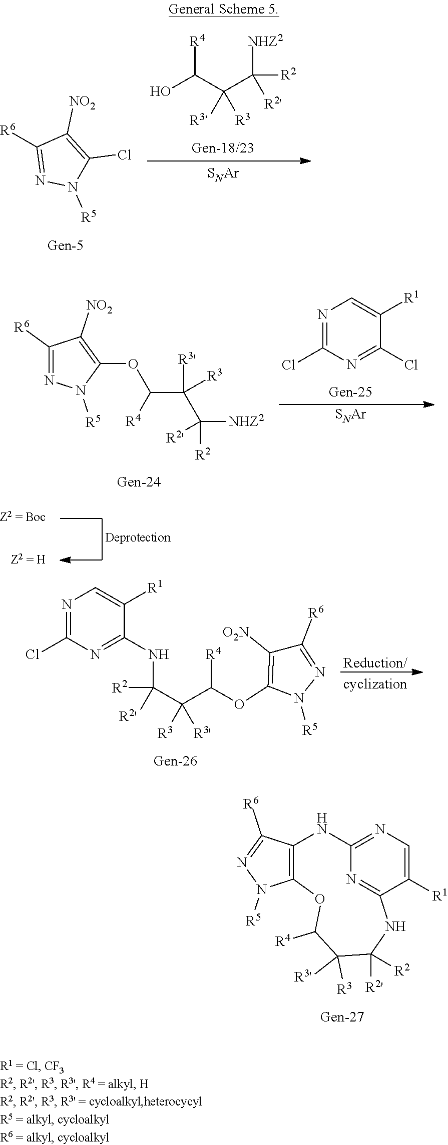

In General Scheme 5, chloropyrazoles of the form Gen-5 were coupled with commercially available or synthetically prepared amino-alcohols of the form Gen-18/Gen 23 through base-mediated S_{N}Ar chemistry to provide Gen-24. In instances of Gen-24 where Z²=Boc, acidic deprotection to the amine was performed. Commercially available pyrimidines of the form Gen-were coupled with amines of the form Gen-24 through S_{N}Ar chemistry to access Gen-26. The nitropyrazole Gen-26 could ultimately be transformed through a tandem reduction of the nitro group and intramolecular S_{N}Ar reaction to afford elaborated compounds of the form Gen-27. Representative preparative examples from each sequence are described in more detail below.

### Preparation of Example 1.1

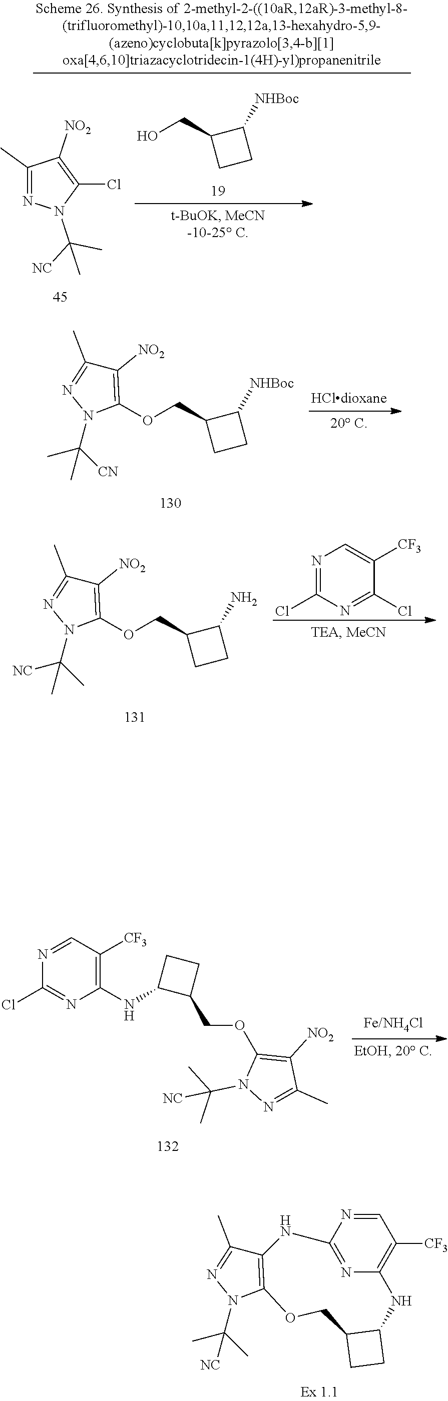

### tert-Butyl ((1R,2R)-2-(((1-(2-cyanopropan-2-yl)-3-methyl-4-nitro-1H-pyrazol-5-yl)oxy)methyl)cyclobutyl)carbamate (130)

To a flask was added 2-(5-chloro-3-methyl-4-nitro-1H-pyrazol-1-yl)-2-methylpropanenitrile 45 (380 mg, 1.66 mmol), tert-butyl ((1R,2R)-2-(hydroxymethyl)cyclobutyl)carbamate 19 (410 mg, 2.04 mmol) and acetonitrile (10 mL). To this solution was added potassium tert-butoxide (373 mg, 3.32 mmol) at −10° C. The mixture was allowed to stir at −10° C. for 3 h, then was quenched with saturated aqueous NH₄Cl and extracted with EtOAc. The combined organic layers were washed with brine and dried over MgSO₄. The solution was filtered and concentrated under reduced pressure. The crude residue was purified by flash chromatography on silica gel (gradient elution of 0-100% EtOAc/hexanes) to afford the title compound 130. MS (ESI): m/z calc'd for C₁₈H₂₇N₅O₅ [M-C₄H₉]⁺: 294, found 294.

### 2-(5-(((1R,2R)-2-aminocyclobutyl)methoxy)-3-methyl-4-nitro-1H-pyrazol-1-yl)-2-methylpropanenitrile (131)

To a solution of tert-butyl ((1R,2R)-2-(((1-(2-cyanopropan-2-yl)-3-methyl-4-nitro-1H-pyrazol-5-

To the vial containing 2-(5-(((1R,2R)-2-aminocyclobutyl)methoxy)-3-methyl-4-nitro-1H-pyrazol-1-yl)-2-methylpropanenitrile, 2TFA (232 mg, 0.445 mmol) 131 was added dioxane (3 mL) and Et₃N (0.700 mL, 5.02 mmol). To this solution was added 2,4-dichloro-5-(trifluoromethyl)pyrimidine (159 mg, 0.733 mmol) at 0° C. The mixture was allowed to stir at RT for 1 h, then was diluted with water and extracted with EtOAc. The combined organic layers were washed with brine and dried over MgSO₄. The solution was filtered and concentrated under reduced pressure. The crude residue was purified by flash chromatography on silica gel (gradient elution of 0-50% EtOAc/hexanes) to afford the title compound 132. MS (ESI): m/z calc'd for C₁₈H₁₉ClF₃N₇O₃ [M+H]⁺: 474, found 474.

### 2-Methyl-2-((10aR,12aR)-3-methyl-8-(trifluoromethyl)-10,10a,11,12,12a,13-hexahydro-5,9-(azeno)cyclobuta[k]pyrazolo[3,4-b][1]oxa[4,6,10]triazacyclotridecin-1 (4H)-yl)propanenitrile (Ex-1.1)

To a flask containing iron (101 mg, 1.81 mmol) was added ammonium chloride (97 mg, 1.80 mmol), water (3 mL) and a solution of 2-(5-(((1R,2R)-2-((2-chloro-5-(trifluoromethyl)pyrimidin-4-yl)amino)cyclobutyl)methoxy)-3-methyl-4-nitro-1H-pyrazol-1-yl)-2-methylpropanenitrile 132 (142.6 mg, 0.301 mmol) in ethanol (15 mL). The mixture was heated to 90° C. and allowed to stir at RT for 4 d. The mixture was diluted with water and extracted with EtOAc. The combined organic layers were washed with brine and dried over MgSO₄. The solution was filtered and concentrated under reduced pressure. The crude residue was purified by flash chromatography on silica gel (gradient elution of 0-40% EtOAc/hexanes) to afford a mixture of regioisomers. The regioisomers could be separated by achiral preparative SFC (Column: AD-H (21×250 mm, 5 μm); Mobile phase A: CO₂; Mobile phase B: 0.1% NH₄OH in MeOH) (t_{R}=1.95 min) to afford the title compound Ex-1.1. MS (ESI): m/z calc'd for C₁₈H₂₀F₃N₇O [M+H]⁺: 408, found 408. ¹H NMR (DMSO-d₆, 600 MHz) δ 8.64 (1H, s), 8.13 (1H, s), 7.50 (1H, d, J=4.3 Hz), 4.52-4.45 (1H, m), 4.37 (1H, dd, J=9.9, 3.5 Hz), 4.13-3.98 (1H, m), 2.22-2.15 (2H, m), 2.13 (3H, s), 2.02 (1H, q, J=8.3 Hz), 1.90 (3H, s), 1.84 (3H, s), 1.81-1.72 (2H, m).

### Preparation of Example 1.2

### Scheme 27. Synthesis of (10aS,13aS)-1-methyl-3,8-bis(trifluoromethyl)-1,4,10a,11,13a,14-hexahydro-10H,13H-5,9-(azeno)furo[3,4-k]pyrazolo[3,4-b][1]oxa[4,6,10]triazacyclotridecine

### NMR (DMSO-d₆, 600 MHz) δ 8.64 (1H, s), 8.13 (1H, s), 7.50 (1H, d, J=4.3 Hz), 4.52-4.45 (1H, m), 4.37 (1H, dd, J=9.9, 3.5 Hz), 4.13-3.98 (1H, m), 2.22-2.15 (2H, m), 2.13 (3H, s), 2.02 (1H, q, J=8.3 Hz), 1.90 (3H, s), 1.84 (3H, s), 1.81-1.72 (2H, m).

### Preparation of Example 1.2

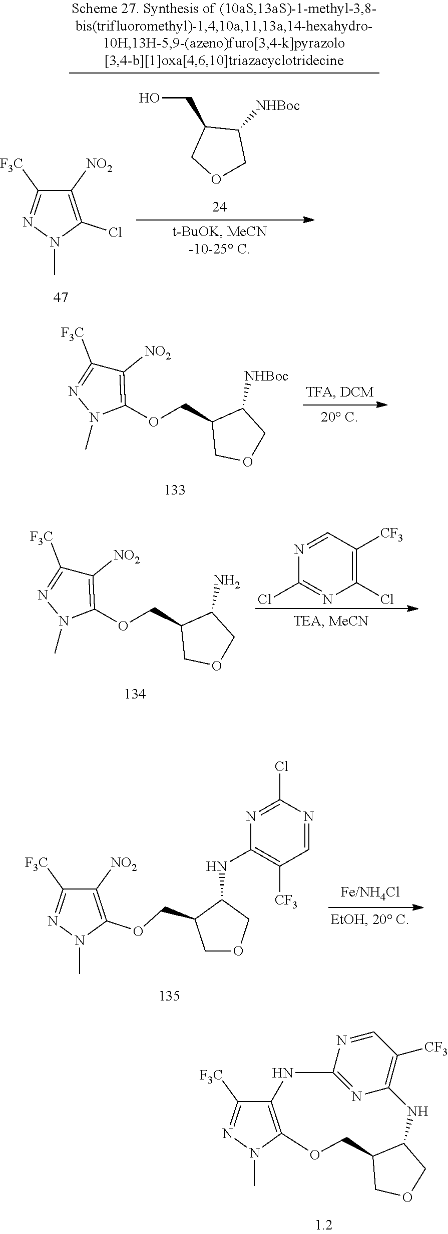

### tert-Butyl ((3S,4S)-4-(((1-methyl-4-nitro-3-(trifluoromethyl)-1H-pyrazol-5-yl)oxy)methyl)tetrahydrofuran-3-yl)carbamate (133)

To a flask was added 5-chloro-1-methyl-4-nitro-3-(trifluoromethyl)-1H-pyrazole 47 (508 mg, 2.213 mmol), tert-butyl ((3S,4R)-4-(hydroxymethyl)tetrahydrofuran-3-yl)carbamate 24 (584 mg, 2.69 mmol) and acetonitrile (10 mL). To this solution was added portion-wise potassium tert-butoxide (373 mg, 3.32 mmol) at −10° C. The mixture was allowed to stir from −10° C. to RT for 40 min, then was quenched with saturated aqueous NH₄Cl and extracted with EtOAc. The combined organic layers were washed with brine and dried over MgSO₄. The solution was filtered and concentrated under reduced pressure. The crude residue was purified by flash chromatography on silica gel (gradient elution of 0-50% EtOAc/hexanes) to afford the title compound 133. MS (ESI): m/z calc'd for C₁₅H₂₁F₃N₄O₆ [M-C₄H₉]⁺: 311, found 311.

### (3S,4S)-4-(((1-Methyl-4-nitro-3-(trifluoromethyl)-1H-pyrazol-5-yl)oxy)methyl)tetrahydrofuran-3-amine, TFA (134)

To the solution of tert-butyl ((3S,4S)-4-(((1-methyl-4-nitro-3-(trifluoromethyl)-1H-pyrazol-5-yl)oxy)methyl)tetrahydrofuran-3-yl)carbamate 133 (672 mg, 1.64 mmol) in DCM (12 mL) was added TFA (0.300 mL, 3.89 mmol). The mixture was allowed to stir at RT for 18 h. To the mixture was added additional TFA (0.600 mL, 7.79 mmol). After stirring at RT for an additional 24 h, the reaction was concentrated under reduced pressure to afford the title compound 134. The product was used in the next step directly without further purification. MS (ESI): m/z calc'd for C₁₀H₁₃F₃N₄O₄ [M+H]⁺: 311, found 311.

### 2-Chloro-N-((3S,4S)-4-(((1-methyl-4-nitro-3-(trifluoromethyl)-1H-pyrazol-5-yl)oxy)methyl)tetrahydrofuran-3-yl)-5-(trifluoromethyl)pyrimidin-4-amine (135)

To the vial containing (3S,4S)-4-(((1-methyl-4-nitro-3-(trifluoromethyl)-1H-pyrazol-5-yl)oxy)methyl)tetrahydrofuran-3-amine, TFA 134 (155 mg, 0.365 mmol) was added dioxane (2000 μL), followed by Et₃N (500 μL, 3.59 mmol) and 2,4-dichloro-5-(trifluoromethyl)pyrimidine (117.8 mg, 0.543 mmol). The mixture was allowed to stir at RT for 2 h, then was diluted with water and extracted with EtOAc. The combined organic layers were washed with brine and dried over MgSO₄. The solution was filtered and concentrated under reduced pressure. The crude residue was purified by flash chromatography on silica gel (gradient elution of 0-80% EtOAc/hexanes) to afford the title compound 135. MS (ESI): m/z calc'd for C₁₅H₁₃ClF₆N₆O₄ [M+H]⁺: 491, found 491.

### (10aS,13aS)-1-Methyl-3,8-bis(trifluoromethyl)-1,4,10a,11,13a,14-hexahydro-10H,13H-5,9-(azeno)furo[3,4-k]pyrazolo[3,4-b][1]oxa[4,6,10]triazacyclotridecine (Ex-1.2)

To the vial containing 2-chloro-N-((3S,4S)-4-(((1-methyl-4-nitro-3-(trifluoromethyl)-1H-pyrazol-5-yl)oxy)methyl)tetrahydrofuran-3-yl)-5-(trifluoromethyl)pyrimidin-4-amine 135 (37.9 mg, 0.077 mmol), was added iron (34.5 mg, 0.618 mmol), ammonium chloride (33.0 mg, 0.618 mmol), water (0.8 mL) and EtOH (4.00 mL). The mixture was heated to 90° C. and allowed to stir overnight, then filtered. The filtrate was diluted with water and EtOAc. The layers were separated. The aqueous layer was extracted with EtOAc. The combined organic layers were washed with brine and dried over MgSO₄. The solution was filtered and concentrated under reduced pressure to afford a residue. The residue was purified by reversed phase HPLC, eluting with water (0.1% NH₄OH)-MeCN to afford the title compound Ex-1.2. MS (ESI): m/z calc'd for C₁₅H₁₄F₆N₆O₂ [M+H]⁺: 425, found 425. ¹H NMR (DMSO-d₆, 500 MHz) δ 8.90 (1H, s), 8.16 (1H, s), 7.03 (1H, d, J=4.8 Hz), 4.48 (1H, d, J=11.3 Hz), 4.36-4.30 (1H, m), 4.29 (1H, s), 4.21 (1H, d, J=9.8 Hz), 4.09 (1H, t, J=9.0 Hz), 3.83 (3H, s), 3.69 (1H, dd, J=9.5, 6.8 Hz), 3.17 (1H, t, J=8.4 Hz), 2.23 (1H, q, J=8.0 Hz).

Compounds in Table 4 below were prepared in accordance with the synthetic sequence illustrated in General Scheme 5 using the corresponding starting materials.

TABLE 4

| | | | Exact Mass |
|---|---|---|---|
| Ex | Structure | Name | [M + H]⁺ |
| Ex-1.3 | 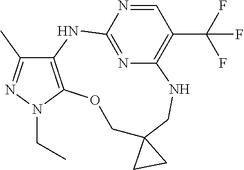 [SMILES: CCn1nc(C)c2c1OCC1(CC1)CNc1nc(ncc1C(F)(F)F)N2] | 1′-ethyl-3′-methyl-8′- (trifluoromethyl)-1′,4′,10′,11′- tetrahydro-13′H-spiro[cyclopropane- 1,12′-[5,9](azeno)pyrazolo[3,4- b][1]oxa[4,6,10]triazacyclotridecine] | Calc'd 369, found 369 |
| Ex-1.4 | 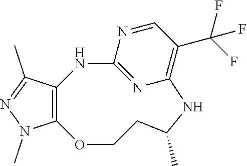 [SMILES: Cc1nn(C)c2c1Nc1ncc(C(F)(F)F)c(n1)N[C@H](C)CCO2] | (R)-1,3,11-trimethyl-8- (trifluoromethyl)-1,4,10,11,12,13- hexahydro-5,9-(azeno)pyrazolo[3,4- b][1]oxa[4,6,10]triazacyclotridecine | Calc'd 343, found 343 |
| Ex-1.5 | 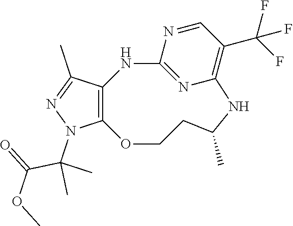 [SMILES: COC(=O)C(C)(C)n1nc(C)c2c1OCC[C@@H](C)Nc1nc(ncc1C(F)(F)F)N2] | methyl (R)-2-(3,11-dimethyl-8- (trifluoromethyl)-10,11,12,13- tetrahydro-5,9-(azeno)pyrazolo[3,4- b][1]oxa[4,6,10]triazacyclotridecin- 1(4H)-yl)-2-methylpropanoate | Calc'd 429, found 429 |
| Ex-1.6 | 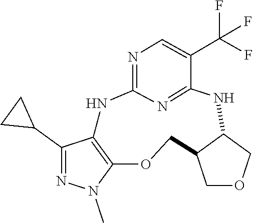 | (10aS,13aS)- or (10aR,13aR)-3- cyclopropyl-1-methyl-8- (trifluoromethyl)-1,4,10a,11,13a,14- hexahydro-10H,13H-5,9- (azeno)furo[3,4-k]pyrazolo[3,4- b][1]oxa[4,6,10]triazacyclotridecine | Calc'd 397, found 397 |
| Ex-1.7 | 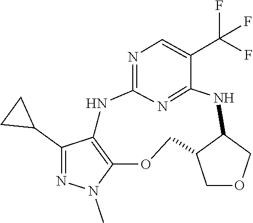 | (10aS,13aS)- or (10aR,13aR)-3- cyclopropyl-1-methyl-8- (trifluoromethyl)-1,4,10a,11,13a,14- hexahydro-10H,13H-5,9- (azeno)furo[3,4-k]pyrazolo[3,4- b][1]oxa[4,6,10]triazacyclotridecine | Calc'd 397, found 397 |
| Ex-1.8 | 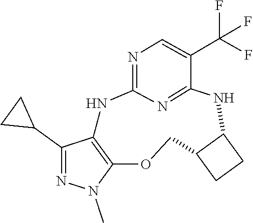 | (10aR,12aS)- or (10aS,12aR)-3- cyclopropyl-1-methyl-8- (trifluoromethyl)- 1,4,10,10a,11,12,12a,13-octahydro- 5,9-(azeno)cyclobuta[k]pyrazolo- [3,4-b][1]oxa[4,6,10]triazacyclo- tridecine | Calc'd 381, found 381 |
| Ex-1.9 | 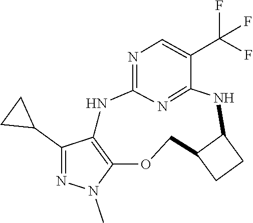 | (10aR,12aS)- or (10aS,12aR)-3- cyclopropyl-1-methyl-8- (trifluoromethyl)- 1,4,10,10a,11,12,12a,13-octahydro- 5,9-(azeno)cyclobuta[k]pyrazolo- [3,4-b][1]oxa[4,6,10]triazacyclo- tridecine | Calc'd 381, found 381 |
| Ex-1.10 | 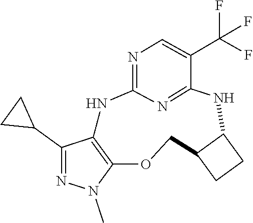 | (10aR,12aR)- or (10aS,12aS)-3- cyclopropyl-1-methyl-8- (trifluoromethyl)- 1,4,10,10a,11,12,12a,13-octahydro- 5,9-(azeno)cyclobuta[k]pyrazolo- [3,4-b][1]oxa[4,6,10]triazacyclo- tridecine | Calc'd 381, found 381 |
| Ex-1.11 | 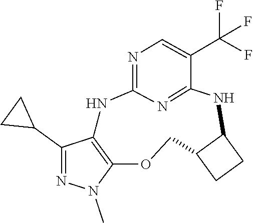 | (10aR,12aR)- or (10aS,12aS)-3- cyclopropyl-1-methyl-8- (trifluoromethyl)-1,4,10,10a,11,12,- 12a,13-octahydro-5,9- (azeno)cyclobuta[k]pyrazolo[3,4- b][1]oxa[4,6,10]triazacyclotridecine | Calc'd 381, found 381 |
| Ex-1.12 | 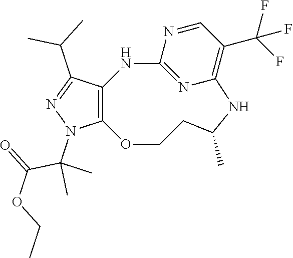 [SMILES: CCOC(=O)C(C)(C)n1nc(C(C)C)c2c1OCC[C@@H](C)Nc1nc(ncc1C(F)(F)F)N2] | ethyl (R)-2-(3-isopropyl-11-methyl- 8-(trifluoromethyl)-10,11,12,13- tetrahydro-5,9-(azeno)pyrazolo[3,4- b][1]oxa[4,6,10]triazacyclotridecin- 1(4H)-yl)-2-methylpropanoate | Calc'd 471, found 471 |
| Ex-1.13 | 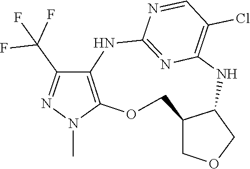 [SMILES: Cn1nc(C(F)(F)F)c2c1OC[C@@H]1COC[C@H]1Nc1nc(ncc1Cl)N2] | (10aS,13aS)-8-chloro-1-methyl-3- (trifluoromethyl)-1,4,10a,11,13a,14- hexahydro-10H,13H-5,9- (azeno)furo[3,4-k]pyrazolo[3,4- b][1]oxa[4,6,10]triazacyclotridecine | Calc'd 391, found 391 |
| Ex-1.14 | 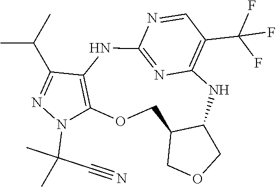 [SMILES: CC(C)c1nn(C(C)(C)C#N)c2c1Nc1ncc(C(F)(F)F)c(n1)N[C@@H]1COC[C@H]1CO2] | 2-((10aS,13aS)-3-isopropyl-8- (trifluoromethyl)-10a,11,13a,14- tetrahydro-10H,13H-5,9- (azeno)furo[3,4-k]pyrazolo[3,4- b][1]oxa[4,6,10]triazacyclotridecin- 1(4H)-yl)-2-methylpropanenitrile | Calc'd 452, found 452 |
| Ex-1.15 | 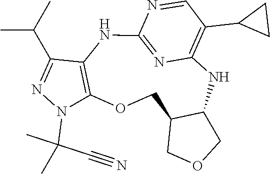 [SMILES: CC(C)c1nn(C(C)(C)C#N)c2c1Nc1ncc(C3CC3)c(n1)N[C@@H]1COC[C@H]1CO2] | 2-((10aS,13aS)-8-cyclopropyl-3- isopropyl-10a,11,13a,14-tetrahydro- 10H,13H-5,9-(azeno)furo[3,4- k]pyrazolo[3,4- b][1]oxa[4,6,10]triazacyclotridecin- 1(4H)-yl)-2-methylpropanenitrile | Calc'd 424, found 424 |
| Ex-1.16 | 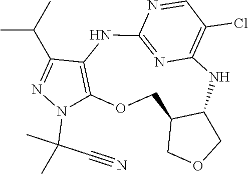 [SMILES: CC(C)c1nn(C(C)(C)C#N)c2c1Nc1ncc(Cl)c(n1)N[C@@H]1COC[C@H]1CO2] | 2-((10aS,13aS)-8-chloro-3- isopropyl-10a,11,13a,14-tetrahydro- 10H,13H-5,9-(azeno)furo[3,4- k]pyrazolo[3,4- b][1]oxa[4,6,10]triazacyclotridecin- 1(4H)-yl)-2-methylpropanenitrile | Calc'd 418, found 418 |
| Ex-1.17 | 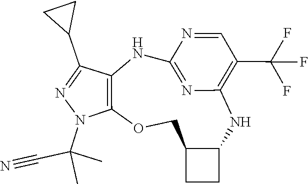 [SMILES: CC(C)(C#N)n1nc(C2CC2)c2c1OC[C@@H]1CC[C@H]1Nc1nc(ncc1C(F)(F)F)N2] | 2-((10aR,12aR)-3-cyclopropyl-8- (trifluoromethyl)- 10,10a,11,12,12a,13-hexahydro-5,9- (azeno)cyclobuta[k]pyrazolo[3,4- b][1]oxa[4,6,10]triazacyclotridecin- 1(4H)-yl)-2-methylpropanenitrile | Calc'd 434, found 434 |
| Ex-1.18 | 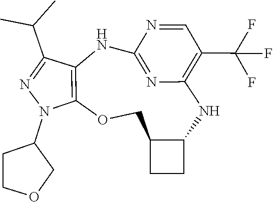 [SMILES: CC(C)c1nn(C2CCOC2)c2c1Nc1ncc(C(F)(F)F)c(n1)N[C@@H]1CC[C@H]1CO2] | (10aR,12aR)-3-isopropyl-1-((R)- or (10aR,12aR)-3-isopropyl-1-((S)- tetrahydrofuran-3-yl)-8- (trifluoromethyl)- 1,4,10,10a,11,12,12a,13-octahydro- 5,9- (azeno)cyclobuta[k]pyrazolo[3,4- b][1]oxa[4,6,10]triazacyclotridecine | Calc'd 439, found 439 |
| Ex-1.19 | 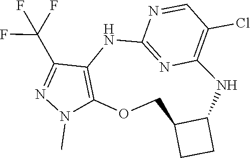 [SMILES: Cn1nc(C(F)(F)F)c2c1OC[C@@H]1CC[C@H]1Nc1nc(ncc1Cl)N2] | (10aR,12aR)-8-chloro-1-methyl-3- (trifluoromethyl)- 1,4,10,10a,11,12,12a,13-octahydro- 5,9- (azeno)cyclobuta[k]pyrazolo[3,4- b][1]oxa[4,6,10]triazacyclotridecine | Calc'd 375, found 375 |
| Ex-1.20 | 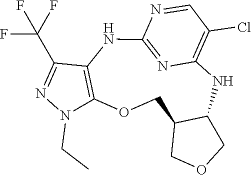 [SMILES: CCn1nc(C(F)(F)F)c2c1OC[C@@H]1COC[C@H]1Nc1nc(ncc1Cl)N2] | (10aS,13aS)-8-chloro-1-ethyl-3- (trifluoromethyl)-1,4,10a,11,13a,14- hexahydro-10H,13H-5,9- (azeno)furo[3,4-k]pyrazolo[3,4- b][1]oxa[4,6,10]triazacyclotridecine | Calc'd 405, found 405 |
| Ex-1.21 | 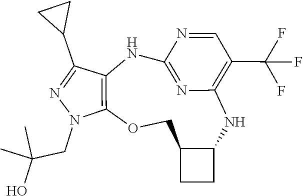 [SMILES: CC(C)(O)Cn1nc(C2CC2)c2c1OC[C@@H]1CC[C@H]1Nc1nc(ncc1C(F)(F)F)N2] | 1-((10aR,12aR)-3-cyclopropyl-8- (trifluoromethyl)- 10,10a,11,12,12a,13-hexahydro-5,9- (azeno)cyclobuta[k]pyrazolo[3,4- b][1]oxa[4,6,10]triazacyclotridecin- 1(4H)-yl)-2-methylpropan-2-ol | Calc'd 439, found 439 |
| Ex-1.22 | 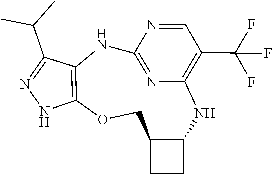 [SMILES: CC(C)c1n[nH]c2c1Nc1ncc(C(F)(F)F)c(n1)N[C@@H]1CC[C@H]1CO2] | (10aR,12aR)-3-isopropyl-8- (trifluoromethyl)- 1,4,10,10a,11,12,12a,13-octahydro- 5,9- (azeno)cyclobuta[k]pyrazolo[3,4- b][1]oxa[4,6,10]triazacyclotridecine | Calc'd 369, found 369 |
| Ex-1.23 | 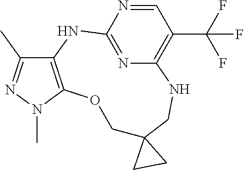 [SMILES: Cc1nn(C)c2c1Nc1ncc(C(F)(F)F)c(n1)NCC1(CC1)CO2] | 1′,3′-dimethyl-8′-(trifluoromethyl)- 1′,4′,10′,11′-tetrahydro-13′H- spiro[cyclopropane-1,12′- [5,9](azeno)pyrazolo[3,4- b][1]oxa[4,6,10]triazacyclotridecine] | Calc'd 3355, found 355 |

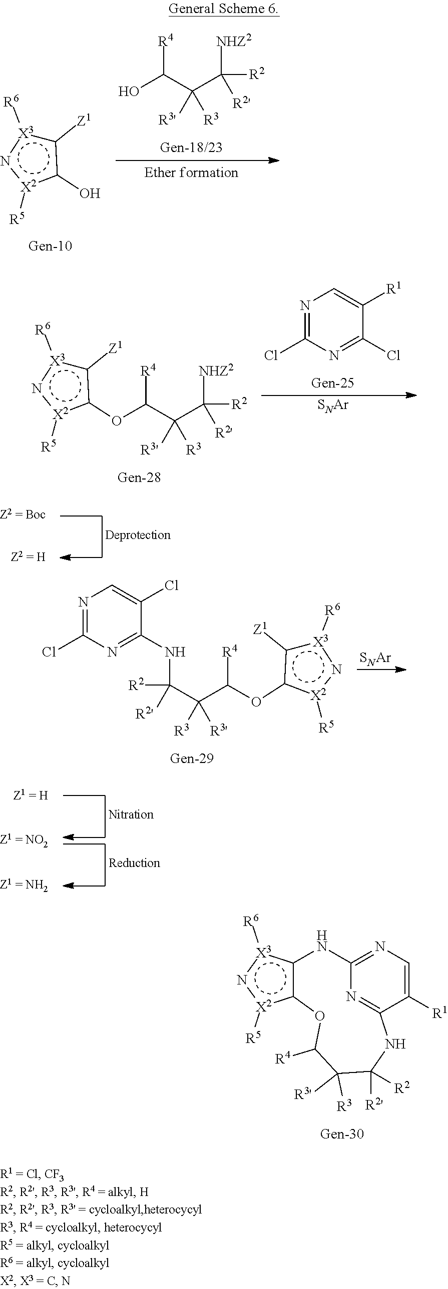

In General Scheme 6, pyrazole alcohols of the form Gen-10 were coupled with commercially available or synthetically prepared amino-alcohols of the form Gen-18 or Gen 23 through Mitsunobu chemistry or derivatives of the Mitsunobu reaction to provide Gen-28. In instances of Gen-28 where Z²=Boc, acidic deprotection to the amine was performed. Commercially available pyrimidines of the form Gen-25 were coupled with amines of the form Gen-1 through S_{N}Ar chemistry to access Gen-29. In instances of Gen-3 where Z¹=H, nitration of the pyrazole was performed. For nitropyrazoles of the form Gen-3 where Z¹=NO₂, reduction to the corresponding amine was performed. The aminopyrazole Gen-29 could ultimately be transformed through intramolecular S_{N}Ar chemistry to afford elaborated compounds of the form Gen-30. Representative preparative examples from each sequence are described in more detail below.

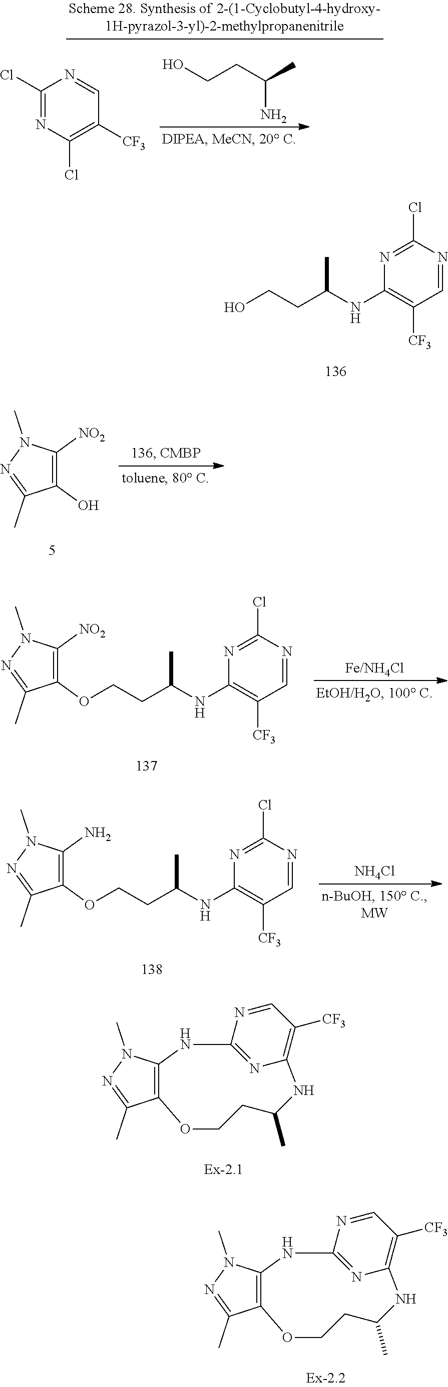

### 3-((2-Chloro-5-(trifluoromethyl)pyrimidin-4-yl)amino) butan-1-ol (136)

To a solution of 2,4-dichloro-5-(trifluoromethyl)pyrimidine (2.00 g, 9.22 mmol) in MeCN (8 mL) was added 3-aminobutan-1-ol (0.886 mL, 9.22 mmol) and DIPEA (3.22 mL, 18.4 mmol). The mixture was allowed to stir at 20° C. for 16 h, then was poured into water (20 mL) and extracted with EtOAc (50 mL×3). The combined organic layers were washed with brine (20 mL×3), dried over Na₂SO₄. The solution was filtered and concentrated under reduced pressure. The mixture was filtered, and the filtrated was concentrated under reduced pressure to give a

### (R)- and (S)-N-(4-((5-amino-1,3-dimethyl-1H-pyrazol-4-yl)oxy) butan-2-yl)-2-chloro-5-(trifluoromethyl)pyrimidin-4-amine (138)

To a solution of ammonia hydrochloride (17.01 mg, 0.318 mmol), (R)- and (S)-2-chloro-N-(4-((1,3-dimethyl-5-nitro-1H-pyrazol-4-yl)oxy) butan-2-yl)-5-(trifluoromethyl)pyrimidin-4-amine 137 (130 mg, 0.318 mmol) in EtOH (10 mL) and water (1 mL) was added iron (17.8 mg, 0.318 mmol). The mixture was allowed to stir at 100° C. for 3 h. The mixture was filtered and concentrated under reduced pressure to afford the title compound 138.

### (R)- and (S)-1,3,7-trimethyl-10-(trifluoromethyl)-1,5,6,7,8,14-hexahydro-9,13-(azeno)pyrazolo[4,3-b][1]oxa[4,6,10]triazacyclotridecine (Ex-2.1 and 2.2)

To a solution of (R)- and (S)-N-(4-((5-amino-1,3-dimethyl-1H-pyrazol-4-yl)oxy) butan-2-yl)-2-chloro-5-(trifluoromethyl)pyrimidin-4-amine 138 (90 mg, 0.238 mmol) in n-butyl alcohol (6 mL) was added NH₄Cl (25.4 mg, 0.475 mmol). The reaction mixture was then heated to 150° C. and allowed to stir for 3 h in the microwave. The reaction mixture was concentrated under reduced pressure. The crude product was purified by reversed phase HPLC, eluting with water (0.1% TFA)-MeCN, to afford the racemic material. The racemic material could be resolved to its component enantiomers by chiral preparative SFC (Column: Chiralpak AS-3, 150×4.6 mm I.D., 3 μm; Mobile phase: A: CO₂ B: ethanol (0.05% DIPEA); Gradient: from 5% to 40% of B in 5 min and from 40% to 5% of B in 0.5 min, hold 5% of B for 1.5 min) to afford the title compounds Ex-2.1 (t_{R}=1.7 min) and 2.2 (t_{R}=2.1 min). MS (ESI): m/z calc'd for C₁₄H₁₇F₃N₆O [M+H]⁺: 343, found 343; ¹H NMR (500 MHz, MeOD) δ 8.06 (s, 1H), 4.38-4.30 (m, 1H), 4.13-4.01 (m, 1H), 3.91-3.70 (m, 1H), 3.58-3.42 (m, 3H), 2.14-2.05 (m, 3H), 1.87-1.76 (m, 1H), 1.72-1.61 (m, 1H), 1.22 (d, J=7.0 Hz, 3H); MS (ESI): m/z calc'd for C₁₄H₁₇F₃N₆O [M+H]⁺: 343, found 343; ¹H NMR (500 MHz, MeOD) δ 8.02 (s, 1H), 4.36-4.28 (m, 1H), 4.06-3.97 (m, 1H), 3.80-3.66 (m, 1H), 3.62-3.46 (m, 3H), 2.14-2.02 (m, 3H), 1.84-1.75 (m, 1H), 1.69-1.61 (m, 1H), 1.20 (d, J=7.2 Hz, 3H).

### Scheme 29. Synthesis of (10aR,12aR)- or (10aS,12aS)-3-cyclobutyl-1-methyl-8-(trifluoromethyl)-3,4,10,10a,11,12,12a,13-octahydro-5,9-(azeno)cyclobuta[k]pyrazolo[4,3-b][1]oxa[4,6,10]triazacyclotridecine

4.01 (m, 1H), 3.91-3.70 (m, 1H), 3.58-3.42 (m, 3H), 2.14-2.05 (m, 3H), 1.87-1.76 (m, 1H), 1.72-1.61 (m, 1H), 1.22 (d, J=7.0 Hz, 3H); MS (ESI): m/z calc'd for C₁₄H₁₇F₃N₆O [M+H]⁺: 343, found 343; ¹H NMR (500 MHz, MeOD) δ 8.02 (s, 1H), 4.36-4.28 (m, 1H), 4.06-3.97 (m, 1H), 3.80-3.66 (m, 1H), 3.62-3.46 (m, 3H), 2.14-2.02 (m, 3H), 1.84-1.75 (m, 1H), 1.69-1.61 (m, 1H), 1.20 (d, J=7.2 Hz, 3H).

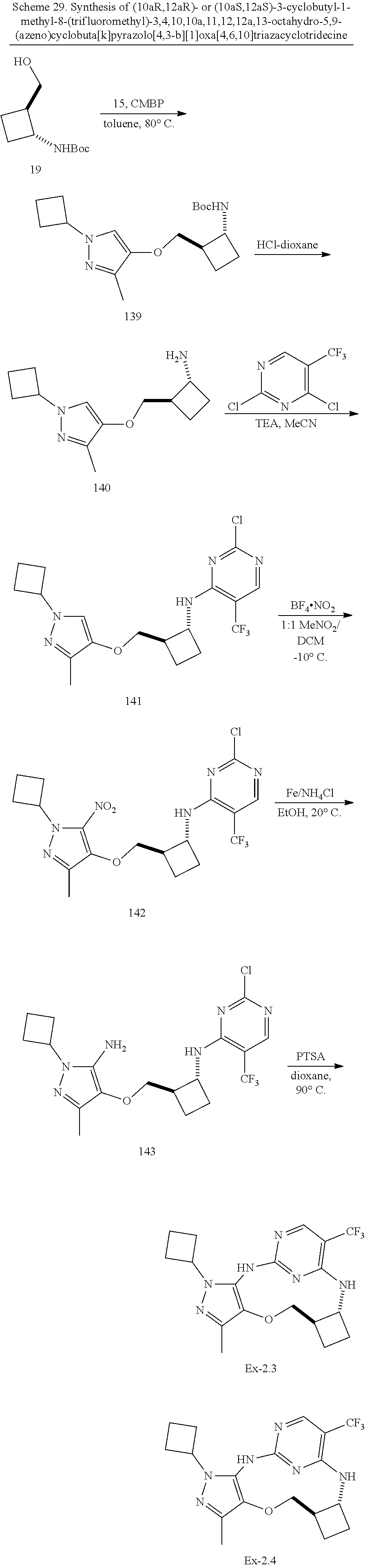

### tert-Butyl ((1R,2R)- and (1S,2S)-2-(((1-cyclobutyl-3-methyl-1H-pyrazol-4-yl)oxy)methyl)cyclobutyl)carbamate (139)

To a solution of 1-cyclobutyl-3-methyl-1H-pyrazol-4-ol 19 (529 mg, 3.48 mmol) and tert-butyl (-2-(hydroxymethyl)cyclobutyl)carbamate 15 (700 mg, 3.48 mmol) in anhydrous Toluene (12 mL) was added (tributylphosphoranylidene) acetonitrile (923 mg, 3.83 mmol), and the reaction mixture was allowed to stir at 80° C. under N₂ for 16 h. Water (40 mL) was added to the mixture, and the mixture was extracted with EtOAc (60 mL×3). The combined organic layers were dried over Na₂SO₄ and filtered. The filtrate was concentrated under reduced pressure. The crude product purified by flash chromatography on silica gel (gradient elution of 0-70% EtOAc/PE) to afford the title compound 139.

To a solution of (1R,2R)- and (1S,2S)-2-(((1-cyclobutyl-3-methyl-1H-pyrazol-4-yl)oxy)methyl)cyclobutan-1-amine 140 (550 mg, 2.337 mmol) and 2,4-dichloro-5-(trifluoromethyl)pyrimidine (507 mg, 2.337 mmol) in acetonitrile (20 mL) was added triethylamine (710 mg, 7.01 mmol) at 20° C. The mixture was allowed to stir for 1 h at 20° C., then was concentrated under reduced pressure. The crude product was purified by reversed phase HPLC, eluting with water (0.1% NH₄OH)-MeCN, to afford the title compound 141.

### 2-Chloro-N-((1R,2R)- and (1S,2S)-2-(((1-cyclobutyl-3-methyl-5-nitro-1H-pyrazol-4-yl)oxy)methyl)cyclobutyl)-5-(trifluoromethyl)pyrimidin-4-amine (142)

To a mixture of 2-chloro-N-((1R,2S)- and (1S,2S)-2-(((1-cyclobutyl-3-methyl-1H-pyrazol-4-yl)oxy)methyl)cyclobutyl)-5-(trifluoromethyl)pyrimidin-4-amine (250 mg, 0.601 mmol) 141 in DCM (4 mL) and MeNO₂ (4.00 mL) was added nitronium tetrafluoroborate (120 mg, 0.902 mmol) at −10° C. The reaction mixture was allowed to stir at −10° C. for 30 min then allowed to stir at 0° C. for 30 min, then quenched with H₂O (20 mL). DCM (30 mL) was added into the mixture, and the organic layer was separated. The aqueous layer was extracted with DCM (30 mL×2). The combined extracts were dried over anhydrous Na₂SO₄ and filtered. The filtrate was concentrated under reduced pressure. The crude product was purified by prep-TLC (2:1 PE/EtOAc) to afford the title compound 142.

### N-((1R,2R)- and (1S,2S)-2-(((5-Amino-1-cyclobutyl-3-methyl-1H-pyrazol-4-yl)oxy)methyl)cyclobutyl)-2-chloro-5-(trifluoromethyl)pyrimidin-4-amine (143)

To a solution of 2-chloro-N-((1R,2R)- and (1S,2S)-2-(((1-cyclobutyl-3-methyl-5-nitro-1H-pyrazol-4-yl)oxy)methyl)cyclobutyl)-5-(trifluoromethyl)pyrimidin-4-amine 142 (140 mg, 0.304 mmol) and ammonia hydrochloride (162 mg, 3.04 mmol) in EtOH (20 mL) and water (2 mL) was added iron (170 mg, 3.04 mmol). The mixture was allowed to stir at 90° C. for 1 h. The solution was filtered and concentrated under reduced pressure. The reaction mixture was poured into water (10 mL) and extracted with EtOAc (20 mL×3). The organic layer was washed with brine (10 mL) and dried over Na₂SO₄. The organic phase was concentrated afford the title compound 143 which was directly used to next step without further purification.

### (10aR,12aR)- or (10aS,12aS)-3-Cyclobutyl-1-methyl-8-(trifluoromethyl)-3,4,10,10a,11,12,12a,13-octahydro-5,9-(azeno)cyclobuta[k]pyrazolo[4,3-b][1]oxa[4,6,10]triazacyclotridecine (Ex-2.3 and Ex-2.4)

To a solution of N-((1R,2R)- and (1S,2S)-2-(((5-Amino-1-cyclobutyl-3-methyl-1H-pyrazol-4-yl)oxy)methyl)cyclobutyl)-2-chloro-5-(trifluoromethyl)pyrimidin-4-amine 143 (100 mg, 0.232 mmol) in 1,4-dioxane (5 mL) was added 4-methylbenzenesulfonic acid (11.99 mg, 0.070 mmol) under N₂. The mixture was allowed to stir at 90° C. for 12 h, then was concentrated under reduced pressure. The crude product was purified by reversed phase HPLC, eluting with water (0.1% TFA)-MeCN, to afford the racemic material. The racemic material was resolved to its component enantiomers by chiral preparative SFC (Column: Chiralpak IC-3 150×4.6 mm I.D., 3 μm. Mobile phase: A: CO₂ B: ethanol (0.05% DEA). Gradient: from 5% to 40% of B in 4 min and hold 40% for 2.5 min, then 5% of B for 1.5 min. Flow rate: 2.5 mL/min. Column temp.: 35° C. ABPR: 1500 psi) to afford the title compounds Ex-2.3 (t_{R}=2.0 min) and Ex-2.4 (t_{R}=2.9 min). MS (ESI): m/z calc'd for C₁₈H₂₁F₃N₆O [M+H]⁺: 395, found 395; ¹H NMR (400 MHz, CDCl₃) δ=8.26-8.03 (m, 1H), 7.98 (s, 1H), 5.30 (br s, 1H), 4.60 (quin, J=8.3 Hz, 1H), 4.37 (m, 1H), 4.02-3.85 (m, 1H), 3.74-3.67 (m, 1H), 2.73 (quin, J=10.0 Hz, 1H), 2.55-2.44 (m, 2H), 2.36-2.25 (m, 6H), 2.03 (q, J=10.2 Hz, 1H), 1.89-1.72 (m, 3H), 1.49-1.31 (m, 1H), 1.28-1.28 (m, 1H), 1.25-1.16 (m, 1H), 1.12-1.12 (m, 1H); MS (ESI): m/z calc'd for C₁₈H₂₁F₃N₆O [M+H]⁺: 395, found 395; ¹H NMR (400 MHz, CDCl₃) δ=8.19 (br s, 1H), 7.96 (s, 1H), 5.26 (br s, 1H), 4.60 (quin, J=8.3 Hz, 1H), 4.36 (m, 1H), 4.00-3.89 (m, 1H), 3.79-3.66 (m, 1H), 2.77-2.60 (m, 1H), 2.60-2.35 (m, 2H), 2.34-2.19 (m, 6H), 2.09-1.99 (m, 1H), 1.85-1.61 (m, 3H), 1.46-1.34 (m, 1H).

Compounds in Table 5 below were prepared in accordance with the synthetic sequence illustrated in General Scheme 6 using the corresponding starting materials.

TABLE 5

| | | | Exact |
|---|---|---|---|
| | | | Mass |
| Ex | Structure | Name | [M + H]⁺ |
| Ex-2.5 | 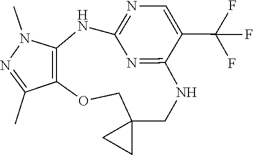 [SMILES: Cc1nn(C)c2c1OCC1(CC1)CNc1nc(ncc1C(F)(F)F)N2] | 1′,3′-dimethyl-10′-(trifluoromethyl)- 1′,7′,8′,14′-tetrahydro-5′H- spiro[cyclopropane-1,6′- [9,13](azeno)pyrazolo[4,3- b][1]oxa[4,6,10]triazacyclotridecine] | Calc'd 355, found 355 |
| Ex-2.6 | 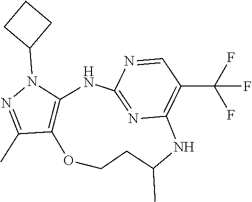 [SMILES: Cc1nn(C2CCC2)c2c1OCCC(C)Nc1nc(ncc1C(F)(F)F)N2] | (R)- or (S)-1-cyclobutyl-3,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 383, found 383 |
| Ex-2.7 | 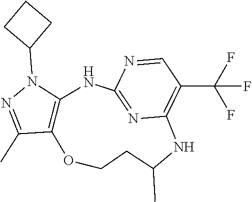 [SMILES: Cc1nn(C2CCC2)c2c1OCCC(C)Nc1nc(ncc1C(F)(F)F)N2] | (R)- or (S)-1-cyclobutyl-3,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 383, found 383 |
| Ex-2.5 | 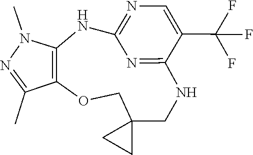 [SMILES: Cc1nn(C)c2c1OCC1(CC1)CNc1nc(ncc1C(F)(F)F)N2] | 1′,3′-dimethyl-10′-(trifluoromethyl)- 1′,7′,8′,14′-tetrahydro-5′H- spiro[cyclopropane-1,6′- [9,13](azeno)pyrazolo[4,3- b][1]oxa[4,6,10]triazacyclotridecine] | Calc'd 355, found 355 |
| Ex-2.6 | 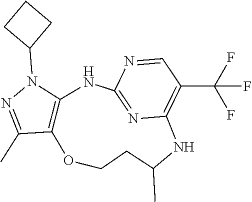 [SMILES: Cc1nn(C2CCC2)c2c1OCCC(C)Nc1nc(ncc1C(F)(F)F)N2] | (R)- or (S)-1-cyclobutyl-3,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 383, found 383 |
| Ex-2.7 | 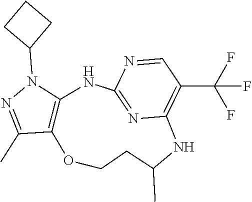 [SMILES: Cc1nn(C2CCC2)c2c1OCCC(C)Nc1nc(ncc1C(F)(F)F)N2] | (R)- or (S)-1-cyclobutyl-3,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 383, found 383 |
| Ex-2.8 | 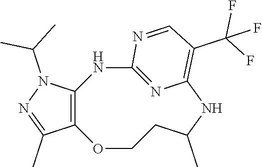 [SMILES: Cc1nn(C(C)C)c2c1OCCC(C)Nc1nc(ncc1C(F)(F)F)N2] | (R)- or (S)-1-isopropyl-3,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 371, found 371 |
| Ex-2.9 | 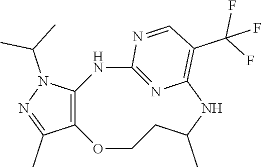 [SMILES: Cc1nn(C(C)C)c2c1OCCC(C)Nc1nc(ncc1C(F)(F)F)N2] | (R)- or (S)-1-isopropyl-3,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 371, found 371 |
| Ex-2.10 | 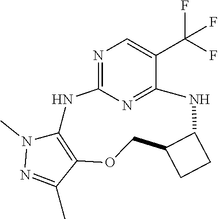 | (10aR,12aR)- or (10aS,12aS)-1,3-dimethyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecine | Calc'd 355, found 355 |
| Ex-2.11 | 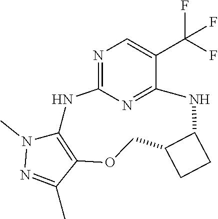 | (10aR,12aS)- or (10aS,12aR)-1,3-dimethyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecine | Calc'd 355, found 355 |
| Ex-2.12 | 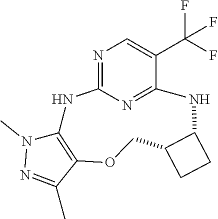 | (10aR,12aS)- or (10aS,12aR)-1,3-dimethyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecine | Calc'd 355, found 355 |
| Ex-2.13 | 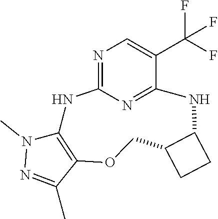 | (10aR,12aS)- or (10aS,12aR)-1,3-dimethyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecine | Calc'd 355, found 355 |
| Ex-2.14 | 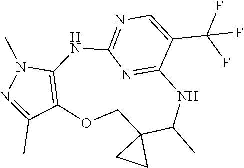 [SMILES: Cc1nn(C)c2c1OCC1(CC1)C(C)Nc1nc(ncc1C(F)(F)F)N2] | (R)- or (S)-1′,3′,7′-trimethyl-10′- (trifluoromethyl)-1′,7′,8',14′-tetrahydro-5′H- spiro[cyclopropane-1,6′- [9,13](azeno)pyrazolo[4,3- b][1]oxa[4,6,10]triazacyclotridecine] | Calc'd 369, found 369 |
| Ex-2.15 | 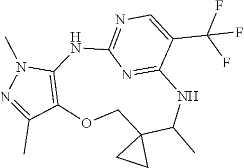 [SMILES: Cc1nn(C)c2c1OCC1(CC1)C(C)Nc1nc(ncc1C(F)(F)F)N2] | (R)- or (S)-1′,3′,7′-trimethyl-10′- (trifluoromethyl)-1′,7′,8′,14′-tetrahydro-5′H- spiro[cyclopropane-1,6′- [9,13](azeno)pyrazolo[4,3- b][1]oxa[4,6,10]triazacyclotridecine] | Calc'd 369, found 369 |
| Ex-2.16 | 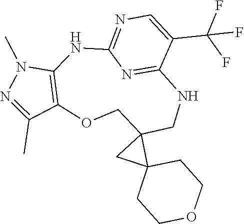 [SMILES: Cc1nn(C)c2c1OCC1(CNc3nc(ncc3C(F)(F)F)N2)CC12CCOCC2] | (R)- or (S)-1″,3″-dimethyl-10″- (trifluoromethyl)-1″,2,3,5,6,7″,8″,14″. octahydro-5″H-dispiro[pyran-4,1′- cyclopropane-2′,6″- [9,13](azeno)pyrazolo[4,3- b][1]oxa[4,6,10]triazacyclotridecine] | Calc'd 425, found 425 |
| Ex-2.17 | 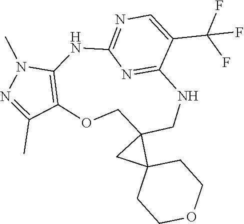 [SMILES: Cc1nn(C)c2c1OCC1(CNc3nc(ncc3C(F)(F)F)N2)CC12CCOCC2] | (R)- or (S)-1″,3″-dimethyl-10″- (trifluoromethyl)-1″,2,3,5,6,7″,8″,14″- octahydro-5″H-dispiro[pyran-4,1′- cyclopropane-2′,6″- [9,13](azeno)pyrazolo[4,3- b][1]oxa[4,6,10]triazacyclotridecine] | Calc'd 425, found 425 |
| Ex-2.18 | 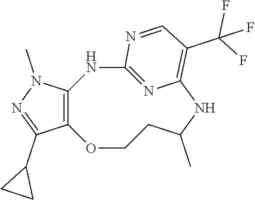 [SMILES: CC1CCOc2c(C3CC3)nn(C)c2Nc2ncc(C(F)(F)F)c(n2)N1] | (R)- or (S)-3-cyclopropyl-1,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 369, found 369 |
| Ex-2.19 | 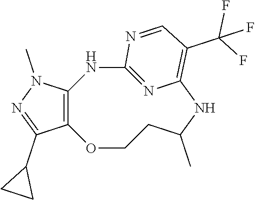 [SMILES: CC1CCOc2c(C3CC3)nn(C)c2Nc2ncc(C(F)(F)F)c(n2)N1] | (R)- or (S)-3-cyclopropyl-1,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 369, found 369 |
| Ex-2.20 | 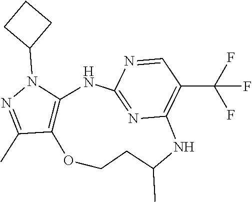 [SMILES: Cc1nn(C2CCC2)c2c1OCCC(C)Nc1nc(ncc1C(F)(F)F)N2] | (R)- or (S)-1-cyclopropyl-3,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 369, found 369 |
| Ex-2.21 | 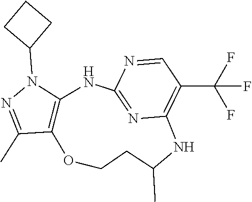 [SMILES: Cc1nn(C2CCC2)c2c1OCCC(C)Nc1nc(ncc1C(F)(F)F)N2] | (R)- or (S)-1-cyclopropyl-3,7-dimethyl-10- (trifluoromethyl)-1,5,6,7,8,14-hexahydro- 9,13-(azeno)pyrazolo[4,3- b][1]oxa[4,6,10]triazacyclotridecine | Calc'd 369, found 369 |
| Ex-2.22 | 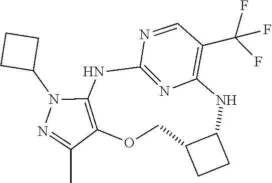 [SMILES: Cc1nn(C2CCC2)c2c1OC[C@H]1CC[C@H]1Nc1nc(ncc1C(F)(F)F)N2] | (10aR,12aS)- or (10aS,12aR)-3-cyclobutyl-1-methyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecine | Calc'd 395, found 395 |
| Ex-2.23 | 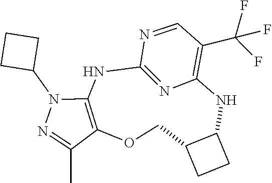 [SMILES: Cc1nn(C2CCC2)c2c1OC[C@H]1CC[C@H]1Nc1nc(ncc1C(F)(F)F)N2] | (10aR,12aS)- or (10aS,12aR)-3-cyclobutyl-1-methyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecine | Calc'd 395, found 395 |
| Ex-2.24 | 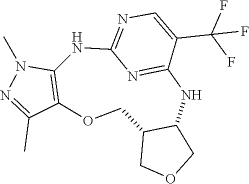 [SMILES: Cc1nn(C)c2c1OC[C@H]1COC[C@H]1Nc1nc(ncc1C(F)(F)F)N2] | (10aS,13aR)- or (10aR,13aS)-1,3-dimethyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 371, found 371 |
| Ex-2,25 | 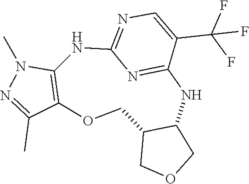 [SMILES: Cc1nn(C)c2c1OC[C@H]1COC[C@H]1Nc1nc(ncc1C(F)(F)F)N2] | (10aS,13aR)- or (10aR,13aS)-1,3-dimethyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 371, found 371 |
| Ex-2.26 | 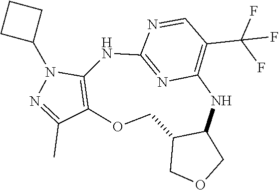 [SMILES: Cc1nn(C2CCC2)c2c1OC[C@H]1COC[C@@H]1Nc1nc(ncc1C(F)(F)F)N2] | (10aS,13aS)- or (10aR,13aR)-3-cyclobutyl-1-methyl-8- (trifluoromethyl)-3,4,10a,11,13a, 14- hexahydro-10H,13H-5,9-(azeno)furo[3,4- k]pyrazolo[4,3- b][1]oxa[4,6,10]triazacyclotridecine | Calc'd 411, found 411 |
| Ex-2.27 | 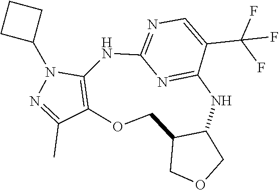 [SMILES: Cc1nn(C2CCC2)c2c1OC[C@@H]1COC[C@H]1Nc1nc(ncc1C(F)(F)F)N2] | (3aR,15aR)- or (3aS,15aS)- 11-cyclobutyl-13-methyl-6- (trifluoromethyl)-2,3,3a,4,10,11,15,15a- octahydro-5,9-(azeno)furo[3,2- k]pyrazolo[4,3- b][1]oxa[4,6,10]triazacyclotridecine | Calc'd 411, found 411 |
| Ex-2.28 | 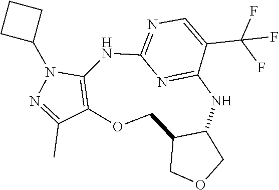 [SMILES: Cc1nn(C2CCC2)c2c1OC[C@@H]1COC[C@H]1Nc1nc(ncc1C(F)(F)F)N2] | (3aR,15aR)- or (3aS,15aS)- 11-cyclobutyl-13-methyl-6- (trifluoromethyl)-2,3,3a,4,10,11,15,15a- octahydro-5,9-(azeno)furo[3,2- k]pyrazolo[4,3- b][1]oxa[4,6,10]triazacyclotridecine | Calc'd 411, found 411 |
| Ex-2.29 | 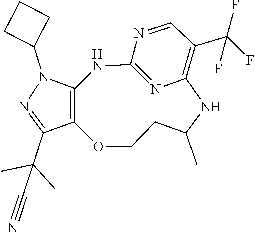 [SMILES: CC1CCOc2c(C(C)(C)C#N)nn(C3CCC3)c2Nc2ncc(C(F)(F)F)c(n2)N1] | (R)- or (S)-butyl-7-methyl-10- (trifluoromethyl)-1,5,6,7,8,14-hexahydro- 9,13-(azeno)pyrazolo[4,3- b][1]oxa[4,6,10]triazacyclotridecin-3-yl)-2- methylpropanenitrile | Calc'd 436, found 436 |
| Ex-2.30 | 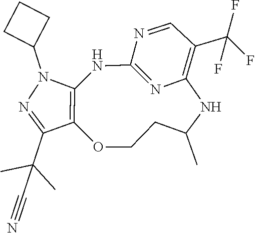 [SMILES: CC1CCOc2c(C(C)(C)C#N)nn(C3CCC3)c2Nc2ncc(C(F)(F)F)c(n2)N1] | (R)- or (S)-butyl-7-methyl-10- (trifluoromethyl)-1,5,6,7,8,14-hexahydro- 9,13-(azeno)pyrazolo[4,3- b][1]oxa[4,6,10]triazacyclotridecin-3-yl)-2- methylpropanenitrile | Calc'd 436, found 436 |
| Ex-2.31 | 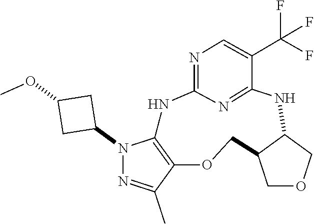 | (10aS,13aS)- or (10aR,13aR)-3-((1R,3S)-3- methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 441, found 441 |
| Ex-2.32 | 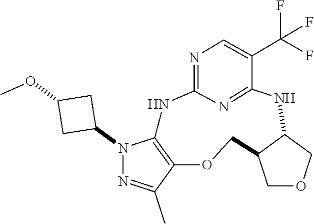 | (10aS,13aS)- or (10aR,13aR)-3-((1R,3S)-3- methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 441 found 441 |
| Ex-2.33 | 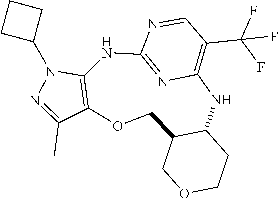 [SMILES: Cc1nn(C2CCC2)c2c1OC[C@@H]1COCC[C@H]1Nc1nc(ncc1C(F)(F)F)N2] | (4aR,16aS)- or (4aS,16aR)-12-cyclobutyl-14-methyl-7- (trifluoromethyl)-3,4,4a,5,11,12,16,16a- octahydro-1H-6,10-(azeno)pyrano[4,3- k]pyrazolo[4,3- b][1]oxa[4,6,10]triazacyclotridecine | Calc'd 425, found 425 |
| Ex-2.34 | 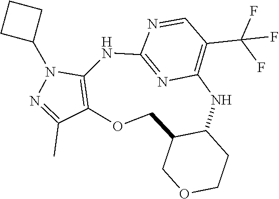 [SMILES: Cc1nn(C2CCC2)c2c1OC[C@@H]1COCC[C@H]1Nc1nc(ncc1C(F)(F)F)N2] | (4aR,16aS)- or (4aS,16aR)-12-cyclobutyl-14-methyl-7- (trifluoromethyl)-3,4,4a,5,11,12,16,16a- octahydro-1H-6,10-(azeno)pyrano[4,3- k]pyrazolo[4,3- b][1]oxa[4,6,10]triazacyclotridecine | Calc'd 425, found 425 |
| Ex-2.35 | 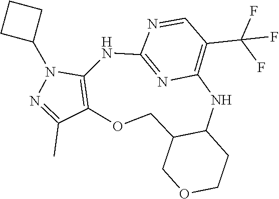 [SMILES: Cc1nn(C2CCC2)c2c1OCC1COCCC1Nc1nc(ncc1C(F)(F)F)N2] | (4aR,16aR)- and (4aS,16aS)-12-cyclobutyl-14-methyl-7- (trifluoromethyl)-3,4,4a,5,11,12,16,16a- octahydro-1H-6,10-(azeno)pyrano[4,3- k]pyrazolo[4,3- b][1]oxa[4,6,10]triazacyclotridecine | Calc'd 425, found 425 |
| Ex-2.36 | 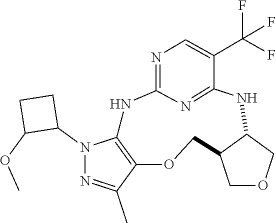 | (10aS,13aS)-3-((1R,2R)- (10aS,13aS)-3-((1S,2R)- (10aS,13aS)-3-((1R,2S)- (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 441, found 441 |
| Ex-2.37 | 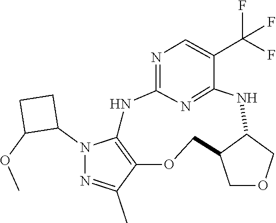 | (10aS,13aS)-3-((1R,2R)- (10aS,13aS)-3-((1S,2R)- (10aS,13aS)-3-((1R,2S)- (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 441, found 441 |
| Ex-2.38 | 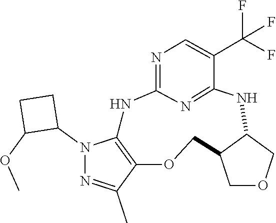 | (10aS,13aS)-3-((1R,2R)- (10aS,13aS)-3-((1S,2R)- (10aS,13aS)-3-((1R,2S)- (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 441, found 441 |
| Ex-2.39 | 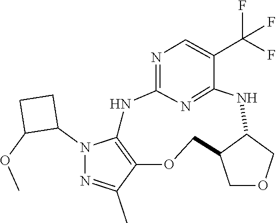 | (10aS,13aS)-3-((1R,2R)- (10aS,13aS)-3-((1S,2R)- (10aS,13aS)-3-((1R,2S)- (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 441, found 441 |
| Ex-2.40 | 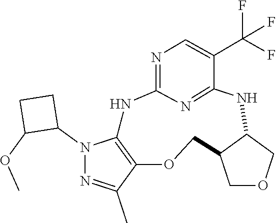 | (10aS,13aS)-3-((1R,2R)- (10aS,13aS)-3-((1S,2R)- (10aS,13aS)-3-((1R,2S)- (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 441, found 441 |
| Ex-2.41 | 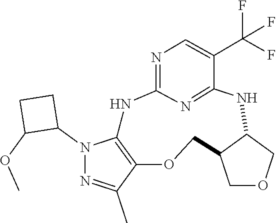 | (10aS,13aS)-3-((1R,2R)- (10aS,13aS)-3-((1S,2R)- (10aS,13aS)-3-((1R,2S)- (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 441, found 441 |
| Ex-2.42 | 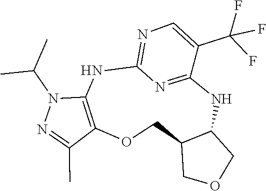 [SMILES: Cc1nn(C(C)C)c2c1OC[C@@H]1COC[C@H]1Nc1nc(ncc1C(F)(F)F)N2] | (10aS,13aS)-3-isopropyl-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 399, found 399 |
| Ex-2.43 | 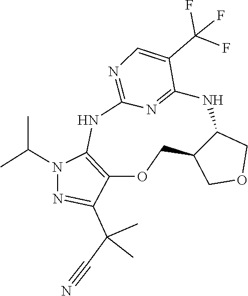 [SMILES: CC(C)n1nc(C(C)(C)C#N)c2c1Nc1ncc(C(F)(F)F)c(n1)N[C@@H]1COC[C@H]1CO2] | 2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1-yl)-2- methylpropanenitrile | Calc'd 452, found 452 |
| Ex-2.44 | 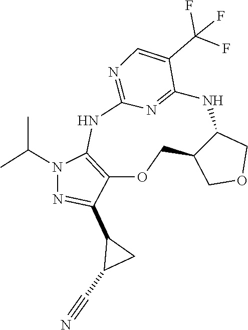 | (1S,2S)- or (1R,2R)-2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carbonitrile | Calc'd 450, found 450 |
| Ex-2.45 | 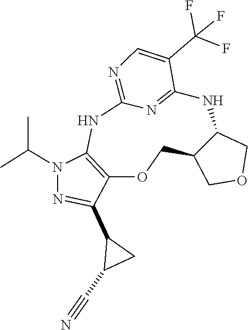 | (1S,2S)- or (1R,2R)-2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carbonitrile | Calc'd 450, found 450 |
| Ex-2.46 | 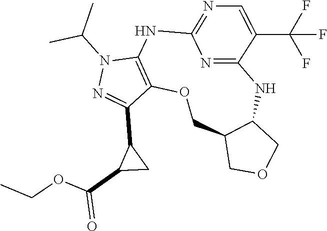 [SMILES: CCOC(=O)[C@@H]1C[C@@H]1c1nn(C(C)C)c2c1OC[C@@H]1COC[C@H]1Nc1nc(ncc1C(F)(F)F)N2] | ethyl (1R,2S)- or ethyl (1S,2R)-2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carboxylate | Calc'd 497, found 497 |
| Ex-2.47 | 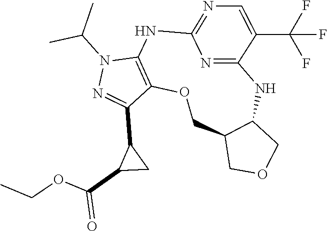 [SMILES: CCOC(=O)[C@@H]1C[C@@H]1c1nn(C(C)C)c2c1OC[C@@H]1COC[C@H]1Nc1nc(ncc1C(F)(F)F)N2] | ethyl (1R,2S)- or ethyl (1S,2R)-2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carboxylate | Calc'd 497, found 497 |
| Ex-2.48 | 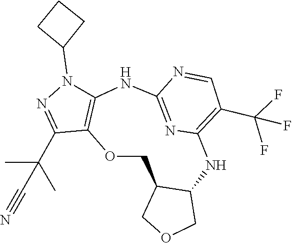 [SMILES: CC(C)(C#N)c1nn(C2CCC2)c2c1OC[C@@H]1COC[C@H]1Nc1nc(ncc1C(F)(F)F)N2] | 2-((10aS,13aS)-3-cyclobutyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1-yl)-2- methylpropanenitrile | Calc'd 464, found 464 |
| Ex-2.49 | 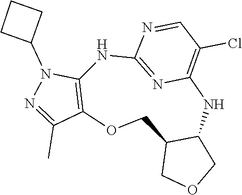 [SMILES: Cc1nn(C2CCC2)c2c1OC[C@@H]1COC[C@H]1Nc1nc(ncc1Cl)N2] | (10aS,13aS)-8-chloro-3-cyclobutyl-1- methyl-3,4,10a,11,13a,14-hexahydro- 10H,13H-5,9-(azeno)furo[3,4- k]pyrazolo[4,3- b][1]oxa[4,6,10]triazacyclotridecine | Calc'd 377, found 377 |
| Ex-2.50 | 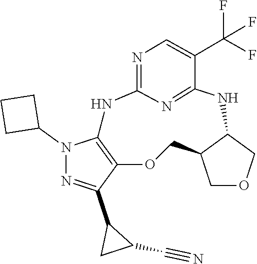 | (1R,2R)- or (1S,2S)-2-((10aS,13aS)-3-cyclobutyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carbonitrile | Calc'd 462, found 462 |
| Ex-2.51 | 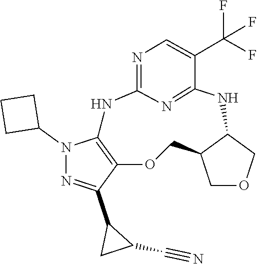 | (1R,2R)- or (1S,2S)-2-((10aS,13aS)-3-cyclobutyl-8- (trifluoromethyl)-3,4,10a,11,13a, 14- hexahydro-10H,13H-5,9-(azeno)furo[3,4- k]pyrazolo[4,3- b][1]oxa[4,6,10]triazacyclotridecin-1- yl)cyclopropane-1-carbonitrile | Calc'd 462, found 462 |
| Ex-2.52 | 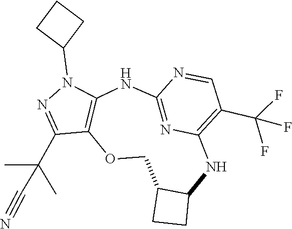 [SMILES: CC(C)(C#N)c1nn(C2CCC2)c2c1OC[C@H]1CC[C@@H]1Nc1nc(ncc1C(F)(F)F)N2] | 2-((10aS,12aS)- or 2-((10aR,12aR)-3-cyclobutyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecin-1-yl)-2- methylpropanenitrile | Calc'd 448, found 448 |
| Ex-2.53 | 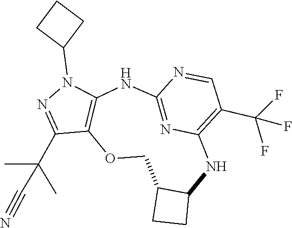 [SMILES: CC(C)(C#N)c1nn(C2CCC2)c2c1OC[C@H]1CC[C@@H]1Nc1nc(ncc1C(F)(F)F)N2] | 2-((10aS,12aS)- or 2-((10aR,12aR)-3-cyclobutyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecin-1-yl)-2- methylpropanenitrile | Calc'd 448, found 448 |
| Ex-2.54 | 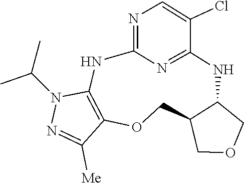 [SMILES: Cc1nn(C(C)C)c2c1OC[C@@H]1COC[C@H]1Nc1nc(ncc1Cl)N2] | (10aS,13aS)-8-chloro-3-isopropyl-1-methyl- 3,4,10a, 11,13a,14--hexahydro-10H,13H- 5,9-(azeno)furo[3,4-k]pyrazolo[4,3- b][1]oxa[4,6,10]triazacyclotridecine | Calc'd 365, found 365 |
| Ex-2.55 | 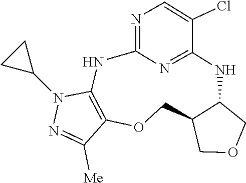 [SMILES: Cc1nn(C2CC2)c2c1OC[C@@H]1COC[C@H]1Nc1nc(ncc1Cl)N2] | (10aS,13aS)-8-chloro-3-cyclopropyl-1- methyl-3,4,10a,11,13a,14-hexahydro- 10H,13H-5,9-(azeno)furo[3,4- k]pyrazolo[4,3- b][1]oxa[4,6,10]triazacyclotridecine | Calc'd 363, found 363 |
| Ex-2.56 | 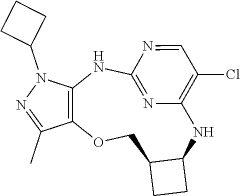 [SMILES: Cc1nn(C2CCC2)c2c1OC[C@@H]1CC[C@@H]1Nc1nc(ncc1Cl)N2] | (10aS,12aR)-8-chloro-3-cyclobutyl-1- methyl-3,4,10,10a,11,12,12a,13-octahydro- 5,9-(azeno)cyclobuta[k]pyrazolo[4,3- b][1]oxa[4,6,10]triazacyclotridecine | Calc'd 361, found 361 |
| Ex-2.57 | 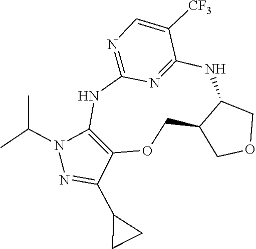 [SMILES: CC(C)n1nc(C2CC2)c2c1Nc1ncc(C(F)(F)F)c(n1)N[C@@H]1COC[C@H]1CO2] | (10aS,13aS)-1-cyclopropyl-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 425, found 425 |
| Ex-2.58 | 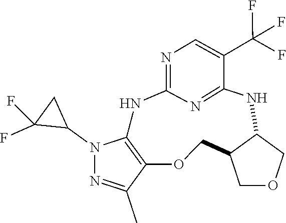 | (10aS,13aS)-3-((R)-2,2- difluorocyclopropyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine (10aS,13aS)-3-((S)-2,2- difluorocyclopropyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 433, found 433 |
| Ex-2.59 | 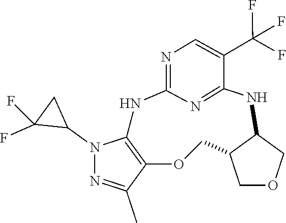 | (10aS,13aS)-3-((R)-2,2- difluorocyclopropyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine (10aS,13aS)-3-((S)-2,2- difluorocyclopropyl)-1-methyl-8- (trifluoromethyl)-3,4,10a,11,13a, 14- hexahydro-10H,13H-5,9-(azeno)furo[3,4- k]pyrazolo[4,3- b\|[1]oxa\|4,6,10]triazacyclotridecine | Calc'd 433, found 433 |
| Ex-2.60 | 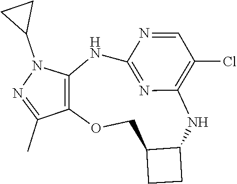 [SMILES: Cc1nn(C2CC2)c2c1OC[C@@H]1CC[C@H]1Nc1nc(ncc1Cl)N2] | (10aR,12aR)-8-chloro-3-cyclopropyl-1- methyl-3,4,10,10a,11,12,12a,13-octahydro- 5,9-(azeno)cyclobuta[k]pyrazolo[4,3- b][1]oxa[4,6,10]triazacyclotridecine | Calc'd 347, found 347 |
| Ex-2.61 | 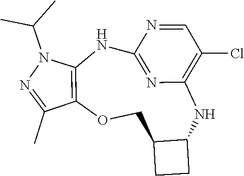 [SMILES: Cc1nn(C(C)C)c2c1OC[C@@H]1CC[C@H]1Nc1nc(ncc1Cl)N2] | (10aR,12aR)-8-chloro-3-isopropyl-1- methyl-3,4,10,10a,11,12,12a,13-octahydro- 5,9-(azeno)cyclobuta[k]pyrazolo[4,3- b][1]oxa[4,6,10]triazacyclotridecine | Calc'd 349, found 349 |
| Ex-2.62 | 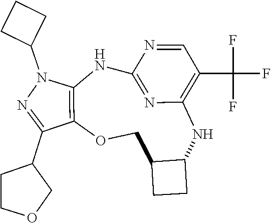 | (10aR,12aR)-3-cyclobutyl-1-((R)- (10aR,12aR)-3-cyclobutyl-1-((S)- (10aS,12aS)-3-cyclobutyl-1-((R)- (10aS,12aS)-3-cyclobutyl-1-((S)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine | Calc'd 451, found 451 |
| Ex-2.63 | 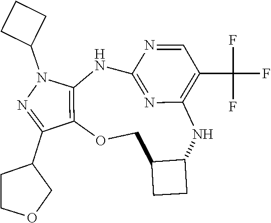 | (10aR,12aR)-3-cyclobutyl-1-((R)- (10aR,12aR)-3-cyclobutyl-1-((S)- (10aS,12aS)-3-cyclobutyl-1-((R)- (10aS,12aS)-3-cyclobutyl-1-((S)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine | Calc'd 451, found 451 |
| Ex-2.64 | 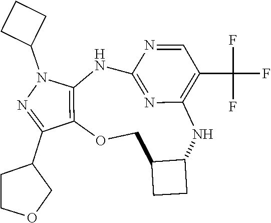 | (10aR,12aR)-3-cyclobutyl-1-((R)- (10aR,12aR)-3-cyclobutyl-1-((S)- (10aS,12aS)-3-cyclobutyl-1-((R)- (10aS,12aS)-3-cyclobutyl-1-((S)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa\|4,6,10]triazacyclotridecine | Calc'd 451, found 451 |
| Ex-2.65 | 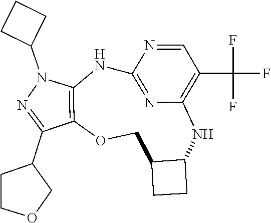 | (10aR,12aR)-3-cyclobutyl-1-((R)- (10aR,12aR)-3-cyclobutyl-1-((S)- (10aS,12aS)-3-cyclobutyl-1-((R)- (10aS,12aS)-3-cyclobutyl-1-((S)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine | Calc'd 451, found 451 |
| Ex-2.66 | 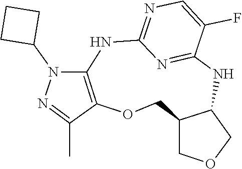 [SMILES: Cc1nn(C2CCC2)c2c1OC[C@@H]1COC[C@H]1Nc1nc(ncc1F)N2] | (10aS,13aS)-3-cyclobutyl-8-fluoro-1- methyl-3,4,10a,11,13a,14-hexahydro- 10H,13H-5,9-(azeno)furo[3,4- k]pyrazolo[4,3- b][1]oxa[4,6,10]triazacyclotridecine | Calc'd 361, found 361 |
| Ex-2.67 | 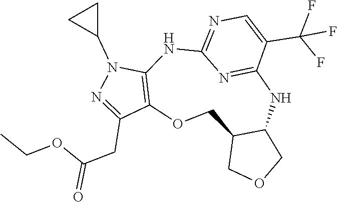 [SMILES: CCOC(=O)Cc1nn(C2CC2)c2c1OC[C@@H]1COC[C@H]1Nc1nc(ncc1C(F)(F)F)N2] | ethyl 2-((10aS,13aS)-3-cyclopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)acetate | Calc'd 469, found 469 |
| Ex-2.68 | 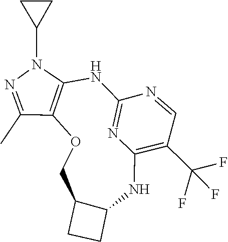 [SMILES: Cc1nn(C2CC2)c2c1OC[C@@H]1CC[C@H]1Nc1nc(ncc1C(F)(F)F)N2] | (10aR,12aR)-3-cyclopropyl-1-methyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecine | Calc'd 381 found 381 |
| Ex-2.69 | 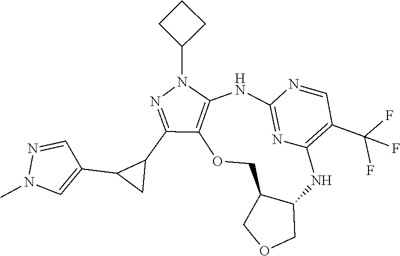 [SMILES: Cn1cc(C2CC2c2nn(C3CCC3)c3c2OC[C@@H]2COC[C@H]2Nc2nc(ncc2C(F)(F)F)N3)cn1] | (10aS,13aS)-3-cyclobutyl-1-((1S,2S)- or (10aS,13aS)-3-cyclobutyl-1-((1R,2R)-2-(1- methyl-1H-pyrazol-4-yl)cyclopropyl)-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 517, found 517 |
| Ex-2.70 | 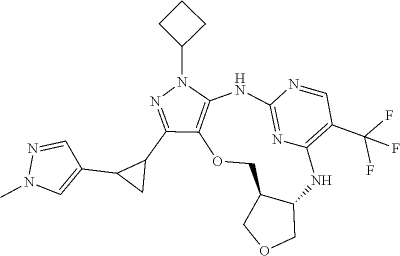 [SMILES: Cn1cc(C2CC2c2nn(C3CCC3)c3c2OC[C@@H]2COC[C@H]2Nc2nc(ncc2C(F)(F)F)N3)cn1] | (10aS,13aS)-3-cyclobutyl-1-((1S,2S)- or (10aS,13aS)-3-cyclobutyl-1-((IR,2R)-2-(1- methyl-1H-pyrazol-4-yl)cyclopropyl)-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 517, found 517 |
| Ex-2.71 | 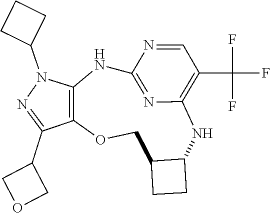 | (10aR,12aR)- or (10aS,12aS)-3-cyclobutyl-1-(oxetan-3-yl)- 8-(trifluoromethyl)- 3,4,10,10a,11,12,12a,13-octahydro-5,9- (azeno)cyclobuta[k]pyrazolo[4,3- b][1]oxa[4,6,10]triazacyclotridecine | Calc'd 437, found 437 |
| Ex-2.72 | 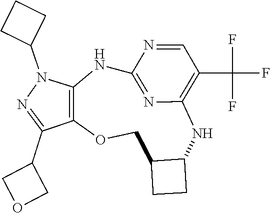 | (10aR,12aR)- or (10aS,12aS)-3-cyclobutyl-1-(oxetan-3-yl)- 8-(trifluoromethyl)- 3,4,10,10a,11,12,12a,13-octahydro-5,9- (azeno)cyclobuta[k]pyrazolo[4,3- b][1]oxa[4,6,10]triazacyclotridecine | Calc'd 437, found 437 |
| Ex-2.73 | 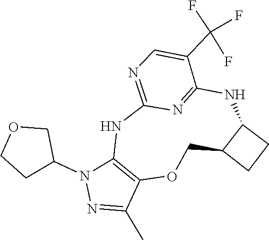 [SMILES: Cc1nn(C2CCOC2)c2c1OC[C@@H]1CC[C@H]1Nc1nc(ncc1C(F)(F)F)N2] | (10aR,12aR)-1-methyl-3-((S)- or (10aR,12aR)-1-methyl-3-((R)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine | Calc'd 411, found 411 |
| Ex-2.74 | 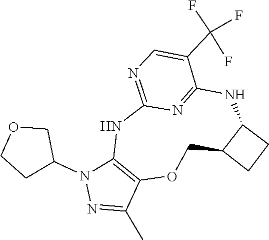 [SMILES: Cc1nn(C2CCOC2)c2c1OC[C@@H]1CC[C@H]1Nc1nc(ncc1C(F)(F)F)N2] | (10aR,12aR)-1-methyl-3-((S)- or (10aR,12aR)-1-methyl-3-((R)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine | Calc'd 411, found 411 |
| Ex-2.75 | 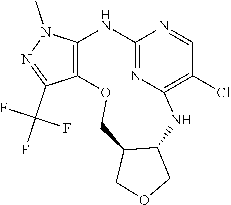 [SMILES: Cn1nc(C(F)(F)F)c2c1Nc1ncc(Cl)c(n1)N[C@@H]1COC[C@H]1CO2] | (10aS,13aS)-8-chloro-3-methyl-1- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 391, found 391 |
| Ex-2.76 | 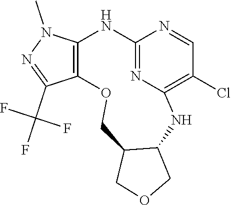 [SMILES: Cn1nc(C(F)(F)F)c2c1Nc1ncc(Cl)c(n1)N[C@@H]1COC[C@H]1CO2] | (10aR,12aR)-8-chloro-3-methyl-1- (trifluoromethyl)-3,4,10,10a,11,12,12a,13- octahydro-5,9- (azeno)cyclobuta[k]pyrazolo[4,3- b][1]oxa[4,6,10]triazacyclotridecine | Calc'd 375 found 375 |
| Ex-2.77 | 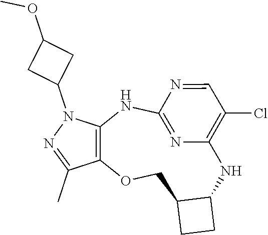 [SMILES: COC1CC(n2nc(C)c3c2Nc2ncc(Cl)c(n2)N[C@@H]2CC[C@H]2CO3)C1] | (10aR,12aR)-8-chloro-3-(3- methoxycyclobutyl)-1-methyl- 3,4,10,10a,11,12,12a,13-octahydro-5,9- (azeno)cyclobuta[k]pyrazolo[4,3- b][1]oxa[4,6,10]triazacyclotridecine | Calc'd 391, found 391 |
| Ex-2.78 | 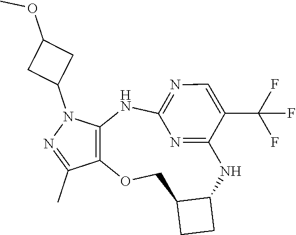 [SMILES: COC1CC(n2nc(C)c3c2Nc2ncc(C(F)(F)F)c(n2)N[C@@H]2CC[C@H]2CO3)C1] | (10aR,12aR)-3-(3-methoxycyclobutyl)-1- methyl-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine | Calc'd 425, found 425 |
| Ex-2.79 | 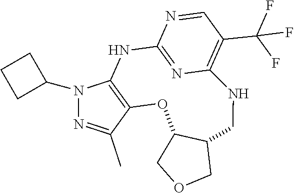 | (11aR,14aR)-3-cyclobutyl-1-methyl-8- (trifluoromethyl)-3,4,10,11,11a,12,14,14a- octahydro-5,9-(azeno)furo[3,4- 1]pyrazolo [4,3- b][1]oxa[4,6,10]triazacyclotridecine (11aS,14aS)-3-cyclobutyl-1-methyl-8- (trifluoromethyl)-3,4,10,11,11a,12,14,14a- octahydro-5,9-(azeno)furo[3,4- 1]pyrazolo[4,3- b][1]oxa[4,6,10]triazacyclotridecine | Calc'd 411 found 411 |
| Ex-2.80 | 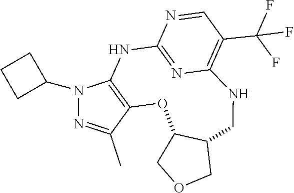 | (11aR,14aR)-3-cyclobutyl-1-methyl-8- (trifluoromethyl)-3,4,10,11,11a,12,14,14a- octahydro-5,9-(azeno)furo[3,4- 1]pyrazolo [4,3- b][1]oxa[4,6,10]triazacyclotridecine (11aS,14aS)-3-cyclobutyl-1-methyl-8- (trifluoromethyl)-3,4,10,11,11a,12,14,14a- octahydro-5,9-(azeno)furo[3,4- 1]pyrazolo[4,3- b][1]oxa[4,6,10]triazacyclotridecine | Calc'd 411, found 411 |
| Ex-2.81 | 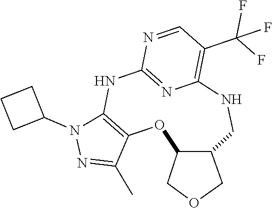 | (11aR,14aS) or (11aS,14aR)-3-cyclobutyl-1-methyl-8- (trifluoromethyl)-3,4,10,11,11a,12,14,14a- octahydro-5,9-(azeno)furo[3,4- 1]pyrazolo[4,3- b][1]oxa[4,6,10]triazacyclotridecine | Calc'd 411, found 411 |
| Ex-2.82 | 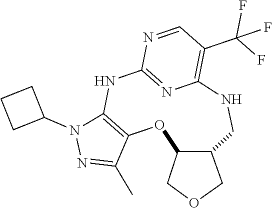 | (11aR,14aS) or (11aS,14aR)-3-cyclobutyl-1-methyl-8- (trifluoromethyl)-3,4,10,11,11a,12,14,14a- octahydro-5,9-(azeno)furo[3,4- 1]pyrazolo[4,3- b][1]oxa[4,6,10]triazacyclotridecine | Calc'd 411, found 411 |
| Ex-2.83 | 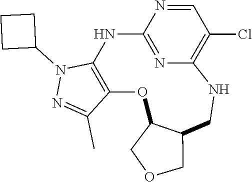 | (11aR,14aR)- or (11aS,14aS) 8-chloro-3-cyclobutyl-1- methyl-3,4,10,11,11a,12,14,14a-octahydro- 5,9-(azeno)furo[3,4-1]pyrazolo[4,3- b][1]oxa[4,6,10]triazacyclotridecine | Calc'd 377, found 377 |
| Ex-2.84 | 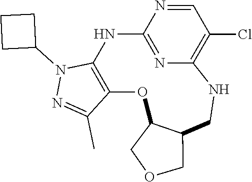 | (11aR,14aR)- or (11aS,14aS) 8-chloro-3-cyclobutyl-1- methyl-3,4,10,11,11a,12,14,14a-octahydro- 5,9-(azeno)furo[3,4-1]pyrazolo[4,3- b][1]oxa[4,6,10]triazacyclotridecine | Calc'd 377, found 377 |
| Ex-2.85 | 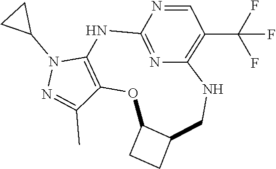 [SMILES: Cc1nn(C2CC2)c2c1O[C@@H]1CC[C@@H]1CNc1nc(ncc1C(F)(F)F)N2] | (11aR,13aR)- or (11aS,13aS)-3-cyclopropyl-1-methyl-8- (trifluoromethyl)-3,4,10,11,11a,12,13,13a- octahydro-5,9- (azeno)cyclobuta[I]pyrazolo[4,3- b][1]oxa[4,6,10]triazacyclotridecine | Calc'd 381, found 381 |
| Ex-2.86 | 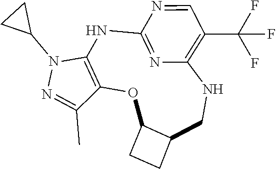 [SMILES: Cc1nn(C2CC2)c2c1O[C@@H]1CC[C@@H]1CNc1nc(ncc1C(F)(F)F)N2] | (11aR,13aR)- or (11aS,13aS)-3-cyclopropyl-1-methyl-8- (trifluoromethyl)-3,4,10,11,11a,12,13,13a- octahydro-5,9- (azeno)cyclobuta[l]pyrazolo[4,3- b][1]oxa[4,6,10]triazacyclotridecine | Calc'd 381, found 381 |
| Ex-2.87 | 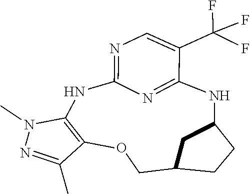 | (5¹S,5³R)-1¹,1³-dimethyl-3⁵- (trifluoromethyl)-1^{1H}-7-oxa-2,4-diaza- 3(2,4)-pyrimidina-1(5,4)-pyrazola-5(1,3)- cyclopentanacycloheptaphane | Calc'd 369, found 369 |
| Ex-2.88 | 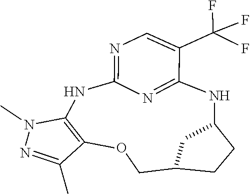 | (5¹R,5³S)-1¹,1³-dimethyl-3⁵- (trifluoromethyl)-1^{1H}-7-oxa-2,4-diaza- 3(2,4)-pyrimidina-1(5,4)-pyrazola-5(1,3)- cyclopentanacycloheptaphane | Calc'd 369, found 369 |

### Preparation of Example 3.1

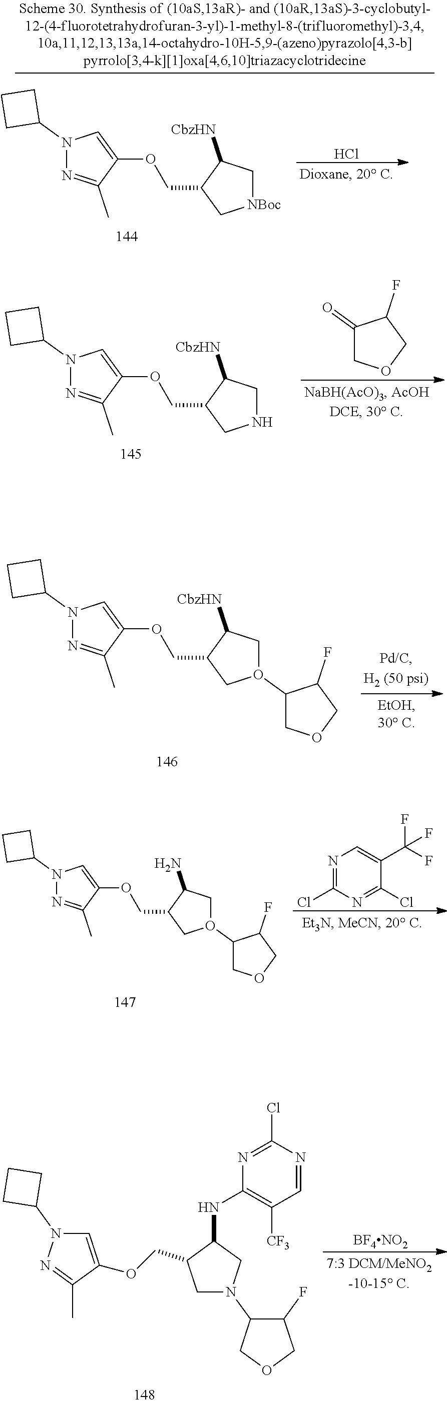

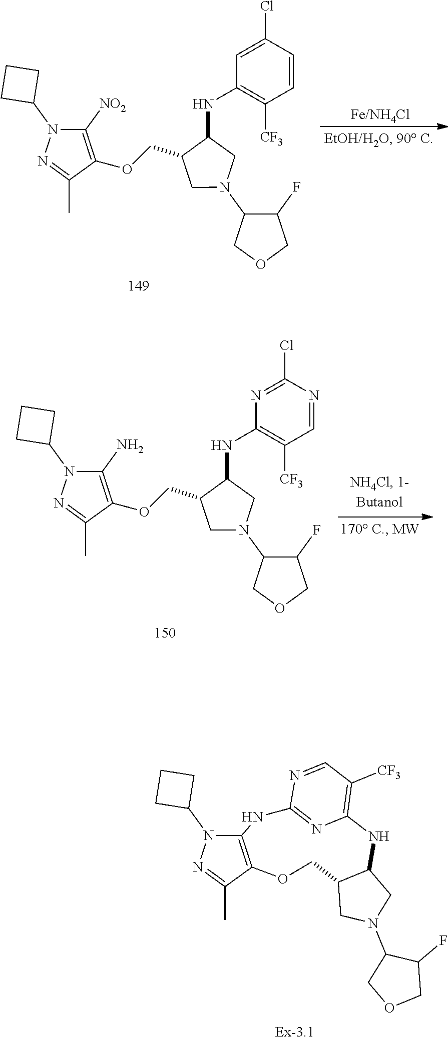 [SMILES: Cc1nn(C2CCC2)c([N+](=O)[O-])c1OC[C@H]1CN(C2COCC2F)C[C@@H]1Nc1cc(Cl)ccc1C(F)(F)F ; "Fe/NH4Cl" ; Cc1nn(C2CCC2)c(N)c1OC[C@H]1CN(C2COCC2F)C[C@@H]1Nc1nc(Cl)ncc1C(F)(F)F ; Cc1nn(C2CCC2)c2c1OC[C@H]1CN(C3COCC3F)C[C@@H]1Nc1nc(ncc1C(F)(F)F)N2]

### Benzyl ((3R,4S)- and (3S,4R)-4-(((1-cyclobutyl-3-methyl-1H-pyrazol-4-yl)oxy)methyl) pyrrolidin-3-yl)carbamate (145)

The starting ether intermediate 144 was prepared by reacting intermediate 15 with the corresponding intermediate 106 in accordance with the synthetic protocol described in General Scheme 6, using procedures analogous to those described above.

To a solution of tert-butyl 3-(((benzyloxy) carbonyl)amino)-4-(((1-cyclobutyl-3-methyl-1H-pyrazol-4-yl)oxy)methyl) pyrrolidine-1-carboxylate 144 (2.6 g, 5.37 mmol) in 1,4-dioxane (10 mL) was added 4M HCl·dioxane (20 mL). The reaction was allowed to stir for 2 h at 25° C. The solution was filtered and concentrated under reduced pressure. The crude product was concentrated under reduced pressure to afford the title compound 145. The product was used in the next step directly without further purification.

### Benzyl ((3R,4S)- and (3S,4R)-4-(((1-cyclobutyl-3-methyl-1H-pyrazol-4-yl)oxy)methyl)-1-(4-fluorotetrahydrofuran-3-yl) pyrrolidin-3-yl)carbamate (146)

To a solution of benzyl (4-(((1-cyclobutyl-3-methyl-1H-pyrazol-4-yl)oxy)methyl) pyrrolidin-3-yl)carbamate 145 (1.7 g, 4.42 mmol) in DCE (30 mL) was added 4-fluorodihydrofuran-3 (2H)-one (1.841 g, 17.69 mmol). The mixture was allowed to stir for 30 min at 30° C., then sodium triacetoxyborohydride (1.874 g, 8.84 mmol) was added. The reaction mixture was allowed to stir at 30° C. for 14 h. The solution was filtered and concentrated under reduced pressure. The crude product was purified by reversed phase HPLC, eluting with water (0.1% TFA)-MeCN, to afford the title compound 146.

### (3R,4S)- and (3S,4R)-4-(((1-cyclobutyl-3-methyl-1H-pyrazol-4-yl)oxy)methyl)-1-(4-fluorotetrahydrofuran-3-yl) pyrrolidin-3-amine (147)

To a solution of benzyl (4-(((1-cyclobutyl-3-methyl-1H-pyrazol-4-yl)oxy)methyl)-1-(4-fluorotetrahydrofuran-3-yl) pyrrolidin-3-yl)carbamate 146 (400 mg, 0.846 mmol) in anhydrous EtOH (10 mL) was added Pd/C (150 mg, 0.141 mmol), and the reaction mixture was allowed to stir at 30° C. under H₂ (50 psi) for 16 h. The solution was filtered and concentrated under reduced pressure. The crude product was purified by reversed phase HPLC, eluting with water (0.1% NH₄OH)-MeCN, to afford the title compound 147. MS (ESI): m/z calc'd for C₁₇H₂₇FN₄O₂ [M+H]⁺: 339, found 399.

### (10aS,13aR)- and (10aR,13aS)-3-cyclobutyl-12-(4-fluorotetrahydrofuran-3-yl)-1-methyl-8-(trifluoromethyl)-3,4,10a,11,12,13,13a,14-octahydro-10H-5,9-(azeno)pyrazolo[4,3-b]pyrrolo[3,4-k][1]oxa[4,6,10]triazacyclotridecine (Ex-3.1)

The title compound Ex-3.1 was prepared in accordance with the synthetic protocol described in Scheme 29 and the accompanying text by substituting intermediate 139 for intermediate 147. MS (ESI): m/z calc'd for C₂₂H₂₇F₄N₇O₂ [M+H]⁺: 498, found 498. ¹H NMR (500 MHz, MeOD) δ=8.16 (d, J=4.1 Hz, 1H), 5.29-5.10 (m, 1H), 4.91 (s, 1H), 4.79 (m, 1H), 4.46-4.41 (m, 1H), 4.36 (m, 1H), 4.15-4.03 (m, 3H), 3.94 (q, J=10.3 Hz, 1H), 3.83-3.75 (m, 1H), 3.50-3.36 (m, 1H), 3.13-3.06 (m, 1H), 2.86-2.73 (m, 2H), 2.66-2.50 (m, 2H), 2.47-2.38 (m, 1H), 2.33 (d, J=2.4 Hz, 4H), 2.13-1.96 (m, 1H), 1.89-1.80 (m, 2H).

### Preparation of Examples 4.1 and 4.2

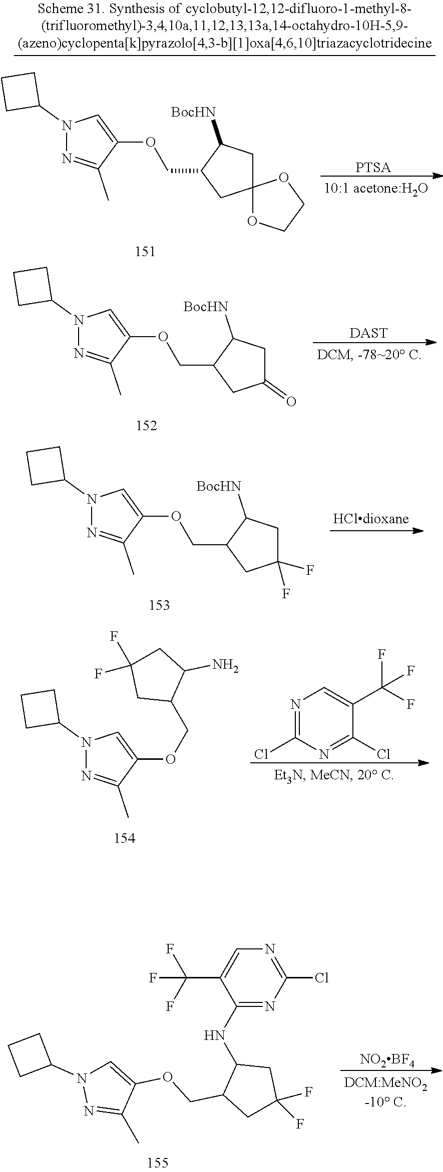

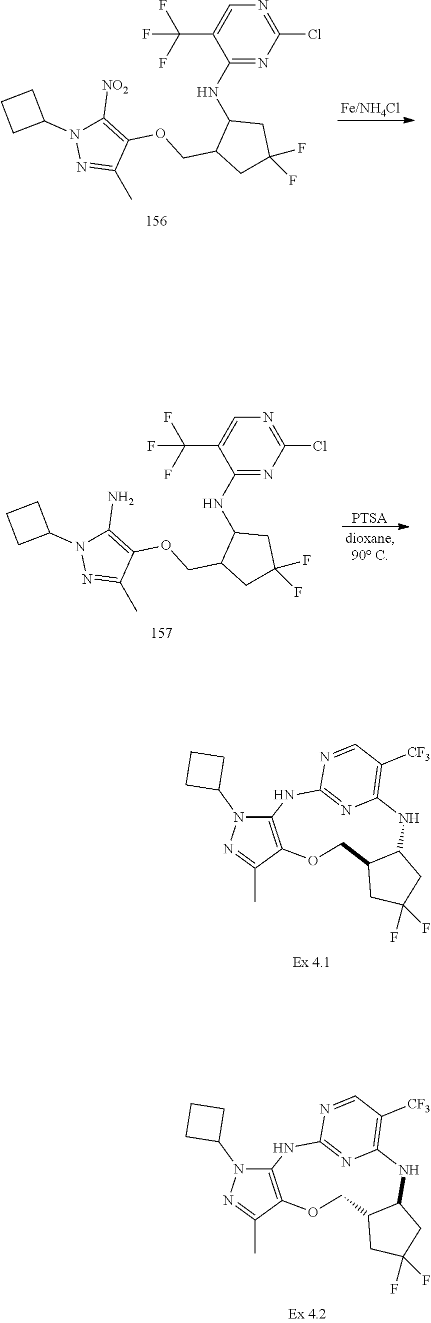

### tert-Butyl (2-(((1-cyclobutyl-3-methyl-1H-pyrazol-4-yl)oxy)methyl)-4-oxocyclopentyl)carbamate (152)

To a solution of tert-butyl (2-(((1-cyclobutyl-3-methyl-1H-pyrazol-4-yl)oxy)methyl)-4-oxocyclopentyl)carbamate 152 (400 mg, 1.101 mmol) in a 20 mL plastic bottle with anhydrous DCM (10 mL) was added N,N-diethyl-1,1,1-trifluoro-14-sulfanamine (0.585 mL, 4.40 mmol) at −78° C. under N₂. The reaction mixture was allowed to stir at 25° C. under N₂ for 5 h, then added to saturated NaHCO₃ (20 mL), extracted with DCM (30 mL×3), dried with anhydrous Na₂SO₄, and filtered. The filtrate was concentrated under reduced pressure. The crude residue was purified by prep-TLC (2:1 PE/EtOAc) to afford the title compound 153.

### 3-Cyclobutyl-12,12-difluoro-1-methyl-8-(trifluoromethyl)-3,4,10a,11,12,13,13a,14-octahydro-10H-5,9-(azeno)cyclopenta[k]pyrazolo[4,3-b][1]oxa[4,6,10]triazacyclotridecine (Ex-4.1 and Ex-4.2)

The title compound Ex-3.1 was prepared in accordance with the synthetic protocol described in Scheme 29 and the accompanying text by substituting intermediate 140 for intermediate 154. The racemic material could be resolved to its component enantiomers by chiral preparative SFC (Column: Chiralpak AD-3 50×4.6 mm I.D., 3 μm; Mobile phase: A: CO₂ B: ethanol (0.05% DEA) Gradient: from 5% to 40% of B in 2 min and hold 40% for 1.2 min, then 5% of B for 0.8 Min. Flow rate: 4 mL/min) to afford Ex-4.1 (t_{R}=1.3 min) and Ex-4.2 (t_{R}=1.8 min). MS (ESI): m/z calc'd for C₁₉H₂₁F₅N₆O [M+H]⁺: 445, found 445; ¹H NMR (400 MHz, CDCl₃) δ=8.13 (s, 1H), 7.52 (br s, 1H), 5.47 (m, 1H), 4.65 (m, 1H), 4.60-4.49 (m, 1H), 4.32 (d, J=10.7 Hz, 1H), 3.94 (dd, J=8.3, 10.8 Hz, 1H), 2.78 (m, 1H), 2.65-2.42 (m, 3H), 2.39-2.31 (m, 6H), 2.17-2.08 (m, 1H), 1.88-1.73 (m, 3H) MS (ESI): m/z calc'd for C₁₉H₂₁F₅N₆O [M+H]⁺: 445, found 445; ¹H NMR (400 MHz, CDCl₃) δ=8.13 (s, 1H), 7.64-7.39 (m, 1H), 5.47 (m, 1H), 4.65 (m, 1H), 4.60-4.48 (m, 1H), 4.32 (d, J=10.7 Hz, 1H), 3.93 (dd, J=8.3, 10.8 Hz, 1H), 2.78 (m, 1H), 2.65-2.47 (m, 3H), 2.39-2.31 (m, 6H), 2.17-2.09 (m, 1H), 1.91-1.77 (m, 3H).

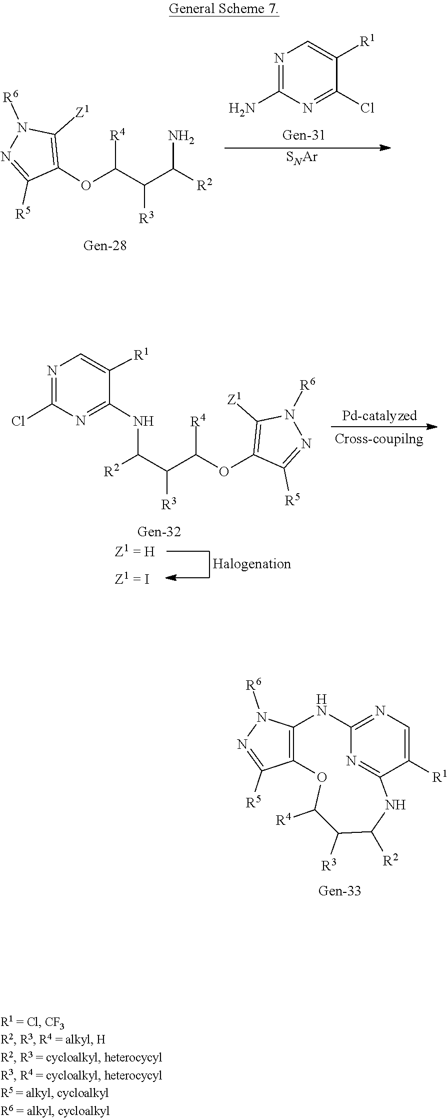

1H), 7.52 (br s, 1H), 5.47 (m, 1H), 4.65 (m, 1H), 4.60-4.49 (m, 1H), 4.32 (d, J=10.7 Hz, 1H), 3.94 (dd, J=8.3, 10.8 Hz, 1H), 2.78 (m, 1H), 2.65-2.42 (m, 3H), 2.39-2.31 (m, 6H), 2.17-2.08 (m, 1H), 1.88-1.73 (m, 3H) MS (ESI): m/z calc'd for C₁₉H₂₁F₅N₆O [M+H]⁺: 445, found 445; ¹H NMR (400 MHz, CDCl₃) δ=8.13 (s, 1H), 7.64-7.39 (m, 1H), 5.47 (m, 1H), 4.65 (m, 1H), 4.60-4.48 (m, 1H), 4.32 (d, J=10.7 Hz, 1H), 3.93 (dd, J=8.3, 10.8 Hz, 1H), 2.78 (m, 1H), 2.65-2.47 (m, 3H), 2.39-2.31 (m, 6H), 2.17-2.09 (m, 1H), 1.91-1.77 (m, 3H).

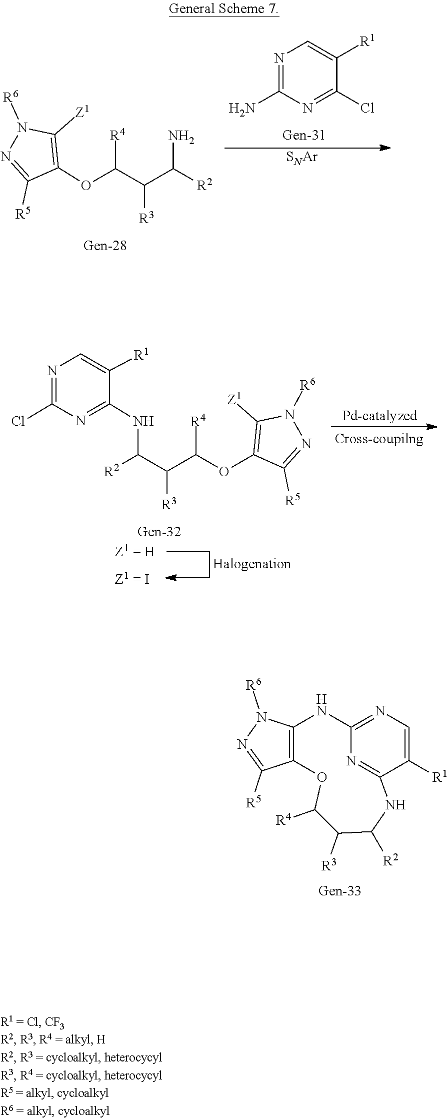

In General Scheme 7, commercially available pyrimidines of the form Gen-31 were coupled with amines of the form Gen-28 through S_{N}Ar chemistry to access Gen-32. In instances of Gen-32 where Z¹=H, iodination of the pyrazole was performed. Iodopyrazoles of the form Gen-32 could ultimately be transformed through intramolecular Pd-catalyzed cross-coupling chemistry to afford elaborated compounds of the form Gen-33. Representative preparative examples from each sequence are described in more detail below.

### Preparation of Example 5.1

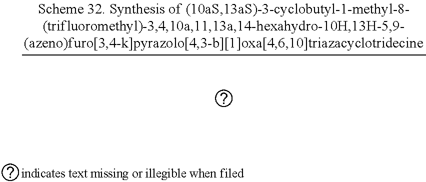

and added to a separatory funnel containing sat. aq. NaCl solution. The layers were separated. The aqueous layers was extracted with EtOAc. The combined organic layers were dried with Na₂SO₄, filtered, and concentrated under reduced pressure. The crude product was elution of 0-100% 3:1 EtOAc:EtOH/hexanes) to afford the title compound 159.

### N⁴-((3S,4S)-4-(((1-cyclobutyl-5-iodo-3-methyl-1H-pyrazol-4-yl)oxy)methyl)tetrahydrofuran-3-yl)-5-(trifluoromethyl)pyrimidine-2,4-diamine (160)

A 250 mL RBF containing N4-((3S,4S)-4-(((1-cyclobutyl-3-methyl-1H-pyrazol-4-yl)oxy)methyl)tetrahydrofuran-3-yl)-5-(trifluoromethyl)pyrimidine-2,4-diamine 159 (5.0 g, 12.12 mmol) was charged with N-iodosuccinimide (4.09 g, 18.19 mmol) and p-toluene sulfonic acid monohydrate (2.54 g, 13.34 mmol). The mixture was dissolved in THF (60.6 mL). The reaction mixture was heated to 60° C. and allowed to stir for 1 h, then cooled, diluted with EtOAc and poured into a separatory funnel containing water. The layers were separated, and the aqueous layer was extracted with EtOAc. The combined organic layers were washed with brine and dried over Na₂SO₄. The solution was filtered and concentrated under reduced pressure. The crude reaction mixture was purified by flash chromatography on silica gel (gradient elution of 0-80% 3:1 EtOAc:EtOH/hexanes). The material was dissolved in DCM and added to a separatory funnel containing sat. aq. Na₂S₂O₃ solution. The combined aqueous layers were extracted once with DCM. The combined organic layers were then washed with sat. aq. Na₂S₂O₃ and brine and dried with Na₂SO₄. The solution was filtered and concentrated under reduced pressure to afford the title compound 160.

### (10aS,13aS)-3-cyclobutyl-1-methyl-8-(trifluoromethyl)-3,4,10a,11,13a,14-hexahydro-10H,13H-5,9-(azeno)furo[3,4-k]pyrazolo[4,3-b][1]oxa[4,6,10]triazacyclotridecine (Ex-5.1)

A 2 L 3-neck flask equipped with a stir bar, thermocouple, and reflux condenser was charged with N⁴-((3S,4S)-4-(((1-cyclobutyl-5-iodo-3-methyl-1H-pyrazol-4-yl)oxy)methyl)tetrahydrofuran-3-yl)-5-(trifluoromethyl)pyrimidine-2,4-diamine 160 (11.25 g, 20.90 mmol), BrettPhos Pd G4 (3.85 g, 4.18 mmol) and potassium phosphate tribasic (22.18 g, 104 mmol). The flask was sealed and inerted 3× vacuum/nitrogen cycles and left under N₂. The solids were then suspended in dioxane (836 mL). The reaction mixture was heated to an internal temperature of 100° C. for 24 h, then cooled to room temperature and filtered to remove solids. The filtrate was concentrated under reduced pressure to remove volatiles. The residue was diluted with EtOAc and added to a separatory funnel containing water. The layers were separated and the aqueous layer was extracted with EtOAc. The combined organic layers washed with brine and dried with Na₂SO₄. The solution was filtered and concentrated under reduced pressure. The crude product was purified by flash chromatography on silica gel (gradient elution of 0-60% 3:1 EtOAc:EtOH/hexanes). The product was recrystallized in iPrOAc (8.8 mL) and hexanes (88 mL) to afford the title compound Ex-5.1. MS (ESI): m/z calc'd for C₁₈H₂₁F₃N₆O₂ [M+H]⁺: 411, found 411. ¹H NMR (DMSO-d₆, 600 MHz) δ 9.55 (1H, s), 8.19 (1H, s), 7.03 (1H, d, J=5.4 Hz), 4.86 (1H, hept, J=6.3 Hz), 4.72 (1H, p, J=8.3 Hz), 4.28 (1H, s), 4.22 (1H, d, J=10.7 Hz), 4.14 (1H, dd, J=9.7, 3.1 Hz), 4.02 (1H, t, J=8.9 Hz), 3.83 (1H, dd, J=11.1, 9.4 Hz), 3.70 (1H, dd, J=9.7, 6.9 Hz), 3.13 (1H, dd, J=9.1, 7.6 Hz), 2.43 (2H, q, J=10.2 Hz), 2.29 (1H, dd, J=9.0, 6.2 Hz), 2.19 (3H, s), 2.07 (1H, q, J=7.7 Hz), 1.76-1.63 (2H, m).

### Preparation of Examples 5.2 and 5.3

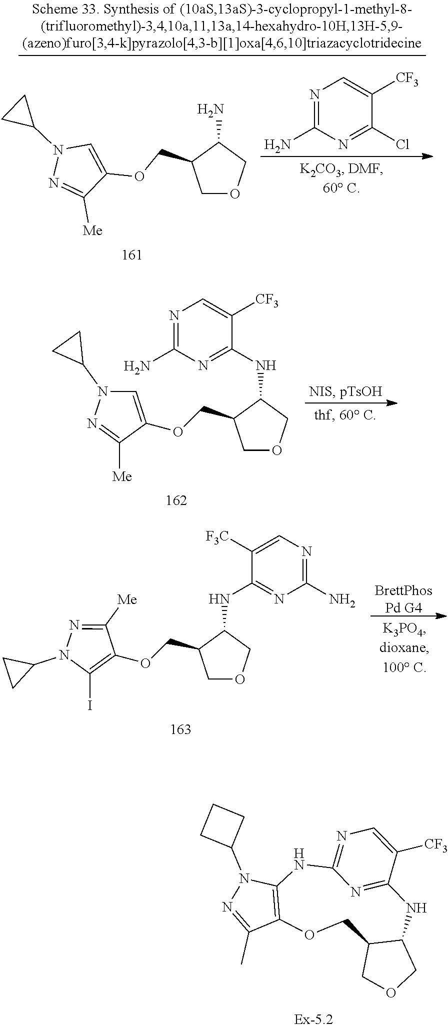

### (10aS,13aS)-3-cyclopropyl-1-methyl-8-(trifluoromethyl)-3,4,10a,11,13a,14-hexahydro-10H,13H-5,9-(azeno)furo[3,4-k]pyrazolo[4,3-b][1]oxa[4,6,10]triazacyclotridecine (Ex-5.2)

The title compound Ex-6.2 was prepared in accordance with the synthetic protocol described in Scheme 32 and the accompanying text by substituting intermediate 158 for intermediate 161.

To a vial containing N4-((3S,4S)-4-(((1-cyclopropyl-5-iodo-3-methyl-1H-pyrazol-4-yl)oxy)methyl)tetrahydrofuran-3-yl)-5-(trifluoromethyl)pyrimidine-2,4-diamine (335.8 mg, 3,4,10a,11,13a,14-hexahydro-10H,13H-5,9-(azeno)furo[3,4-k]pyrazolo[4,3-b][1]oxa[4,6,10]triazacyclotridecine

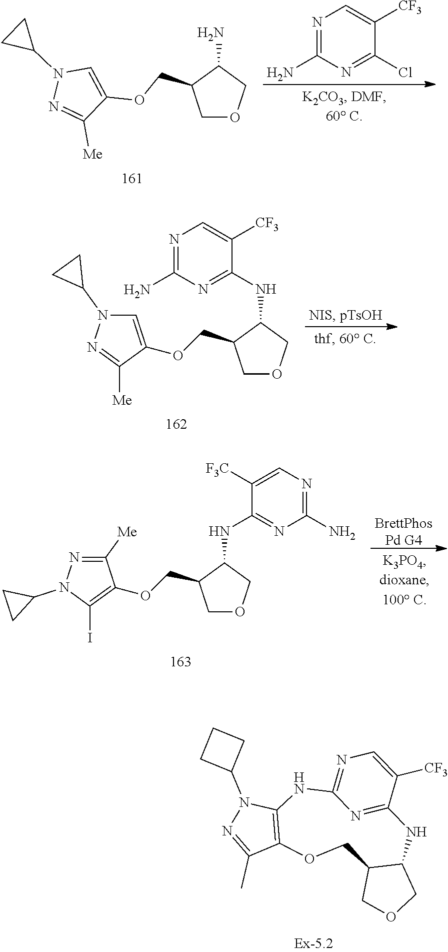

### (10aS,13aS)-3-cyclopropyl-1-methyl-8-(trifluoromethyl)-3,4,10a,11,13a,14-hexahydro-10H,13H-5,9-(azeno)furo[3,4-k]pyrazolo[4,3-b][1]oxa[4,6,10]triazacyclotridecine (Ex-5.2)

The title compound Ex-6.2 was prepared in accordance with the synthetic protocol described in Scheme 32 and the accompanying text by substituting intermediate 158 for intermediate 161.

To a vial containing N⁴-((3S,4S)-4-(((1-cyclopropyl-5-iodo-3-methyl-1H-pyrazol-4-yl)oxy)methyl)tetrahydrofuran-3-yl)-5-(trifluoromethyl)pyrimidine-2,4-diamine (335.8 mg, 0.640 mmol) was added BrettPhos Pd G4 (59.0 mg, 0.064 mmol) and tripotassium phosphate (680 mg, 3.20 mmol). The reaction mixture was sealed and inerted 3× vacuum/nitrogen cycles and left under N₂. To the reaction was added 1,4-dioxane (25.6 mL) under N₂. The reaction mixture was heated to 100° C. and allowed to stir for 10 h, then concentrated under reduced pressure. The crude product was dissolved in methanol and filtered. The solution was purified by reversed phase HPLC, eluting with water (0.1% NH₄OH)-MeCN, to afford the title compound Ex-5.2. MS (ESI): m/z calc'd for C₁₇H₁₉F₃N₆O₂ [M+H]⁺: 397, found 397; ¹H NMR (DMSO-d₆, 500 MHz) δ 9.54 (1H, s), 8.20 (1H, s), 7.05 (1H, d, J=4.9 Hz), 4.28 (1H, s), 4.21 (1H, d, J=11.2 Hz), 4.14 (1H, d, J=9.8 Hz), 4.02 (1H, t, J=8.9 Hz), 3.81 (1H, t, J=10.3 Hz), 3.71 (1H, t, J=8.3 Hz), 3.43 (1H, s), 3.13 (1H, t, J=8.2 Hz), 2.12 (3H, s), 2.09 (1H, d, J=8.3 Hz), 1.11 (1H, d, J=4.6 Hz), 0.87 (2H, tq, J=16.0, 9.2, 8.5 Hz), 0.75-0.64 (1H, m).

Compounds in Table 6 below were prepared in accordance with the synthetic sequence illustrated in General Scheme 7 using the corresponding starting materials.

TABLE 6

| | | | Exact Mass |
|---|---|---|---|
| Ex | Structure | Name | [M + H]⁺ |
| Ex-5.3 | 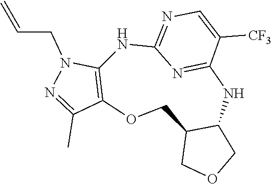 [SMILES: C=CCn1nc(C)c2c1Nc1ncc(C(F)(F)F)c(n1)N[C@@H]1COC[C@H]1CO2] | (10aS,13aS)-3-allyl-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine | Calc'd 397, found 397 |
| Ex-5.4 | 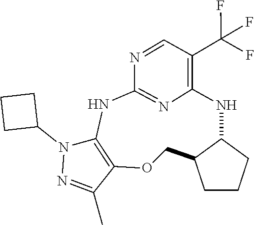 | (10aR,13aR)- or (10aS,13aS)-3-cyclobutyl-1-methyl-8- (trifluoromethyl)-3,4,10a,11,12,13,13a,14- octahydro-10H-5,9- (azeno)cyclopenta[k]pyrazolo[4,3- b][1]oxa[4,6,10]triazacyclotridecine | Calc'd 409, found 409 |
| Ex-5.5 | 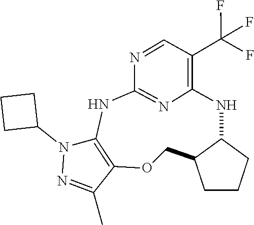 | (10aR,13aR)- or (10aS,13aS)-3-cyclobutyl-1-methyl-8- (trifluoromethyl)-3,4,10a,11,12,13,13a,14- octahydro-10H-5,9- (azeno)cyclopenta[k]pyrazolo[4,3- b][1]oxa[4,6, 10]triazacyclotridecine | Calc'd 409, found 409 |
| Ex-5.6 | 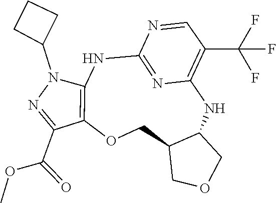 [SMILES: COC(=O)c1nn(C2CCC2)c2c1OC[C@@H]1COC[C@H]1Nc1nc(ncc1C(F)(F)F)N2] | methyl (10aS,13aS)-3-cyclobutyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine-1- carboxylate | Calc'd 455, found 455 |

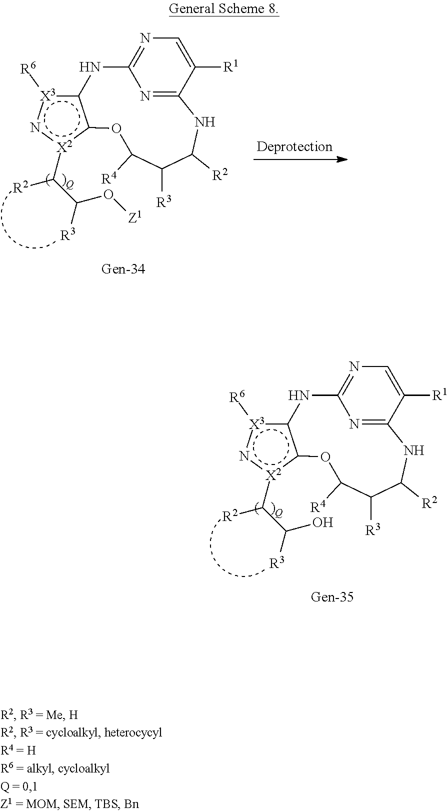

In General Scheme 8, synthetically prepared intermediates with protected alcohols of the form Gen-34 could be transformed through removal of the protecting groups using a number of deprotection methods commonly known to those skilled in the art, including, but not limited to, acid-mediated cleavage or hydrogenation, to compounds of the form Gen-35. Representative preparative examples from each sequence are described in more detail below.

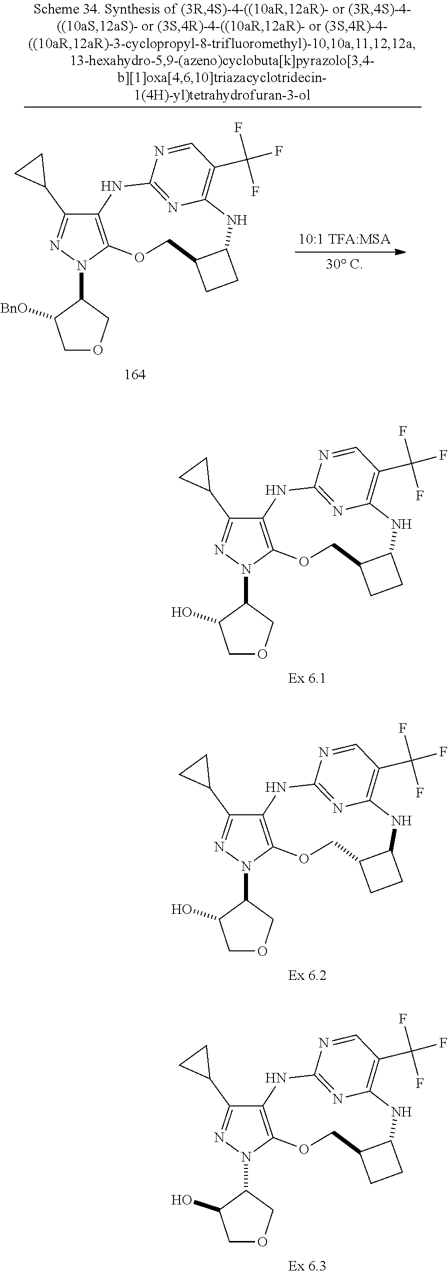

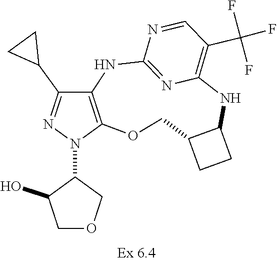

### (3R,4S)-4-((10aR,12aR)- or (3R,4S)-4-((10aS,12aS)- or (3S,4R)-4-((10aR,12aR)- or (3S,4R)-4-((10aR,12aR)-3-cyclopropyl-8-(trifluoromethyl)-10,10a,11,12,12a,13-hexahydro-5,9-(azeno)cyclobuta[k]pyrazolo[3,4-b][1]oxa[4,6,10]triazacyclotridecin-1 (4H)-yl) tetrahydrofuran-3-ol (Ex-6.1, Ex-6.2, Ex-6.3 and Ex-6.4)

To a solution of (10aR,12aR)-1-((3S,4R)- and (10aS,12aS)-1-((3S,4R)- and (10aR,12aR)-1-((3R,4S)- and (10aR,12aR)-1-((3R,4S)-4-(benzyloxy)tetrahydrofuran-3-yl)-3-cyclopropyl-8-(trifluoromethyl)-1,4,10,10a,11,12,12a, 13-octahydro-5,9-(azeno)cyclobuta[k]pyrazolo[3,4-b][1]oxa[4,6,10]triazacyclotridecine 164 (200 mg, 0.369 mmol) in TFA (5 mL, 64.9 mmol) was added methanesulfonic acid (0.5 mL, 7.70 mmol). The solution was allowed to stir for 3 h at 30° C. The crude product was purified by reversed phase HPLC, eluting with water (0.1% TFA)-1H), 5.32 (br s, 1H), 5.19 (m, 1H), 4.68 (m, 1H), 4.49-4.39 (m, 2H), 4.21-4.05 (m, 4H), 4.01-3.96 (m, 2H), 2.51 (m, 1H), 2.28 (q, J=9.0 Hz, 1H), 2.06 (q, J=9.8 Hz, 1H), 1.86 (m, 1H), 1.71-1.68 (m, 1H), 1.61-1.52 (m, 1H), 0.98-0.91 (m, 1H), 0.90-0.86 (m, 2H), 0.75-0.64 (m, 1H).

Compounds in Table 7 below were prepared in accordance with the synthetic sequence illustrated in General Scheme 5 or General Scheme 7 and subsequent deprotection illustrated in General Scheme 8 using the corresponding starting materials.

TABLE 7

| | | | Exact Mass |
|---|---|---|---|
| Ex | Structure | Name | [M + H]⁺ |
| Ex-6.5 | 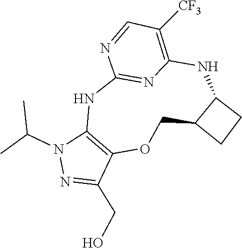 [SMILES: CC(C)n1nc(CO)c2c1Nc1ncc(C(F)(F)F)c(n1)N[C@@H]1CC[C@H]1CO2] | ((10aR,12aR)-3-isopropyl-8- (trifluoromcthyl)-3,4,10,10a,11,12,12a,13- octahydro-5,9- (azeno)cyclobuta[k]pyrazolo[4,3- b][1]oxa[4,6,10]triazacyclotridecin-1- yl)methanol | Calc'd 399, found 399 |
| Ex-6.6 | 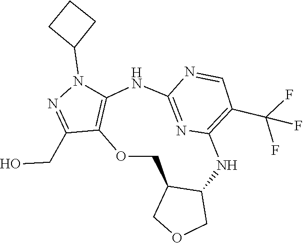 [SMILES: OCc1nn(C2CCC2)c2c1OC[C@@H]1COC[C@H]1Nc1nc(ncc1C(F)(F)F)N2] | ((10aS,13aS)-3-cyclobutyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)methanol | Calc'd 427, found 427 |
| Ex-6.7 | 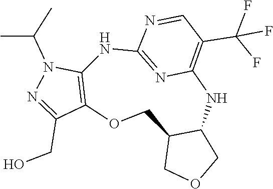 [SMILES: CC(C)n1nc(CO)c2c1Nc1ncc(C(F)(F)F)c(n1)N[C@@H]1COC[C@H]1CO2] | ((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)methanol | Calc'd 415, found 415 |
| Ex-6.8 | 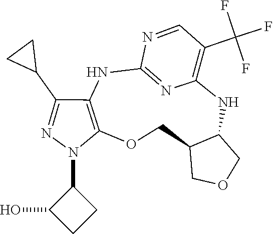 [SMILES: O[C@H]1CC[C@@H]1n1nc(C2CC2)c2c1OC[C@@H]1COC[C@H]1Nc1nc(ncc1C(F)(F)F)N2] | (1R,2R)- or (1S,2S)-2-((10aS,13aS)-3- cyclopropyl-8-(trifluoromethyl)- 10a,11,13a,14-tetrahydro-10H,13H-5,9- (azeno)furo[3,4-k]pyrazolo[3,4- b][1]oxa[4,6,10]triazacyclotridecin-1(4H)- yl)cyclobutan-1-ol | Calc'd 453, found 453 |
| Ex-6.5 | 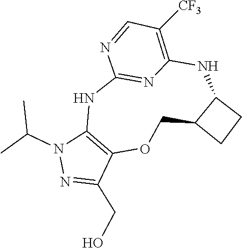 [SMILES: CC(C)n1nc(CO)c2c1Nc1ncc(C(F)(F)F)c(n1)N[C@@H]1CC[C@H]1CO2] | ((10aR,12aR)-3-isopropyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecin-1- yl)methanol | Calc'd 399, found 399 |
| Ex-6.6 | 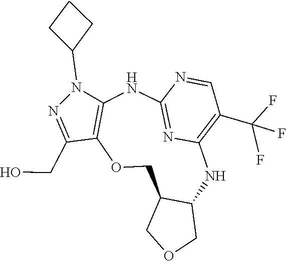 [SMILES: OCc1nn(C2CCC2)c2c1OC[C@@H]1COC[C@H]1Nc1nc(ncc1C(F)(F)F)N2] | ((10aS,13aS)-3-cyclobutyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)methanol | Calc'd 427, found 427 |
| Ex-6.7 | 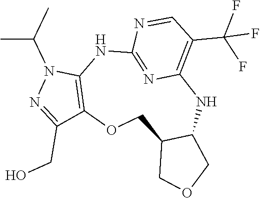 [SMILES: CC(C)n1nc(CO)c2c1Nc1ncc(C(F)(F)F)c(n1)N[C@@H]1COC[C@H]1CO2] | ((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)methanol | Calc'd 415, found 415 |
| Ex-6.8 | 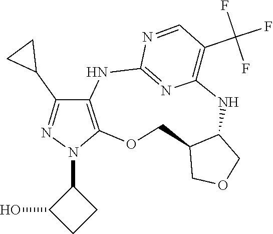 [SMILES: O[C@H]1CC[C@@H]1n1nc(C2CC2)c2c1OC[C@@H]1COC[C@H]1Nc1nc(ncc1C(F)(F)F)N2] | (1R,2R)- or (1S,2S)-2-((10aS,13aS)-3- cyclopropyl-8-(trifluoromethyl)- 10a,11,13a,14-tetrahydro-10H,13H-5,9- (azeno)furo[3,4-k]pyrazolo[3,4- b][1]oxa[4,6,10]triazacyclotridecin-1(4H)- yl)cyclobutan-1-ol | Calc'd 453, found 453 |
| Ex-6.9 | 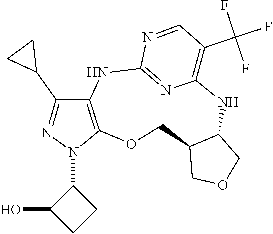 [SMILES: O[C@@H]1CC[C@H]1n1nc(C2CC2)c2c1OC[C@@H]1COC[C@H]1Nc1nc(ncc1C(F)(F)F)N2] | (1R,2R)- or (1S,2S)-2-((10aS,13aS)-3- cyclopropyl-8-(trifluoromethyl)- 10a,11,13a, 14-tetrahydro-10H,13H-5,9- (azeno)furo[3,4-k]pyrazolo[3,4- b][1]oxa[4,6,10]triazacyclotridecin-1(4H)- yl)cyclobutan-1-ol | Calc'd 453, found 453 |

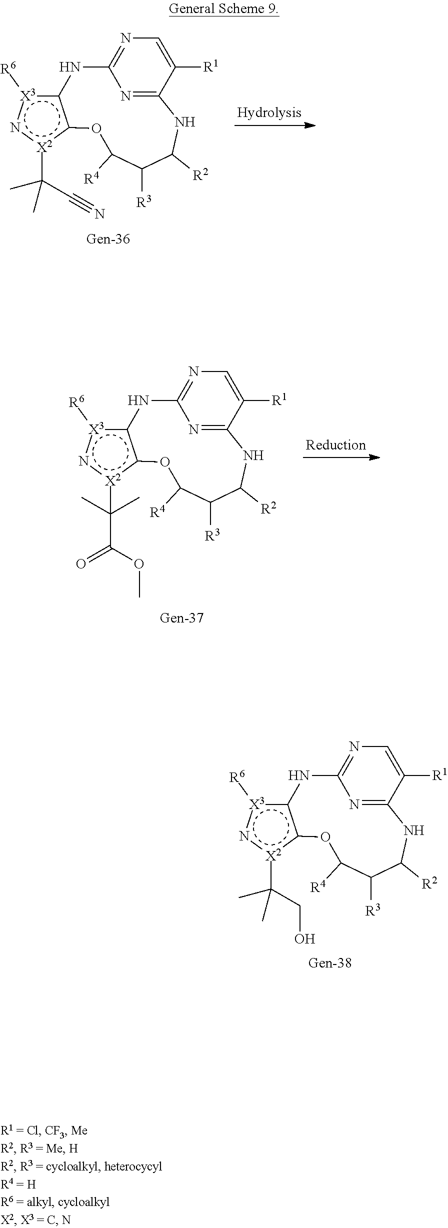

In General Scheme 9, synthetically prepared compounds of the form Gen-36 could be transformed by acid-mediated hydrolysis to esters of the form Gen-37. Esters Gen-37 could be ultimately transformed through reduction of the carbonyl to the elaborated compounds of the form Gen-38. Representative preparative examples from each sequence are described in more detail below.

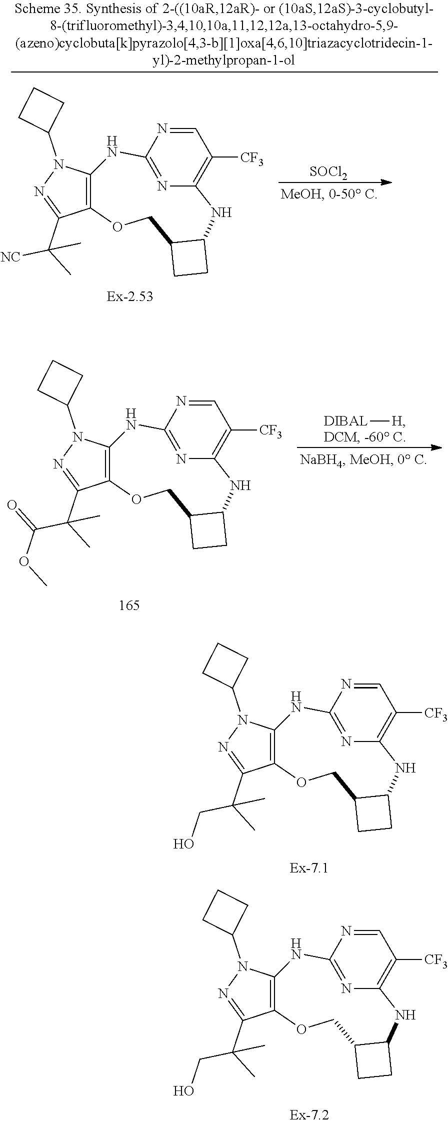

### Methyl 2-((10aR,12aR)- and (10aS,12aS)-3-cyclobutyl-8-(trifluoromethyl)-3,4,10,10a,11,12,12a,13-octahydro-5,9-(azeno)cyclobuta[k]pyrazolo[4,3-b][1]oxa[4,6,10]triazacyclotridecin-1-yl)-2-methylpropanoate (165)

A solution of SOCl₂ (2 mL, 27.4 mmol) in MeOH (4 mL) was allowed to stir at 0° C. for 30 min. To the reaction mixture was added 2-((10aR,12aR)- and ((10aS,12aS)-3-cyclobutyl-8-(trifluoromethyl)-3,4,10,10a, 11,12,12a, 13-octahydro-5,9-(azeno)cyclobuta[k]pyrazolo[4,3-b][1]oxa[4,6,10]triazacyclotridecin-1-yl)-2-methylpropanenitrile Ex-2.53 (50 mg, 0.112 mmol). (m, 1H), 1.41 (s, 3H), 1.35 (s, 3H); MS (ESI): m/z calc'd for C₂₁H₂₇F₃N₆O₂ [M+H]⁺: 453, found 453; ¹H NMR (500 MHz, MeOD) δ=8.00 (s, 1H), 4.67 (m, 1H), 4.18 (m, 1H), 4.11-3.90 (m, 1H), 3.75 (m, 1H), 3.64 (d, J=10.8 Hz, 1H), 3.59-3.54 (m, 1H), 2.58-2.38 (m, 2H), 2.34-2.18 (m, 3H), 2.15-2.03 (m, 1H), 1.98-1.84 (m, 2H), 1.80-1.68 (m, 2H), 1.55-1.38 (m, 1H), 1.29 (s, 3H), 1.23 (s, 3H).

Compounds in Table 8 below were prepared in accordance with the synthetic sequence illustrated in General Scheme 4 or General Scheme 7 and subsequent deprotection illustrated in General Scheme 9 using the corresponding starting materials.

TABLE 8

| | | | Exact Mass |
|---|---|---|---|
| Ex | Structure | Name | [M + H]⁺ |
| Ex-7.3 | 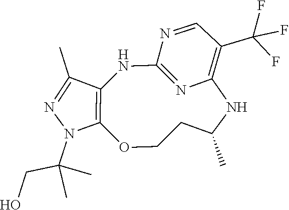 [SMILES: Cc1nn(C(C)(C)CO)c2c1Nc1ncc(C(F)(F)F)c(n1)N[C@H](C)CCO2] | (R)-2-(3,11-dimethyl-8-(trifluoromethyl)- 10,11,12,13-tetrahydro-5,9- (azeno)pyrazolo[3,4- b][1]oxa[4,6,10]triazacyclotridecin-1(4H)- yl)-2-methylpropan-1-ol | Calc'd 401, found 401 |
| Ex-7.4 | 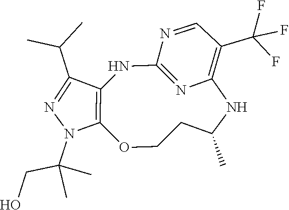 [SMILES: CC(C)c1nn(C(C)(C)CO)c2c1Nc1ncc(C(F)(F)F)c(n1)N[C@H](C)CCO2] | (R)-2-(3-isopropyl-11-methyl-8- (trifluoromethyl)-10,11,12,13-tetrahydro- 5,9-(azeno)pyrazolo[3,4- b][1]oxa[4,6,10]triazacyclotridecin-1(4H)- yl)-2-methylpropan-1-ol | Calc'd 429, found 429 |
| Ex-7.5 | 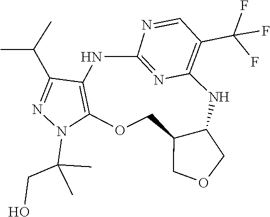 [SMILES: CC(C)c1nn(C(C)(C)CO)c2c1Nc1ncc(C(F)(F)F)c(n1)N[C@@H]1COC[C@H]1CO2] | 2-((10aS,13aS)-3-isopropyl-8- (trifluoromethyl)-10a,11,13a,14- tetrahydro-10H,13H-5,9-(azeno)furo[3,4- k]pyrazolo[3,4- b][1]oxa[4,6,10]triazacyclotridecin-1(4H)- yl)-2-methylpropan-1-ol | Calc'd 457, found 457 |

### Preparation of Example 8.1

### Scheme 36. Synthesis of (R)-2-methyl-2-(3,8,11-trimethyl-10,11,12,13-tetrahydro-5,9-(azeno)pyrazolo[3,4-b][1]oxa[4,6,10]triazacyclotridecin-1 (4H)-yl)propan-1-ol

Compounds in Table 8 below were prepared in accordance with the synthetic sequence illustrated in General Scheme 4 or General Scheme 7 and subsequent deprotection illustrated in General Scheme 9 using the corresponding starting materials.

TABLE 8

| | | | Exact Mass |
|---|---|---|---|
| Ex | Structure | Name | [M + H]⁺ |
| Ex-7.3 | 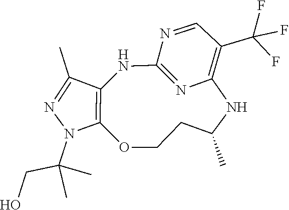 [SMILES: Cc1nn(C(C)(C)CO)c2c1Nc1ncc(C(F)(F)F)c(n1)N[C@H](C)CCO2] | (R)-2-(3,11-dimethyl-8-(trifluoromethyl)- 10,11,12,13-tetrahydro-5,9- (azeno)pyrazolo[3,4- b][1]oxa[4,6,10]triazacyclotridecin-1(4H)- yl)-2-methylpropan-1-ol | Calc'd 401, found 401 |
| Ex-7.4 | 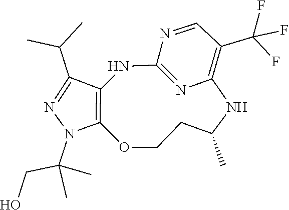 [SMILES: CC(C)c1nn(C(C)(C)CO)c2c1Nc1ncc(C(F)(F)F)c(n1)N[C@H](C)CCO2] | (R)-2-(3-isopropyl-11-methyl-8- (trifluoromethyl)-10,11,12,13-tetrahydro- 5,9-(azeno)pyrazolo[3,4- b][1]oxa[4,6,10]triazacyclotridecin-1(4H)- yl)-2-methylpropan-1-ol | Calc'd 429, found 429 |
| Ex-7.5 | 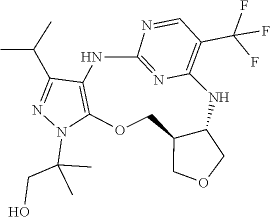 [SMILES: CC(C)c1nn(C(C)(C)CO)c2c1Nc1ncc(C(F)(F)F)c(n1)N[C@@H]1COC[C@H]1CO2] | 2-((10aS,13aS)-3-isopropyl-8- (trifluoromethyl)-10a,11,13a,14- tetrahydro-10H,13H-5,9-(azeno)furo[3,4- k]pyrazolo[3,4- b][1]oxa[4,6,10]triazacyclotridecin-1(4H)- yl)-2-methylpropan-1-ol | Calc'd 457, found 457 |

### Preparation of Example 8.1

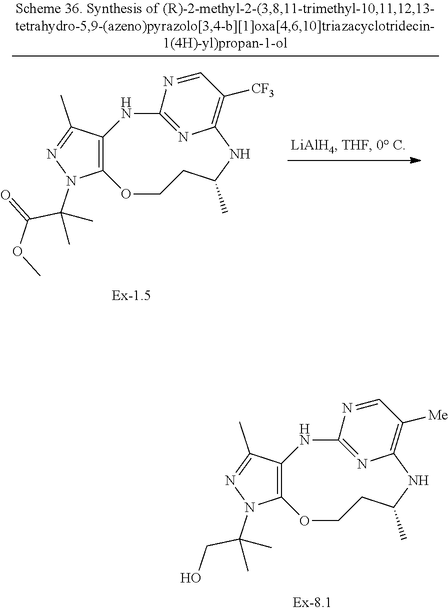 [SMILES: COC(=O)C(C)(C)n1nc(C)c2c1OCC[C@@H](C)Nc1nc(ncc1C(F)(F)F)N2 ; Cc1cnc2nc1N[C@H](C)CCOc1c(c(C)nn1C(C)(C)CO)N2]

### (R)-2-methyl-2-(3,8,11-trimethyl-10,11,12,13-tetrahydro-5,9-(azeno)pyrazolo[3,4-b][1]oxa[4,6,10]triazacyclotridecin-1 (4H)-yl)propan-1-ol (Ex-8.1)

To a vial containing methyl (R)-2-(3,11-dimethyl-8-(trifluoromethyl)-10,11,12,13-tetrahydro-5,9-(azeno)pyrazolo[3,4-b][1]oxa[4,6,10]triazacyclotridecin-1 (4H)-yl)-2-methylpropanoate Ex-1.5 (21 mg, 0.049 mmol) was added THF (500 μL). To this solution was added LiAlH₄ in THF (50 μL, 0.100 mmol) at 0° C. dropwise. The mixture was allowed to stir at RT for 2 h, then was quenched with saturated aqueous NH₄Cl and extracted with EtOAc. The combined organic layers were washed with brine, dried over MgSO₄, filtered and concentrated under reduced pressure to afford a residue. The residue was purified by reversed phase HPLC, eluting with water (0.1% TFA)-MeCN, to afford the title compound Ex-8.1. MS (ESI): m/z calc'd for C₁₇H₂₆N₆O₂ [M+H]⁺: 347, found 347; ¹H NMR (DMSO-d₆, 600 MHz) δ 7.55 (1H, s), 7.32 (1H, s), 6.22 (1H, d, J=5.3 Hz), 4.73 (1H, t, J=6.0 Hz), 4.51-4.38 (1H, m), 4.38-4.26 (1H, m), 4.05-3.96 (1H, m), 3.67 (1H, dd, J=10.7, 5.9 Hz), 3.58 (1H, dd, J=10.7, 6.2 Hz), 2.07 (3H, s), 1.95-1.91 (1H, m), 1.90 (3H, s), 1.58-1.49 (1H, m), 1.43 (5H, s), 1.29 (3H, d, J=7.0 Hz).

### Preparation of Example 9.1

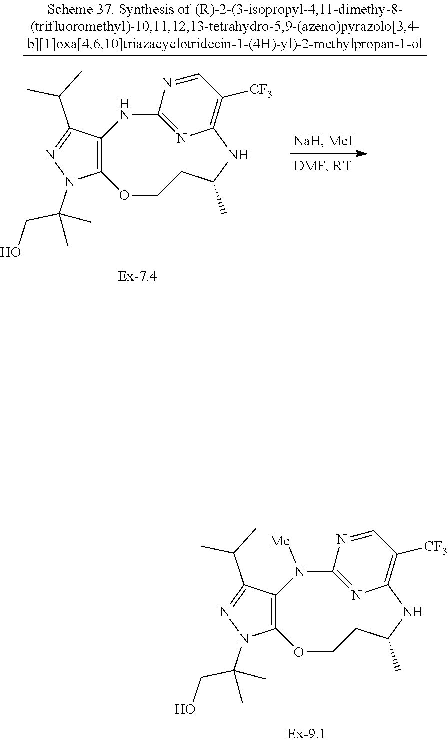 [SMILES: CC(C)c1nn(C(C)(C)CO)c2c1Nc1ncc(C(F)(F)F)c(n1)N[C@H](C)CCO2 ; CC(C)c1nn(C(C)(C)CO)c2c1N(C)c1ncc(C(F)(F)F)c(n1)N[C@H](C)CCO2]

### (R)-2-(3-isopropyl-4,11-dimethyl-8-(trifluoromethyl)-10,11,12,13-tetrahydro-5,9-(azeno)pyrazolo[3,4-b][1]oxa[4,6,10]triazacyclotridecin-1 (4H)-yl)-2-methylpropan-1-ol (Ex-9.1)

To a vial was added (R)-2-(3-isopropyl-11-methyl-8-(trifluoromethyl)-10,11,12,13-tetrahydro-5,9-(azeno)pyrazolo[3,4-b][1]oxa[4,6,10]triazacyclotridecin-1 (4H)-yl)-2-methylpropan-1-ol Ex-7.4 (13.8 mg, 0.032 mmol) and DMF (400 μL). To this solution was added NaH (2.9 mg, 0.073 mmol) at RT. The reaction mixture was allowed to stir at RT for 30 min, then Mel (4 μL, 0.06 mmol) was added. The mixture was quenched with NH₄Cl and extracted with EtOAc. The combined organic layers were washed with brine and dried over MgSO₄. The solution was filtered and concentrated under reduced pressure. The crude product was purified by chiral preparative SFC (Column & Dimensions: (R,R)-Whelk-O, 21×250 mm, 5 μm, Mobile phase A: CO₂; Mobile phase B: 15% MeOH w/0.1% NH₄OH·IPA) to afford the title compound Ex-9.1. MS (ESI): m/z calc'd for C₂₀H₂₉F₃N₆O₂ [M+H]⁺: 443, found 443; ¹H NMR (DMSO-d₆, 600 MHz) δ 8.17 (1H, s), 6.70 (1H, s), 4.79 (1H, s), 4.51 (1H, s), 4.37 (1H, s), 4.10 (1H, d, J=10.3 Hz), 3.70 (1H, dd, J=10.7, 5.7 Hz), 3.68-3.61 (1H, m), 3.28 (3H, s), 2.83 (1H, dt, J=13.6, 6.8 Hz), 2.06 (1H, m), 1.67 (1H, m), 1.45 (6H, d, J=18.1 Hz), 1.18 (6H, t, J=7.0 Hz), 1.00 (3H, s).

### Preparation of Example 10.1

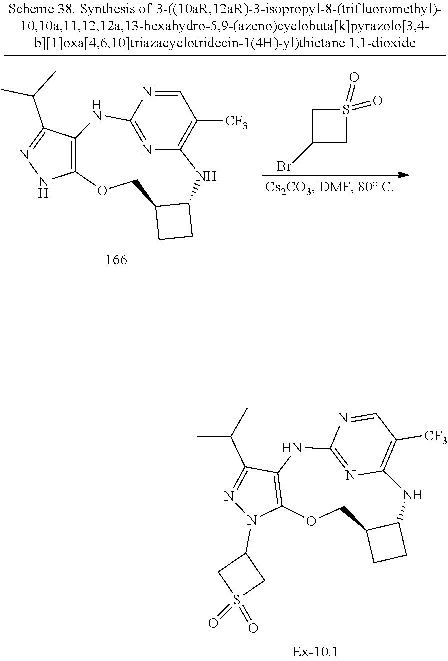

### 3-((10aR,12aR)-3-isopropyl-8-(trifluoromethyl)-10,10a,11,12,12a,13-hexahydro-5,9-(azeno)cyclobuta[k]pyrazolo[3,4-b][1]oxa[4,6,10]triazacyclotridecin-1 (4H)-yl) thietane 1,1-dioxide (Ex-10.1)

To a vial containing (10aR,12aR)-3-isopropyl-8-(trifluoromethyl)-1,4,10,10a,11,12,12a,13-octahydro-5,9-(azeno)cyclobuta[k]pyrazolo[3,4-b][1]oxa[4,6,10]triazacyclotridecine (38.2 mg, 0.104 mmol) were added 3-bromothietane 1,1-dioxide (29 mg, 0.157 mmol), Cs₂CO₃ (101 mg, 0.311 mmol) and DMF (1000 μL). The mixture was allowed to stir at 80° C. for 2.5 h, then was filtered and purified by reversed phase HPLC, then eluted with water (0.1% NH₄OH)-MeCN, to afford the title compound Ex-10.1. MS (ESI): m/z calc'd for C₁₉H₂₃F₃N₆O₃S [M+H]⁺: 473, found 473; ¹H NMR (DMSO-d₆, 600 MHz) δ 8.70 (1H, s), 8.08 (1H, s), 6.81 (1H, s), 5.29-5.18 (1H, m), 4.78 (2H, dd, J=10.5, 4.4 Hz), 4.65 (1H, td, J=10.0, 8.9, 4.4 Hz), 4.56 (1H, dd, J=13.5, 6.1 Hz), 4.50 (1H, d, J=10.2 Hz), 3.92 (1H, d, J=7.1 Hz), 3.91-3.85 (1H, m), 3.30-3.28 (1H, m), 3.13 (1H, hept, J=6.6 Hz), 2.37 (1H, p, J=8.3 Hz), 2.16 (1H, q, J=10.4 Hz), 2.06 (1H, q, J=9.0 Hz), 1.94 (1H, q, J=10.1 Hz), 1.45 (1H, q, J=10.0 Hz), 1.21 (2H, d, J=6.8 Hz), 1.09 (3H, d, J=6.9 Hz).

### Preparation of Examples 11.1 and 11.2

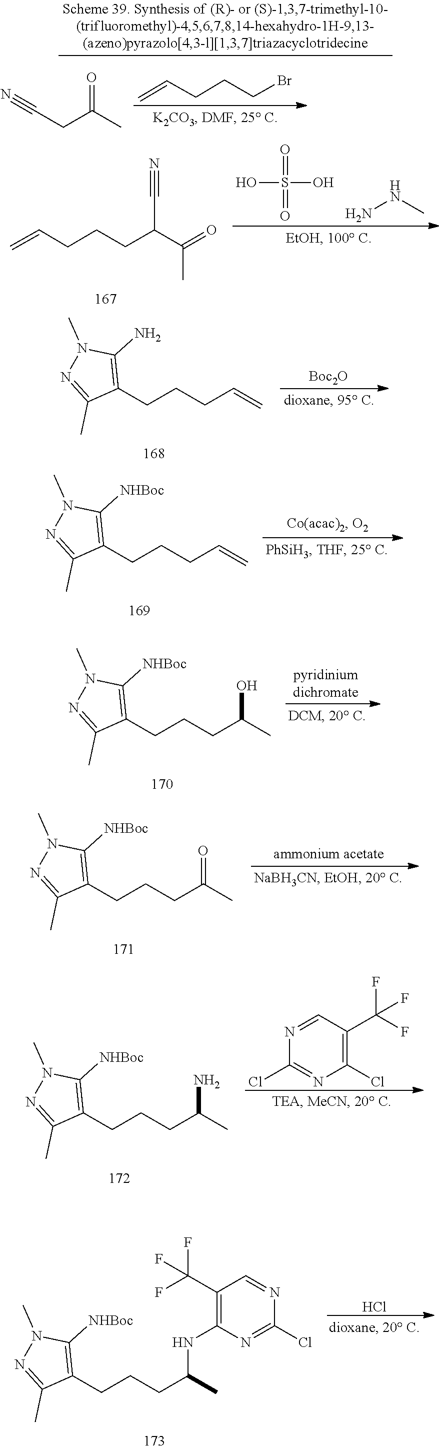

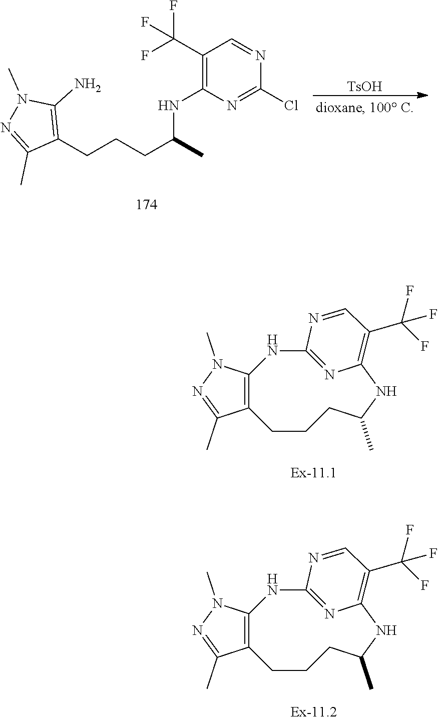 [SMILES: Cc1nn(C)c(N)c1CCC[C@H](C)Nc1nc(Cl)ncc1C(F)(F)F ; "TsOH" ; Cc1nn(C)c2c1CCC[C@@H](C)Nc1nc(ncc1C(F)(F)F)N2 ; Cc1nn(C)c2c1CCC[C@H](C)Nc1nc(ncc1C(F)(F)F)N2]

### 2-Acetylhept-6-enenitrile (167)

To a solution of 3-oxobutanenitrile (10 g, 120 mmol) in anhydrous DMF (300 mL) was added K₂CO₃ (18.30 g, 132 mmol) and 5-bromopent-1-ene (19.73 g, 132 mmol) and the reaction mixture was allowed to stir at 25° C. under for 16 h. The reaction mixture was poured into water (300 mL) and extracted with EtOAc (300 mL×3). The combined organic layers were washed with water (200 mL×2), dried over Na₂SO₄. The solution was filtered and concentrated under reduced pressure. The crude product was purified by flash chromatography on silica gel (gradient elution of 0-10% EtOAc/PE) to afford the title compound 167.

### 1,3-Dimethyl-4-(pent-4-en-1-yl)-1H-pyrazol-5-amine (168)

To a solution of 2-acetylhept-6-enenitrile 167 (14 g, 93 mmol) in anhydrous EtOH (200 mL) was added methylhydrazine sulfate (19.74 g, 139 mmol), and the reaction mixture was allowed to stir

### tert-Butyl (R)- and (S)-(4-(4-aminopentyl)-1,3-dimethyl-1H-pyrazol-5-yl)carbamate (172)

To a solution of tert-butyl (1,3-dimethyl-4-(4-oxopentyl)-1H-pyrazol-5-yl)carbamate 171 (400 mg, 1.354 mmol) in anhydrous EtOH (5 mL) was added ammonium acetate (313 mg, 4.06 mmol) and NaBH₃CN (128 mg, 2.031 mmol), and the reaction mixture was allowed to stir at 20° C. for 14 h. The reaction mixture was poured into water (15 mL) and extracted with EtOAc (15 mL×3), and dried over Na₂SO₄. The mixture was filtered and concentrated under reduced pressure to afford the title compound 172. The product was used in the next step directly without further purification.

### tert-Butyl (R)- and (S)-(4-(4-((2-chloro-5-(trifluoromethyl)pyrimidin-4-yl)amino) pentyl)-1,3-dimethyl-1H-pyrazol-5-yl)carbamate (173)

To a solution of tert-butyl (R)- and (S)-(4-(4-((2-chloro-5-(trifluoromethyl)pyrimidin-4-yl)amino) pentyl)-1,3-dimethyl-1H-pyrazol-5-yl)carbamate 172 (150 mg, 0.315 mmol, 26.6% yield) in anhydrous MeCN (10 mL) was added 2,4-dichloro-5-(trifluoromethyl)pyrimidine (384 mg, 1.771 mmol) and TEA (0.5 mL, 3.54 mmol), and the reaction mixture was allowed to stir at 20° C. for 1 h. The solution was filtered and concentrated under reduced pressure. The crude product was purified by reversed-phase HPLC, eluting with water (0.1% TFA)-MeCN, to afford the title compound 173.

### (R)- and (S)-N-(5-(5-Amino-1,3-dimethyl-1H-pyrazol-4-yl) pentan-2-yl)-2-chloro-5-(trifluoromethyl)pyrimidin-4-amine (174)

To a solution of tert-butyl (R)- and (S)-(4-(4-((2-chloro-5-(trifluoromethyl)pyrimidin-4-yl)amino) pentyl)-1,3-dimethyl-1H-pyrazol-5-yl)carbamate 173 (150 mg, 0.315 mmol) in 1,4-dioxane (4 mL) was added 4 M HCl dioxane (4 mL), and the reaction mixture was allowed to stir at 20° C. for 1 h. The reaction mixture was adjusted pH to 7-8 using saturated NaHCO₃ (20 mL) and extracted with EtOAc (20 mL×3), and dried over Na₂SO₄. The mixture was filtered and concentrated to afford the title compound 174. The product was used in the next step directly without further purification.

### (R)- or (S)-1,3,7-Trimethyl-10-(trifluoromethyl)-4,5,6,7,8,14-hexahydro-1H-9,13-(azeno)pyrazolo[4,3-l][1,3,7]triazacyclotridecine (Ex-11.1 and Ex-11.2)

To a solution of (R)- and (S)-N-(5-(5-amino-1,3-dimethyl-1H-pyrazol-4-yl) pentan-2-yl)-2-chloro-5-(trifluoromethyl)pyrimidin-4-amine 174 (80 mg, 0.212 mmol) in anhydrous 1,4-dioxane (1.5 mL) was added 4-methylbenzenesulfonic acid (18.28 mg, 0.106 mmol) and the reaction mixture was allowed to stir at 100° C. for 14 h. The solution was filtered and concentrated under reduced pressure. The residue was purified by reversed-phase HPLC, eluting with water (0.1% TFA)-MeCN, to afford the racemic material. The racemic material was resolved to its component enantiomers by chiral preparative SFC (Column: Chiralpak AD-3, 250 mm×30 mm, 10 μm; Mobile phase A: CO₂; Mobile phase B: 30% EtOH/IPA) to afford the title compounds 11.1 (t_{R}=4.1 min) and 11.2 (t_{R}=4.7 min). MS (ESI): m/z calc'd for C₁₅H₁₉F₃N₆ [M+H]⁺: 341, found 341; ¹H NMR (400 MHz, CDCl₃) δ 8.11 (br s, 1H), 7.21 (br s, 1H), 5.06 (br s, 1H), 3.73-3.81 (m, 1H), 3.71 (s, 3H), 3.15 (m, 1H), 2.27-2.35 (m, 1H), 2.20 (s, 3H), 1.89-2.01 (m, 2H), 1.54-1.63 (m, 1H), 1.43-1.52 (m, 1H), 1.29 (d, J=6.65 Hz, 3H); MS (ESI): m/z calc'd for C₁₅H₁₉F₃N₆ [M+H]⁺: 341, found 341; ¹H NMR (400 MHz, CDCl₃) δ 8.11 (br s, 1H), 7.21 (br s, 1H), 5.06 (br s, 1H), 3.73-3.80 (m, 1H), 3.71 (s, 3H), 3.15 (m, 1H), 2.27-2.34 (m, 1H), 2.20 (s, 3H), 1.89-2.02 (m, 2H), 1.54-1.62 (m, 1H), 1.44-1.52 (m, 1H), 1.29 (d, J=7.04 Hz, 3H).

### Preparation of Examples 12.1 and 12.2

### Scheme 40. Synthesis of (10aS,13aS)- or (10aR,13aR)-3-cyclobutyl-1-methyl-8-(trifluoromethyl)-3,4,10a,11,13,13a,14,15-octahydro-10H-5,9-(azeno)furo[3,4-h]pyrazolo[4,3-1][1,3,7]triazacyclotridecine

with water (0.1% TFA)-MeCN, to afford the racemic material. The racemic material was resolved to its component enantiomers by chiral preparative SFC (Column: Chiralpak AD-3, 250 mm×30 mm, 10 μm; Mobile phase A: CO₂; Mobile phase B: 30% EtOH/IPA) to afford the title compounds 11.1 (t_{R}=4.1 min) and 11.2 (t_{R}=4.7 min). MS (ESI): m/z calc'd for C₁₅H₁₉F₃N₆ [M+H]⁺: 341, found 341; ¹H NMR (400 MHz, CDCl₃) δ 8.11 (br s, 1H), 7.21 (br s, 1H), 5.06 (br s, 1H), 3.73-3.81 (m, 1H), 3.71 (s, 3H), 3.15 (m, 1H), 2.27-2.35 (m, 1H), 2.20 (s, 3H), 1.89-2.01 (m, 2H), 1.54-1.63 (m, 1H), 1.43-1.52 (m, 1H), 1.29 (d, J=6.65 Hz, 3H); MS (ESI): m/z calc'd for C₁₅H₁₉F₃N₆ [M+H]⁺: 341, found 341; ¹H NMR (400 MHz, CDCl₃) δ 8.11 (br s, 1H), 7.21 (br s, 1H), 5.06 (br s, 1H), 3.73-3.80 (m, 1H), 3.71 (s, 3H), 3.15 (m, 1H), 2.27-2.34 (m, 1H), 2.20 (s, 3H), 1.89-2.02 (m, 2H), 1.54-1.62 (m, 1H), 1.44-1.52 (m, 1H), 1.29 (d, J=7.04 Hz, 3H).

### Preparation of Examples 12.1 and 12.2

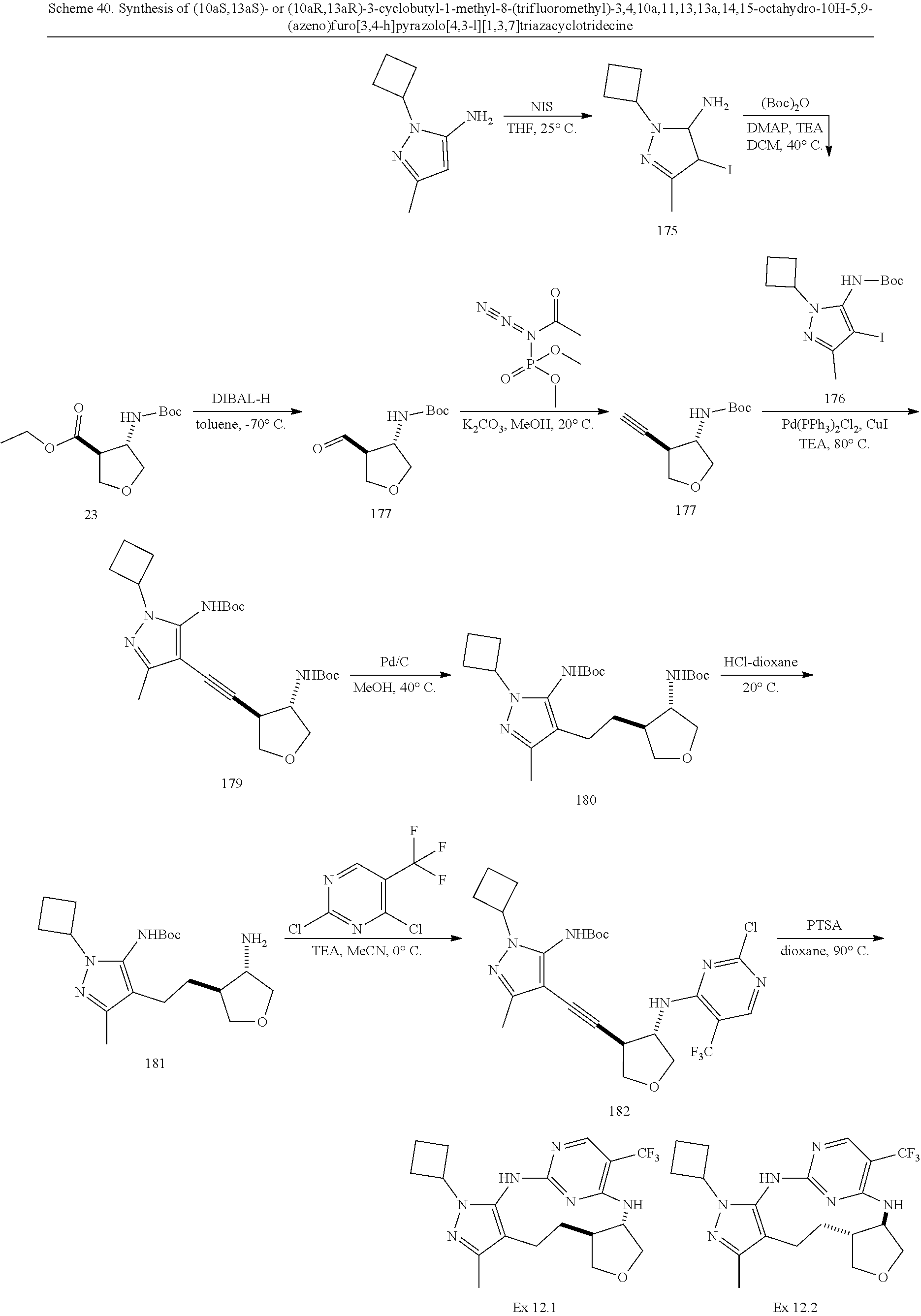

### 1-Cyclobutyl-4-iodo-3-methyl-1H-pyrazol-5-amine (175)

### tert-Butyl ((3S,4S)- and (3R,4R)-4-formyltetrahydrofuran-3-yl)carbamate (177)

To a solution of ethyl (3S,4S)- or (3R,4R)-4-((tert-butoxycarbonyl)amino)tetrahydrofuran-3-carboxylate (5 g, 19.28 mmol) in toluene (50 mL) was added diisobutylaluminum hydride (1 M in toluene, 23.1 mL, 23.1 mmol) at −78° C. The mixture was allowed to stir at −78° C. for 1 h, then MeOH (25 mL) was added and the mixture was warmed to RT. Brine (30 mL) was added, and the reaction mixture was filtered, poured into water (200 mL) and extracted with EtOAc (3×100 mL). The combined organic layers were dried over Na₂SO₄. The solution was filtered and concentrated under reduced pressure. The mixture was concentrated to afford the title compound 177. The product was used in the next step directly without further purification.

### tert-Butyl ((3S,4S)- or (3R,4R)-4-ethynyltetrahydrofuran-3-yl)carbamate (178)

To a solution of tert-Butyl ((3S,4S)- or (3R,4R)-4-formyltetrahydrofuran-3-yl)carbamate 177 (4 g, 18.58 mmol) in anhydrous MeOH (50 mL) was added dimethyl (1-diazo-2-oxopropyl) phosphonate (10.71 g, 55.7 mmol) at 0° C. followed by potassium carbonate (10.3 g, 74.3 mmol). The reaction mixture was allowed to stir at 20° C. for 16 h. The reaction mixture was poured into water (100 mL) and extracted with EtOAc (3×50 mL). The combined organic layers were washed with brine (3×30 mL), dried over Na₂SO₄, filtered and concentrated under reduced pressure. The crude product was purified by flash chromatography on silica gel (gradient elution of 0-30% EtOAc/PE) to afford the title compound 178. ¹H NMR (400 MHz, CDCl₃) § 4.83 (br s, 1H), 4.27 (br s, 1H), 4.17-4.09 (m, 1H), 4.03 (dd, J=5.6, 9.4 Hz, 1H), 3.80-3.73 (m, 1H), 3.67 (m, 1H), 2.91 (br s, 1H), 2.22 (d, J=2.5 Hz, 1H), 1.45 (br s, 9H).

### tert-butyl ((3S,4S)- or (3R,4R)-4-((5-((tert-butoxycarbonyl)amino)-1-cyclobutyl-3-methyl-1H-pyrazol-4-yl) ethynyl)tetrahydrofuran-3-yl)carbamate (179)

A mixture of tert-butyl (4-ethynyltetrahydrofuran-3-yl)carbamate 176 (350 mg, 1.657 mmol), tert-butyl ((3S,4S)- and (3R,4R)-4-ethynyltetrahydrofuran-3-yl)carbamate 178 (500 mg, 1.325 mmol), bis(triphenylphosphine) palladium (II) dichloride (11.63 mg, 0.017 mmol) and copper (I) iodide (31.6 mg, 0.166 mmol) in TEA (3 mL) was allowed to stir at 80° C. under N₂ for 16 h. The solution was filtered and concentrated under reduced pressure. The residue was purified by flash chromatography on silica gel (gradient elution of 0-35% EtOAc/PE) to afford the title compound 179.

### tert-Butyl ((3S,4S)- and (3R,4R)-4-(2-(5-((tert-butoxycarbonyl)amino)-1-cyclobutyl-3-methyl-1H-pyrazol-4-yl)ethyl)tetrahydrofuran-3-yl)carbamate (180)

To a solution of tert-butyl ((3S,4S)- and (3R,4R)-4-((5-((tert-butoxycarbonyl)amino)-1-cyclobutyl-3-methyl-1H-pyrazol-4-yl) ethynyl)tetrahydrofuran-3-yl)carbamate 179 (280 mg, 0.608 mmol) in anhydrous MeOH (10 mL) was added Pd/C (129 mg, 0.122 mmol), and the reaction mixture was allowed to stir at 40° C. under H₂ (15 psi) for 1 h. The mixture was filtered and concentrated to afford the title compound 180. The product was used in the next step directly without further purification.

### 4-(2-((3S,4S)- and (3R,4R)-4-Aminotetrahydrofuran-3-yl)ethyl)-1-cyclobutyl-3-methyl-1H-pyrazol-5-amine (181)

A solution of tert-butyl ((3S,4S)- and (3R,4R)-4-(2-(5-((tert-butoxycarbonyl)amino)-1-cyclobutyl-3-methyl-1H-pyrazol-4-yl)ethyl)tetrahydrofuran-3-yl)carbamate 180 (475 mg, 1.02 mmol) in HCl-dioxane (5 mL) at 25° C. was allowed to stir at 20° C. for 2 h. Aqueous NaHCO₃ (IM) was added to adjust pH to 8-9. The solution was filtered and concentrated under reduced pressure to afford the title compound 181. The product was used in the next step directly without further purification.

### N-((3S,4S)- and (3R,4R)-4-(2-(5-Amino-1-cyclobutyl-3-methyl-1H-pyrazol-4-yl)ethyl)tetrahydrofuran-3-yl)-2-chloro-5-(trifluoromethyl)pyrimidin-4-amine (182)

The mixture of 4-(2-((3S,4S)- and (3R,4R)-4-aminotetrahydrofuran-3-yl)ethyl)-1-cyclobutyl-3-methyl-1H-pyrazol-5-amine 181 (260 mg, 0.983 mmol), 2,4-dichloro-5-(trifluoromethyl) pyrimidine (213 mg, 0.983 mmol) and TEA (0.685 mL, 4.92 mmol) in anhydrous MeCN (4 mL) was allowed to stir at 0° C. for 1 h. The mixture was filtered and concentrated under reduced pressure to give the crude product which was purified by flash chromatography on silica gel (gradient elution of 0-50% EtOAc/PE) to afford the title compound 182.

### (10aS,13aS)- or (10aR,13aR)-3-Cyclobutyl-1-methyl-8-(trifluoromethyl)-3,4,10a,11,13,13a,14,15-octahydro-10H-5,9-(azeno)furo[3,4-h]pyrazolo[4,3-l][1,3,7]triazacyclotridecine (Ex 12.1 and 12.2)

To a solution of N-((3S,4S)- and (3R,4R)-4-(2-(5-Amino-1-cyclobutyl-3-methyl-1H-pyrazol-4-yl)ethyl)tetrahydrofuran-3-yl)-2-chloro-5-(trifluoromethyl)pyrimidin-4-amine 182 (60 mg, 0.135 mmol) in dioxane (2 mL) was added 4-methylbenzenesulfonic acid (6.97 mg, 0.040 mmol) under nitrogen. The mixture was allowed to stir at 90° C. for 16 h. The reaction mixture was concentrated, and the residue was purified by reversed phase HPLC, eluting with water (0.1% TFA)-MeCN, to afford the title product as a racemic mixture. The racemic mixture was resolved by chiral preparative SFC (Column: Chiralpak AD-3 50×4.6 mm I.D., 3 μm, Mobile phase: A: CO₂ B: isopropanol (0.05% DEA), Gradient: from 5% to 40% of B in 2 min and hold 40% for 1.2 min, then 5% of B for 0.8 min) to afford the title compounds Ex-12.1 (Rt=1.5 min) and Ex-12.2. (Rt=1.8 min). MS (ESI): m/z calc'd for C₁₉H₂₃F₃N₆O [M+H]⁺: 409, found 409; ¹H NMR (400 MHz, MeOD-d₄) δ 8.10 (s, 1H), 4.76 (J=8.5 Hz, 1H), 4.11 (t, J=8.5 Hz, 1H), 4.04 (m, 1H), 3.97 (d, J=10.3 Hz, 1H), 3.73 (dd, J=6.0, 10.3 Hz, 1H), 3.12 (dd, J=7.0, 8.9 Hz, 1H), 2.82 (m, 1H), 2.50 (J=3.1, 10.0 Hz, 2H), 2.38-2.30 (m, 2H), 2.29-2.22 (m, 1H), 2.19 (s, 3H), 2.15-2.04 (m, 1H), 1.88-1.73 (m, 3H), 1.70-1.59 (m, 1H); MS (ESI): m/z calc'd for C₁₉H₂₃F₃N₆O [M+H]⁺: 409, found 409; ¹H NMR (400 MHz, MeOD-d₄) δ 8.14 (s, 1H), 4.76 (J=8.4 Hz, 1H), 4.12 (t, J=8.5 Hz, 1H), 4.07-4.02 (m, 1H), 3.99 (d, J=10.4 Hz, 1H), 3.71 (dd, J=6.0, 10.3 Hz, 1H), 3.11 (dd, J=7.2, 8.9 Hz, 1H), 2.83-2.73 (m, 1H), 2.50 (J=10.0 Hz, 2H), 2.38-2.30 (m, 2H), 2.29-2.22 (m, 1H), 2.19 (s, 3H), 2.14-2.04 (m, 1H), 1.89-1.82 (m, 1H), 1.82-1.74 (m, 2H), 1.70-1.58 (m, 1H).

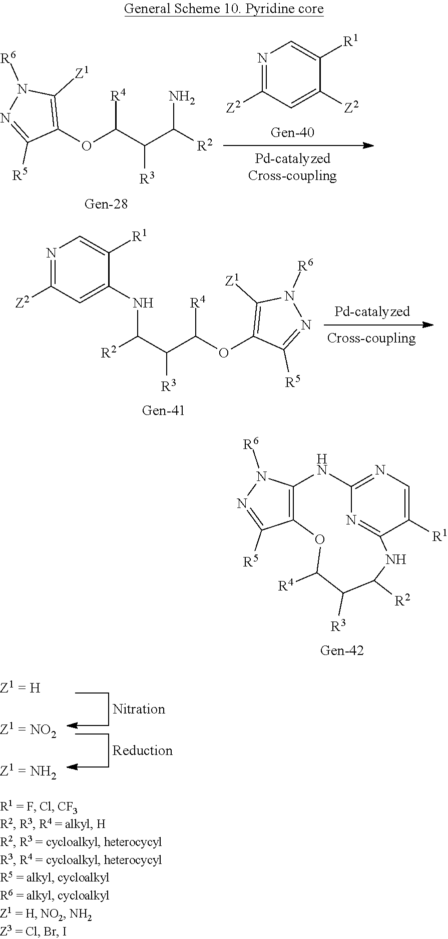

In General Scheme 10, commercially available pyridines of the form Gen-40 were coupled with amines of the form Gen-28 through Pd-catalyzed cross-coupling chemistry to access Gen-41. In instances of Gen-41 where Z¹=H, nitration of the pyrazole was performed. For nitropyrazoles of the form Gen-41 where Z¹=NO₂, reduction to the corresponding amine was performed. The aminopyrazole Gen-41 could ultimately be transformed through intramolecular Pd-catalyzed

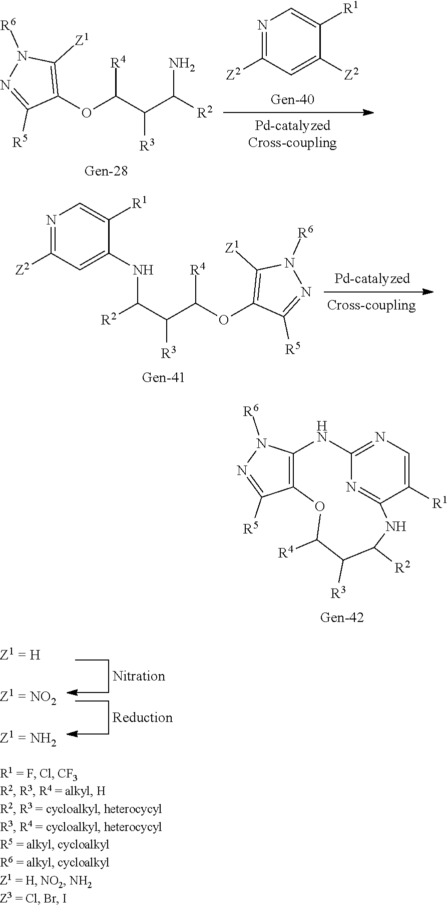

In General Scheme 10, commercially available pyridines of the form Gen-40 were coupled with amines of the form Gen-28 through Pd-catalyzed cross-coupling chemistry to access Gen-41. In instances of Gen-41 where Z¹=H, nitration of the pyrazole was performed. For nitropyrazoles of the form Gen-41 where Z¹=NO₂, reduction to the corresponding amine was performed. The aminopyrazole Gen-41 could ultimately be transformed through intramolecular Pd-catalyzed cross-coupling chemistry to afford compounds of the form Gen-42. Representative preparative examples from each sequence are described in more detail below.

### Preparation of Example 13.1

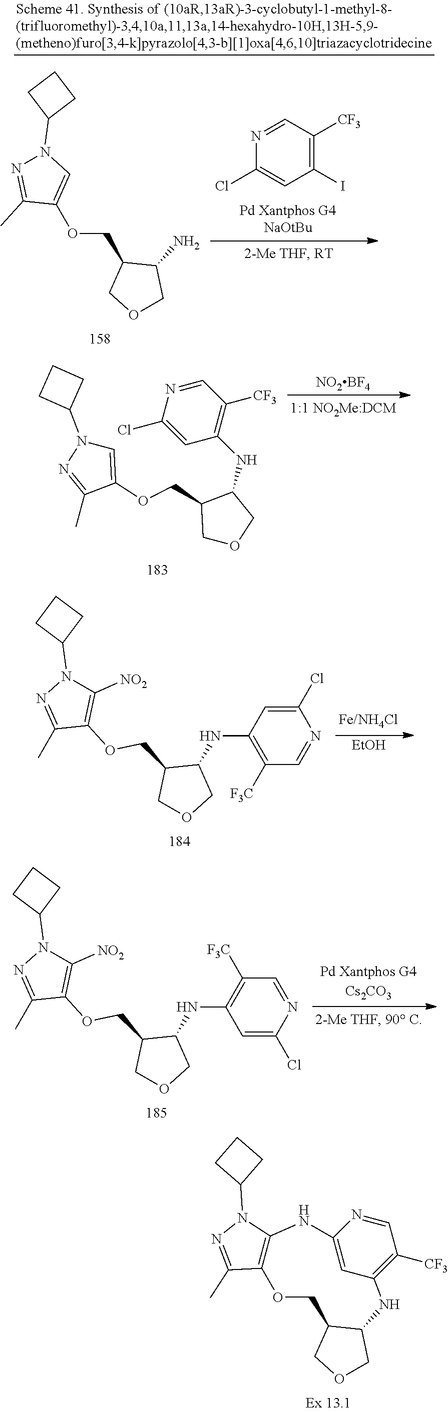

### 2-Chloro-N-((3S,4S)-4-(((1-cyclobutyl-3-methyl-1H-pyrazol-4-yl)oxy)methyl)tetrahydrofuran-3-yl)-5-(trifluoromethyl)pyridin-4-amine (183)

### 2-Chloro-N-((3R,4R)-4-(((1-cyclobutyl-3-methyl-5-nitro-1H-pyrazol-4-yl)oxy)methyl)tetrahydrofuran-3-yl)-5-(trifluoromethyl)pyridin-4-amine (184)

Nitronium tetrafluoroborate (100 μL, 0.957 mmol) was added to a vial in a glovebox. The vial was sealed and removed from the glovebox and all additional steps were performed under nitrogen in the hood. 5 mL of 1:1 DCM:nitromethane was added to the vial, and the vial was chilled to −10° C. 2-chloro-N-((3S,4S)-4-(((1-cyclobutyl-3-methyl-1H-pyrazol-4-yl)oxy)methyl)tetrahydrofuran-3-yl)-5-(trifluoromethyl)pyridin-4-amine 183 (275 mg, 0.638 mmol) was dissolved in 13 mL of 1:1 DCM:nitromethane and was added slowly to the solution of nitronium tetrafluoroborate. The reaction mixture was allowed to stir at −10° C. for 2 h, then allowed to stir overnight, eventually warming to 10° C. The reaction was quenched with MeOH. The reaction mixture was diluted with DCM and washed twice with water and once with brine. The combined organic fractions were dried over anhydrous sodium sulfate, filtered, and concentrated under reduced pressure. The residue was purified by flash chromatography on silica gel (gradient elution of 0-100% EtOAc/hexanes) to afford the title compound 184. MS (ESI): m/z calc'd for C₁₉H₂₁ClF₃N₅O₄ [M+H]⁺: 476, found 476.

### N-((3R,4R)-4-(((5-Amino-1-cyclobutyl-3-methyl-1H-pyrazol-4-yl)oxy)methyl)tetrahydrofuran-3-yl)-2-chloro-5-(trifluoromethyl)pyridin-4-amine (185)

2-chloro-N-((3R,4R)-4-(((1-cyclobutyl-3-methyl-5-nitro-1H-pyrazol-4-yl)oxy)methyl)tetrahydrofuran-3-yl)-5-(trifluoromethyl)pyridin-4-amine 184 (95 mg, 0.200 mmol), iron (111 mg, 1.996 mmol) and ammonium chloride (107 mg, 1.996 mmol) were added to a vial. Ethanol (1815 μL) and water (181 μL) were added, and the reaction mixture was allowed to stir 2 h at 90° C. The reaction mixture was diluted with DCM and filtered. The filtrate was concentrated under reduced pressure. The reaction mixture was diluted with DCM and washed with water. The biphasic mixture was passed through a phase separator cartridge and concentrated under reduced pressure to afford the title compound 185. The product was used in the next step directly without further purification. MS (ESI): m/z calc'd for C₁₉H₂₃ClF₃N₅O₂ [M+H]⁺: 446, found 446.

### (10aR,13aR)-3-Cyclobutyl-1-methyl-8-(trifluoromethyl)-3,4,10a,11,13a,14-hexahydro-10H,13H-5,9-(metheno)furo[3,4-k]pyrazolo[4,3-b][1]oxa[4,6,10]triazacyclotridecine, TFA (Ex-13.1)

Cesium carbonate (248 mg, 0.763 mmol) and XantPhos Pd G4 (29.4 mg, 0.031 mmol) were added to a vial. The vial was sealed, and its contents were placed under an inert atmosphere by performing 3 vacuum/nitrogen cycles. A solution of N-((3R,4R)-4-(((5-amino-1-cyclobutyl-3-methyl-1H-pyrazol-4-yl)oxy)methyl)tetrahydrofuran-3-yl)-2-chloro-5-(trifluoromethyl)pyridin-4-amine 185 (68 mg, 0.153 mmol) in 2-methyltetrahydrofuran (6100 μL) was added through the septum, and the reaction mixture was allowed to stir for 72 h at 90° C. The crude reaction mixture was scavenged for 1 h with Si-DMT. The reaction mixture was filtered and purified by HPLC, eluting acetonitrile/water gradient with 0.1% TFA modifier, linear gradient and lyophilized to afford the title compound 13.1. MS (ESI): m/z calc'd for C₁₉H₂₂F₃N₅O₂·TFA [M+H]+: 410, found 410; ¹H NMR (DMSO-d₆, 500 MHz) δ 9.63 (1H, s), 8.14 (1H, s), 6.68 (1H, s), 5.87 (1H, s), 4.74 (1H, p, J=8.1 Hz), 4.38 (1H, d, J=11.2 Hz), 4.33 (1H, d, J=9.8 Hz), 4.23 (1H, s), 4.14 (1H, t, J=9.0 Hz), 4.03-3.96 (2H, m), 3.74-3.69 (8H₈H₂H₈H, m), 3.17 (1H, t, J=9.2 Hz), 2.50 (7H, s), 2.43 (1H, q, J=10.0 Hz), 2.32 (1H, s), 2.22 (4H4H3H4H, s), 2.00-1.89 (1H, m), 1.72 (2H, dt, J=18.1, 8.9 Hz).

Compounds in Table 9 below were prepared in accordance with the synthetic sequence illustrated in General Scheme 6 and General Scheme 10 using the corresponding starting materials.

TABLE 9

| | | | Exact Mass |
|---|---|---|---|
| Ex | Structure | Name | [M + H]⁺ |
| Ex-13.2 | 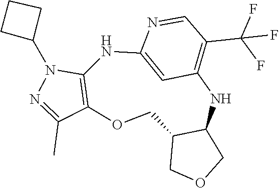 [SMILES: Cc1nn(C2CCC2)c2c1OC[C@H]1COC[C@@H]1Nc1cc(ncc1C(F)(F)F)N2] | (10aR,13aR)-3-cyclobuty1-1- methyl-8-(trifluoromethyl)- 3,4,10a,11,13a,14-hexahydro- 10H,13H-5,9-(metheno)furo[3,4- k]pyrazolo[4,3- b][1]oxa[4,6,10]triazacyclotridecine | Calc'd 410, found 410 |
| Ex-13.3 | 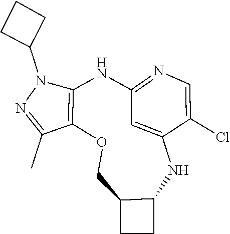 [SMILES: Cc1nn(C2CCC2)c2c1OC[C@@H]1CC[C@H]1Nc1cc(ncc1Cl)N2] | (10aR,12aR)-8-chloro-3-cyclobutyl- 1-methyl-3,4,10,10a,11,12,12a,13- octahydro-5,9- (metheno)cyclobuta[k]pyrazolo[4,3- b][1]oxa[4,6,10]triazacyclotridecine | Calc'd 360, found 360 |
| Ex-13.2 | 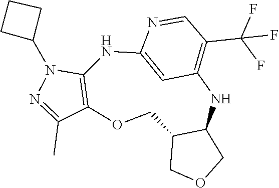 [SMILES: Cc1nn(C2CCC2)c2c1OC[C@H]1COC[C@@H]1Nc1cc(ncc1C(F)(F)F)N2] | (10aR,13aR)-3-cyclobutyl-1- methyl-8-(trifluoromethyl)- 3,4,10a,11,13a,14-hexahydro- 10H,13H-5,9-(metheno)furo[3,4- k]pyrazolo[4,3- b][1]oxa[4,6,10]triazacyclotridecine | Calc'd 410, found 410 |
| Ex-13.3 | 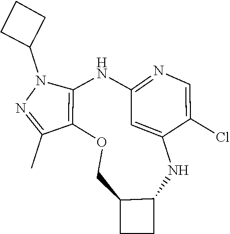 [SMILES: Cc1nn(C2CCC2)c2c1OC[C@@H]1CC[C@H]1Nc1cc(ncc1Cl)N2] | (10aR,12aR)-8-chloro-3-cyclobutyl- 1-methyl-3,4,10,10a,11,12,12a,13- octahydro-5,9- (metheno)cyclobuta[k]pyrazolo[4,3- b][1]oxa[4,6,10]triazacyclotridecine | Calc'd 360, found 360 |
| Ex-13.4 | 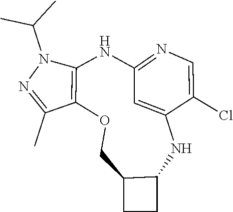 [SMILES: Cc1nn(C(C)C)c2c1OC[C@@H]1CC[C@H]1Nc1cc(ncc1Cl)N2] | (10aR,12aR)-8-chloro-3-isopropyl- 1-methyl-3,4,10,10a,11,12,12a,13- octahydro-5,9- (metheno)cyclobuta[k]pyrazolo[4,3- b][1]oxa[4,6,10]triazacyclotridecine | Calc'd 348, found 348 |
| Ex-13.5 | 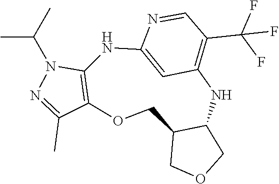 [SMILES: Cc1nn(C(C)C)c2c1OC[C@@H]1COC[C@H]1Nc1cc(ncc1C(F)(F)F)N2] | (10aS,13aS)-3-isopropyl-1-methyl- 8-(trifluoromethyl)- 3,4,10a,11,13a,14-hexahydro- 10H,13H-5,9-(metheno)furo[3,4- k]pyrazolo[4,3- b][1]oxa[4,6,10]triazacyclotridecine | Calc'd 398, found 398 |
| Ex-13.6 | 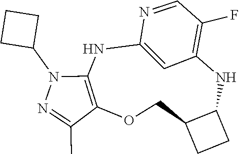 [SMILES: Cc1nn(C2CCC2)c2c1OC[C@@H]1CC[C@H]1Nc1cc(ncc1F)N2] | (10aR,12aR)-3-cyclobutyl-8-fluoro- 1-methyl-3,4,10,10a,11,12,12a,13- octahydro-5,9- (metheno)cyclobuta[k]pyrazolo[4,3- b][1]oxa[4,6,10]triazacyclotridecine | Calc'd 344, found 344 |
| Ex-13.7 | 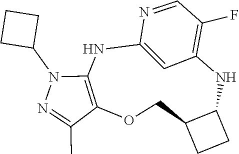 [SMILES: Cc1nn(C2CCC2)c2c1OC[C@@H]1CC[C@H]1Nc1cc(ncc1F)N2] | (10aR,12aR)-3-cyclobutyl-8-fluoro- 1-methyl-3,4,10,10a,11,12,12a,13- octahydro-5,9- (metheno)cyclobuta[k]pyrazolo[4,3- b][1]oxa[4,6,10]triazacyclotridecine | Calc'd 344, found 344 |
| Ex-13.8 | 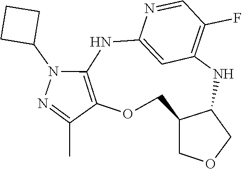 [SMILES: Cc1nn(C2CCC2)c2c1OC[C@@H]1COC[C@H]1Nc1cc(ncc1F)N2] | (10aR,13aR)-3-cyclobutyl-8-fluoro- 1-methyl-3,4,10a,11,13a,14- hexahydro-10H,13H-5,9- (metheno)furo[3,4-k]pyrazolo[4,3- b][1]oxa[4,6,10]triazacyclotridecine | Calc'd 360, found 360 |
| Ex-13.9 | 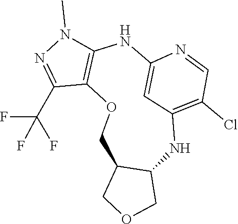 [SMILES: Cn1nc(C(F)(F)F)c2c1Nc1cc(c(Cl)cn1)N[C@@H]1COC[C@H]1CO2] | (10aS,13aS)-8-chloro-3-methyl-1- (trifluoromethyl)- 3,4,10a,11,13a,14-hexahydro- 10H,13H-5,9-(metheno)furo[3,4- k]pyrazolo[4,3- b][1]oxa[4,6,10]triazacyclotridecine | Calc'd 390, found 390 |

### Preparation of Examples 14.1 and 14.2

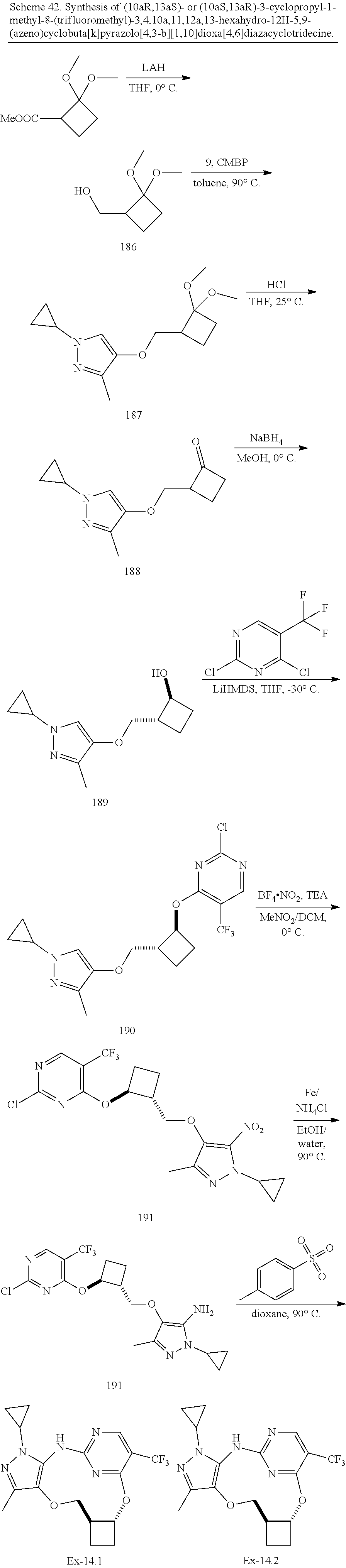

### (2,2-Dimethoxycyclobutyl)methanol (186)

To a solution of methyl 2,2-dimethoxycyclobutane-1-carboxylate (1 g, 5.74 mmol) in THF (10 mL), cooled to 0° C., was added LAH (8.61 mL, 8.61 mmol). The reaction was allowed to stir for 1 h at 0° C. Ice water (2 mL) was added to the mixture. The suspension was filtered, and the filtrate was concentrated under reduced pressure to afford the title compound 186, which was used directly for the next step without further purification. ¹H NMR (400 MHz, CDCl₃) δ 3.72-3.68 (m, 2H), 3.58-3.20 (m, 6H), 2.67-2.60 (m, 1H), 2.60-2.41 (m, 1H), 2.25-2.16 (m, 1H), 2.05-2.00 (m, 1H), 1.83-1.73 (m, 1H), 1.56-1.44 (m, 1H).

### 1-Cyclopropyl-4-((2,2-dimethoxycyclobutyl)methoxy)-3-methyl-1H-pyrazole (187)

To a solution of (2,2-dimethoxycyclobutyl)methanol 186 (800 mg, 5.47 mmol) and 1-cyclopropyl-3-methyl-1H-pyrazol-4-ol 186 (756 mg, 5.47 mmol) in toluene (10 mL) was added 2-(tributyl-15-phosphaneylidene) acetonitrile (1981 mg, 8.21 mmol) in a glove box. The reaction was allowed to stir for 16 h at 90° C. The reaction solution was concentrated under reduced pressure. The crude product was purified by flash chromatography on silica gel (gradient elution of 0-25% EtOAc/PE) to afford the title compound 187. ¹H NMR (400 MHz, CDCl₃) δ 7.03 (s, 1H), 4.03 (dd, J=7.7, 9.6 Hz, 1H), 3.70 (dd, J=6.6, 9.7 Hz, 1H), 3.53-3.39 (m, 6H), 2.80 (q, J=7.7 Hz, 1H), 2.23-2.15 (m, 1H), 2.15-2.09 (m, 4H), 2.07-1.99 (m, 1H), 1.88 (dtd, J=4.5, 9.3, 11.0 Hz, 1H), 1.49-1.39 (m, 1H), 1.06-1.01 (m, 2H), 0.96-0.90 (m, 2H).

### 2-(((1-Cyclopropyl-3-methyl-1H-pyrazol-4-yl)oxy)methyl)cyclobutan-1-one (188)

To a solution of 1-cyclopropyl-4-((2,2-dimethoxycyclobutyl)methoxy)-3-methyl-1H-pyrazole 187 (1.15 g, 4.32 mmol) in THF (5 mL) was added aq. HCl (1 mL, 12.18 mmol), and the reaction mixture was allowed to stir at 25° C. for 1 h. Aqueous NaHCO₃ was added to adjust pH to 7. The reaction mixture was poured into water (10 mL) and extracted with EtOAc (10 mL×3). The combined organic layers were dried over MgSO₄. The solution was filtered and concentrated under reduced pressure. The crude product was purified by flash chromatography on silica gel (gradient elution of 0-30% EtOAc/PE) to afford the title compound 188.

### (1R,2S)- and (1S,2R)-2-(((1-Cyclopropyl-3-methyl-1H-pyrazol-4-yl)oxy)methyl)cyclobutan-1-ol (189)

To a solution of 2-(((1-cyclopropyl-3-methyl-1H-pyrazol-4-yl)oxy)methyl)cyclobutan-1-one 188 (820 mg, 3.72 mmol) in anhydrous MeOH (10 mL) was added NaBH₄ (282 mg, 7.45 mmol) at 0° C., and the reaction mixture was allowed to stir at 0° C. for 2 h. The reaction mixture was poured into ice water (10 mL) and extracted with EtOAc (30 mL×3). The organic layer was washed with water (10 mL×3) and dried over Na₂SO₄. The solution was filtered and concentrated under reduced pressure. The crude product was purified by flash chromatography on silica gel (gradient elution of 0-30% EtOAc/PE) to afford the title compound 189.

with water (10 mL×3) and dried over Na₂SO₄. The solution was filtered and concentrated under reduced pressure. The crude product was purified by flash chromatography on silica gel (gradient elution of 0-30% EtOAc/PE) to afford the title compound 190.

### 2-chloro-4-((1R,2S)- and (1S,2R)-2-(((1-Cyclopropyl-3-methyl-5-nitro-1H-pyrazol-4-yl)oxy)methyl)cyclobutoxy)-5-(trifluoromethyl)pyrimidine (191)

To a solution of 2-chloro-4-((1R,2S)- and (1S,2R)-2-(((1-cyclopropyl-3-methyl-1H-pyrazol-4-yl)oxy)methyl)cyclobutoxy)-5-(trifluoromethyl)pyrimidine (190 (400 mg, 0.993 mmol) in DCM (30 mL) and MeNO₂ (30 mL) was added TEA (0.415 mL, 2.98 mmol). The mixture was allowed to stir at −5° C. for 10 min. The solution was added to another vial charged with nitronium tetrafluoroborate (0.312 mL, 2.98 mmol) at −5° C. The reaction mixture was allowed to stir at −5° C. for 30 min. The reaction mixture was poured into ice-water (30 mL) and extracted with EtOAc (10 mL×3). The organic layer was washed with water (30 mL×3) and dried over MgSO₄. The solution was filtered and concentrated under reduced pressure. The crude product was purified by reversed phase HPLC, eluting with water (0.1% TFA)-MeCN to afford the title compound 191.

### 4-(((1R,2S)- and (1S,2R)-2-((2-Chloro-5-(trifluoromethyl)pyrimidin-4-yl)oxy)cyclobutyl)methoxy)-1-cyclopropyl-3-methyl-1H-pyrazol-5-amine (192)

A solution of 2-chloro-4-((1R,2S)- and (1S,2R)-2-(((1-cyclopropyl-3-methyl-5-nitro-1H-pyrazol-4-yl)oxy)methyl)cyclobutoxy)-5-(trifluoromethyl)pyrimidine 191 (150 mg, 0.335 mmol) and ammonia hydrochloride (90 mg, 1.675 mmol) in ethanol (2 ml) and water (0.2 mL) was added iron (94 mg, 1.675 mmol). The mixture was allowed to stir at 90° C. for 1 h. The reaction mixture was filtered, then poured into water (5 mL) and extracted with EtOAc (5 mL×3). The organic layer was washed with brine (5 mL×3) and dried over MgSO₄. The solution was filtered and concentrated under reduced pressure to afford the title compound 192, which was used directly for the next step without further purification.

### (10aR,13aS)- or (10aS,13aR)-3-Cyclopropyl-1-methyl-8-(trifluoromethyl)-3,4,10a,11,12a,13-hexahydro-12H-5,9-(azeno)cyclobuta[k]pyrazolo[4,3-b][1,10]dioxa[4,6]diazacyclotridecine (Ex-14.1 and Ex-14.2)

To a solution of 4-(((1R,2S)- and (1S,2R)-2-((2-chloro-5-(trifluoromethyl)pyrimidin-4-yl)oxy)cyclobutyl)methoxy)-1-cyclopropyl-3-methyl-1H-pyrazol-5-amine 192 (120 mg, 0.287 mmol) in dioxane (1 mL) was added p-tolyl-27-sulfanetrione (24.58 mg, 0.144 mmol)) in a glove box. The reaction was allowed to stir for 2 h at 90° C. The reaction mixture was poured into water (10 mL) and extracted with EtOAc (30 mL×3). The organic layer was washed with water (10 mL×3) and dried over Na₂SO₄. The solution was filtered and concentrated under reduced pressure. The crude product was purified by reversed phase HPLC, eluting with water (0.1% NH₄OH)-MeCN to afford the racemic material. The racemic material was resolved to its component enantiomers by chiral preparative SFC (Column: Chiralpak AD-3 50×4.6 mm I.D., 3 μm Mobile phase: A: CO₂ B: isopropanol (0.05% DEA) Gradient: from 5% to 40% of B in 2 min and hold 40% for 1.2 min, then 5% of B for 0.8 min) to afford the title compounds Ex-14.1 (t_{R}=1.6 min) and Ex-14.2 (t_{R}=2.6 min). MS (ESI): m/z calc'd for C₁₇H₁₈F₃N₅O₂ [M+H]⁺: 382, found 382; ¹H NMR (400 MHz, CDCl₃) δ=8.22 (s, 1H), 4.81-4.69 (m, 1H), 4.36-4.26 (m, 1H), 4.02-3.96 (m, 1H), 3.18-3.08 (m, 1H), 2.70-2.58 (m, 1H), 2.78-2.10 (m, 5H), 2.04-1.96 (m, 2H), 1.23-1.13 (m, 2H), 1.01-0.93 (m, 2H); MS (ESI): m/z calc'd for C₁₇H₁₈F₃N₅O₂ [M+H]⁺: 382, found 382; ¹H NMR (400 MHz, CDCl₃) δ 8.34 (s, 1H), 7.32 (s, 1H), 4.83 (q, J=7.4 Hz, 1H), 4.40 (d, J=9.5 Hz, 1H), 3.80 (t, J=10.3 Hz, 1H), 3.21 (tt, J=3.6, 6.9 Hz, 1H), 2.83-2.70 (m, 1H), 2.36-2.27 (m, 2H), 2.26 (s, 3H), 2.15-2.00 (m, 2H), 1.06-0.98 (m, 2H), 0.90-0.84 (m, 2H).

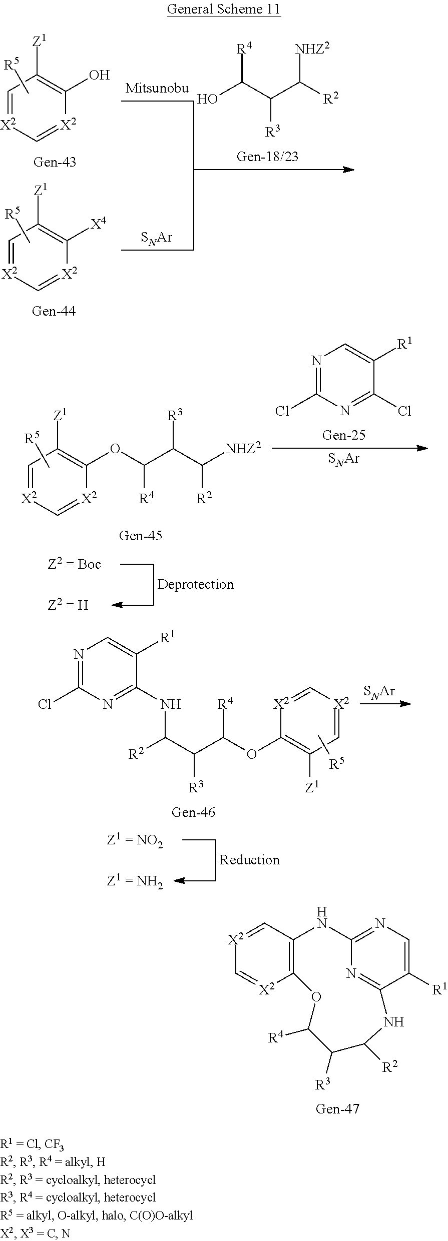

In General Scheme 11, aryl and heteroaryl alcohols of the form Gen-43 were coupled with commercially available or synthetically prepared amino-alcohols of the form Gen-18 or Gen 23 through Mitsunobu chemistry or derivatives of the Mitsunobu reaction to provide Gen-45. Alternatively, aryls and heteroaryl halides of the form Gen-44 could be coupled with

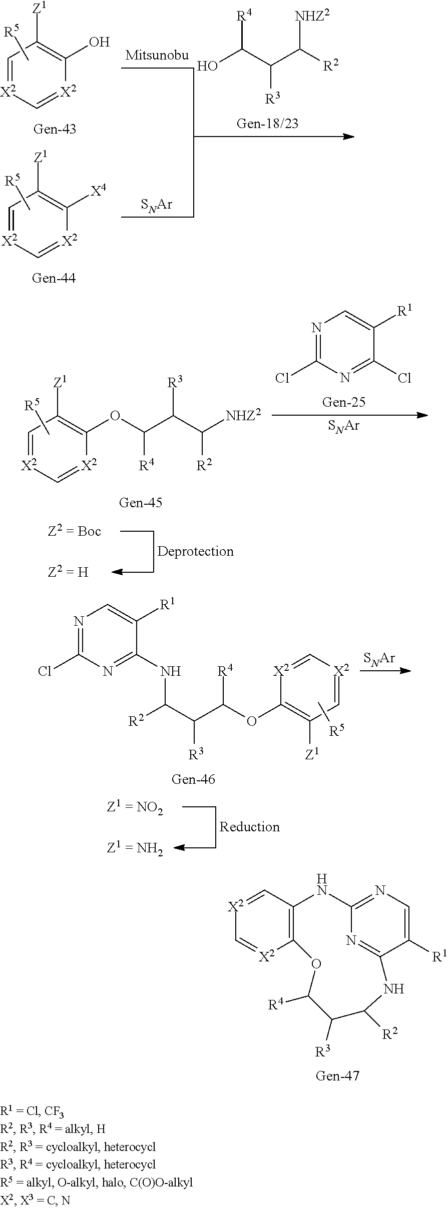

In General Scheme 11, aryl and heteroaryl alcohols of the form Gen-43 were coupled with commercially available or synthetically prepared amino-alcohols of the form Gen-18 or Gen 23 through Mitsunobu chemistry or derivatives of the Mitsunobu reaction to provide Gen-45. Alternatively, aryls and heteroaryl halides of the form Gen-44 could be coupled with commercially available or synthetically prepared amino-alcohols of the form Gen-18 or Gen-23 through base-mediated S_{N}Ar chemistry known to those skilled in the art. In instances of Gen-45 where Z²=Boc, acidic deprotection to the amine was performed. Commercially available pyrimidines of the form Gen-25 were coupled with amines of the form Gen-45 through S_{N}Ar chemistry to access Gen-46. Nitro-aryls or heteroaryls of the form Gen-46 where Z¹=NO₂, reduction to the corresponding amine was performed. The aminopyrazole Gen-46 could ultimately be transformed through intramolecular S_{N}Ar chemistry to afford elaborated compounds of the form Gen-47. Representative preparative examples from each sequence are described in more detail below.

### Preparation of Example 15.1

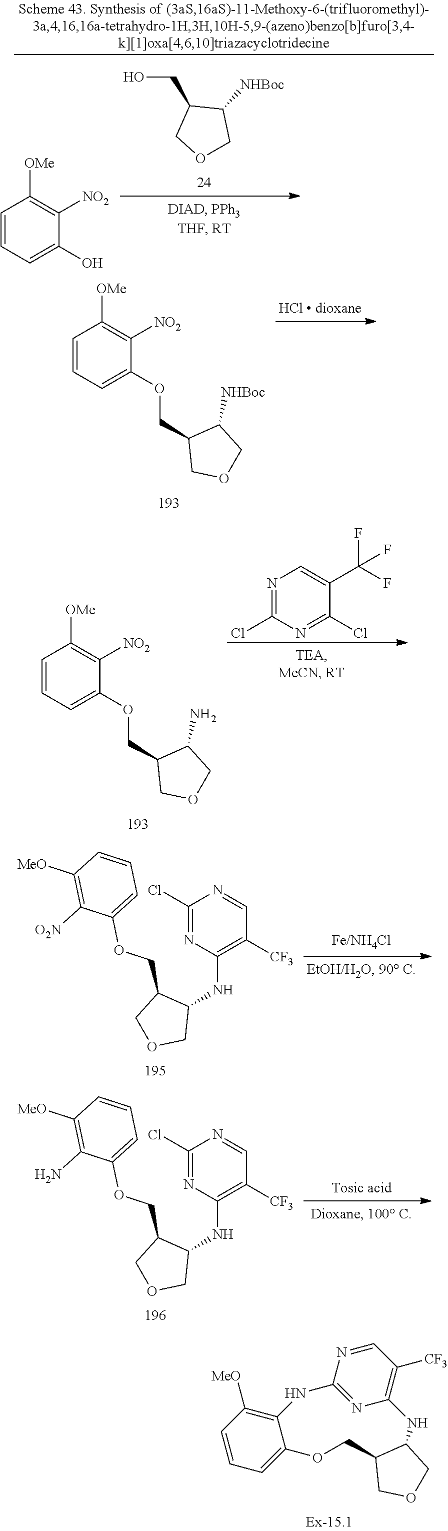

### tert-Butyl ((3S,4S)-4-((3-methoxy-2-nitrophenoxy)methyl)tetrahydrofuran-3-yl)carbamate (193)

To a vial was added 3-methoxy-2-nitrophenol (183 mg, 1.08 mmol), tert-butyl (4-(hydroxymethyl)tetrahydrofuran-3-yl)carbamate 24 (470 mg, 2.16 mmol) and triphenylphosphine (568 mg, 2.16 mmol). The solids were dissolved in tetrahydrofuran (1.08E+04 μL) and diisopropyl azodicarboxylate (421 μL, 2.16 mmol) was added. The reaction was allowed to stir at room temperature overnight. The reaction was washed with water and extracted with ethyl acetate. The combined organic layers were combined and concentrated under reduced pressure. The crude product was purified by flash chromatography on silica gel (gradient elution of 0-100% EtOAc/hexanes) to afford the title compound 193.

### (3S,4S)-4-((3-Methoxy-2-nitrophenoxy)methyl)tetrahydrofuran-3-amine (194)

To a vial was added tert-butyl (4-((3-methoxy-2-nitrophenoxy)methyl)tetrahydrofuran-3-yl)carbamate (399 mg, 1.08 mmol) and was dissolved in 1,4-dioxane hydrochloride (2700 μL, 10.8 mmol). The reaction was allowed to stir for 1 h and then concentrated under reduced pressure to afford the title compound 194, which was used directly for the next step without further purification.

### 2-Chloro-N-((3S,4S)-4-((3-methoxy-2-nitrophenoxy)methyl)tetrahydrofuran-3-yl)-5-(trifluoromethyl)pyrimidin-4-amine (195)

product was purified by reversed phase HPLC, eluting with water (0.1% TFA)-MeCN, to afford the title compound Ex-15.1. MS (ESI): m/z calc'd for C₁₇H₁₇F₃N₄O₃ [M+H]⁺: 383, found 383; ¹H NMR (500 MHz, CDCl₃) δ 10.78 (1H, s), 8.07 (1H, s), 7.19 (1H, t, J=8.4 Hz), 6.61 (2H, d, J=8.4 Hz), 6.06 (1H, d, J=5.3 Hz), 4.42 (1H, d, J=5.9 Hz), 4.25 (2H, q, J=10.1, 9.2 Hz), 4.13 (1H, t, J=10.8 Hz), 3.97-3.90 (2H, m), 3.89 (3H, s), 3.26 (1H, t, J=8.9 Hz), 2.45 (1H, d, J=10.0 Hz).

Compounds in Table 10 below were prepared in accordance with the synthetic sequence illustrated in General Scheme 11 using the corresponding starting materials.

TABLE 10

| | | | Exact |
|---|---|---|---|
| | | | Mass |
| Ex | Structure | Name | [M + H]⁺ |
| Ex- 15.2 | 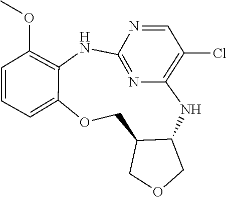 [SMILES: COc1cccc2c1Nc1ncc(Cl)c(n1)N[C@@H]1COC[C@H]1CO2] | (3aS,16aS)-6-chloro-11-methoxy- 3a,4,16,16a-tetrahydro-1H,3H,10H- 5,9-(azeno)benzo[b]furo[3,4- k][1]oxa[4,6,10]triazacyclotridecine | Calc'd 349, found 349 |
| Ex- 15.3 | 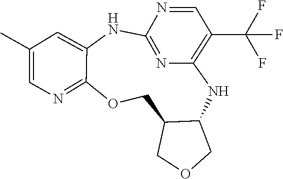 [SMILES: Cc1cnc2c(c1)Nc1ncc(C(F)(F)F)c(n1)N[C@@H]1COC[C@H]1CO2] | (11aS,14aS)-3-methyl-9- (trifluoromethyl)-11a,12,14a,15- tetrahydro-5H,11H,14H6,10- (azeno)furo[3,4-k]pyrido[2,3- b][1]oxa[4,6,10]triazacyclotridecine | Calc'd 368, found 368 |
| Ex- 15.4 | 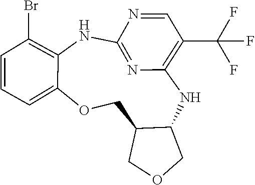 [SMILES: FC(F)(F)c1cnc2nc1N[C@@H]1COC[C@H]1COc1cccc(Br)c1N2] | (3aS,16aS)-11-bromo-6- (trifluoromethyl)-3a,4,16,16a- tetrahydro-1H,3H,10H-5,9- (azeno)benzo[b]furo[3,4- k\|[1]oxa[4,6,10]triazacyclotridecine | Calc'd 433, found 433 |
| Ex- 15.5 | 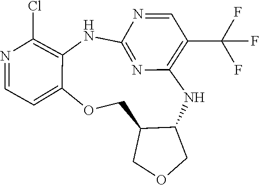 [SMILES: FC(F)(F)c1cnc2nc1N[C@@H]1COC[C@H]1COc1ccnc(Cl)c1N2] | (3aS,16aS)-11-chloro-6- (trifluoromethyl)-3a,4,16,16a- tetrahydro-1H,3H,10H-5,9- (azeno)furo[3,4-k]pyrido[4,3- b][1]oxa[4,6,10]triazacyclotridecine | Calc'd 388, found 388 |
| Ex- 15.2 | 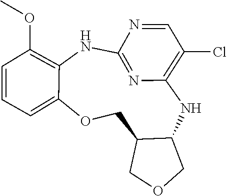 [SMILES: COc1cccc2c1Nc1ncc(Cl)c(n1)N[C@@H]1COC[C@H]1CO2] | (3aS,16aS)-6-chloro-11-methoxy- 3a,4,16,16a-tetrahydro-1H,3H,10H- 5,9-(azeno)benzo[b]furo[3,4- k][1]oxa[4,6,10]triazacyclotridecine | Calc'd 349, found 349 |
| Ex- 15.3 | 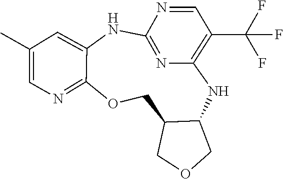 [SMILES: Cc1cnc2c(c1)Nc1ncc(C(F)(F)F)c(n1)N[C@@H]1COC[C@H]1CO2] | (11aS,14aS)-3-methyl-9- (trifluoromethyl)-11a,12,14a,15- tetrahydro-5H,11H,14H6,10- (azeno)furo[3,4-k]pyrido[2,3- b][1]oxa[4,6,10]triazacyclotridecine | Calc'd 368, found 368 |
| Ex- 15.4 | 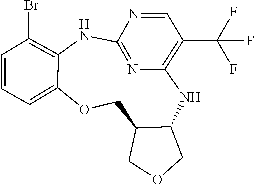 [SMILES: FC(F)(F)c1cnc2nc1N[C@@H]1COC[C@H]1COc1cccc(Br)c1N2] | (3aS,16aS)-11-bromo-6- (trifluoromethyl)-3a,4,16,16a- tetrahydro-1H,3H,10H-5,9- (azeno)benzo[b]furo[3,4- k][1]oxa[4,6,10]triazacyclotridecine | Calc'd 433, found 433 |
| Ex- 15.5 | 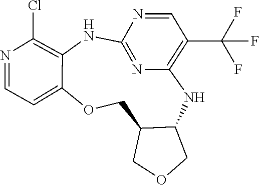 [SMILES: FC(F)(F)c1cnc2nc1N[C@@H]1COC[C@H]1COc1ccnc(Cl)c1N2] | (3aS,16aS)-11-chloro-6- (trifluoromethyl)-3a,4,16,16a- tetrahydro-1H,3H,10H-5,9- (azeno)furo[3,4-k]pyrido[4,3- b][1]oxa[4,6,10]triazacyclotridecine | Calc'd 388, found 388 |
| Ex- 15.6 | 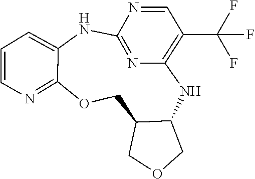 [SMILES: FC(F)(F)c1cnc2nc1N[C@@H]1COC[C@H]1COc1ncccc1N2] | (11aS,14aS)-9-(trifluoromethyl)- 11a,12,14a,15-tetrahydro- 5H,11H,14H6,10-(azeno)furo[3,4- k]pyrido[2,3- b][1]oxa[4,6,10]triazacyclotridecine | Calc'd 354, found 354 |
| Ex- 15.7 | 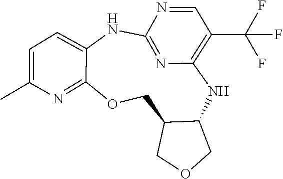 [SMILES: Cc1ccc2c(n1)OC[C@@H]1COC[C@H]1Nc1nc(ncc1C(F)(F)F)N2] | (11aS,14aS)-2-methyl-9- (trifluoromethyl)-11a,12,14a,15- tetrahydro-5H,11H,14H6,10- (azeno)furo[3,4-k]pyrido[2,3- b][1]oxa[4,6,10]triazacyclotridecine | Calc'd 368, found 368 |
| Ex- 15.8 | 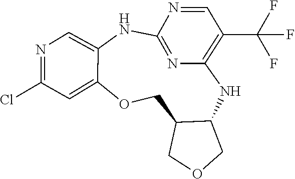 [SMILES: FC(F)(F)c1cnc2nc1N[C@@H]1COC[C@H]1COc1cc(Cl)ncc1N2] | (3aS,16aS)-13-chloro-6- (trifluoromethyl)-3a,4,16,16a- tetrahydro-1H,3H,10H-5,9- (azeno)furo[3,4-k]pyrido[4,3- b][1]oxa[4,6,10]triazacyclotridecine | Calc'd 388, found 388 |
| Ex- 15.9 | 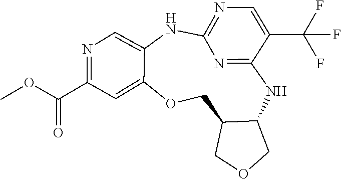 [SMILES: COC(=O)c1cc2c(cn1)Nc1ncc(C(F)(F)F)c(n1)N[C@@H]1COC[C@H]1CO2] | ethyl (3aS,16aS)-6-(trifluoromethyl)- 3a,4,16,16a-tetrahydro-1H,3H,10H- 5,9-(azeno)furo[3,4-k]pyrido[4,3- b][1]oxa[4,6,10]triazacyclotridecine- 13-carboxylate | Calc'd 426, found 426 |

### Preparation of Examples 16.1 and 16.2

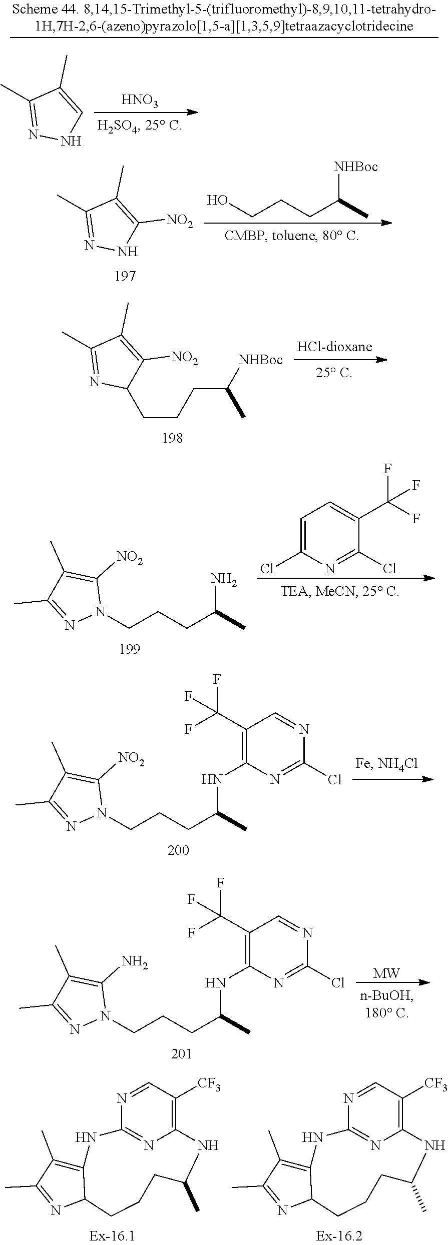

### 3,4-Dimethyl-5-nitro-1H-pyrazole (197)

To a mixture of 3,4-dimethyl-1H-pyrazole (2.00 g, 20.8 mmol) in anhydrous sulfuric acid (10 mL, 20.8 mmol) was added nitric acid (2.00 mL, 32.0 mmol) at 0° C. The reaction mixture was allowed to stir at 25° C. for 1 h. The mixture was added into the water (20 mL) and adjusted to pH=8 with the saturated NaHCO₃ solution. EtOAc (20 mL) was added into the mixture. The organic layer was separated. The aqueous was extracted with EtOAc (20 mL×3). The mixture was dried by anhydrous Na₂SO₄, filtered and concentrated under reduced pressure. The crude product was purified by flash chromatography on silica gel (2:1 PE/EtOAc) to afford the title compound 197. 199, which was used in next step without further purification.

### 2-Chloro-N-(5-(3,4-dimethyl-5-nitro-1H-pyrazol-1-yl) pentan-2-yl)-5-(trifluoromethyl)pyrimidin-4-amine (200)

To a vial containing 5-(3,4-dimethyl-5-nitro-1H-pyrazol-1-yl) pentan-2-amine 199 (150 mg, 0.663 mmol) was added 2,4-dichloro-5-(trifluoromethyl)pyrimidine (158 mg, 0.729 mmol) and TEA (0.277 ml, 1.989 mmol) in MeOH (5.0 mL). The reaction mixture was allowed to stir at 25° C. for 1 h. The mixture was concentrated under reduced pressure. The crude product was purified by flash chromatography on silica gel (5:1 PE/EtOAc) to afford the title compound 200. ¹HNMR (400 MHz, CDCl₃) § 8.25 (s, 1H), 4.58-4.47 (m, 2H), 4.46-4.36 (m, 1H), 2.27 (s, 3H), 2.22 (s, 3H), 1.91 (q, J=7.5 Hz, 2H), 1.65-1.59 (m, 2H), 1.26 (d, J=6.6 Hz, 3H).

### N-(5-(5-Amino-3,4-dimethyl-1H-pyrazol-1-yl) pentan-2-yl)-2-chloro-5-(trifluoromethyl)pyrimidin-4-amine (201)

To solution of ammonium chloride (52.6 mg, 0.983 mmol) and iron (54.9 mg, 0.983 mmol) in ethanol (5 mL) and water (1 mL) was added 2-chloro-N-(5-(3,4-dimethyl-5-nitro-1H-pyrazol-1-yl) pentan-2-yl)-5-(trifluoromethyl)pyrimidin-4-amine 200 (80 mg, 0.197 mmol). The mixture was allowed to stir at 100° C. for 2 h. The mixture was filtered. The filtrate was concentrated under reduced pressure to afford the title compound 201, which was used directly for the next step.

### 8,14,15-Trimethyl-5-(trifluoromethyl)-8,9,10,11-tetrahydro-1H,7H-2,6-(azeno)pyrazolo[1,5-a][1,3,5,9]tetraazacyclotridecine (Ex-16.1 and Ex-16.2)

A solution of N-(5-(5-amino-3,4-dimethyl-1H-pyrazol-1-yl) pentan-2-yl)-2-chloro-5-(trifluoromethyl)pyrimidin-4-amine 201 (30 mg, 0.074 mmol) in n-BuOH (2.0 mL) was heated to 180° C. and allowed to stir for 8 h. The mixture was concentrated under reduced pressure to give a residue, which was purified by reversed-phase HPLC, eluting with water (0.1% TFA)-MeCN, to afford the racemic material. The racemic material could be resolved to its component enantiomers by chiral preparative SFC (Column: Chiralpak AD-3 150×4.6 mm I.D., 3 μm Mobile phase: A: CO₂; B: isopropanol (0.05% DEA); Gradient: from 5-40% of B in 5 min and hold 40% for 2.5 min, then 5% of B for 2.5 min) to afford the title compounds Ex-16.1 (t_{R}=3.5 min) and Ex-16.2 (t_{R}=5.1 min). MS (ESI): m/z calc'd for C₁₅H₁₉F₃N₆ [M+H]⁺: 341, found 341; ¹H NMR (500 MHz, CDCl₃) δ: 8.15 (s, 1H), 6.76 (s, 1H), 5.14 (s, 1H), 4.57 (s, 1H), 3.94-3.83 (m, 1H), 3.63 (d, J=5.5 Hz, 1H), 2.43 (s, 1H), 2.18 (s, 3H), 2.03-1.92 (m, 1H), 1.90 (s, 3H), 1.87-1.73 (m, 1H), 1.67-1.61 (m, 1H), 1.29 (d, J=6.9 Hz, 3H); MS (ESI): m/z calc'd for C₁₅H₁₉F₃N₆ [M+H]⁺: 341, found 341; ¹H NMR (500 MHz, CDCl₃) δ: 8.15 (s, 1H), 6.68 (s, 1H), 5.14 (s, 1H), 4.56 (s, 1H), 3.93-3.85 (m, 1H), 3.63 (d, J=4.7 Hz, 1H), 2.44 (s, 1H), 2.19-2.17 (m, 1H), 2.18 (s, 2H), 2.04-1.73 (m, 5H), 1.62 (s, 1H), 1.30 (d, J=6.9 Hz, 3H).

### Preparation of Examples 17.1 and 17.2

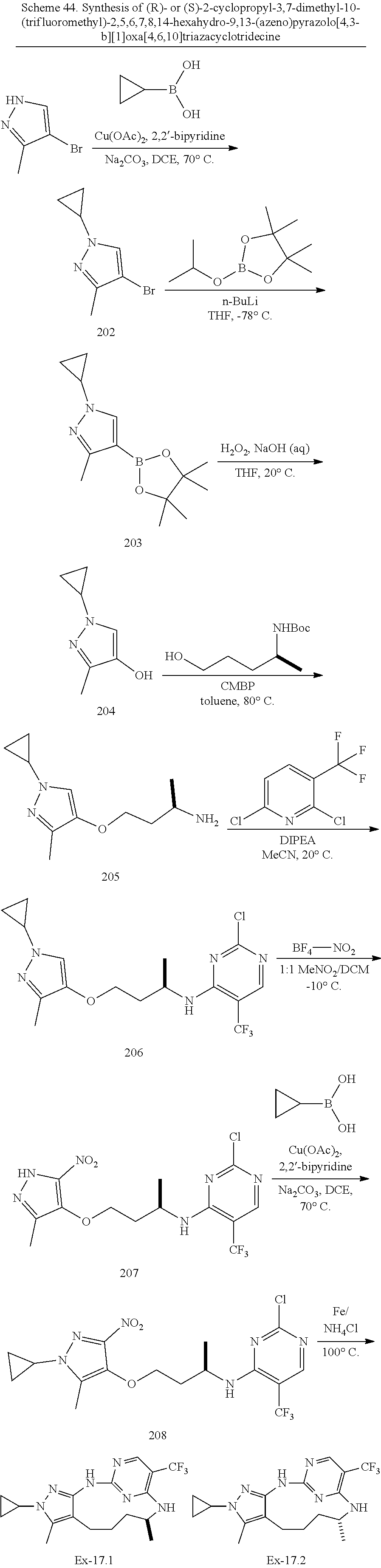

### 4-Bromo-1-cyclopropyl-3-methyl-1H-pyrazole (202)

To a solution of copper (II) acetate (54.2 g, 298 mmol) in anhydrous DCE (200 mL) was added 2,2′-bipyridine (46.6 g, 298 mmol), and the mixture was allowed to stir at 50° C. for 30 min under N2. Na₂CO₃ (79 g, 745 mmol), 4-bromo-5-methyl-1H-pyrazole (40 g, 248 mmol) and cyclopropylboronic acid (27.7 g, 323 mmol) in DCE (200 mL) was added into the mixture. The final mixture was allowed to stir at 70° C. for 16 h. The reaction mixture was filtered and concentrated under reduced pressure. The resulting solution was poured into water (300 mL) and extracted with DCM (300 mL×3). The combined organic layers were washed with water, dried over Na₂SO₄. After filtration and concentration, crude product was purified by flash chromatography on silica gel (gradient elution of 0-10% EtOAc/PE) to afford a mixture of regioisomers. The mixture could be resolved to its component enantiomers by chiral preparative SFC (Column: Chiralpak AD-3 150*4.6 mm I.D., 3 μm. Mobile phase: A: CO₂; B: ethanol (0.05% DEA); Gradient: from 5% to 40% of B in 2 min and hold 40% for 1.2 min, then 5% of B for 0.8 min) to afford the title compound 202 (t_{R}=1.6 min).

### 1-Cyclopropyl-3-methyl-4-(4,4,5,5-tetramethyl-1,3,2-dioxaborolan-2-yl)-1H-pyrazole (203)

To a solution of 4-bromo-1-cyclopropyl-3-methyl-1H-pyrazole 202 (7 g, 34.8 mmol) in THF (90 mL) was added dropwise n-butyllithium (2.5 M in hexanes, 3.12 g, 48.7 mmol) at −78° C. The resulting solution was allowed to stir for 30 min. To the mixture was added a solution of 2-isopropoxy-4,4,5,5-tetramethyl-1,3,2-dioxaborolane (14.3 g, 77.0 mmol) in THF (10 mL). The mixture was allowed to warm to RT and stir for 30 min. The reaction was quenched with saturated aqueous NH₄Cl solution. The mixture was extracted with EtOAc (100 mL×3), washed with brine, dried over MgSO₄, filtered and concentrated under reduced pressure. The crude product was purified by flash chromatography on silica gel (gradient elution of 0-40% EtOAc/PE) to afford the title compound 203.

### 1-Cyclopropyl-3-methyl-1H-pyrazol-4-ol (204)

To a solution of 1-cyclopropyl-3-methyl-4-(4,4,5,5-tetramethyl-1,3-dioxolan-2-yl)-1H-pyrazole 203 (6 g, 23.97 mmol) in THF (20 mL) was added sodium hydroxide (2.0 N in water, 36.0 mL, 71.9 mmol) and hydrogen peroxide (30% w/w in H₂O, 5.43 g, 47.9 mmol) at 0° C. The solution was allowed to stir for 2 h at 20° C. The mixture was quenched by adding saturated aqueous Na₂SO₃. The mixture was adjusted to pH of 4 with 2 M aqueous HCl. The mixture was concentrated and the residue was dissolved in MeOH (40 mL). The mixture was filtered and the filtrate was concentrated. The crude product was purified by reversed phase HPLC, eluting with water (0.1% NH₄OH)-MeCN, to afford the title compound 204.

### (R)- and (S)-4-((1-cyclopropyl-3-methyl-1H-pyrazol-4-yl)oxy) butan-2-amine (205)

To a solution of 1-cyclopropyl-3-methyl-1H-pyrazol-4-ol 204 (2 g, 14.47 mmol) and 3-aminobutan-1-ol (1.290 g, 14.47 mmol) in anhydrous toluene (15 mL) was added (tributylphosphoranylidene) acetonitrile (5.24 g, 21.71 mmol). The reaction mixture was allowed to stir at 80° C. under N₂ for 16 h. The mixture was concentrated to afford the title compound 205. The product was used in the next step directly without further purification.

### (R)- and (S)-2-chloro-N-(4-((1-cyclopropyl-3-methyl-1H-pyrazol-4-yl)oxy) butan-2-yl)-5-(trifluoromethyl)pyrimidin-4-amine (206)

To a solution of 4-((1-cyclopropyl-3-methyl-1H-pyrazol-4-yl)oxy) butan-2-amine 205 (2.5 g, 11.94 mmol) and 2,4-dichloro-5-(trifluoromethyl)pyrimidine (3.11 g, 14.33 mmol) in acetonitrile (30 mL) was added DIPEA (6.26 mL, 35.8 mmol) at 20° C. The mixture was allowed to stir for 1 h at 20° C. The mixture was concentrated under reduced pressure. The crude product was purified by reversed phase HPLC, eluting with water (0.1% NH₄OH)-MeCN, to afford the title compound 206.

### (R)- and (S)-2-chloro-N-(4-((3-methyl-5-nitro-1H-pyrazol-4-yl)oxy) butan-2-yl)-5-(trifluoromethyl)pyrimidin-4-amine (207)

To a mixture of 2-chloro-N-(4-((1-cyclopropyl-3-methyl-1H-pyrazol-4-yl)oxy) butan-2-yl)-5-(trifluoromethyl)pyrimidin-4-amine 206 (300 mg, 0.770 mmol) in MeNO₂ (5 mL) and DCM (5 mL) was added nitronium tetrafluoroborate (307 mg, 2.309 mmol) at −10° C. The reaction mixture was allowed to stir at −10° C. for 1 h. The reaction was quenched with H₂O (10 mL). DCM (20 mL) was added into the mixture. The organic layer was separated. The aqueous layer was extracted with DCM (20 mL×2). The mixture was dried over anhydrous Na₂SO₄, filtered and concentrated under reduced pressure. The crude product was purified by flash chromatography on silica gel (gradient elution of 0-10% EtOAc/PE) to afford the title compound 207.

### (R)- and (S)-2-chloro-N-(4-((1-cyclopropyl-5-methyl-3-nitro-1H-pyrazol-4-yl)oxy) butan-2-yl)-5-(trifluoromethyl)pyrimidin-4-amine (208)

To a solution of copper (II) acetate (11.04 mg, 0.061 mmol) in anhydrous DCE (3 mL) was added 2,2′-bipyridine (9.50 mg, 0.061 mmol), and the mixture was allowed to stir at 50° C. for 30 min under N₂. Na₂CO₃ (16.11 mg, 0.152 mmol), (R)- or (S)-2-chloro-N-(4-((3-methyl-5-nitro-1H-pyrazol-4-yl)oxy) butan-2-yl)-5-(trifluoromethyl)pyrimidin-4-amine 207 (20 mg, 0.051 mmol) and cyclopropylboronic acid (5.66 mg, 0.066 mmol) in DCE (3 mL) was added into the mixture. The final mixture was allowed to stir at 70° C. for 12 h. The mixture was filtered and concentrated under reduced pressure. The reaction mixture was poured into water (20 mL) and extracted with EtOAc (30 mL×3). The organic layer was washed with water (10 mL), dried over Na₂SO₄, filtered and concentrated under reduced pressure. The crude product was purified by reversed phase HPLC, eluting with water (0.1% NH₄OH)-MeCN, to afford the title compound 208.

### (R)- or (S)-2-cyclopropyl-3,7-dimethyl-10-(trifluoromethyl)-2,5,6,7,8,14-hexahydro-9,13-(azeno)pyrazolo[4,3-b][1]oxa[4,6,10]triazacyclotridecine (Ex-17.1 and Ex-17.2)

To a solution of 2-chloro-N-(4-((1-cyclopropyl-5-methyl-3-nitro-1H-pyrazol-4-yl)oxy) butan-2-yl)-5-(trifluoromethyl)pyrimidin-4-amine 208 (30 mg, 0.069 mmol) and ammonia hydrochloride (36.9 mg, 0.690 mmol) in EtOH (10 mL) and water (1 mL) was added iron (38.5 mg, 0.690 mmol). The mixture was allowed to stir at 90° C. for 12 h. The reaction mixture was filtered and concentrated under reduced pressure. The crude product was purified by reversed-phase HPLC, eluting with water (0.1% TFA)-MeCN, to afford the racemic material. The racemic material could be resolved to its component enantiomers by chiral preparative SFC (Column: Chiralpak AD-3 150*4.6 mm I.D., 3 μm. Mobile phase: A: CO₂ B: ethanol (0.05% DEA). Gradient: from 5% to 40% of B in 2 min and hold 40% for 1.2 min, then 5% of B for 0.8 min) to afford the title compounds Ex-17.1 (t_{R}=1.4 min) and Ex-17.2 (t_{R}=2.4 min). MS (ESI): m/z calc'd for C₁₆H₁₉F₃N₆O [M+H]⁺: 369, found 369; ¹H NMR (400 MHz, CDCl₃) δ 9.48 (br s, 1H), 8.42 (s, 1H), 4.91 (br s, 1H), 4.47 (br s, 1H), 4.08 (m, 1H), 3.85 (m, 1H), 3.20 (m, 1H), 2.26 (s, 3H), 2.09-1.96 (m, 1H), 1.62-1.54 (m, 1H), 1.32 (d, J=7.1 Hz, 3H), 1.09-0.91 (m, 4H), 1.55-−0.02 (m, 1H). MS (ESI): m/z calc'd for C₁₆H₁₉F₃N₆O [M+H]⁺: 369, found 369; ¹H NMR (400 MHz, CDCl₃) δ 9.55 (br s, 1H), 8.45-8.37 (m, 1H), 4.99-4.83 (m, 1H), 4.47 (br s, 1H), 4.08 (m, 1H), 3.85 (m, 1H), 3.20 (m, 1H), 2.29-2.17 (m, 3H), 2.09-1.99 (m, 1H), 1.62-1.53 (m, 1H), 1.32 (d, J=6.8 Hz, 3H), 1.10-0.90 (m, 4H).

### Preparation of Example 18.1

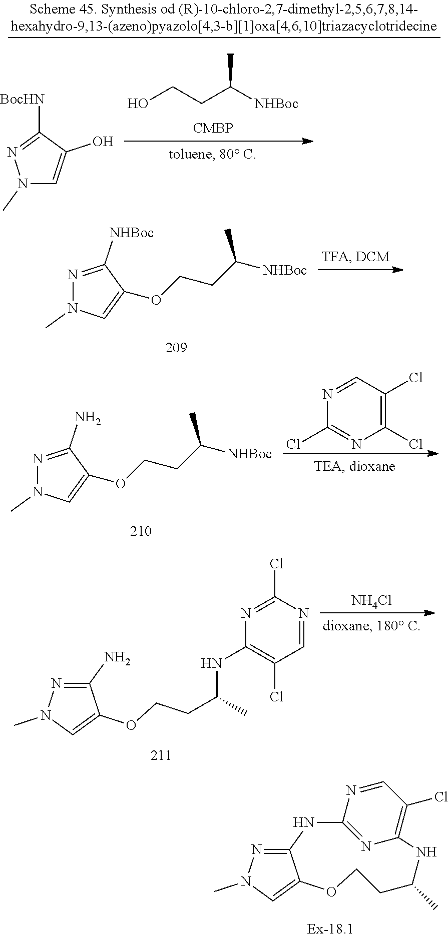

### tert-butyl (R)-(4-((3-((tert-butoxycarbonyl)amino)-1-methyl-1H-pyrazol-4-yl)oxy) butan-2-yl)carbamate (209)

To a solution of (R)-tert-butyl (4-hydroxybutan-2-yl)carbamate (110 mg, 0.58 mmol) and tert-butyl (4-hydroxy-1-methyl-1H-pyrazol-3-yl)carbamate (150 mg, 0.70 mmol) in anhydrous Toluene (3.0 mL) was added (tributylphosphoranylidene) acetonitrile (203 mg, 0.84 mmol), and the reaction mixture was allowed to stir at 80° C. under N₂ for 16 h. The reaction was cooled to RT then directly loaded and purified by flash chromatography on silica gel (gradient elution of 0-100% EtOAc/DCM) to afford the title compound 209.

### (R)-4-(3-aminobutoxy)-1-methyl-1H-pyrazol-3-amine, TFA (210)

Trifluoroacetic acid (0.1 ml, 1.3 mmol) was added dropwise to a solution of tert-butyl (R)-(4-(3-((tert-butoxycarbonyl)amino) butoxy)-1-methyl-1H-pyrazol-3-yl)carbamate 209 (84 mg, 0.22 mmol) in Dichloromethane (1.0 mL). The reaction mixture was allowed to stir at RT under N₂ for 18 h. Trifluoroacetic acid (0.05 ml, 0.65 mmol) was added, and the mixture was allowed to stir an additional 3 h. The mixture was concentrated to afford the title compound 210. The product was used in the next step directly without further purification.

### (R)-N-(4-((3-amino-1-methyl-1H-pyrazol-4-yl)oxy) butan-2-yl)-2,5-dichloropyrimidin-4-amine (211)

To a mixture of (R)-4-(3-aminobutoxy)-1-methyl-1H-pyrazol-3-amine 210 (40 mg, 0.22 mmol) and triethylamine (0.12 ml, 0.87 mmol) in dioxane (2.0 mL) was added 2,4,5-trichloropyrimidine (0.025 ml, 0.218 mmol). The reaction mixture was allowed to stir at RT overnight. The reaction mixture was diluted with water and extracted three times with CHCl₃:IPA (3:1 v/v). The 0-100% EtOAc/DCM) to afford the title compound 209.

### (R)-4-(3-aminobutoxy)-1-methyl-1H-pyrazol-3-amine, TFA (210)

Trifluoroacetic acid (0.1 ml, 1.3 mmol) was added dropwise to a solution of tert-butyl (R)-(4-(3-((tert-butoxycarbonyl)amino) butoxy)-1-methyl-1H-pyrazol-3-yl)carbamate 209 (84 mg, 0.22 mmol) in Dichloromethane (1.0 mL). The reaction mixture was allowed to stir at RT under N₂ for 18 h. Trifluoroacetic acid (0.05 ml, 0.65 mmol) was added, and the mixture was allowed to stir an additional 3 h. The mixture was concentrated to afford the title compound 210. The product was used in the next step directly without further purification.

### (R)-N-(4-((3-amino-1-methyl-1H-pyrazol-4-yl)oxy) butan-2-yl)-2,5-dichloropyrimidin-4-amine (211)

To a mixture of (R)-4-(3-aminobutoxy)-1-methyl-1H-pyrazol-3-amine 210 (40 mg, 0.22 mmol) and triethylamine (0.12 ml, 0.87 mmol) in dioxane (2.0 mL) was added 2,4,5-trichloropyrimidine (0.025 ml, 0.218 mmol). The reaction mixture was allowed to stir at RT overnight. The reaction mixture was diluted with water and extracted three times with CHCl₃:IPA (3:1 v/v). The combined organic fractions were dried over anhydrous sodium sulfate, filtered, and concentrated under reduced pressure. The residue was purified by flash chromatography on silica gel (gradient elution of 0-100% EtOAc:EtOH (3:1 v/v)/hexanes) to afford the title compound 211.

### (R)-10-chloro-2,7-dimethyl-2,5,6,7,8,14-hexahydro-9,13-(azeno)pyrazolo[4,3-b][1]oxa[4,6,10]triazacyclotridecine, TFA (Ex-18.1)

Ammonium chloride (7.1 mg, 0.13 mmol) and (R)-N-(4-((3-amino-1-methyl-1H-pyrazol-4-yl)oxy) butan-2-yl)-2,5-dichloropyrimidin-4-amine 211 (22 mg, 0.07 mmol) were suspended in dioxane (0.5 ml) in a microwave vial. The vial was sealed and subjected to microwave heating at 150° C. for 1 h followed by microwave heating at 180° C. for 7 h. The resulting solution was concentrated under reduced pressure. The crude residue was purified by reversed-phase HPLC, eluting with water (0.1% TFA)-MeCN, to afford the title compound Ex-18.1. MS (ESI): m/z calc'd for C₁₂H₁₆ClN₆O [M+H]⁺: 295, found 295; ¹H NMR (DMSO-d₆, 500 MHz) δ 9.96 (s, 1H), 8.03 (s, 1H), 7.50 (s, 1H), 4.28 (dd, J=9.8, 5.7 Hz, 1H), 4.03 (dt, J=6.8, 3.1 Hz, 1H), 3.79-3.68 (m, 1H), 3.67 (s, 3H), 1.86-1.75 (m, 1H), 1.72-1.58 (m, 1H), 1.29 (d, J=7.1 Hz, 3H).

### Preparation of Example 19.1

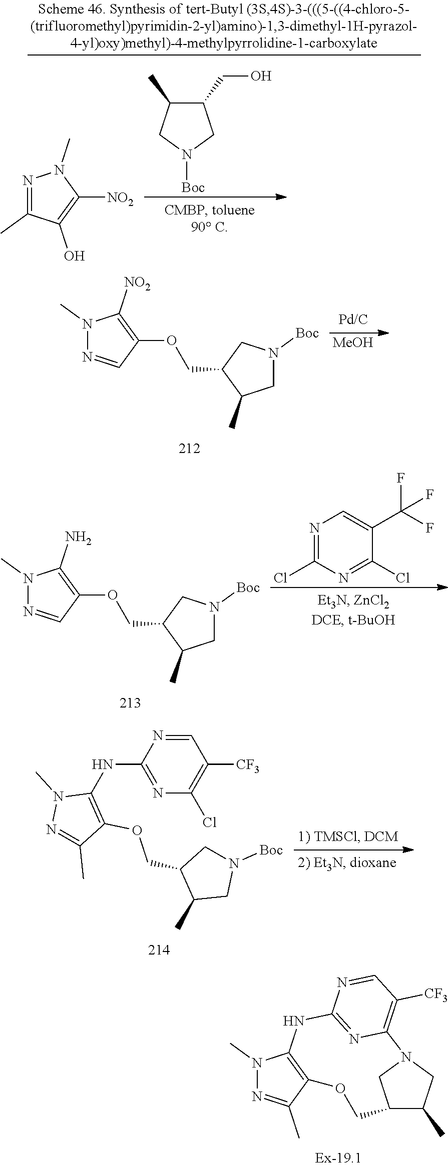

### tert-Butyl (3S,4S)-3-(((1,3-dimethyl-5-nitro-1H-pyrazol-4-yl)oxy)methyl)-4-methylpyrrolidine-1-carboxylate (212)

To a vial was added 1,3-dimethyl-5-nitro-1H-pyrazol-4-ol (350 mg, 2.227 mmol), tert-butyl (3S,4S)-3-(hydroxymethyl)-4-methylpyrrolidine-1-carboxylate (528.7 mg, 2.456 mmol) and toluene (8 ml). To the solution was added CMBP (791 mg, 3.28 mmol). The mixture was heated at 90° C. for 2 h. The mixture was diluted with water and extracted with EtOAc. The combined organic layers were washed with brine, dried over MgSO₄, filtered and concentrated under reduced pressure. The crude product was purified by flash chromatography on silica gel (gradient elution of 0-50% EtOAc/PE) to afford the title compound 212. MS (ESI): m/z calc'd for C₁₆H₂₆N₄O₅ [M+23]⁺: 377, found 377.

### tert-Butyl (3S,4S)-3-(((5-amino-1,3-dimethyl-1H-pyrazol-4-yl)oxy)methyl)-4-methylpyrrolidine-1-carboxylate (213)

To the solution of tert-butyl (3S,4S)-3-(((1,3-dimethyl-5-nitro-1H-pyrazol-4-yl)oxy)methyl)-4-methylpyrrolidine-1-carboxylate 212 (396 mg, 1.12 mmol) in MeOH (4000 μL) was added 10% Pd/C (119 mg, 0.112 mmol). The mixture was evacuated and backfilled with N₂ for 3 times. The reaction mixture was then allowed to stir under an atmosphere of H₂ at RT for 5 days. The mixture was filtered over a pad of Celite and concentrated under reduced pressure. The crude product was purified by flash chromatography on silica gel (gradient elution of 0-80% tert-Butyl (3S,4S)-3-(((5-((4-chloro-5-(trifluoromethyl)pyrimidin-2-yl)amino)-1,3-dimethyl-1H-pyrazol-4-yl)oxy)methyl)-4-methylpyrrolidine-1-carboxylate (Ex-19.1) To the vial containing tert-butyl (3S,4S)-3-(((5-((4-chloro-5-(trifluoromethyl)pyrimidin-2-yl)amino)-1,3-dimethyl-1H-pyrazol-4-yl)oxy)methyl)-4-methylpyrrolidine-1-carboxylate 214 (41.1 mg, 0.081 mmol) was added DCM (900 μL) and TMSI (60 μL, 0.441 mmol). The mixture was diluted with dioxane (4000 μL) and to the solution was added Et₃N (150 μL, 1.076 mmol). The mixture was allowed to stir at RT for 2 h. The reaction mixture was diluted with water and extracted with EtOAc. The combined organic layers were washed with brine, dried over MgSO₄, filtered and concentrated under reduced pressure. The crude residue was purified by reversed-phase HPLC, eluting with water (0.1% TFA)-MeCN, to afford the title compound Ex-19.1. MS (ESI): m/z calc'd for C₁₆H₁₉F₃N₆O [M+1]⁺: 369, found 369. ¹H NMR (600 MHz, DMSO-d₆) δ 9.73 (s, 1H), 8.45 (s, 1H), 4.44 (dd, J=11.5, 2.3 Hz, 1H), 4.32 (d, J=12.4 Hz, 1H), 4.02 (d, J=11.4 Hz, 1H), 3.60 (s, 3H), 3.49 (dd, J=11.4, 3.9 Hz, 1H), 3.14-3.00 (m, 1H), 2.93 (t, J=11.4 Hz, 1H), 2.18 (s, 3H), 1.83 (dp, J=22.7, 8.7, 7.5 Hz, 2H), 1.00 (d, J=6.1 Hz, 3H).

### Preparation of Examples 20.1 and 20.2

### Scheme 47. Synthesis of (6S,7S)- and (6R,7R)-1-Cyclobutyl-3,7-dimethyl-11-(trifluoromethyl)-1,5,6,7,8,15-hexahydro-10,14-(azeno)-6,9-methanopyrazolo[4,3-b][1]oxa[4,6,10]triazacyclotetradecine

### Preparation of Examples 20.1 and 20.2

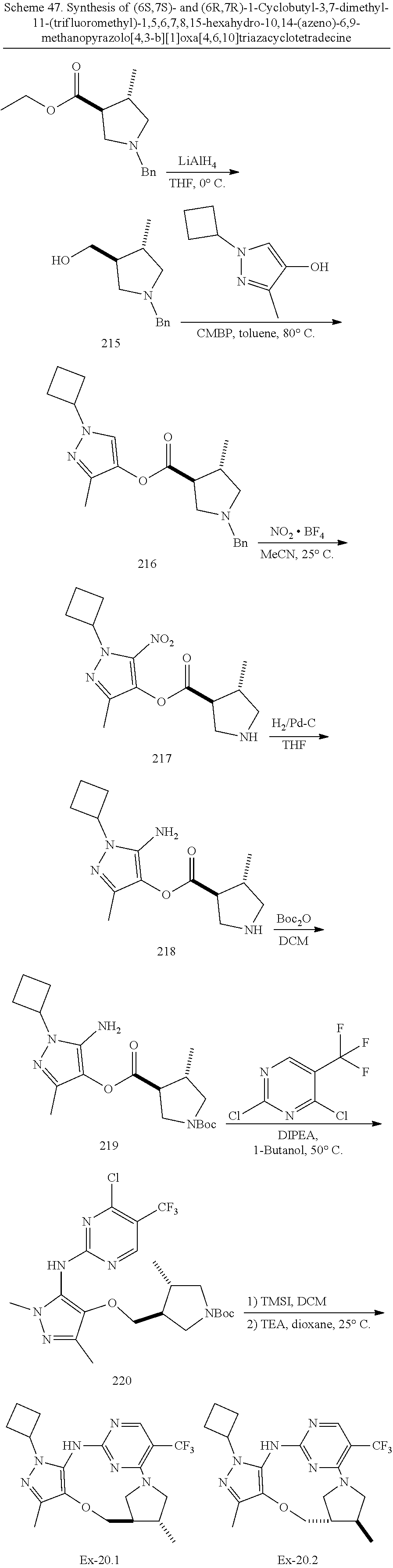

((3S,4S)- and (3R,4R)-1-benzyl-4-methylpyrrolidin-3-yl)methanol (215) To a suspension of LiAlH₄ (0.460 g, 12.13 mmol) in anhydrous THF (20 mL) was added dropwise trans-ethyl 1-benzyl-4-methylpyrrolidine-3-carboxylate (3 g, 12.13 mmol) in THF (30 mL) at 0° C., and the reaction mixture was allowed to stir at 0° C. for 1 h. The reaction mixture was quenched with water (5 mL). The mixture was filtered and concentrated under reduced pressure to afford the title compound 215, which was used directly for the next step without purification.

### 4-(((3S,4S)- and (3R,4R)-1-benzyl-4-methylpyrrolidin-3-yl)methoxy)-1-cyclobutyl-3-methyl-1H-pyrazole (216)

### 1-Cyclobutyl-3-methyl-4-(((3S,4S)- and (3R,4R)-4-methylpyrrolidin-3-yl)methoxy)-5-nitro-1H-pyrazole (217)

The solution of 4-(((3S,4S)- and (3R,4R)-1-benzyl-4-methylpyrrolidin-3-yl)methoxy)-1-cyclobutyl-3-methyl-1H-pyrazole 216 (2.7 g, 7.95 mmol) in MeCN (30 mL) was allowed to stir at 25° C. for 10 min. Nitronium tetrafluoroborate (2.495 mL, 23.9 mmol) was added to the mixture. The reaction mixture was allowed to stir at 25° C. for 1 h. The mixture was poured into the ice water (50 mL), and then extracted with EtOAc (50 mL). The combined organic layers were dried over Na₂SO₄ and filtered. The filtrate was concentrated under reduced pressure. The crude product was purified by flash chromatography on silica gel (1:1 EtOAc/PE) to afford the title compound 217. MS (ESI) m/z calc'd for C₂₁H₂₈N₄O₃ [M+H]⁺: 385, found 385.

### 1-Cyclobutyl-3-methyl-4-(((3S,4S)- and (3R,4R)-4-methylpyrrolidin-3-yl)methoxy)-1H-pyrazol-5-amine (218)

To a solution of 1-cyclobutyl-3-methyl-4-(((3S,4S)- and (3R,4R)-4-methylpyrrolidin-3-yl)methoxy)-5-nitro-1H-pyrazole 217 (600 mg, 1.561 mmol) in anhydrous THF (20 mL) was added Pd/C (1848 mg, 1.561 mmol). The reaction mixture was allowed to stir at 50° C. under H₂ at 15 psi for 12 h. The mixture was filtered and concentrated under reduced pressure to afford the title compound 218, which was directly used to next step without further purification. MS (ESI) m/z calc'd for C₁₄H₂₄N₄O [M+H]⁺: 265, found 265.

### tert-Butyl (3S,4S)- and (3R,4R)-3-(((5-amino-1-cyclobutyl-3-methyl-1H-pyrazol-4-yl)oxy)methyl)-4-methylpyrrolidine-1-carboxylate (219)

1-Cyclobutyl-3-methyl-4-(((3S,4S)- and (3R,4R)-4-methylpyrrolidin-3-yl)methoxy)-1H-pyrazol-5-amine 218 (400 mg, 1.513 mmol) and Boc₂O (1.054 mL, 4.54 mmol) in DCM (10 mL) was added TEA (0.633 ml, 4.54 mmol) at 25° C. The mixture was allowed to stir at 25° C. for 12 h. The mixture was concentrated under reduced pressure. The crude product was purified by flash chromatography on silica gel (1:1 EtOAc/PE) to afford the title compound 219. MS (ESI) m/z calc'd for C₁₉H₃₂N₄O₃ [M+H]⁺: 365, found 365.

### tert-Butyl (3S,4S)- and (3R,4R)-3-(((5-((4-chloro-5-(trifluoromethyl)pyrimidin-2-yl)amino)-1-cyclobutyl-3-methyl-1H-pyrazol-4-yl)oxy)methyl)-4-methylpyrrolidine-1-carboxylate (220)

To a solution of tert-butyl (3S,4S)- and (3R,4R)-3-(((5-amino-1-cyclobutyl-3-methyl-1H-pyrazol-4-yl)oxy)methyl)-4-methylpyrrolidine-1-carboxylate 219 (200 mg, 0.549 mmol) in n-butyl alcohol (6 mL) was added 2,4-dichloro-5-(trifluoromethyl)pyrimidine (476 mg, 2.195 mmol). The reaction mixture was allowed to stir at 50° C. for 36 h. The reaction mixture was concentrated under reduced pressure. The crude product was purified by flash chromatography on silica gel (1:1 EtOAc/PE) to afford the title compound 220. MS (ESI) m/z calc'd for C₂₄H₃₂ClF₃N₆O₃ [M+H]⁺: 545, found 545.

### (6S,7S)- or (6R,7R)-1-Cyclobutyl-3,7-dimethyl-11-(trifluoromethyl)-1,5,6,7,8,15-hexahydro-10,14-(azeno)-6,9-methanopyrazolo[4,3-b][1]oxa[4,6,10]triazacyclotetradecine (Ex-20.1 and Ex-20.2)

To the vial containing tert-butyl (3S,4S)- or (3R,4R)-3-(((5-((4-chloro-5-(trifluoromethyl)pyrimidin-2-yl)amino)-1-cyclobutyl-3-methyl-1H-pyrazol-4-yl)oxy)methyl)-4-methylpyrrolidine-1-carboxylate 220 (100 mg, 0.183 mmol) was added DCM (4 mL) and iodotrimethylsilane (220 mg, 1.101 mmol). The mixture was diluted with dioxane (4 mL), then TEA (0.332 mL, 2.385 mmol) was added. The mixture was allowed to stir at 25° C. for 2 h. The reaction mixture was concentrated under reduced pressure. The crude product was purified by reversed-phase HPLC, eluting with water (0.1% TFA)-MeCN, to afford the racemic material. The racemic material could be resolved to its component enantiomers by chiral preparative SFC (Column: Chiralpak AD-3 50*4.6 mm I.D., 3 μm; Mobile phase: A: CO₂; B: Methanol (0.05% DEA); Gradient: from 5% to 40% of B in 2 min and hold 40% for 1.2 min, then 5% of B for 0.8 Min.) to afford the title compounds Ex-20.1 (t_{R}=2.2 min) and Ex-20.2 (t_{R}=3.3 min). MS (ESI) m/z calc'd for C₁₉H₂₃F₃N₆O [M+H]⁺: 409, found 409; ¹H NMR (500 MHz, CDCl₃) δ: 8.22 (s, 1H), 7.64 (s, 1H), 4.66 (m, 1H), 4.60-4.53 (m, 1H), 4.42 (m, 1H), 4.12-4.04 (m, 1H), 3.68-3.59 (m, 1H), 3.12 (m, 1H), 2.93 (t, J=11.7 Hz, 1H), 2.76 (m, 1H), 2.59 (m, 1H), 2.37-2.32 (m, 4H), 2.06-1.93 (m, 1H), 1.87-1.72 (m, 4H), 1.04 (d, J=6.6 Hz, 3H). MS (ESI) m/z calc'd for C₁₉H₂₃F₃N₆O [M+H]⁺: 409, found 409; ¹H NMR (500 MHz, CDCl₃) δ:8.23 (s, 1H), 7.54 (s, 1H), 4.66 (m, 1H), 4.60-4.52 (m, 1H), 4.42 (m, 1H), 4.08 (m, 1H), 3.63 (m, 1H), 3.12 (m, 1H), 2.93 (t, J=11.7 Hz, 1H), 2.77 (m, 1H), 2.58 (m, 1H), 2.37-2.33 (m, 4H), 2.06-1.93 (m, 1H), 1.87-1.70 (m, 4H), 1.04 (d, J=6.6 Hz, 3H).

### General Scheme 12

### 1H), 4.66 (m, 1H), 4.60-4.52 (m, 1H), 4.42 (m, 1H), 4.08 (m, 1H), 3.63 (m, 1H), 3.12 (m, 1H), 2.93 (t, J=11.7 Hz, 1H), 2.77 (m, 1H), 2.58 (m, 1H), 2.37-2.33 (m, 4H), 2.06-1.93 (m, 1H), 1.87-1.70 (m, 4H), 1.04 (d, J=6.6 Hz, 3H).

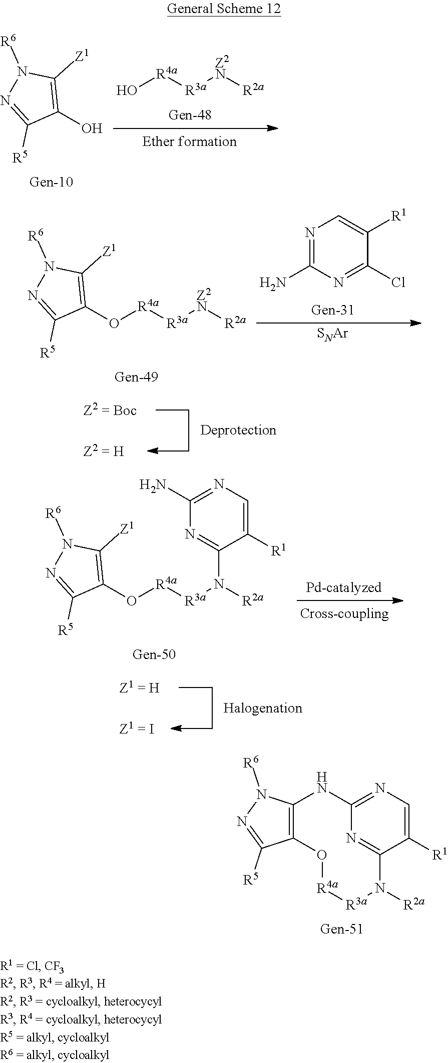

In General Scheme 12, pyrazole alcohols of the form Gen-10 were coupled with commercially available or synthetically prepared amino-alcohols of the form Gen48 through Mitsunobu chemistry or derivatives of the Mitsunobu reaction to provide Gen-49. In instances of Gen-49 where Z²=Boc, acidic deprotection to the amine was performed. Commercially available pyrimidines of the form Gen-31 were coupled with amines of the form Gen-1 through S_{N}Ar chemistry to access Gen-50. In instances of Gen-50 where Z¹=H, iodination of the pyrazole was performed. Iodopyrazoles of the form Gen-50 could ultimately be transformed through intramolecular Pd-catalyzed cross-coupling chemistry to afford elaborated compounds of the form Gen-51. Representative preparative examples from each sequence are described in more detail below.

### Preparation of Examples 21.1 and 21.2

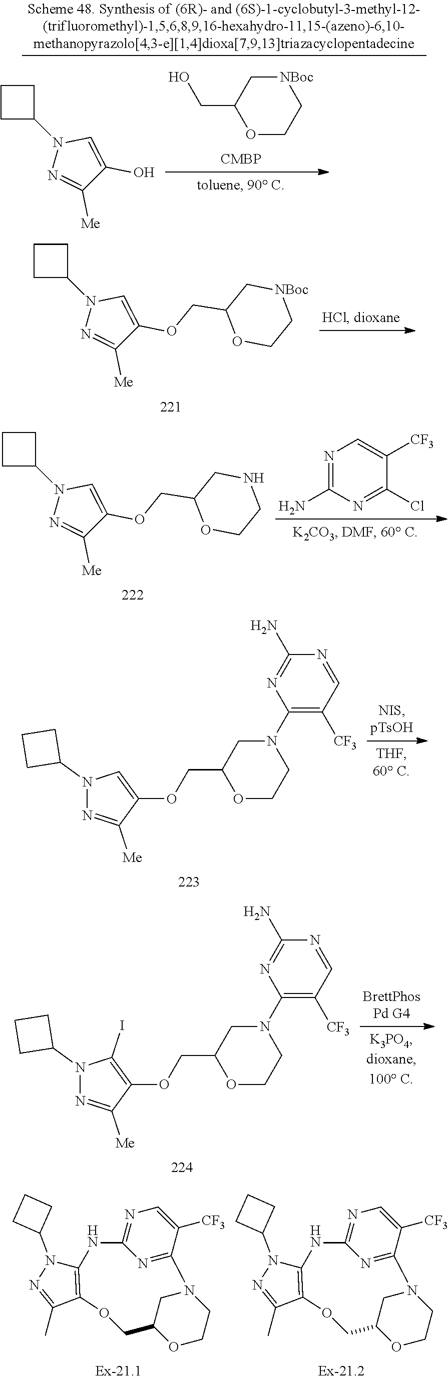

### tert-butyl (R) or (S)-2-(((1-cyclobutyl-3-methyl-1H-pyrazol-4-yl)oxy)methyl) morpholine-4-carboxylate (221)

To a solution of (rac)-tert-butyl 2-(hydroxymethyl) morpholine-4-carboxylate (286 mg, 1.31 mmol) and 1-cyclobutyl-3-methyl-1H-pyrazol-4-ol (200 mg, 1.31 mmol) in anhydrous Toluene (10 mL) was added (tributylphosphoranylidene) acetonitrile (476 mg, 2.0 mmol), and the reaction mixture was allowed to stir at 90° C. under N₂ for 18 h. The reaction was cooled to RT, diluted with water, and extracted three times with EtOAc. The combined organic extract was dried over Na₂SO₄, filtered and concentrated under reduced pressure. The resulting residue was purified by flash chromatography on silica gel (gradient elution of 0-100% EtOAc/hexanes) to afford the title compound 221.

### (R) and (S)-2-(((1-cyclobutyl-3-methyl-1H-pyrazol-4-yl)oxy)methyl) morpholine (222)

Hydrochloric acid (1.6 mL, 4.0M in dioxane, 6.4 mmol) was added dropwise to a solution of tert-butyl (R) or (S)-2-(((1-cyclobutyl-3-methyl-1H-pyrazol-4-yl)oxy)methyl) morpholine-4-carboxylate 221 (457 mg, 1.3 mmol) in dioxane (5.0 mL). The reaction was allowed to stir for 5 h then concentrated under reduced pressure to afford the title compound 222.

### (R) and (S)-4-(2-(((1-cyclobutyl-3-methyl-1H-pyrazol-4-yl)oxy)methyl) morpholino)-5-

Potassium carbonate (539 mg, 3.9 mmol), 4-chloro-5-(trifluoromethyl)pyrimidin-2-amine (257 mg, 1.3 mmol), and (R) and (S)-2-(((1-cyclobutyl-3-methyl-1H-pyrazol-4-yl)oxy)methyl) morpholine 222 (327 mg, 1.3 mmol) were suspended in DMF (3.0 ml) in a 20 mL vial, and the reaction mixture was allowed to stir at 60° C. under N₂ for 18 h. The reaction was cooled to RT, diluted with water, and extracted with EtOAc. The combined organic extract was washed with water, brine, and dried over Na₂SO₄, filtered and concentrated under reduced pressure. The resulting residue was purified by flash chromatography on silica gel (gradient elution of 0-100% EtOAc:EtOH (3:1 v/v)/hexanes) to afford the title compound 223.

### (R) and (S)-4-(2-(((1-cyclobutyl-5-iodo-3-methyl-1H-pyrazol-4-yl)oxy)methyl) morpholino)-5-(trifluoromethyl)pyrimidin-2-amine (224)

N-iodosuccinimide (290 mg, 1.3 mmol), tosic Acid (180 mg, 0.95 mmol), and (R) and (S)-4-(2-(((1-cyclobutyl-3-methyl-1H-pyrazol-4-yl)oxy)methyl) morpholino)-5-(trifluoromethyl)pyrimidin-2-amine 223 (355 mg, 0.86 mmol) were added to a vial and purged with N2. THF (8.0 ml) was added, and the reaction was allowed to stir at 60° C. under N₂ for 45 min. The reaction was cooled to RT, diluted with saturated aqueous sodium bicarbonate, and extracted with EtOAc. The combined organic layers were washed with saturated sodium thiosulfate, brine, dried over Na₂SO₄, filtered and concentrated under reduced pressure. The resulting residue was purified by flash chromatography on silica gel (gradient elution of 0-100% EtOAc:EtOH (3:1 v/v)/hexanes) to afford the title compound 224.

### (6R)- or (6S)-1-cyclobutyl-3-methyl-12-(trifluoromethyl)-1,5,6,8,9,16-hexahydro-11,15-(azeno)-6,10-methanopyrazolo[4,3-e][1,4]dioxa[7,9,13]triazacyclopentadecine (Ex-21.1 and Ex-21.2)

Potassium phosphate tribasic (99 mg, 0.46 mmol), Brettphos Pd G4 (17 mg, 0.02 mmol), and (R) and (S)-4-(2-(((1-cyclobutyl-5-iodo-3-methyl-1H-pyrazol-4-yl)oxy)methyl) morpholino)-5-(trifluoromethyl)pyrimidin-2-amine 224 (50 mg, 0.09 mmol) were weighed into a microwave vial. The vial was sealed and purged with N2. Dioxane (2.0 mL) was added, and the reaction was allowed to stir at 100° C. 18 h. The reaction was cooled to RT, diluted with water, and extracted with EtOAc. The combined organic layers were washed with brine, dried over Na₂SO₄, filtered and concentrated under reduced pressure. The resulting residue was purified by flash chromatography on silica gel (gradient elution of 0-100% EtOAc:EtOH (3:1 v/v)/hexanes). The crude product was purified by reversed-phase HPLC, eluting with water (0.1% TFA)-MeCN, to crude product was purified by reversed-phase HPLC, eluting with water (0.1% TFA)-MeCN, to afford the racemic material as a TFA salt solution. The solution was neutralized with saturated aqueous sodium bicarbonate, extracted with EtOAc and concentrated under reduced pressure to afford the racemic material. The racemic material could be resolved to its component enantiomers by chiral preparative SFC (Column: Lux-2 21×250 mm I.D., 5 μm. Mobile phase: A: CO₂; B: methanol (0.1% NH₄OH)) to afford the title compounds Ex-21.1 (t_{R}=3.5 min) and Ex-21.2 (t_{R}=3.7 min). MS (ESI): m/z calc'd for C₁₈H₂₂F₃N₆O₂ [M+H]⁺: 411, found 411; ¹H NMR (DMSO-d₆, 500 MHz) δ 9.51 (s, 1H), 8.40 (s, 1H), 4.97-4.83 (m, 1H), 4.38 (d, J=13.7 Hz, 1H), 4.27-4.13 (m, 1H), 3.94 (dd, J=9.8, 3.6 Hz, 1H), 3.91-3.75 (m, 1H), 3.75-3.63 (m, 1H), 3.62-3.42 (m, 3H), 3.22 (dd, J=13.7, 6.4 Hz, 1H), 2.49-2.32 (m, 2H), 2.39-2.19 (m, 2H), 2.15 (s, 3H), 1.81-1.57 (m, 2H).

Compounds in Table 12 below were prepared in accordance with the synthetic sequence illustrated above using the corresponding starting materials.

| | | | Exact Mass |
|---|---|---|---|
| Ex | Structure | Name | [M + H\|⁺ |
| Ex-21.3 | 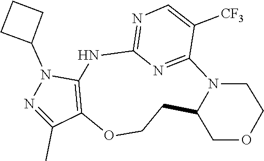 [SMILES: Cc1nn(C2CCC2)c2c1OCC[C@@H]1COCCN1c1nc(ncc1C(F)(F)F)N2] | (R) or (S)-3-cyclobutyl-1-methyl-8- (trifluoromethyl)- 3,4,11,12,14,14a,15,16-octahydro- 5,9-(azeno)[1,4]oxazino[4,3- j]pyrazolo[4,3- b][1]oxa[4,6,10]triazacyclotridecine | Calc'd 425, found 425 |
| Ex-21.4 | 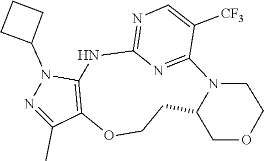 [SMILES: Cc1nn(C2CCC2)c2c1OCC[C@H]1COCCN1c1nc(ncc1C(F)(F)F)N2] | (R) or (S)-3-cyclobutyl-1-methyl-8- (trifluoromethyl)- 3,4,11,12,14,14a,15,16-octahydro- 5,9-(azeno)[1,4]oxazino[4,3- j]pyrazolo[4,3- b][1]oxa[4,6,10]triazacyclotridecine | Calc'd 425, found 425 |

### Biological Assay: LRRK2 Km ATP LanthaScreen™ Assay

The LRRK2 kinase activity reported herein as IC₅₀ values was determined with LanthaScreen™ technology from Life Technologies Corporation (Carlsbad, CA) using GST-tagged truncated human mutant G2019S LRRK2 in the presence of the fluorescein-labeled peptide substrate LRRKtide, also from Life Technologies. The data presented for the Km ATP reasonable deviations depending on the specific conditions and reagents used. Assays were performed in the presence of 134 μM ATP (Km ATP). Upon completion, the assay was stopped and phosphorylated substrate detected with a terbium (Tb)-labeled anti-pERM antibody (cat. no. PV4898). The compound dose response was prepared by diluting a 10 mM stock of compound to a maximum concentration of 9.99 μM in 100% dimethylsulfoxide followed by custom fold serial dilution in dimethylsulfoxide nine times. Twenty nanoliters of each dilution was spotted via a Labcyte Echo onto a 384-well black-sided plate (Corning 3575) followed by 15 μl of a 1.25 nM enzyme solution in 1× assay buffer (50 mM Tris pH 8.5, 10 mM MgCl2, 0.01% Brij-35, 1 mM EGTA, 2 mM dithiothreitol, 0.05 mM sodium orthovanadate). Following a 15-minute incubation at room temperature, the kinase reaction was started with the addition of 5 μl of 400 nM fluorescein-labeled LRRKtide peptide substrate and 134 μM ATP solution in 1× assay buffer. The reaction was allowed to progress at ambient temperature for 90 minutes. The reaction was then stopped by the addition of 20 μl of TR-FRET Dilution Buffer (Life Technologies, Carlsbad, CA) containing 2 nM Tb-labeled anti-phospho LRRKtide antibody and 10 mM EDTA (Life Technologies, Carlsbad, CA). After an incubation of 1 h at room temperature, the plate was read on an EnVision multimode plate reader (Perkin Elmer, Waltham, MA) with an excitation wavelength of 337 nm (Laser) and a reading emission at both 520 and 495 nm. Compound IC50s were interpolated from nonlinear regression best fits of the log of the final compound concentration, plotted as a function of the 520/495-nm emission ratio using Activity base. Abase uses a 4 parameter (4P) logistic fit based on the Levenberg-Marquardt algorithm.

TABLE 13

| Ex | LRRK2 pIC₅₀ |
|---|---|
| Ex-1.1 | >10.09 |
| Ex-1.2 | >10.09 |
| Ex-1.3 | 9.07 |
| Ex-1.4 | 8.93 |
| Ex-1.5 | 8.73 |
| Ex-1.6 | 6.92 |
| Ex-1.7 | >10.09 |
| Ex-1.8 | 5.94 |
| Ex-1.9 | >10.09 |
| Ex-1.10 | 6.58 |
| Ex-1.11 | >10.09 |
| Ex-1.12 | <5.00 |
| Ex-1.13 | 7.99 |
| Ex-1.14 | 8.66 |
| Ex-1.15 | 8.27 |
| Ex-1.16 | 9.76 |
| Ex-1.17 | >10.09 |
| Ex-1.18 | >10.09 |
| Ex-1.19 | 9.87 |
| Ex-1.20 | 9.42 |
| Ex-1.21 | >10.09 |
| Ex-1.22 | >10.09 |
| Ex-1.23 | 9.47 |
| Ex-2.1 | 6.64 |
| Ex-2.2 | 5.45 |
| Ex-2.3 | 9.12 |
| Ex-2.4 | 6.66 |
| Ex-2.5 | >10.09 |
| Ex-2.6 | 9.17 |
| Ex-2.7 | 7.21 |
| Ex-2.8 | 9.48 |
| Ex-2.9 | 6.31 |
| Ex-2.10 | >10.09 |
| Ex-2.11 | >10.09 |
| Ex-2.12 | 7.61 |
| Ex-2.13 | >10.09 |
| Ex-2.14 | 6.73 |
| Ex-2.15 | 7.44 |
| Ex-2.16 | 7.93 |
| Ex-2.17 | 8.42 |
| Ex-2.18 | 6.65 |
| Ex-2.19 | 8.78 |
| Ex-2.20 | 6.71 |
| Ex-2.21 | 9.00 |
| Ex-2.22 | 5.76 |
| Ex-2.23 | >10.09 |
| Ex-2.24 | 6.05 |
| Ex-2.25 | 7.55 |
| Ex-2.26 | 6.77 |
| Ex-2.27 | 6.47 |
| Ex-2.28 | 9.64 |
| Ex-2.29 | 5.70 |
| Ex-2.30 | 10.03 |
| Ex-2.31 | 6.54 |
| Ex-2.32 | 10.04 |
| Ex-2.33 | 9.11 |
| Ex-2.34 | 8.00 |
| Ex-2.35 | 8.56 |
| Ex-2.36 | 6.31 |
| Ex-2.37 | 6.00 |
| Ex-2.38 | 6.13 |
| Ex-2.39 | 6.32 |
| Ex-2.40 | 8.96 |
| Ex-2.41 | 6.51 |
| Ex-2.42 | 9.93 |
| Ex-2.43 | >10.09 |
| Ex-2.44 | 8.80 |
| Ex-2.45 | 8.32 |
| Ex-2.46 | 6.66 |
| Ex-2.47 | >10.09 |
| Ex-2.48 | >10.09 |
| Ex-2.49 | >10.09 |
| Ex-2.50 | 9.77 |
| Ex-2.51 | 9.05 |
| Ex-2.52 | 5.96 |
| Ex-2.53 | 9.64 |
| Ex-2.54 | 8.57 |
| Ex-2.55 | 8.94 |
| Ex-2.56 | >10.09 |
| Ex-2.57 | 9.98 |
| Ex-2.58 | 8.78 |
| Ex-2.59 | 9.71 |
| Ex-2.60 | 9.41 |
| Ex-2.61 | 9.60 |
| Ex-2.62 | 6.88 |
| Ex-2.63 | >10.09 |
| Ex-2.64 | >10.09 |
| Ex-2.65 | 6.59 |
| Ex-2.66 | 8.43 |
| Ex-2.67 | 9.45 |
| Ex-2.68 | >10.09 |
| Ex-2.69 | >10.09 |
| Ex-2.70 | 9.35 |
| Ex-2.71 | 8.24 |
| Ex-2.72 | >10.09 |
| Ex-2.73 | >10.09 |
| Ex-2.74 | >10.09 |
| Ex-2.75 | 9.50 |
| Ex-2.76 | 9.47 |
| Ex-2.77 | >10.09 |
| Ex-2.78 | >10.09 |
| Ex-2.79 | 6.87 |
| Ex-2.80 | 7.57 |
| Ex-2.81 | 5.99 |
| Ex-2.82 | 9.60 |
| Ex-2.83 | 5.76 |
| Ex-2.84 | 8.77 |
| Ex-2.85 | 8.74 |
| Ex-2.86 | 8.13 |
| Ex-2.87 | 6.84 |
| Ex-2.88 | 7.64 |
| Ex-3.1 | >10.09 |
| Ex-4.1 | 6.25 |
| Ex-4.2 | 9.51 |
| Ex-5.1 | >10.09 |
| Ex-5.2 | 9.46 |
| Ex-5.3 | 7.30 |
| Ex-5.4 | 6.78 |
| Ex-5.5 | 10.06 |
| Ex-5.6 | 9.80 |
| Ex-6.1 | 7.94 |
| Ex-6.2 | >10.09 |
| Ex-6.3 | 7.29 |
| Ex-6.4 | >10.09 |
| Ex-6.5 | >10.09 |
| Ex-6.6 | >10.09 |
| Ex-6.7 | >10.09 |
| Ex-6.8 | >10.09 |
| Ex-6.9 | >10.09 |
| Ex-7.1 | 6.62 |
| Ex-7.2 | 10.03 |
| Ex-7.3 | >10.09 |
| Ex-7.4 | >10.09 |
| Ex-7.5 | >10.09 |
| Ex-8.1 | >10.09 |
| Ex-9.1 | 6.11 |
| Ex-10.1 | >10.09 |
| Ex-11.1 | 8.49 |
| Ex-11.2 | 8.50 |
| Ex-12.1 | >10.09 |
| Ex-12.2 | 9.62 |
| Ex-13.1 | >10.09 |
| Ex-13.2 | 7.01 |
| Ex-13.3 | >10.09 |
| Ex-13.4 | 9.61 |
| Ex-13.5 | >10.09 |
| Ex-13.6 | <5.00 |
| Ex-13.7 | 5.02 |
| Ex-13.8 | <5.00 |
| Ex-13.9 | 8.49 |
| Ex-14.1 | 8.78 |
| Ex-14.2 | 5.16 |
| Ex-15.1 | >10.09 |
| Ex-15.2 | 9.65 |
| Ex-15.3 | 9.15 |
| Ex-15.4 | 9.12 |
| Ex-15.5 | 9.90 |
| Ex-15.6 | 9.08 |
| Ex-15.7 | 10.01 |
| Ex-15.8 | 9.04 |
| Ex-15.9 | 8.58 |
| Ex-16.1 | 7.70 |
| Ex-16.2 | 9.20 |
| Ex-17.1 | 6.63 |
| Ex-17.2 | 8.24 |
| Ex-18.1 | 7.94 |
| Ex-19.1 | >10.09 |
| Ex-20.1 | 5.84 |
| Ex-20.2 | 9.39 |
| Ex-21.1 | 6.09 |
| Ex-21.2 | 8.07 |
| Ex-21.3 | 7.64 |
| Ex-21.4 | 5.61 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A compound of structural Formula (I) or Formula Ia: 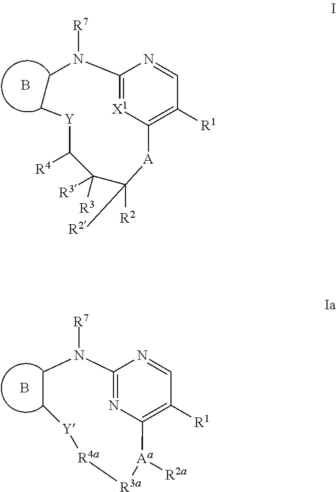
or a pharmaceutically acceptable salt thereof, wherein,
B is a C₅₋₆ heteroaryl or C₆₋₁₀ aryl, said heteroaryl and aryl optionally substituted with 1 to 3 groups of R^{x};
Y is O, OCH₂ or CH₂;
Y¹ is O, OCH₂, or CH₂;
X¹ is N or CH;
R¹ is selected from C₁₋₆alkyl, C₃₋₆cycloalkyl, halogen, C₁₋₃haloalkyl,
R² is selected from hydrogen, and C₁₋₆alkyl, said alkyl optionally substituted from 1 to 3 groups selected from C₁₋₆ alkyl, CF₃, and CN,
R^{2a} is hydrogen
R³ is selected from hydrogen, and C₁₋₆alkyl, said alkyl optionally substituted from 1 to 3 groups selected from C₁₋₆ alkyl, CF₃, and CN,
R^{3a} is CHR^{4b};
R^{4a} is (CH₂)ₙ; or
R^{3a} and R^{4a} combine to form a C₃₋₁₀ cycloalkyl, or C₃₋₁₀ heterocyclyl, said cycloalkyl and heterocyclyl optionally substituted with 1 to 3 groups of R^{b};
R^{4b} is selected from hydrogen and C₁₋₃alkyl,
R^{2′} and R^{3′} are independently hydrogen, or
R^{2′} and R² together form a spiro-C₃₋₆cycloalkyl, or a spiro-C₃₋₁₀heterocyclyl; said cycloalkyl and heterocyclyl optionally substituted with 1 to 3 groups of C₁₋₆alkyl, C₃₋₁₀heterocyclyl, and halogen, or
R^{3′} and R³ together form a spiro-C₃₋₆cycloalkyl, or a spiro-C₃₋₁₀heterocyclyl; said cycloalkyl and heterocyclyl optionally substituted with 1 to 3 groups of C₁₋₆alkyl, C₃₋₁₀heterocyclyl, and halogen, or
R² and R³ can combine with the atoms to which they are attached to form a cyclic group selected from C₃₋₆cycloalkyl and C₃₋₁₀heterocyclyl, said cycloalkyl and heterocyclyl optionally substituted with 1 to 3 groups of C₁₋₆alkyl, C₃₋₁₀heterocyclyl, and halogen, said heterocyclyl optionally substituted with 1 to 3 halogen substituents;
R⁴ is hydrogen; or
R³ and R⁴ can combine with the atoms to which they are attached to form a cyclic group selected from C₃₋₆cycloalkyl and C₃₋₁₀heterocyclyl, said cycloalkyl and heterocyclyl optionally substituted with 1 to 3 groups of C₁₋₆alkyl, C₃₋₁₀heterocyclyl, and halogen, said heterocyclyl optionally substituted with 1 to 3 groups of halogen;
R^{x} is selected from C₁₋₆alkyl, C₂₋₆alkenyl, C(O)OC₁₋₆alkyl, C₃₋₆cycloalkyl, C₁₋₃haloalkyl, and C₃₋₁₀heterocyclyl, said alkyl, alkenyl, cycloalkyl and heterocyclyl optionally substituted with 1 to 3 R^{a} substituents each independently selected from hydrogen, C₁₋₆ alkyl, —OC₁₋₆alkyl, CN, SO₂, OH, C(O)OC₁₋₆alkyl, C₁₋₃haloalkyl, C₃₋₁₀ heteroaryl, C₃₋₁₀ heterocyclyl, and halogen, said heteroaryl and heterocyclyl of R^{a} optionally substituted with 1 to 3 R^{a′} substituents wherein each R^{a′} is independently selected from C₁₋₆ alkyl, CF₃, and CN;
A is O, NH or CH₂;
A^{a} is N or CH; or
A^{a}, R^{2a} and R^{3a} combine with the atoms to which they are attached to form a C₃₋₁₀ heterocyclyl, said heterocyclyl optionally substituted with 1 to 3 groups of R^{b};
R^{b} is selected from C₁₋₆ alkyl, OC₁₋₆ alkyl, CN, SO₂, OH, C(O)OC₁₋₆alkyl, C₁₋₃haloalkyl, C₃₋₁₀ cycloalkyl, and halogen;
R⁷ is hydrogen or C₁₋₆alkyl, and
n is 0, 1, 2, 4, or 4.

2. The compound according to claim 1 represented by Formula 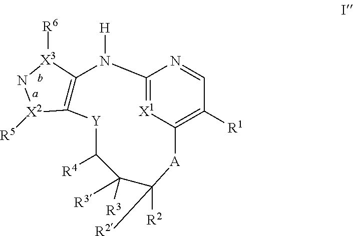
or a pharmaceutically acceptable salt thereof, wherein:
A, Y, X¹, R¹, R², R^{2′} R³, R^{3′} and R⁴ are as described,
X² and X³ are independently selected from C and N;
bond “a” is a double bond when X² is C
bond “b” is a double bond when X³ is C; provided that only one of X² or X³ is carbon at the same time;
R⁵ is selected from hydrogen, C₁₋₆alkyl, C(O)OC₁₋₆alkyl, C₃₋₆cycloalkyl, C₁₋₃haloalkyl, and C₃₋₁₀heterocyclyl, said alkyl, cycloalkyl and heterocyclyl optionally substituted with 1 to 3 R^{a} substituents each independently selected from hydrogen, C₁₋₆ alkyl, CN, SO₂, OH, C(O)OC₁₋₆alkyl, C₁₋₃haloalkyl, C₃₋₁₀ heteroaryl, C₃₋₁₀ heterocyclyl, and halogen, said heteroaryl and heterocyclyl of R^{a} optionally substituted with 1 to 3 R^{a′} substituents wherein each R^{a′} is independently selected from C₁₋₆ alkyl, CF₃, and CN; and
R⁶ is selected from C₁₋₆alkyl, C₂₋₆alkenyl, C₃₋₆cycloalkyl, C₁₋₃haloalkyl, and C₃₋₁₀heterocyclyl, said alkyl, alkenyl, cycloalkyl and heterocyclyl optionally substituted with 1 to 3 groups of R^{b} selected from hydrogen, C₁₋₆ alkyl, OH, —OC₁₋₆alkyl, and halogen.

3. The compound according to claim 1 wherein A and A^{a} are independently selected from NH or CH₂.

4. The compound according to claim 1 wherein Y and Y′ are independently O.

5. The compound according to claim 2 wherein X¹, X² and X³ are N, C and N, respectively, and bond a is a double bond and bond b is a single bond.

6. The compound according to claim 4 wherein X¹, X² and X³ are N, N and C, respectively, and bond a is a single bond and bond b is a double bond.

7. The compound according to claim 1 wherein R¹ is selected from methyl, ethyl, propyl, butyl, pentyl, hexyl, cyclopropyl, cyclobutyl, chlorine, fluorine, CF₃, CHF₂, and CH₂F.

8. The compound according to claim 1 wherein R^{3′} and R⁴ are hydrogen, and R² and R³ are independently selected from hydrogen, methyl, ethyl, propyl, butyl, hexyl, cyclopropyl, and cyclobutyl.

9. The compound according to claim 1 wherein or R² is selected from hydrogen, methyl, ethyl, propyl, butyl, hexyl, cyclopropyl, and cyclobutyl, R⁴ is hydrogen, and R^{3′} and R³ together form a spiro-C₃₋₆cycloalkyl or a spiro-C₃₋₁₀heterocyclyl.

10. The compound according to claim 1 wherein R^{3′} and R⁴ are hydrogen, and R² and R³ combine with the atoms to which they are attached to form a cyclic group selected from optionally substituted cyclopropyl, cyclobutyl, cyclopentyl, cyclohexyl, tetrahydrofuranyl, tetrahydropyranyl, oxetanyl, azetidinyl, piperazinyl, piperidinyl, and pyrrolidinyl.

11. The compound according to claim 1 wherein R³ and R⁴ combine with the atoms to which they are attached to form a group selected from optionally substituted cyclopropyl, cyclobutyl, cyclopentyl, cyclohexyl, tetrahydrofuranyl, tetrahydropyranyl, oxetanyl, azetidinyl, piperazinyl, piperidinyl, and pyrrolidinyl.

12. The compound according to claim 2 wherein R⁵ is selected from optionally substituted —CH₂R^{a}, —CH₂(CH₃)₂R^{a}, —CH₃, —CH₂CH₃, —C(CH₃)₂R^{a}, —C(CH₃)₂CH₂R^{a}, CF₃, CHF₂, CH₂F, optionally substituted cyclopropyl, cyclobutyl, tetrahydrofuranyl, azetidinly, piperazinyl, piperidinyl, oxetanyl, and thietanyl sulfoxide.

13. The compound according to claim 2 wherein R⁶ is —CH₂R^{b}, —CH(CH₃)₂, —CH₂(CH₃)₂R^{b}, —CH₃, —CH₂CH₃, —C(CH₃)₂R^{b}, —C(CH₃)₂CH₂R^{b}, CF₃, CHF₂, CH₂F, and optionally substituted cyclopropyl and cyclobutyl.

14. The compound according to Formula Ia of claim 1 represented by Formula Ia″ 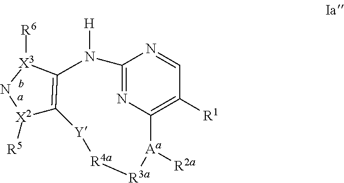
or a pharmaceutically acceptable salt thereof, wherein Y′, A^{a}, R¹, R^{2a}, R^{3a}, and R^{4a}, are as described;
X² an X³ are independently selected from C and N;
bond “a” is a double bond when X² is C
bond “b” is a double bond when X³ is C; provided that only one of X² or X³ is carbon at the same time;
R⁵ is selected from hydrogen, C₁₋₆alkyl, C(O)OC₁₋₆alkyl, C₃₋₆cycloalkyl, C₁₋₃haloalkyl, and C₃₋₁₀heterocyclyl, said alkyl, cycloalkyl and heterocyclyl optionally substituted with 1 to 3 R^{a} substituents each independently selected from hydrogen, C₁₋₆ alkyl, CN, SO₂, OH, C(O)OC₁₋₆alkyl, C₁₋₃haloalkyl, C₃₋₁₀ heteroaryl, C₃₋₁₀ heterocyclyl, and halogen, said heteroaryl and heterocyclyl of R^{a} optionally substituted with 1 to 3 R^{a′} substituents wherein each R^{a′} is independently selected from C₁₋₆ alkyl, CF₃, and CN; and
R⁶ is selected from C₁₋₆alkyl, C₂₋₆alkenyl, C₃₋₆cycloalkyl, C₁₋₃haloalkyl, and C₃₋₁₀heterocyclyl, said alkyl, alkenyl, cycloalkyl and heterocyclyl optionally substituted with 1 to 3 groups of R^{b} selected from hydrogen, C₁₋₆ alkyl, OH, —OC₁₋₆alkyl, and halogen.

15. The compound according to Formula I of claim 1 represented by formulas II and III: 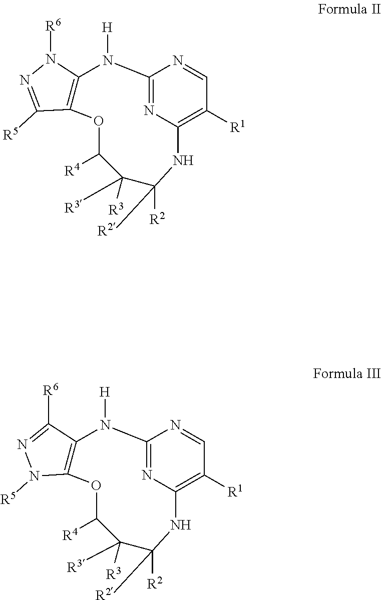
wherein R¹, R², R³, R⁴, are as described;
R⁵ is selected from hydrogen, C₁₋₆alkyl, C(O)OC₁₋₆alkyl, C₃₋₆cycloalkyl, C₁₋₃haloalkyl, and C₃₋₁₀heterocyclyl, said alkyl, cycloalkyl and heterocyclyl optionally substituted with 1 to 3 R^{a} substituents each independently selected from hydrogen, C₁₋₆ alkyl, CN, SO₂, OH, C(O)OC₁₋₆alkyl, C₁₋₃haloalkyl, C₃₋₁₀ heteroaryl, C₃₋₁₀ heterocyclyl, and halogen, said heteroaryl and heterocyclyl of R^{a} optionally substituted with 1 to 3 R^{a′} substituents wherein each R^{a′} is independently selected from C₁₋₆ alkyl, CF₃, and CN; and
R⁶ is selected from C₁₋₆alkyl, C₂₋₆alkenyl, C₃₋₆cycloalkyl, C₁₋₃haloalkyl, and C₃₋₁₀heterocyclyl, said alkyl, alkenyl, cycloalkyl and heterocyclyl optionally substituted with 1 to 3 groups of R^{b} selected from hydrogen, C₁₋₆ alkyl, OH, —OC₁₋₆alkyl, and halogen.

16. A compound selected from:

| Ex | Structure | Name |
|---|---|---|
| Ex-1.1 | 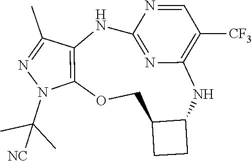 [SMILES: Cc1nn(C(C)(C)C#N)c2c1Nc1ncc(C(F)(F)F)c(n1)N[C@@H]1CC[C@H]1CO2] | 2-Methyl-2-((10aR,12aR)-3-methyl-8- (trifluoromethyl)-10,10a,11,12,12a,13- hexahydro-5,9- (azeno)cyclobuta[k]pyrazolo[3,4- b][1]oxa[4,6,10]triazacyclotridecin-1(4H)- yl)propanenitrile |
| Ex-1.2 | 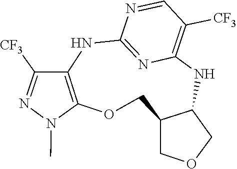 [SMILES: Cn1nc(C(F)(F)F)c2c1OC[C@@H]1COC[C@H]1Nc1nc(ncc1C(F)(F)F)N2] | (10aS,13aS)-1-Methyl-3,8- bis(trifluoromethyl)-1,4,10a,11,13a,14- hexahydro-10H,13H-5,9-(azeno)furo[3,4- k]pyrazolo[3,4- b][1]oxa[4,6,10]triazacyclotridecine |
| Ex-1.3 | 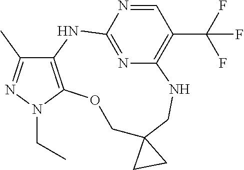 [SMILES: CCn1nc(C)c2c1OCC1(CC1)CNc1nc(ncc1C(F)(F)F)N2] | 1′-ethyl-3′-methyl-8′-(trifluoromethyl)- 1′,4′,10′,11′-tetrahydro-13′H- spiro[cyclopropane-1,12′- [5,9](azeno)pyrazolo[3,4- b][1]oxa[4,6,10]triazacyclotridecine] |
| Ex-1.4 | 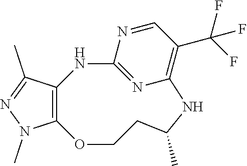 [SMILES: Cc1nn(C)c2c1Nc1ncc(C(F)(F)F)c(n1)N[C@H](C)CCO2] | (R)-1,3,11-trimethyl-8-(trifluoromethyl)- 1,4,10,11,12,13-hexahydro-5,9- (azeno)pyrazolo[3,4- b][1]oxa[4,6,10]triazacyclotridecine |
| Ex-1.5 | 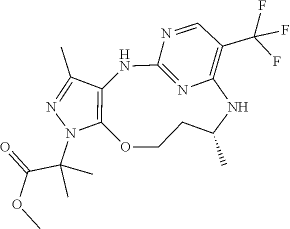 [SMILES: COC(=O)C(C)(C)n1nc(C)c2c1OCC[C@@H](C)Nc1nc(ncc1C(F)(F)F)N2] | methyl(R)-2-(3,11-dimethyl-8- (trifluoromethyl)-10,11,12,13-tetrahydro-5,9- (azeno)pyrazolo[3,4- b][1]oxa[4,6,10]triazacyclotridecin-1(4H)- yl)-2-methylpropanoate |
| Ex-1.6 | 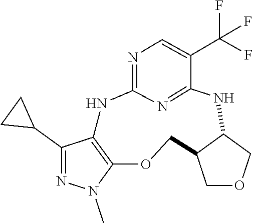 | (10aS,13aS)- or (10aR,13aR)- 3-cyclopropyl-1-methyl-8-(trifluoromethyl)- 1,4,10a,11,13a,14-hexahydro-10H,13H-5,9- (azeno)furo[3,4-k]pyrazolo[3,4- b][1]oxa[4,6,10]triazacyclotridecine |
| Ex-1.7 | 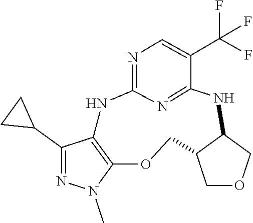 | (10aS,13aS)- or (10aR,13aR)- 3-cyclopropyl-1-methyl-8-(trifluoromethyl)- 1,4,10a,11,13a,14-hexahydro-10H,13H-5,9- (azeno)furo[3,4-k]pyrazolo[3,4- b][1]oxa[4,6,10]triazacyclotridecine |
| Ex-1.8 | 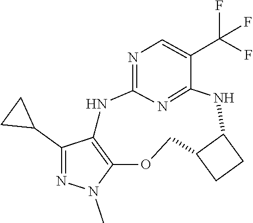 | (10aR,12aS)- or (10aS,12aR)- 3-cyclopropyl-1-methyl-8-(trifluoromethyl)- 1,4,10,10a,11,12,12a,13-octahydro-5,9- (azeno)cyclobuta[k]pyrazolo-[3,4- b][1]oxa[4,6,10]triazacyclo-tridecine |
| Ex-1.9 | 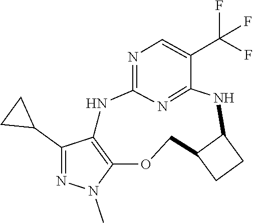 | (10aR,12aS)- or (10aS,12aR)- 3-cyclopropyl-1-methyl-8-(trifluoromethyl)- 1,4,10,10a,11,12,12a,13-octahydro-5,9- (azeno)cyclobuta[k]pyrazolo-[3,4- b][1]oxa[4,6,10]triazacyclo-tridecine |
| Ex-1.10 | 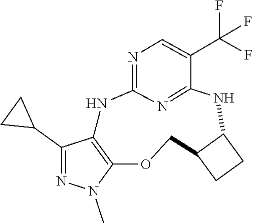 | (10aR,12aR)- or (10aS, 12aS)- 3-cyclopropyl-1-methyl-8-(trifluoromethyl)- 1,4,10,10a,11,12,12a,13-octahydro-5,9- (azeno)cyclobuta[k]pyrazolo-[3,4- b][1]oxa[4,6,10]triazacyclo-tridecine |
| Ex-1.11 | 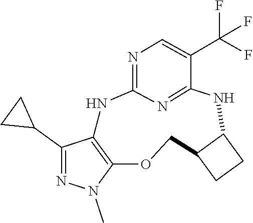 | (10aR,12aR)- or (10aS,12aS)- 3-cyclopropyl-1-methyl-8-(trifluoromethyl)- 1,4,10,10a,11,12,-12a,13-octahydro-5,9- (azeno)cyclobuta[k]pyrazolo[3,4- b][1]oxa[4,6,10]triazacyclotridecine |
| Ex-1.12 | 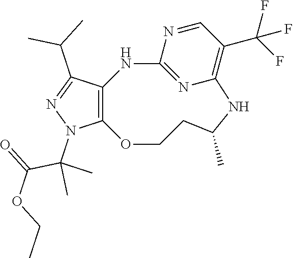 [SMILES: CCOC(=O)C(C)(C)n1nc(C(C)C)c2c1OCC[C@@H](C)Nc1nc(ncc1C(F)(F)F)N2] | ethyl (R)-2-(3-isopropyl-11-methyl-8- (trifluoromethyl)-10,11,12,13-tetrahydro-5,9- (azeno)pyrazolo[3,4-b][1]oxa[4,6,10] triazacyclotridecin-1(4H)-yl)-2- methylpropanoate |
| Ex-1.13 | 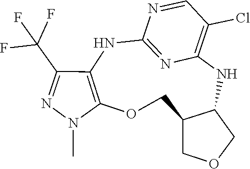 [SMILES: Cn1nc(C(F)(F)F)c2c1OC[C@@H]1COC[C@H]1Nc1nc(ncc1Cl)N2] | (10aS,13aS)-8-chloro-1-methyl-3- (trifluoromethyl)-1,4,10a,11,13a,14- hexahydro-10H,13H-5,9-(azeno)furo[3,4- k]pyrazolo[3,4- b][1]oxa[4,6,10]triazacyclotridecine |
| Ex-1.14 | 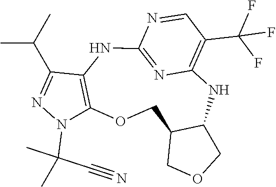 [SMILES: CC(C)c1nn(C(C)(C)C#N)c2c1Nc1ncc(C(F)(F)F)c(n1)N[C@@H]1COC[C@H]1CO2] | 2-((10aS,13aS)-3-isopropyl-8- (trifluoromethyl)-10a,11,13a,14-tetrahydro- 10H,13H-5,9-(azeno)furo[3,4- b][1]oxa[4,6,10]triazacyclotridecin-1(4H)- yl)-2-methylpropanenitrile |
| Ex-1.15 | 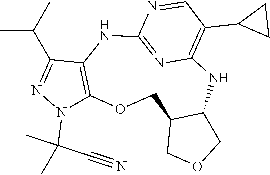 [SMILES: CC(C)c1nn(C(C)(C)C#N)c2c1Nc1ncc(C3CC3)c(n1)N[C@@H]1COC[C@H]1CO2] | 2-((10aS,13aS)-8-cyclopropyl-3-isopropyl- 10a,11,13a,14-tetrahydro-10H,13H-5,9- (azeno)furo[3,4-k]pyrazolo[3,4- b][1]oxa[4,6,10]triazacyclotridecin-1(4H)- yl)-2-methylpropanenitrile |
| Ex-1.16 | 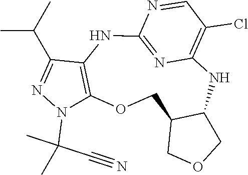 [SMILES: CC(C)c1nn(C(C)(C)C#N)c2c1Nc1ncc(Cl)c(n1)N[C@@H]1COC[C@H]1CO2] | 2-((10aS,13aS)-8-chloro-3-isopropyl- 10a,11,13a,14-tetrahydro-10H,13H-5,9- (azeno)furo[3,4-k]pyrazolo[3,4- b][1]oxa[4,6,10]triazacyclotridecin-1(4H)- yl)-2-methylpropanenitrile |
| Ex-1.17 | 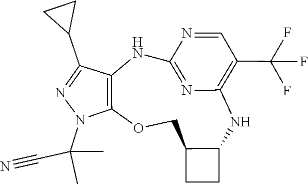 [SMILES: CC(C)(C#N)n1nc(C2CC2)c2c1OC[C@@H]1CC[C@H]1Nc1nc(ncc1C(F)(F)F)N2] | 2-((10aR,12aR)-3-cyclopropyl-8- (trifluoromethyl)-10,10a,11,12,12a,13- hexahydro-5,9-(azeno)cyclobuta[k]pyrazolo- [3,4-b][1]oxa[4,6,10]triazacyclotridecin- 1(4H)-yl)-2-methylpropanenitrile |
| Ex-1.18 | 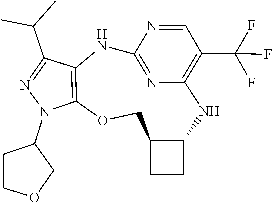 [SMILES: CC(C)c1nn(C2CCOC2)c2c1Nc1ncc(C(F)(F)F)c(n1)N[C@@H]1CC[C@H]1CO2] | (10aR,12aR)-3-isopropyl-1-((R)- or- (10aR,12aR)-3-isopropyl-1-((S)- tetrahydrofuran-3-yl)-8-(trifluoromethyl)- 1,4,10,10a,11,12,12a,13-octahydro-5,9- (azeno)cyclobuta[k]pyrazolo-[3,4- b][1]oxa[4,6,10]triazacyclotridecine |
| Ex-1.19 | 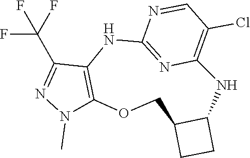 [SMILES: Cn1nc(C(F)(F)F)c2c1OC[C@@H]1CC[C@H]1Nc1nc(ncc1Cl)N2] | (10aR,12aR)-8-chloro-1-methyl-3- (trifluoromethyl)-1,4,10,10a,11,12,12a,13- octahydro-5,9- (azeno)cyclobuta[k]pyrazolo[3,4- b][1]oxa[4,6,10]triazacyclotridecine |
| Ex-1.20 | 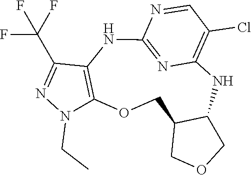 [SMILES: CCn1nc(C(F)(F)F)c2c1OC[C@@H]1COC[C@H]1Nc1nc(ncc1Cl)N2] | (10aS,13aS)-8-chloro-1-ethyl-3- (trifluoromethyl)-1,4,10a,11,13a,14- hexahydro-10H,13H-5,9-(azeno)furo[3,4- b][1]oxa[4,6,10]triazacyclotridecine |
| Ex-1.21 | 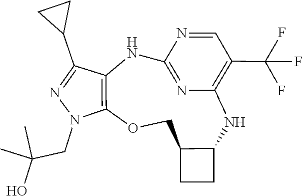 [SMILES: CC(C)(O)Cn1nc(C2CC2)c2c1OC[C@@H]1CC[C@H]1Nc1nc(ncc1C(F)(F)F)N2] | 1-((10aR,12aR)-3-cyclopropyl-8- (trifluoromethyl)-10,10a,11,12,12a,13- hexahydro-5,9- (azeno)cyclobuta[c]pyrazolo[3,4- b][1]oxa[4,6,10]triazacyclotridecin-1(4H)- yl)-2-methylpropan-2-ol |
| Ex-1.22 | 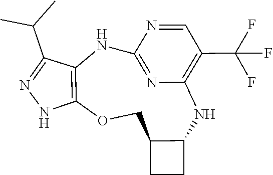 [SMILES: CC(C)c1n[nH]c2c1Nc1ncc(C(F)(F)F)c(n1)N[C@@H]1CC[C@H]1CO2] | (10aR,12aR)-3-isopropyl-8-(trifluoromethyl)- 1,4,10,10a,11,12,12a,13-octahydro-5,9- (azeno)cyclobuta[k]pyrazolo[3,4- b][1]oxa[4,6,10]triazacyclotridecine |
| Ex-1.23 | 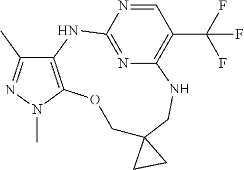 [SMILES: Cc1nn(C)c2c1Nc1ncc(C(F)(F)F)c(n1)NCC1(CC1)CO2] | 1′,3′-dimethyl-8′-(trifluoromethyl)- 1′,4′,10′,11′-tetrahydro-13′H- spiro[cyclopropane-1,12′- [5,9](azeno)pyrazolo[3,4- b][1]oxa[4,6,10]triazacyclotridecine] |
| Ex-2.1 | 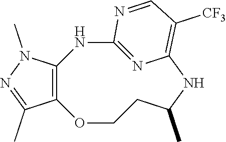 [SMILES: Cc1nn(C)c2c1OCC[C@H](C)Nc1nc(ncc1C(F)(F)F)N2] | (R)- or (S)-1,3,7-trimethyl-10- (trifluoromethyl)-1,5,6,7,8,14-hexahydro- 9,13-(azeno)pyrazolo[4,3-b][1]oxa[4,6,10] triazacyclotridecine |
| Ex-2.2 | 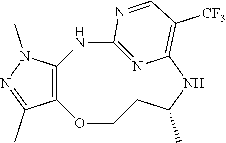 [SMILES: Cc1nn(C)c2c1OCC[C@@H](C)Nc1nc(ncc1C(F)(F)F)N2] | (R)- or (S)-1,3,7-trimethyl-10- (trifluoromethyl)-1,5,6,7,8,14-hexahydro- 9,13-(azeno)pyrazolo[4,3-b][1]oxa[4,6,10] triazacyclotridecine |
| Ex-2.3 | 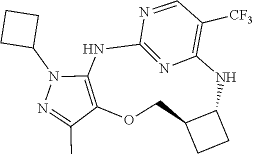 [SMILES: Cc1nn(C2CCC2)c2c1OC[C@@H]1CC[C@H]1Nc1nc(ncc1C(F)(F)F)N2] | (10aR,12aR)- or (10aS,12aS)- 3-Cyclobutyl-1-methyl-8-(trifluoromethyl)- 3,4,10,10a,11,12,-12a,13-octahydro-5,9- (azeno)cyclobuta[k]pyrazolo[4,3- b][1]oxa[4,6,10]triazacyclotridecine |
| Ex-2.4 | 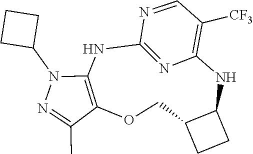 [SMILES: Cc1nn(C2CCC2)c2c1OC[C@H]1CC[C@@H]1Nc1nc(ncc1C(F)(F)F)N2] | (10aR,12aR)- or (10aS,12aS)- 3-Cyclobutyl-1-methyl-8-(trifluoromethyl)- 3,4,10,10a,11,12,-12a,13-octahydro-5,9- (azeno)cyclobuta[k]pyrazolo[4,3- b][1]oxa[4,6,10]triazacyclotridecine |
| Ex-2.5 | 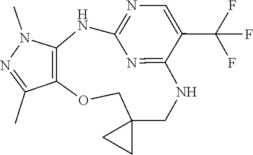 [SMILES: Cc1nn(C)c2c1OCC1(CC1)CNc1nc(ncc1C(F)(F)F)N2] | 1′,3′-dimethyl-10′-(trifluoromethyl)- 1′,7′,8′,14′-tetrahydro-5′H- spiro[cyclopropane-1,6′- [9,13](azeno)pyrazolo[4,3- b][1]oxa[4,6,10]triazacyclotridecine] |
| Ex-2.6 | 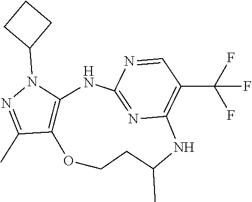 [SMILES: Cc1nn(C2CCC2)c2c1OCCC(C)Nc1nc(ncc1C(F)(F)F)N2] | (R)- or (S)-1-cyclobutyl-3,7-dimethyl-10- (trifluoromethyl)-1,5,6,7,8,14-hexahydro- 9,13-(azeno)pyrazolo[4,3- b][1]oxa[4,6,10]triazacyclotridecine |
| Ex-2.7 | 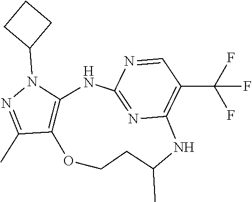 [SMILES: Cc1nn(C2CCC2)c2c1OCCC(C)Nc1nc(ncc1C(F)(F)F)N2] | (R)- or (S)-1-cyclobutyl-3,7-dimethyl-10- (trifluoromethyl)-1,5,6,7,8,14-hexahydro- 9,13-(azeno)pyrazolo[4,3- b][1]oxa[4,6,10]triazacyclotridecine |
| Ex-2.8 | 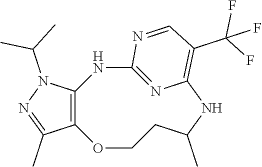 [SMILES: Cc1nn(C(C)C)c2c1OCCC(C)Nc1nc(ncc1C(F)(F)F)N2] | (R)- or (S)-1-isopropyl-3,7-dimethyl-10- (trifluoromethyl)-1,5,6,7,8,14-hexahydro- 9,13-(azeno)pyrazolo[4,3- b][1]oxa[4,6,10]triazacyclotridecine |
| Ex-2.9 | 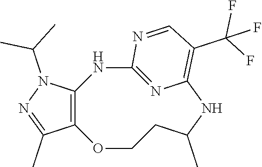 [SMILES: Cc1nn(C(C)C)c2c1OCCC(C)Nc1nc(ncc1C(F)(F)F)N2] | (R)- or (S)-1-isopropyl-3,7-dimethyl-10- (trifluoromethyl)-1,5,6,7,8,14-hexahydro- 9,13-(azeno)pyrazolo[4,3- b][1]oxa[4,6,10]triazacyclotridecine |
| Ex-2.10 | 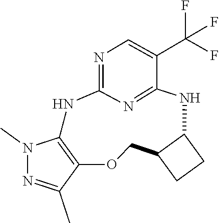 | (10aR,12aR)- or (10aS,12aS)- 1,3-dimethyl-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.11 | 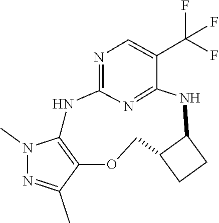 | (10aR,12aS)- or (10aS,12aR)- 1,3-dimethyl-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.12 | 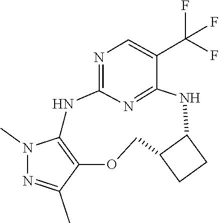 | (10aR,12aS)- or (10aS,12aR)- 1,3-dimethyl-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.13 | 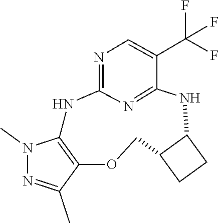 | (10aR,12aS)- or (10aS,12aR)- 1,3-dimethyl-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.14 | 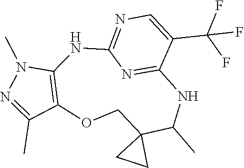 [SMILES: Cc1nn(C)c2c1OCC1(CC1)C(C)Nc1nc(ncc1C(F)(F)F)N2] | (R)- or (S)-1′,3′,7′-trimethyl-10′- (trifluoromethyl)-1′,7′,8′,14′-tetrahydro-5′H- spiro[cyclopropane-1,6′- [9,13](azeno)pyrazolo[4,3- b][1]oxa[4,6,10]triazacyclotridecine] |
| Ex-2.15 | 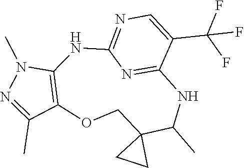 [SMILES: Cc1nn(C)c2c1OCC1(CC1)C(C)Nc1nc(ncc1C(F)(F)F)N2] | (R)- or (S)-1′,3′,7′-trimethyl-10′- (trifluoromethyl)-1′,7′,8′,14′-tetrahydro-5′H- spiro[cyclopropane-1,6′- [9,13](azeno)pyrazolo[4,3- b][1]oxa[4,6,10]triazacyclotridecine] |
| Ex-2.16 | 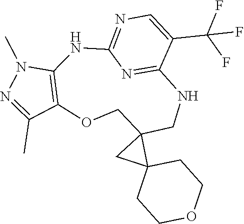 [SMILES: Cc1nn(C)c2c1OCC1(CNc3nc(ncc3C(F)(F)F)N2)CC12CCOCC2] | (R)- or (S)-1″,3″-dimethyl-10″- (trifluoromethyl)-1″,2,3,5,6,7″,8″,14″- octahydro-5″H-dispiro[pyran-4,1′- cyclopropane-2′,6″- [9,13](azeno)pyrazolo[4,3- b][1]oxa[4,6,10]triazacyclotridecine] |
| Ex-2.17 | 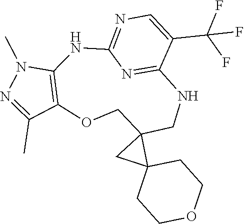 [SMILES: Cc1nn(C)c2c1OCC1(CNc3nc(ncc3C(F)(F)F)N2)CC12CCOCC2] | (R)- or (S)-1″,3″-dimethyl-10″- (trifluoromethyl)-1″,2,3,5,6,7″,8″,14″- octahydro-5″H-dispiro[pyran-4,1′- cyclopropane-2′,6″- [9,13](azeno)pyrazolo[4,3- b][1]oxa[4,6,10]triazacyclotridecine] |
| Ex-2.18 | 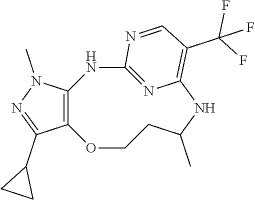 [SMILES: CC1CCOc2c(C3CC3)nn(C)c2Nc2ncc(C(F)(F)F)c(n2)N1] | (R)- or (S)-3-cyclopropyl-1,7-dimethyl-10- (trifluoromethyl)-1,5,6,7,8,14-hexahydro- 9,13-(azeno)pyrazolo[4,3- b][1]oxa[4,6,10]triazacyclotridecine |
| Ex-2.19 | 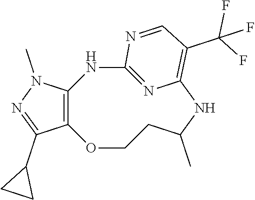 [SMILES: CC1CCOc2c(C3CC3)nn(C)c2Nc2ncc(C(F)(F)F)c(n2)N1] | (R)- or (S)-3-cyclopropyl-1,7-dimethyl-10- (trifluoromethyl)-1,5,6,7,8,14-hexahydro- 9,13-(azeno)pyrazolo[4,3- b][1]oxa[4,6,10]triazacyclotridecine |
| Ex-2.20 | 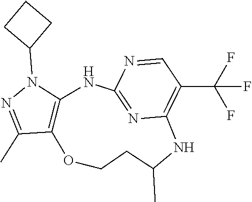 [SMILES: Cc1nn(C2CCC2)c2c1OCCC(C)Nc1nc(ncc1C(F)(F)F)N2] | (R)- or (S)-1-cyclopropyl-3,7-dimethyl-10- (trifluoromethyl)-1,5,6,7,8,14-hexahydro- 9,13-(azeno)pyrazolo[4,3- b][1]oxa[4,6,10]triazacyclotridecine |
| Ex-2.21 | 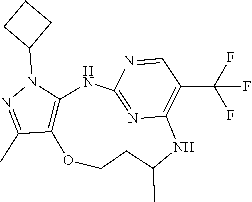 [SMILES: Cc1nn(C2CCC2)c2c1OCCC(C)Nc1nc(ncc1C(F)(F)F)N2] | (R)- or (S)-1-cyclopropyl-3,7-dimethyl-10- (trifluoromethyl)-1,5,6,7,8,14-hexahydro- 9,13-(azeno)pyrazolo[4,3- b][1]oxa[4,6,10]triazacyclotridecine |
| Ex-2.22 | 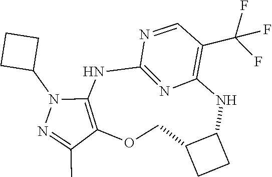 [SMILES: Cc1nn(C2CCC2)c2c1OC[C@H]1CC[C@H]1Nc1nc(ncc1C(F)(F)F)N2] | (10aR,12aS)- or (10aS,12aR)- 3-cyclobutyl-1-methyl-8-(trifluoromethyl)- 3,4,10,10a,-11,12,12a,13-octahydro-5,9- (azeno)cyclobuta[k]pyrazolo[4,3- b][1]oxa[4,6,10]triazacyclotridecine |
| Ex-2.23 | 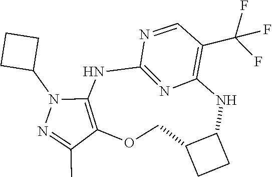 [SMILES: Cc1nn(C2CCC2)c2c1OC[C@H]1CC[C@H]1Nc1nc(ncc1C(F)(F)F)N2] | (10aR,12aS)- or (10aS,12aR)- 3-cyclobutyl-1-methyl-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.24 | 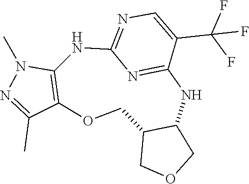 [SMILES: Cc1nn(C)c2c1OC[C@H]1COC[C@H]1Nc1nc(ncc1C(F)(F)F)N2] | (10aS,13aR)- or (10aR,13aS)- 1,3-dimethyl-8-(trifluoromethyl)- 3,4,10a,11,13a,14-hexahydro-10H,13H-5,9- (azeno)furo[3,4-k]pyrazolo[4,3- b][1]oxa[4,6,10]triazacyclotridecine |
| Ex-2.25 | 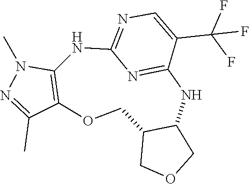 [SMILES: Cc1nn(C)c2c1OC[C@H]1COC[C@H]1Nc1nc(ncc1C(F)(F)F)N2] | (10aS,13aR)- or (10aR,13aS)-1,3-dimethyl-8- (trifluoromethyl)-3,4,10a,11,13a,14- hexahydro-10H,13H-5,9-(azeno)furo[3,4-k] pyrazolo[4,3- b][1]oxa[4,6,10]triazacyclotridecine |
| Ex-2.26 | 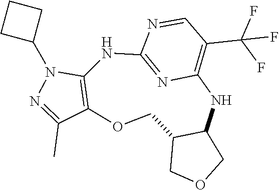 [SMILES: Cc1nn(C2CCC2)c2c1OC[C@H]1COC[C@@H]1Nc1nc(ncc1C(F)(F)F)N2] | (10aS,13aS)- or (10aR,13aR)-3-cyclobutyl-1- methyl-8-(trifluoromethyl)- 3,4,10a,11,13a,14-hexahydro-10H,13H-5,9- (azeno)furo[3,4-k]pyrazolo[4,3- b][1]oxa[4,6,10]triazacyclotridecine |
| Ex-2.27 | 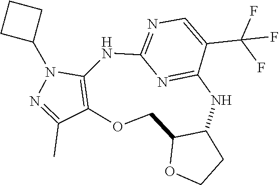 [SMILES: Cc1nn(C2CCC2)c2c1OC[C@@H]1OCC[C@H]1Nc1nc(ncc1C(F)(F)F)N2] | (3aR,15aR)- or (3aS,15aS)- 11-cyclobutyl-13-methyl-6-(trifluoromethyl)- 2,3,3a,4,10,11,15,15a-octahydro-5,9- (azeno)furo[3,2-k]pyrazolo[4,3- b][1]oxa[4,6,10]triazacyclotridecine |
| Ex-2.28 | 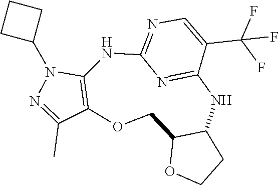 [SMILES: Cc1nn(C2CCC2)c2c1OC[C@@H]1OCC[C@H]1Nc1nc(ncc1C(F)(F)F)N2] | (3aR,15aR)- or (3aS,15aS)- 11-cyclobutyl-13-methyl-6-(trifluoromethyl)- 2,3,3a,4,10,11,15,15a-octahydro-5,9- (azeno)furo[3,2-k]pyrazolo[4,3- b][1]oxa[4,6,10]triazacyclotridecine |
| Ex-2.29 | 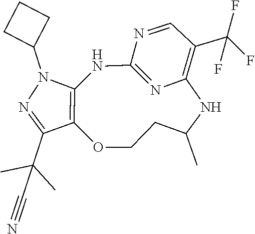 [SMILES: CC1CCOc2c(C(C)(C)C#N)nn(C3CCC3)c2Nc2ncc(C(F)(F)F)c(n2)N1] | (R)- or (S)- butyl-7-methyl-10-(trifluoromethyl)- 1,5,6,7,8,14-hexahydro-9,13- (azeno)pyrazolo[4,3- b][1]oxa[4,6,10]triazacyclotridecin-3-yl)-2- methylpropanenitrile |
| Ex-2.30 | 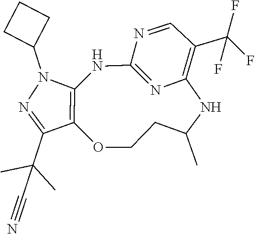 [SMILES: CC1CCOc2c(C(C)(C)C#N)nn(C3CCC3)c2Nc2ncc(C(F)(F)F)c(n2)N1] | (R)- or (S)- butyl-7-methyl-10-(trifluoromethyl)- 1,5,6,7,8,14-hexahydro-9,13- (azeno)pyrazolo[4,3- b][1]oxa[4,6,10]triazacyclotridecin-3-yl)-2- methylpropanenitrile |
| Ex-2.31 | 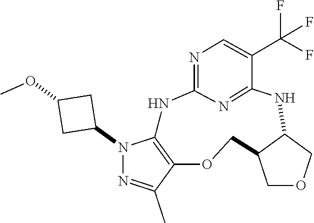 | (10aS,13aS)- or (10aR,13aR)- 3-((1R,3S)-3-methoxycyclobutyl)-1-methyl- 8-(trifluoromethyl)-3,4,10a,11,13a,14- hexahydro-10H,13H-5,9-(azeno)furo[3,4- k]pyrazolo[4,3-b][1]oxa[4,6,10] triazacyclotridecine |
| Ex-2.32 | 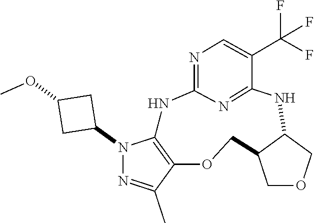 | (10aS,13aS)- or (10aR,13aR)- 3-((1R,3S)-3-methoxycyclobutyl)-1-methyl- 8-(trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.33 | 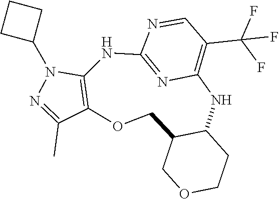 [SMILES: Cc1nn(C2CCC2)c2c1OC[C@@H]1COCC[C@H]1Nc1nc(ncc1C(F)(F)F)N2] | (4aR,16aS)- or (4aS,16aR)- 12-cyclobutyl-14-methyl-7- (trifluoromethyl)-3,4,4a,5,11,12,16,16a- octahydro-1H-6,10-(azeno)pyrano[4,3- k]pyrazolo[4,3- b][1]oxa[4,6,10]triazacyclotridecine |
| Ex-2.34 | 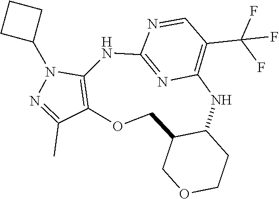 [SMILES: Cc1nn(C2CCC2)c2c1OC[C@@H]1COCC[C@H]1Nc1nc(ncc1C(F)(F)F)N2] | (4aR,16aS)- or (4aS,16aR)- 12-cyclobutyl-14-methyl-7- (trifluoromethyl)-3,4,4a,5,11,12,16,16a- octahydro-1H-6,10-(azeno)pyrano[4,3- k]pyazolo[4,3- b][1]oxa[4,6,10]triazacyclotridecine |
| Ex-2.35 | 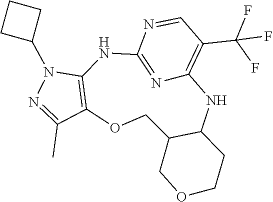 [SMILES: Cc1nn(C2CCC2)c2c1OCC1COCCC1Nc1nc(ncc1C(F)(F)F)N2] | (4aR,16aR)- or (4aS,16aS)- 12-cyclobutyl-14-methyl-7- (trifluoromethyl)-3,4,4a,5,11,12,16,16a- octahydro-1H-6,10-(azeno)pyrano[4,3- k]pyrazolo[4,3- b][1]oxa[4,6,10]triazacyclotridecine |
| Ex-2.36 | 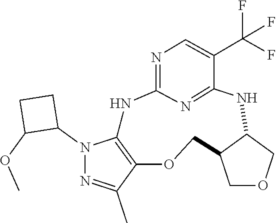 | (10aS,13aS)-3-((1R,2R)- or (10aS,13aS)-3-((1S,2R)- or (10aS,13aS)-3-((1R,2S)- or (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.37 | 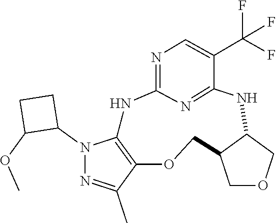 | (10aS,13aS)-3-((1R,2R)- or (10aS,13aS)-3-((1S,2R)- or (10aS,13aS)-3-((1R,2S)- or (10aS,13aS)-3-((1R,2R)- or (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.38 | 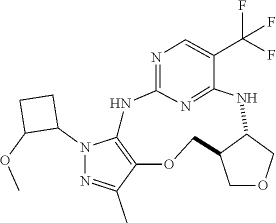 | (10aS,13aS)-3-((1R,2R)- or (10aS,13aS)-3-((1S,2R)- or (10aS,13aS)-3-((1R,2S)- or (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.39 | 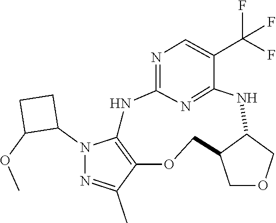 | (10aS,13aS)-3-((1R,2R)- or (10aS,13aS)-3-((1S,2R)- or (10aS,13aS)-3-((1R,2S)- or (10aS,13aS)-3-((1R,2R)- or (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.40 | 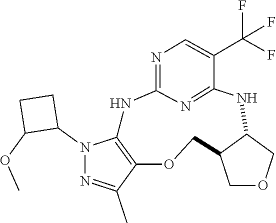 | (10aS,13aS)-3-((1R,2R)- or (10aS,13aS)-3-((1S,2R)- or (10aS,13aS)-3-((1R,2S)- or (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.41 | 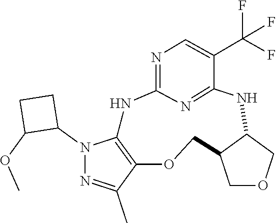 | (10aS,13aS)-3-((1R,2R)- or (10aS,13aS)-3-((1S,2R)- or (10aS,13aS)-3-((1R,2S)- or (10aS,13aS)-3-((1R,2R)- (10aR,13aR)-3-((1S,2R)-(10aR,13aR)-3- ((1R,2S)-2-methoxycyclobutyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.42 | 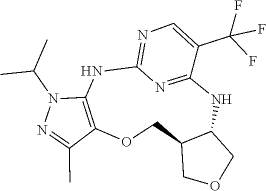 [SMILES: Cc1nn(C(C)C)c2c1OC[C@@H]1COC[C@H]1Nc1nc(ncc1C(F)(F)F)N2] | (10aS,13aS)-3-isopropyl-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.43 | 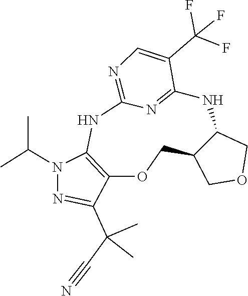 [SMILES: CC(C)n1nc(C(C)(C)C#N)c2c1Nc1ncc(C(F)(F)F)c(n1)N[C@@H]1COC[C@H]1CO2] | 2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1-yl)-2- methylpropanenitrile |
| Ex-2.44 | 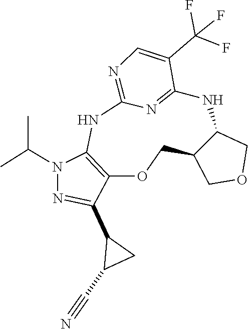 | (1S,2S)- or (1R,2R)- 2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carbonitrile |
| Ex-2.45 | 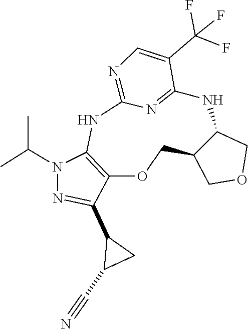 | (1S,2S)- or (1R,2R)- 2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carbonitrile |
| Ex-2.46 | 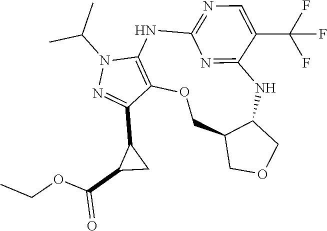 [SMILES: CCOC(=O)[C@@H]1C[C@@H]1c1nn(C(C)C)c2c1OC[C@@H]1COC[C@H]1Nc1nc(ncc1C(F)(F)F)N2] | ethyl (1R,2S)- or ethyl (1S,2R)- 2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carboxylate |
| Ex-2.47 | 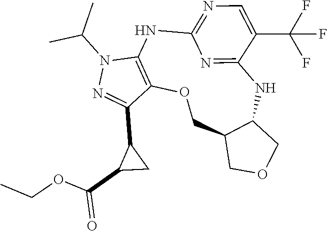 [SMILES: CCOC(=O)[C@@H]1C[C@@H]1c1nn(C(C)C)c2c1OC[C@@H]1COC[C@H]1Nc1nc(ncc1C(F)(F)F)N2] | ethyl (1R,2S)- or ethyl (1S,2R)- 2-((10aS,13aS)-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carboxylate |
| Ex-2.48 | 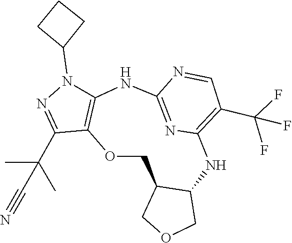 [SMILES: CC(C)(C#N)c1nn(C2CCC2)c2c1OC[C@@H]1COC[C@H]1Nc1nc(ncc1C(F)(F)F)N2] | 2-((10aS,13aS)-3-cyclobutyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1-yl)-2- methylpropanenitrile |
| Ex-2.49 | 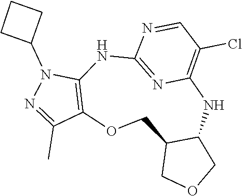 [SMILES: Cc1nn(C2CCC2)c2c1OC[C@@H]1COC[C@H]1Nc1nc(ncc1Cl)N2] | (10aS,13aS)-8-chloro-3-cyclobutyl-1-methyl- 3,4,10a,11,13a,14-hexahydro-10H,13H-5,9- (azeno)furo[3,4-k]pyrazolo[4,3- b][1]oxa[4,6,10]triazacyclotridecine |
| Ex-2.50 | 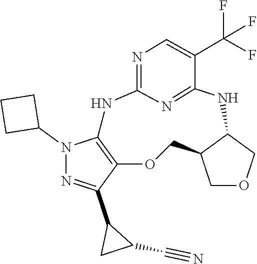 | (1R,2R)- or (1S,2S)- 2-((10aS,13aS)-3-cyclobutyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carbonitrile |
| Ex-2.51 | 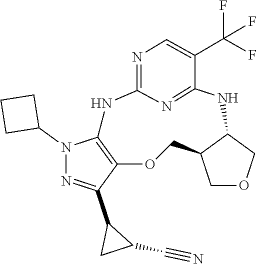 | (1R,2R)- or (1S,2S)- 2-((10aS,13aS)-3-cyclobutyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)cyclopropane-1-carbonitrile |
| Ex-2.52 | 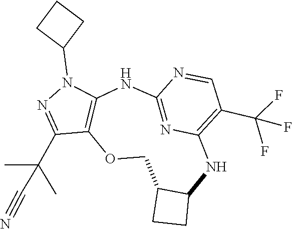 [SMILES: CC(C)(C#N)c1nn(C2CCC2)c2c1OC[C@H]1CC[C@@H]1Nc1nc(ncc1C(F)(F)F)N2] | 2-((10aS,12aS)- or 2-((10aR,12aR)- 3-cyclobutyl-8-(trifluoromethyl)- 3,4,10,10a,11,12,12a,13-octahydro-5,9- (azeno)cyclobuta[k]pyrazolo[4,3- b][1]oxa[4,6,10]triazacyclotridecin-1-yl)-2- methylpropanenitrile |
| Ex-2.53 | 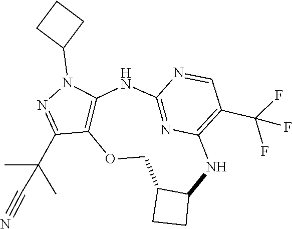 [SMILES: CC(C)(C#N)c1nn(C2CCC2)c2c1OC[C@H]1CC[C@@H]1Nc1nc(ncc1C(F)(F)F)N2] | 2-((10aS,12aS)- or 2-((10aR,12aR)- 3-cyclobutyl-8-(trifluoromethyl)- 3,4,10,10a,11,12,12a,13-octahydro-5,9- (azeno)cyclobuta[k]pyrazolo[4,3- b][1]oxa[4,6,10]triazacyclotridecin-1-yl)-2- methylpropanenitrile |
| Ex-2.54 | 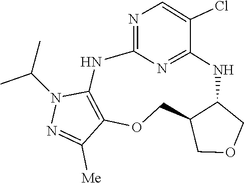 [SMILES: Cc1nn(C(C)C)c2c1OC[C@@H]1COC[C@H]1Nc1nc(ncc1Cl)N2] | (10aS,13aS)-8-chloro-3-isopropyl-1-methyl- 3,4,10a,11,13a,14--hexahydro-10H,13H-5,9- (azeno)furo[3,4-k]pyrazolo[4,3- b][1]oxa[4,6,10]triazacyclotridecine |
| Ex-2.55 | 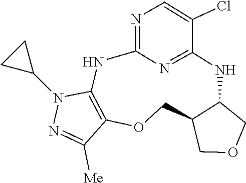 [SMILES: Cc1nn(C2CC2)c2c1OC[C@@H]1COC[C@H]1Nc1nc(ncc1Cl)N2] | (10aS,13aS)-8-chloro-3-cyclopropyl-1- methyl-3,4,10a,11,13a,14-hexahydro- 10H,13H-5,9-(azeno)furo[3,4- k]pyrazolo[4,3- b][1]oxa[4,6,10]triazacyclotridecine |
| Ex-2.56 | 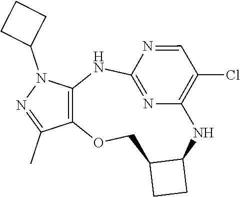 [SMILES: Cc1nn(C2CCC2)c2c1OC[C@@H]1CC[C@@H]1Nc1nc(ncc1Cl)N2] | (10aS,12aR)-8-chloro-3-cyclobutyl-1- methyl-3,4,10,10a,11,12,12a,13-octahydro- 5,9-(azeno)cyclobuta[k]pyrazolo[4,3- b][1]oxa[4,6,10]triazacyclotridecine |
| Ex-2.57 | 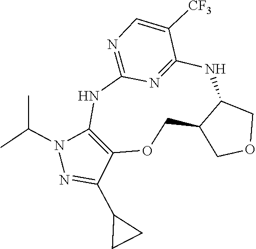 [SMILES: CC(C)n1nc(C2CC2)c2c1Nc1ncc(C(F)(F)F)c(n1)N[C@@H]1COC[C@H]1CO2] | (10aS,13aS)-1-cyclopropyl-3-isopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.58 | 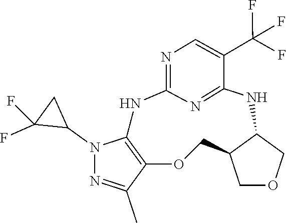 [SMILES: Cc1nn(C2CC2(F)F)c2c1OC[C@@H]1COC[C@H]1Nc1nc(ncc1C(F)(F)F)N2] | (10aS,13aS)-3-((R)-2,2- or (10aS,13aS)-3- ((S)-2,2- difluorocyclopropyl)-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.59 | 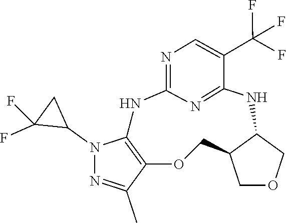 [SMILES: Cc1nn(C2CC2(F)F)c2c1OC[C@@H]1COC[C@H]1Nc1nc(ncc1C(F)(F)F)N2] | (10aS,13aS)-3-((R)-2,2- or (10aS,13aS)-3- ((S)-2,2- difluorocyclopropyl)-1-methyl-8- (trifluoromethyl)-3,4,10a,11,13a,14- hexahydro-10H,13H-5,9-(azeno)furo[3,4-k] pyrazolo[4,3- b][1]oxa[4,6,10]triazacyclotridecine |
| Ex-2.60 | 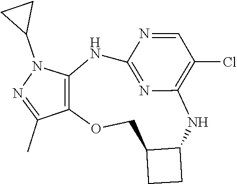 [SMILES: Cc1nn(C2CC2)c2c1OC[C@@H]1CC[C@H]1Nc1nc(ncc1Cl)N2] | (10aR,12aR)-8-chloro-3-cyclopropyl-1- methyl-3,4,10,10a,11,12,12a,13-octahydro- 5,9-(azeno)cyclobuta[k]pyrazolo[4,3- b][1]oxa[4,6,10]triazacyclotridecine |
| Ex-2.61 | 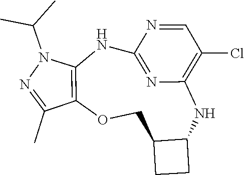 [SMILES: Cc1nn(C(C)C)c2c1OC[C@@H]1CC[C@H]1Nc1nc(ncc1Cl)N2] | (10aR,12aR)-8-chloro-3-isopropyl-1-methyl- 3,4,10,10a,11,12,12a,13-octahydro-5,9- (azeno)cyclobuta[k]pyrazolo[4,3- b][1]oxa[4,6,10]triazacyclotridecine |
| Ex-2.62 | 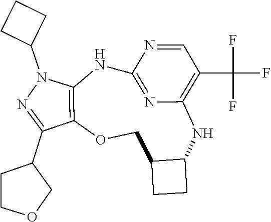 | (10aR,12aR)-3-cyclobutyl-1-((R)- or (10aR,12aR)-3-cyclobutyl-1-((S)- or (10aS,12aS)-3-cyclobutyl-1-((R)- or (10aS,12aS)-3-cyclobutyl-1-((S)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.63 | 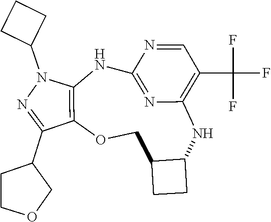 | (10aR,12aR)-3-cyclobutyl-1-((R)- or (10aR,12aR)-3-cyclobutyl-1-((S)- or (10aS,12aS)-3-cyclobutyl-1-((R)- or (10aS,12aS)-3-cyclobutyl-1-((S)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.64 | 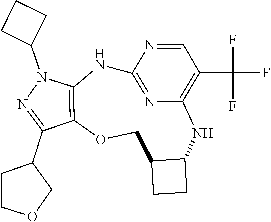 | (10aR,12aR)-3-cyclobutyl-1-((R)- or (10aR,12aR)-3-cyclobutyl-1-((S)- or (10aS,12aS)-3-cyclobutyl-1-((R)- or (10aS,12aS)-3-cyclobutyl-1-((S)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.65 | 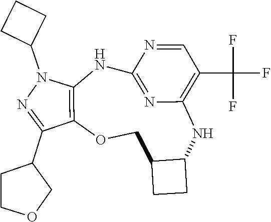 | (10aR,12aR)-3-cyclobutyl-1-((R)- or (10aR,12aR)-3-cyclobutyl-1-((S)- or (10aS,12aS)-3-cyclobutyl-1-((R)- or (10aS,12aS)-3-cyclobutyl-1-((S)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.66 | 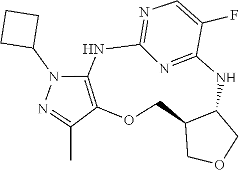 [SMILES: Cc1nn(C2CCC2)c2c1OC[C@@H]1COC[C@H]1Nc1nc(ncc1F)N2] | (10aS,13aS)-3-cyclobutyl-8-fluoro-1-methyl- 3,4,10a,11,13a,14-hexahydro-10H,13H-5,9- (azeno)furo[3,4-k]pyrazolo[4,3- b][1]oxa[4,6,10]triazacyclotridecine |
| Ex-2.67 | 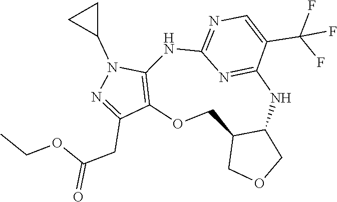 [SMILES: CCOC(=O)Cc1nn(C2CC2)c2c1OC[C@@H]1COC[C@H]1Nc1nc(ncc1C(F)(F)F)N2] | ethyl 2-((10aS,13aS)-3-cyclopropyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecin-1- yl)acetate |
| Ex-2.68 | 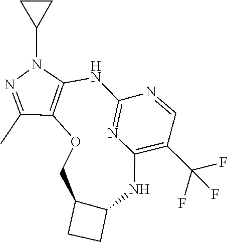 [SMILES: Cc1nn(C2CC2)c2c1OC[C@@H]1CC[C@H]1Nc1nc(ncc1C(F)(F)F)N2] | (10aR,12aR)-3-cyclopropyl-1-methyl-8- (trifluoromethyl)-3,4,10,10a,11,12,12a,13- octahydro-5,9- (azeno)cyclobuta[k]pyrazolo[4,3- b][1]oxa[4,6,10]triazacyclotridecine |
| Ex-2.69 | 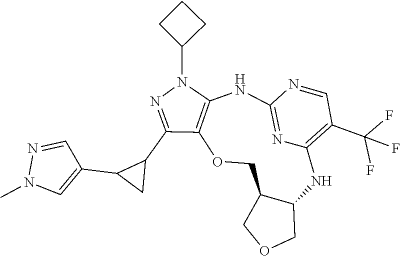 [SMILES: Cn1cc(C2CC2c2nn(C3CCC3)c3c2OC[C@@H]2COC[C@H]2Nc2nc(ncc2C(F)(F)F)N3)cn1] | (10aS,13aS)-3-cyclobutyl-1-((1S,2S)- or (10aS,13aS)-3-cyclobutyl-1-((1R,2R)- 2-(1-methyl-1H-pyrazol-4-yl)cyclopropyl)- 8-(trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.70 | 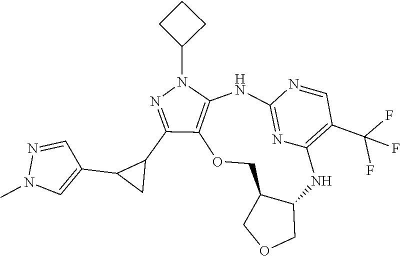 [SMILES: Cn1cc(C2CC2c2nn(C3CCC3)c3c2OC[C@@H]2COC[C@H]2Nc2nc(ncc2C(F)(F)F)N3)cn1] | (10aS,13aS)-3-cyclobutyl-1-((1S,2S)- or (10aS,13aS)-3-cyclobutyl-1-((1R,2R)- 2-(1-methyl-1H-pyrazol-4-yl)cyclopropyl)- 8-(trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.71 | 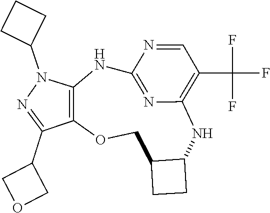 | (10aR,12aR)- or (10aS,12aS)- 3-cyclobutyl-1-(oxetan-3-yl)-8- (trifluoromethyl)-3,4,10,10a,11,12,-12a,13- octahydro-5,9- (azeno)cyclobuta[k]pyrazolo[4,3- b][1]oxa[4,6,10]triazacyclotridecine |
| Ex-2.72 | 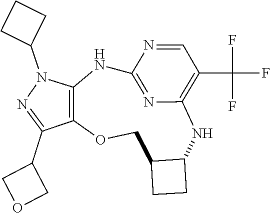 | (10aR,12aR)- or (10aS,12aS)- 3-cyclobutyl-1-(oxetan-3-yl)-8- (trifluoromethyl)-3,4,10,10a,11,12,12a,13- octahydro-5,9- (azeno)cyclobuta[k]pyrazolo[4,3- b][1]oxa[4,6,10]triazacyclotridecine |
| Ex-2.73 | 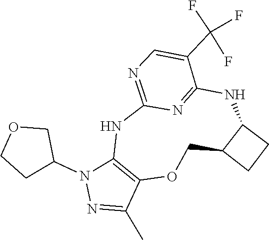 [SMILES: Cc1nn(C2CCOC2)c2c1OC[C@@H]1CC[C@H]1Nc1nc(ncc1C(F)(F)F)N2] | (10aR,12aR)-1-methyl-3-((S)- or (10aR,12aR)-1-methyl-3-((R)- tetrahydrofuran-3-yl)-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.74 | 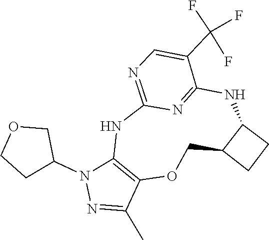 [SMILES: Cc1nn(C2CCOC2)c2c1OC[C@@H]1CC[C@H]1Nc1nc(ncc1C(F)(F)F)N2] | (10aR,12aR)-1-methyl-3-((S)- or (10aR,12aR)-1-methyl-3-((R)- tetrahydrofuran-3-yl)-8-(trifluoromethyl)- 3,4,10,10a,11,12,-12a,13-octahydro-5,9- (azeno)cyclobuta[k]pyrazolo[4,3- b][1]oxa[4,6,10]triazacyclotridecine |
| Ex-2.75 | 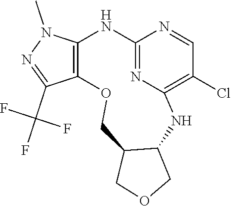 [SMILES: Cn1nc(C(F)(F)F)c2c1Nc1ncc(Cl)c(n1)N[C@@H]1COC[C@H]1CO2] | (10aS,13aS)-8-chloro-3-methyl-1- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-2.76 | 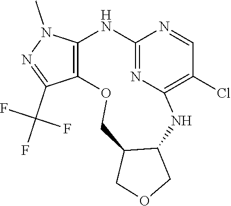 [SMILES: Cn1nc(C(F)(F)F)c2c1Nc1ncc(Cl)c(n1)N[C@@H]1COC[C@H]1CO2] | (10aR,12aR)-8-chloro-3-methyl-1- (trifluoromethyl)-3,4,10,10a,11,12,12a,13- octahydro-5,9- (azeno)cyclobuta[k]pyrazolo[4,3- b][1]oxa[4,6,10]triazacyclotridecine |
| Ex-2.77 | 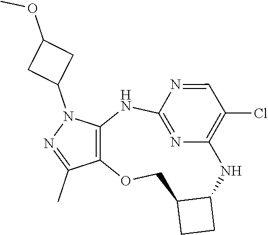 [SMILES: COC1CC(n2nc(C)c3c2Nc2ncc(Cl)c(n2)N[C@@H]2CC[C@H]2CO3)C1] | (10aR,12aR)-8-chloro-3-(3- methoxycyclobutyl)-1-methyl- 3,4,10,10a,11,12,12a,13-octahydro-5,9- (azeno)cyclobuta[k]pyrazolo[4,3- b][1]oxa[4,6,10]triazacyclotridecine |
| Ex-2.78 | 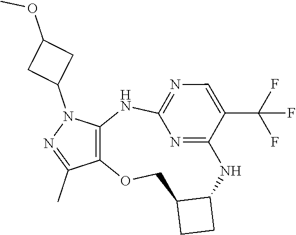 [SMILES: COC1CC(n2nc(C)c3c2Nc2ncc(C(F)(F)F)c(n2)N[C@@H]2CC[C@H]2CO3)C1] | (10aR,12aR)-3-(3-methoxycyclobutyl)-1- methyl-8-(trifluoromethyl)- 3,4,10,10a,11,12,12a,13-octahydro-5,9- (azeno)cyclobuta[k]pyrazolo[4,3- b][1]oxa[4,6,10]triazacyclotridecine |
| Ex-2.79 | 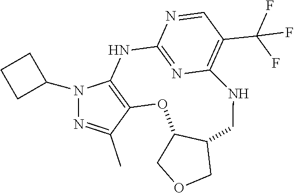 | (11aR,14aR) or (11aS,14aS)- 3-cyclobutyl-1-methyl-8-(trifluoromethyl)- 3,4,10,11,11a,12,14,14a-octahydro-5,9- (azeno)furo[3,4-l]pyrazolo[4,3- b][1]oxa[4,6,10]triazacyclotridecine |
| Ex-2.80 | 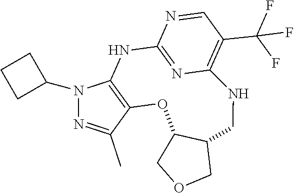 | (11aR,14aR) or (11aS,14aS)- 3-cyclobutyl-1-methyl-8-(trifluoromethyl)- 3,4,10,11,11a,12,14,14a-octahydro-5,9- (azeno)furo[3,4-l]pyrazolo[4,3- b][1]oxa[4,6,10]triazacyclotridecine |
| Ex-2.81 | 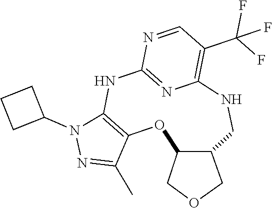 | (11aR,14aR) or (11aS,14aR)- 3-cyclobutyl-1-methyl-8-(trifluoromethyl)- 3,4,10,11,11a,12,14,14a-octahydro-5,9- (azeno)furo[3,4-l]pyrazolo[4,3- b][1]oxa[4,6,10]triazacyclotridecine |
| Ex-2.82 | 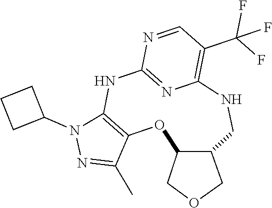 | (11aR,14aS) or (11aS,14aR)- 3-cyclobutyl-1-methyl-8-(trifluoromethyl)- 3,4,10,11,11a,12,14,14a-octahydro-5,9- (azeno)furo[3,4-l]pyrazolo[4,3- b][1]oxa[4,6,10]triazacyclotridecine |
| Ex-2.83 | 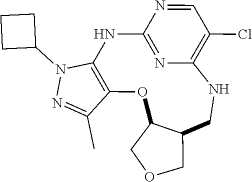 | (11aR,14aR)- or (11aS,14aS)-8-chloro-3- cyclobutyl-1-methyl- 3,4,10,11,11a,12,14,14a-octahydro-5,9- (azeno)furo[3,4-l]pyrazolo[4,3- b][1]oxa[4,6,10]triazacyclotridecine |
| Ex-2.84 | 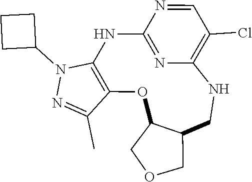 | (11aR,14aR)- or (11aS,14aS)- 8-chloro-3-cyclobutyl-1-methyl- 3,4,10,11,11a,12,14,14a-octahydro-5,9- (azeno)furo[3,4-l]pyrazolo[4,3- b][1]oxa[4,6,10]triazacyclotridecine |
| Ex-2.85 | 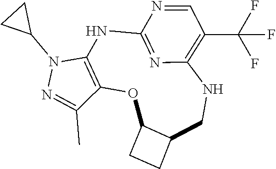 [SMILES: Cc1nn(C2CC2)c2c1O[C@@H]1CC[C@@H]1CNc1nc(ncc1C(F)(F)F)N2] | (11aR,13aR)- or (11aS,13aS)- 3-cyclopropyl-1-methyl-8-(trifluoromethyl)- 3,4,10,11,11a,12,-13,13a-octahydro-5,9- (azeno)cyclobuta[l]pyrazolo[4,3- b][1]oxa[4,6,10]triazacyclotridecine |
| Ex-2.86 | 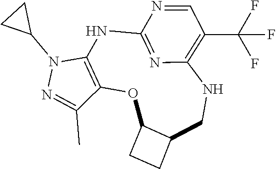 [SMILES: Cc1nn(C2CC2)c2c1O[C@@H]1CC[C@@H]1CNc1nc(ncc1C(F)(F)F)N2] | (11aR,13aR)- or (11aS,13aS)- 3-cyclopropyl-1-methyl-8-(trifluoromethyl)- 3,4,10,11,11a,12,13,13a-octahydro-5,9- (azeno)cyclobuta[l]pyrazolo[4,3- b][1]oxa[4,6,10]triazacyclotridecine |
| Ex-3.1 | 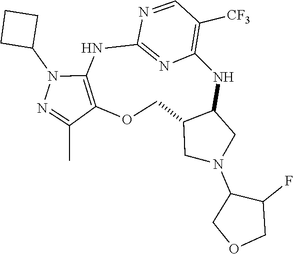 [SMILES: Cc1nn(C2CCC2)c2c1OC[C@H]1CN(C3COCC3F)C[C@@H]1Nc1nc(ncc1C(F)(F)F)N2] | (10aS,13aR)- and (10aR,13aS)- 3-cyclobutyl-12-(4-fluorotetrahydrofuran-3- yl)-1-methyl-8-(trifluoromethyl)- 3,4,10a,11,12,13,13a,-14-octahydro-10H-5,9- (azeno)pyrazolo[4,3-b]pyrrolo[3,4-k][1]oxa [4,6,10]-triazacyclotridecine |
| Ex-4.1 | 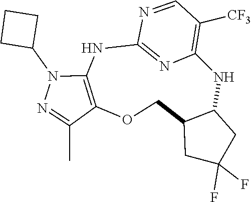 [SMILES: Cc1nn(C2CCC2)c2c1OC[C@@H]1CC(F)(F)C[C@H]1Nc1nc(ncc1C(F)(F)F)N2] | 3-Cyclobutyl-12,12-difluoro-1-methyl-8- (trifluoromethyl)-3,4,10a,11,12,13,13a,14- octahydro-10H-5,9-(azeno)cyclopenta- [k]pyrazolo[4,3- b][1]oxa[4,6,10]triazacyclotridecine |
| Ex-4.2 | 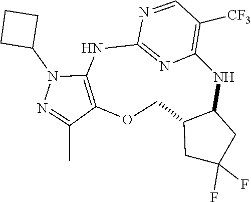 [SMILES: Cc1nn(C2CCC2)c2c1OC[C@H]1CC(F)(F)C[C@@H]1Nc1nc(ncc1C(F)(F)F)N2] | 3-Cyclobutyl-12,12-difluoro-1-methyl-8- (trifluoromethyl)-3,4,10a,11,12,13,13a,14- octahydro-10H-5,9- (azeno)cyclopenta[k]pyrazolo[4,3- b][1]oxa[4,6,10]triazacyclotridecine |
| Ex-5.1 | 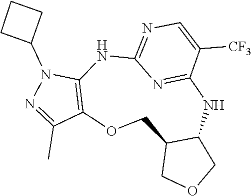 [SMILES: Cc1nn(C2CCC2)c2c1OC[C@@H]1COC[C@H]1Nc1nc(ncc1C(F)(F)F)N2] | (10aS,13aS)-3-cyclobutyl-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-5.2 | 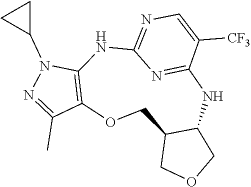 [SMILES: Cc1nn(C2CC2)c2c1OC[C@@H]1COC[C@H]1Nc1nc(ncc1C(F)(F)F)N2] | (10aS,13aS)-3-cyclopropyl-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-5.3 | 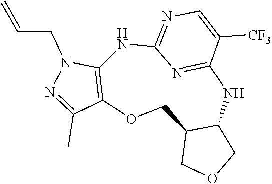 [SMILES: C=CCn1nc(C)c2c1Nc1ncc(C(F)(F)F)c(n1)N[C@@H]1COC[C@H]1CO2] | (10aS,13aS)-3-allyl-1-methyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine |
| Ex-5.4 | 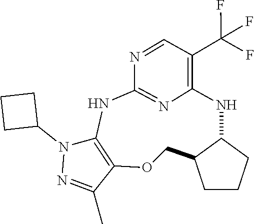 | (10aR,13aR)- or (10aS,13aS)- 3-cyclobutyl-1-methyl-8-(trifluoromethyl)- 3,4,10a,11,12,13,13a,14-octahydro-10H- 5,9-(azeno)-cyclopenta[k]pyrazolo [4,3-b][1]oxa[4,6,10]-triazacyclotridecine |
| Ex-5.5 | 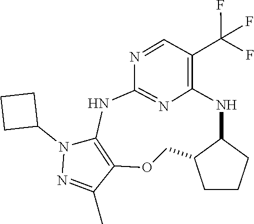 | (10aR,13aR)- or (10aS,13aS)- 3-cyclobutyl-1-methyl-8-(trifluoromethyl)- 3,4,10a,11,12,13,13a,14-octahydro-10H- 5,9-(azeno)-cyclopenta[k]pyrazolo[4,3- b][1]oxa[4,6,10]triazacyclotridecine |
| Ex-5.6 | 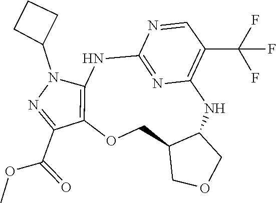 [SMILES: COC(=O)c1nn(C2CCC2)c2c1OC[C@@H]1COC[C@H]1Nc1nc(ncc1C(F)(F)F)N2] | methyl (10aS,13aS)-3-cyclobutyl-8- (trifluoromethyl)-3,4,10a,11,13a,14- hexahydro-10H,13H-5,9-(azeno)furo[3,4- k]pyrazolo[4,3- b][1]oxa[4,6,10]triazacyclotridecine-1- carboxylate |
| Ex-6.1 | 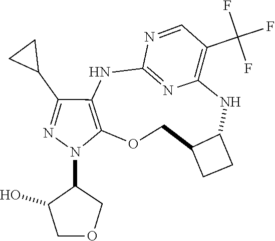 [SMILES: O[C@H]1COC[C@@H]1n1nc(C2CC2)c2c1OC[C@@H]1CC[C@H]1Nc1nc(ncc1C(F)(F)F)N2] | (3R,4S)-4-((10aR,12aR)- or (3R,4S)-4- ((10aS,12aS)- or (3S,4R)-4-((10aR,12aR)- or (3S,4R)-4-((10aR,12aR)- 3-cyclopropyl-8-(trifluoromethyl)- 10,10a,11,12,12a,13-hexahydro-5,9- (azeno)cyclobuta[k]pyrazolo-[3,4- b][1]oxa[4,6,10]-triazacyclotridecin-1(4H)- yl)tetrahydrofuran-3-ol |
| Ex-6.2 | 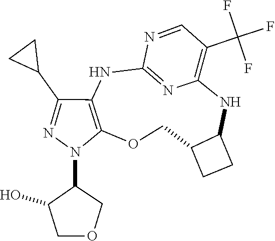 [SMILES: O[C@H]1COC[C@@H]1n1nc(C2CC2)c2c1OC[C@H]1CC[C@@H]1Nc1nc(ncc1C(F)(F)F)N2] | (3R,4S)-4-((10aR,12aR)- or (3R,4S)-4- ((10aS,12aS)- or (3S,4R)-4-((10aR,12aR)- or (3S,4R)-4-((10aR,12aR)- 3-cyclopropyl-8-(trifluoromethyl)- 10,10a,11,12,12a,13-hexahydro-5,9- (azeno)cyclobuta[k]pyrazolo[3,4- b][1]oxa[4,6,10]-triazacyclotridecin-1(4H)- yl)tetrahydrofruan-3-ol |
| Ex-6.3 | 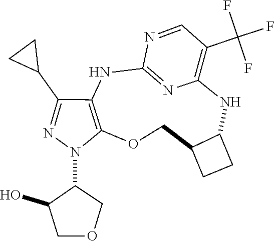 [SMILES: O[C@@H]1COC[C@H]1n1nc(C2CC2)c2c1OC[C@@H]1CC[C@H]1Nc1nc(ncc1C(F)(F)F)N2] | (3R,4S)-4-((10aR,12aR)- or (3R,4S)-4- ((10aS,12aS)- or (3S,4R)-4-((10aR,12aR)- or (3S,4R)-4-((10aR,12aR)- 3-cyclopropyl-8-(trifluoromethyl)- 10,10a,11,12,12a,13-hexahydro-5,9- (azeno)cyclobuta[k]pyrazolo-[3,4- b][1]oxa[4,6,10]-triazacyclotridecin-1(4H)- yl)tetrahydrofuran-3-ol |
| Ex-6.4 | 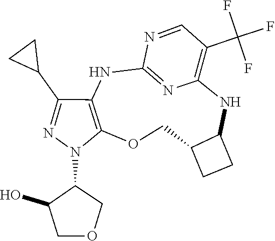 [SMILES: O[C@@H]1COC[C@H]1n1nc(C2CC2)c2c1OC[C@H]1CC[C@@H]1Nc1nc(ncc1C(F)(F)F)N2] | (3R,4S)-4-((10aR,12aR)- or (3R,4S)-4- ((10aS,12aS)- or (3S,4R)-4-((10aR,12aR)- or (3S,4R)-4-((10aR,12aR)-3- cyclopropyl-8-(trifluoromethyl)- 10,10a,11,12,12a,13-hexahydro-5,9- (azeno)cyclobuta[k]pyrazolo-[3,4- b][1]oxa[4,6,10]-triazacyclotridecin-1(4H)- yl)tetrahydrofuran-3-ol |
| Ex-6.5 | 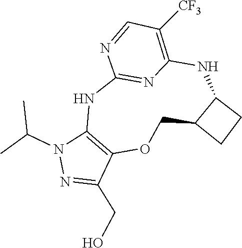 [SMILES: CC(C)n1nc(CO)c2c1Nc1ncc(C(F)(F)F)c(n1)N[C@@H]1CC[C@H]1CO2] | ((10aR,12aR)-3-isopropyl-8- (trifluoromethyl)-3,4,10,10a,11,12,12a,13- octahydro-5,9-(azeno)cyclobuta[k]pyrazolo- [4,3-b][1]oxa[4,6,10]triazacyclo-tridecin-1- yl)methanol |
| Ex-6.6 | 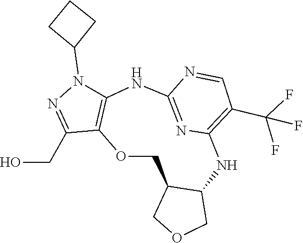 [SMILES: OCc1nn(C2CCC2)c2c1OC[C@@H]1COC[C@H]1Nc1nc(ncc1C(F)(F)F)N2] | ((10aS,13aS)-3-cyclobutyl-8- (trifluoromethyl)-3,4,10a,11,13a,-14- hexahydro-10H,13H-5,9-(azeno)furo[3,4- k]pyrazolo[4,3- b][1]oxa[4,6,10]triazacyclotridecin-1- yl)methanol |
| Ex-6.7 | 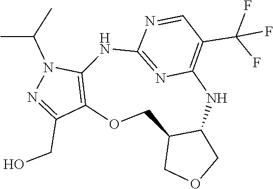 [SMILES: CC(C)n1nc(CO)c2c1Nc1ncc(C(F)(F)F)c(n1)N[C@@H]1COC[C@H]1CO2] | ((10aS,13aS)-3-isopropyl-8- (trifluoromethyl)-3,4,10a,11,13a,-14- hexahydro-10H,13H-5,9-(azeno)furo[3,4- k]pyrazolo[4,3- b][1]oxa[4,6,10]triazacyclotridecin-1- yl)methanol |
| Ex-6.8 | 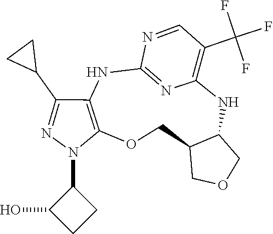 [SMILES: O[C@H]1CC[C@@H]1n1nc(C2CC2)c2c1OC[C@@H]1COC[C@H]1Nc1nc(ncc1C(F)(F)F)N2] | (1R,2R)- or (1S,2S)- 2-((10aS,13aS)-3-cyclopropyl-8- (trifluoromethyl)-10a,11,13a,14-tetrahydro- 10H,13H-5,9-(azeno)furo[3,4- k]pyrazolo[3,4- b][1]oxa[4,6,10]triazacyclotridecin-1(4H)- yl)cyclobutan-1-ol |
| Ex-6.9 | 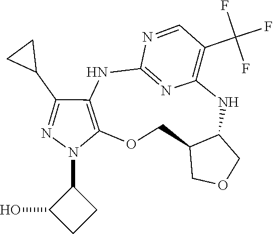 [SMILES: O[C@H]1CC[C@@H]1n1nc(C2CC2)c2c1OC[C@@H]1COC[C@H]1Nc1nc(ncc1C(F)(F)F)N2] | (1R,2R)- or (1S,2S)- 2-((10aS,13aS)-3-cyclopropyl-8- (trifluoromethyl)-10a,11,13a,14-tetrahydro- 10H,13H-5,9-(azeno)furo[3,4- k]pyrazolo[3,4- b][1]oxa[4,6,10]triazacyclotridecin-1(4H)- yl)cyclobutan-1-ol |
| Ex-7.1 | 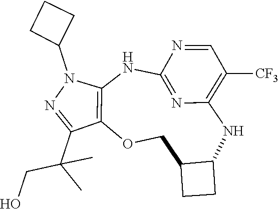 [SMILES: CC(C)(CO)c1nn(C2CCC2)c2c1OC[C@@H]1CC[C@H]1Nc1nc(ncc1C(F)(F)F)N2] | 2-((10aR,12aR)- or (10aS,12aS)- 3-cyclobutyl-8-(trifluoromethyl)- 3,4,10,10a,11,12,12a,13-octahydro-5,9- (azeno)cyclobuta-[k]pyrazolo[4,3- b][1]oxa[4,6,10]triazacyclotridecin-1-yl)-2- methylpropan-1-ol |
| Ex-7.2 | 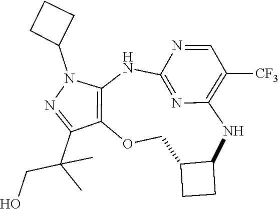 [SMILES: CC(C)(CO)c1nn(C2CCC2)c2c1OC[C@H]1CC[C@@H]1Nc1nc(ncc1C(F)(F)F)N2] | 2-((10aR,12aR)- or (10aS,12aS)- 3-cyclobutyl-8-(trifluoromethyl)- 3,4,10,10a,11,12,12a,13-octahydro-5,9- (azeno)cyclobuta-[k]pyrazolo[4,3- b][1]oxa[4,6,10]triazacyclotridecin-1-yl)-2- methylpropan-1-ol |
| Ex-7.3 | 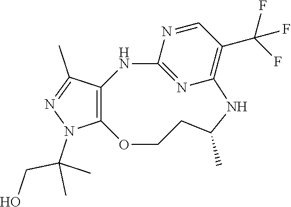 [SMILES: Cc1nn(C(C)(C)CO)c2c1Nc1ncc(C(F)(F)F)c(n1)N[C@H](C)CCO2] | (R)-2-(3,11-dimethyl-8-(trifluoromethyl)- 10,11,12,13-tetrahydro-5,9- (azeno)pyrazolo[3,4- b][1]oxa[4,6,10]triazacyclotridecin-1(4H)- yl)-2-methylpropan-1-ol |
| Ex-7.4 | 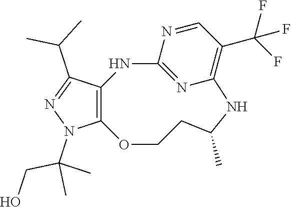 [SMILES: CC(C)c1nn(C(C)(C)CO)c2c1Nc1ncc(C(F)(F)F)c(n1)N[C@H](C)CCO2] | (R)-2-(3-isopropyl-11-methyl-8- (trifluoromethyl)-10,11,12,13-tetrahydro- 5,9-(azeno)pyrazolo[3,4- b][1]oxa[4,6,10]triazacyclotridecin-1(4H)- yl)-2-methylpropan-1-ol |
| Ex-7.5 | 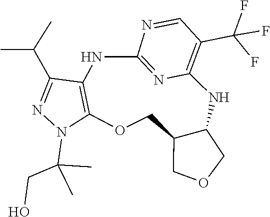 [SMILES: CC(C)c1nn(C(C)(C)CO)c2c1Nc1ncc(C(F)(F)F)c(n1)N[C@@H]1COC[C@H]1CO2] | 2-((10aS,13aS)-3-isopropyl-8- (trifluoromethyl)-10a,11,13a,14-tetrahydro- 10H,13H-5,9-(azeno)furo[3,4- k]pyrazolo[3,4- b][1]oxa[4,6,10]triazacyclotridecin-1(4H)- yl)-2-methylpropan-1-ol |
| Ex-8.1 | 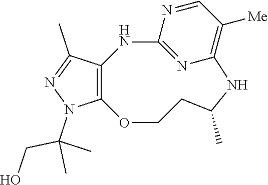 [SMILES: Cc1cnc2nc1N[C@H](C)CCOc1c(c(C)nn1C(C)(C)CO)N2] | (R)-2-methyl-2-(3,8,11-trimethyl- 10,11,12,13-tetrahydro-5,9- (azeno)pyrazolo[3,4- b][1]oxa[4,6,10]triazacyclotridecin-1(4H)- yl)propan-1-ol |
| Ex-9.1 | 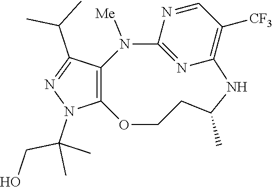 [SMILES: CC(C)c1nn(C(C)(C)CO)c2c1N(C)c1ncc(C(F)(F)F)c(n1)N[C@H](C)CCO2] | (R)-2-(3-isopropyl-4,11-dimethyl-8- (trifluoromethyl)-10,11,12,13-tetrahydro- 5,9-(azeno)pyrazolo[3,4- b][1]oxa[4,6,10]triazacyclotridecin-1(4H)- yl)-2-methylpropan-1-ol |
| Ex-10.1 | 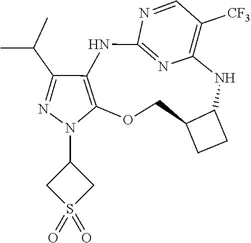 | 3-((10aR,12aR)-3-isopropyl-8- (trifluoromethyl)-10,10a,11,12,12a,13- hexahydro-5,9-(azeno)cyclobuta[k]pyrazolo- [3,4-b][1]oxa[4,6,10]triazacyclo-tridecin- 1(4H)-yl)thietane1,1-dioxide |
| Ex-11.1 | 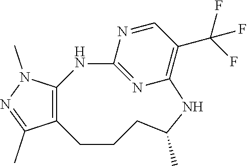 [SMILES: Cc1nn(C)c2c1CCC[C@@H](C)Nc1nc(ncc1C(F)(F)F)N2] | (R)- or (S)- 1,3,7-Trimethyl-10-(trifluoromethyl)- 4,5,6,7,8,14-hexahydro-1H-9,13- (azeno)pyrazolo[4,3-l][1,3,7] triazacyclotridecine |
| Ex-11.2 | 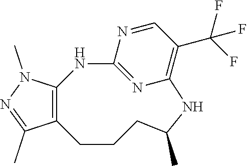 [SMILES: Cc1nn(C)c2c1CCC[C@H](C)Nc1nc(ncc1C(F)(F)F)N2] | (R)- or (S)- 1,3,7-Trimethyl-10-(trifluoromethyl)- 4,5,6,7,8,14-hexahydro-1H-9,13- (azeno)pyrazolo[4,3-l][1,3,7] triazacyclotridecine |
| Ex-12.1 | 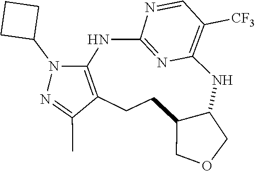 [SMILES: Cc1nn(C2CCC2)c2c1CC[C@@H]1COC[C@H]1Nc1nc(ncc1C(F)(F)F)N2] | (10aS,13aS)- or (10aR,13aR)- 3-Cyclobutyl-1-methyl-8-(trifluoromethyl)- 3,4,10a,11,13,13a,14,15-octahydro-10H-5,9- (azeno)furo[3,4-h]pyrazolo[4,3-l][1,3,7] triazacyclotridecine |
| Ex-12.2 | 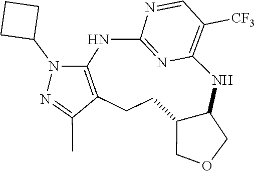 [SMILES: Cc1nn(C2CCC2)c2c1CC[C@H]1COC[C@@H]1Nc1nc(ncc1C(F)(F)F)N2] | (10aS,13aS)- or (10aR,13aR)- 3-Cyclobutyl-1-methyl-8-(trifluoromethyl)- 3,4,10a,11,13,13a,14,15-octahydro-10H-5,9- (azeno)furo[3,4-h]pyrazolo[4,3-l][1,3,7] triazacyclotridecine |
| Ex-13.1 | 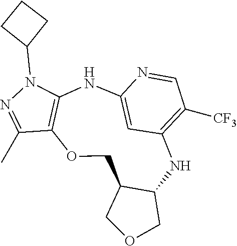 [SMILES: Cc1nn(C2CCC2)c2c1OC[C@@H]1COC[C@H]1Nc1cc(ncc1C(F)(F)F)N2] | (10aR,13aR)-3-Cyclobutyl-1-methyl-8- (trifluoromethyl)-3,4,10a,11,13a,14- hexahydro-10H,13H-5,9-(metheno)furo[3,4- k]pyrazolo[4,3- b][1]oxa[4,6,10]triazacyclotridecine |
| Ex-13.2 | 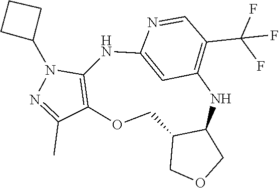 [SMILES: Cc1nn(C2CCC2)c2c1OC[C@H]1COC[C@@H]1Nc1cc(ncc1C(F)(F)F)N2] | (10aS,13aS)- or (10aR,13aR)- 3-cyclobutyl-1-methyl-8-(trifluoromethyl)- 3,4,10a,11,13a,14-hexahydro-10H,13H-5,9- (metheno)furo[3,4-k]pyrazolo[4,3- b][1]oxa[4,6,10]triazacyclotridecine |
| Ex-13.3 | 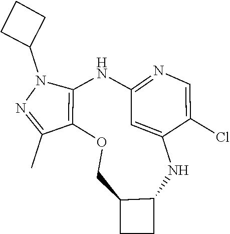 [SMILES: Cc1nn(C2CCC2)c2c1OC[C@@H]1CC[C@H]1Nc1cc(ncc1Cl)N2] | (10aR,12aR)-8-chloro-3-cyclobutyl-1- methyl-3,4,10,10a,11,12,12a,13-octahydro- 5,9-(metheno)cyclobuta[k]pyrazolo[4,3- b][1]oxa[4,6,10]triazacyclotridecine |
| Ex-13.4 | 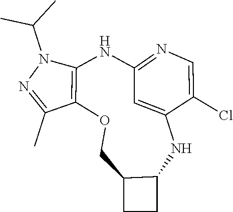 [SMILES: Cc1nn(C(C)C)c2c1OC[C@@H]1CC[C@H]1Nc1cc(ncc1Cl)N2] | (10aR,12aR)-8-chloro-3-isopropyl-1-methyl- 3,4,10,10a,11,12,12a,13-octahydro-5,9- (metheno)cyclobuta[k]pyrazolo[4,3- b][1]oxa[4,6,10]triazacyclotridecine |
| Ex-13.5 | 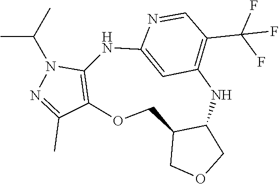 [SMILES: Cc1nn(C(C)C)c2c1OC[C@@H]1COC[C@H]1Nc1cc(ncc1C(F)(F)F)N2] | (10aS,13aS)-3-isopropyl-1-methyl-8- (trifluoromethyl)-3,4,10a,11,13a,14- hexahydro-10H,13H-5,9-(metheno)furo[3,4- k]pyrazolo[4,3- b][1]oxa[4,6,10]triazacyclotridecine |
| Ex-13.6 | 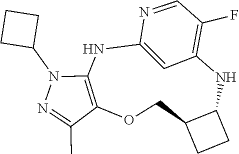 [SMILES: Cc1nn(C2CCC2)c2c1OC[C@@H]1CC[C@H]1Nc1cc(ncc1F)N2] | (10aR,12aR)-3-cyclobutyl-8-fluoro-1- methyl-3,4,10,10a,11,12,12a,13-octahydro- 5,9-(metheno)cyclobuta-[k]pyrazolo[4,3- b][1]oxa[4,6,10]-triazacyclotridecine |
| Ex-13.7 | 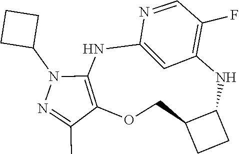 [SMILES: Cc1nn(C2CCC2)c2c1OC[C@@H]1CC[C@H]1Nc1cc(ncc1F)N2] | (10aR,12aR)-3-cyclobutyl-8-fluoro-1- methyl-3,4,10,10a,11,12,12a,13-octahydro- 5,9-(metheno)cyclobuta-[k]pyrazolo[4,3- b][1]oxa[4,6,10]-triazacyclotridecine |
| Ex-13.8 | 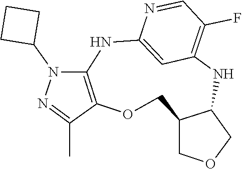 [SMILES: Cc1nn(C2CCC2)c2c1OC[C@@H]1COC[C@H]1Nc1cc(ncc1F)N2] | (10aR,13aR)-3-cyclobutyl-8-fluoro-1- methyl-3,4,10a,11,13a,14-hexahydro- 10H,13H-5,9-(metheno)furo[3,4- k]pyrazolo[4,3- b][1]oxa[4,6,10]triazacyclotridecine |
| Ex-13.9 | 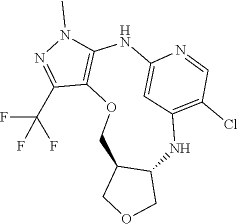 [SMILES: Cn1nc(C(F)(F)F)c2c1Nc1cc(c(Cl)cn1)N[C@@H]1COC[C@H]1CO2] | (10aS,13aS)-8-chloro-3-methyl-1- (trifluoromethyl)-3,4,10a,11,13a,14- hexahydro-10H,13H-5,9-(metheno)furo[3,4- k]pyrazolo[4,3- b][1]oxa[4,6,10]triazacyclotridecine |
| Ex-14.1 | 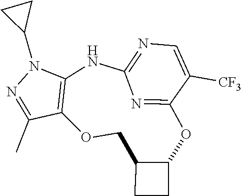 [SMILES: Cc1nn(C2CC2)c2c1OC[C@@H]1CC[C@H]1Oc1nc(ncc1C(F)(F)F)N2] | (10aR,13aS)- or (10aS,13aR)- 3-Cyclopropyl-1-methyl-8- (trifluoromethyl)-3,4,10a,11,12a,-13- hexahydro-12H-5,9-(azeno)- cyclobuta[k]yrazolo[4,3-b][1,10] dioxa[4,6]diazacyclotridecine and |
| Ex-14.2 | 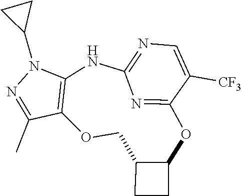 [SMILES: Cc1nn(C2CC2)c2c1OC[C@H]1CC[C@@H]1Oc1nc(ncc1C(F)(F)F)N2] | (10aR,13aS)- or (10aS,13aR)- 3-Cyclopropyl-1-methyl-8- (trifluoromethyl)-3,4,10a,11,12a,13- hexahydro-12H-5,9- (azeno)cyclobuta[k]pyrazolo- [4,3-b][1,10]dioxa[4,6]-diazacyclotridecine |
| Ex-15.1 | 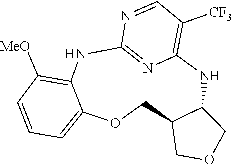 [SMILES: COc1cccc2c1Nc1ncc(C(F)(F)F)c(n1)N[C@@H]1COC[C@H]1CO2] | (3aS,16aS)-11-Methoxy-6-(trifluoromethyl)- 3a,4,16,16a-tetrahydro-1H,3H,10H-5,9- (azeno)benzo[b]furo[3,4- k][1]oxa[4,6,10]triazacyclotridecine |
| Ex-15.2 | 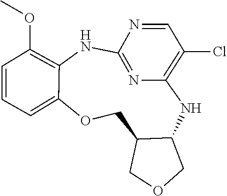 [SMILES: COc1cccc2c1Nc1ncc(Cl)c(n1)N[C@@H]1COC[C@H]1CO2] | (3aS,16aS)-6-chloro-11-methoxy- 3a,4,16,16a-tetrahydro-1H,3H,10H-5,9- (azeno)benzo[b]furo[3,4- k][1]oxa[4,6,10]triazacyclotridecine |
| Ex-15.3 | 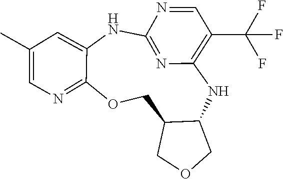 [SMILES: Cc1cnc2c(c1)Nc1ncc(C(F)(F)F)c(n1)N[C@@H]1COC[C@H]1CO2] | (11aS,14aS)-3-methyl-9-(trifluoromethyl)- 11a,12,14a,15-tetrahydro-5H,11H,14H6,10- (azeno)furo[3,4-k]pyrido[2,3- b][1]oxa[4,6,10]triazacyclotridecine |
| Ex-15.4 | 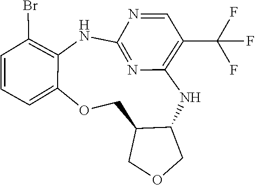 [SMILES: FC(F)(F)c1cnc2nc1N[C@@H]1COC[C@H]1COc1cccc(Br)c1N2] | (3aS,16aS)-11-bromo-6-(trifluoromethyl)- 3a,4,16,16a-tetrahydro-1H,3H,10H-5,9- (azeno)benzo[b]furo[3,4- k][1]oxa[4,6,10]triazacyclotridecine |
| Ex-15.5 | 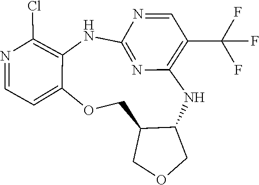 [SMILES: FC(F)(F)c1cnc2nc1N[C@@H]1COC[C@H]1COc1ccnc(Cl)c1N2] | (3aS,16aS)-11-chloro-6-(trifluoromethyl)- 3a,4,16,16a-tetrahydro-1H,3H,10H-5,9- (azeno)furo[3,4-k]pyrido[4,3- b][1]oxa[4,6,10]triazacyclotridecine |
| Ex-15.6 | 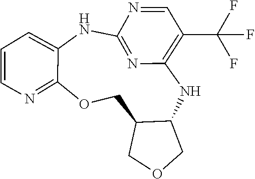 [SMILES: FC(F)(F)c1cnc2nc1N[C@@H]1COC[C@H]1COc1ncccc1N2] | (11aS,14aS)-9-(trifluoromethyl)- 11a,12,14a,15-tetrahydro-5H,11H,14H6,10- (azeno)furo[3,4-k]pyrido[2,3- b][1]oxa[4,6,10]triazacyclotridecine |
| Ex-15.7 | 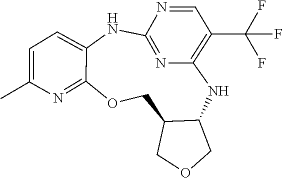 [SMILES: Cc1ccc2c(n1)OC[C@@H]1COC[C@H]1Nc1nc(ncc1C(F)(F)F)N2] | (11aS,14aS)-2-methyl-9-(trifluoromethyl)- 11a,12,14a,15-tetrahydro-5H,11H,14H6,10- (azeno)furo[3,4-k]pyrido[2,3- b][1]oxa[4,6,10]triazacyclotridecine |
| Ex-15.8 | 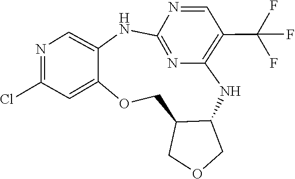 [SMILES: FC(F)(F)c1cnc2nc1N[C@@H]1COC[C@H]1COc1cc(Cl)ncc1N2] | (3aS,16aS)-13-chloro-6-(trifluoromethyl)- 3a,4,16,16a-tetrahydro-1H,3H,10H-5,9- (azeno)furo[3,4-k]pyrido[4,3- b][1]oxa[4,6,10]triazacyclotridecine |
| Ex-15.9 | 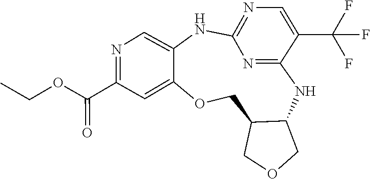 [SMILES: CCOC(=O)c1cc2c(cn1)Nc1ncc(C(F)(F)F)c(n1)N[C@@H]1COC[C@H]1CO2] | ethyl (3aS,16aS)-6-(trifluoromethyl)- 3a,4,16,16a-tetrahydro-1H,3H,10H-5,9- (azeno)furo[3,4-k]pyrido[4,3- b][1]oxa[4,6,10]triazacyclotridecine-13- carboxylate |
| Ex-16.1 | 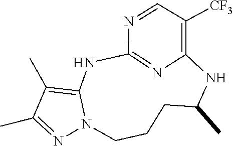 | 8,14,15-Trimethyl-5-(trifluoromethyl)- 8,9,10,11-tetrahydro-1H,7H-2,6- (azeno)pyrazolo[1,5- a][1,3,5,9]tetraazacyclotridecine |
| Ex-16.2 | 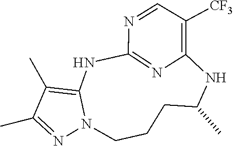 | 8,14,15-Trimethyl-5-(trifluoromethyl)- 8,9,10,11-tetrahydro-1H,7H-2,6- (azeno)pyrazolo[1,5- a][1,3,5,9]tetraazacyclotridecine |
| Ex-17.1 | 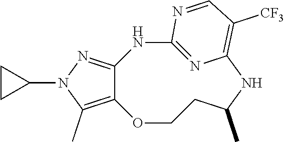 [SMILES: Cc1c2c(nn1C1CC1)Nc1ncc(C(F)(F)F)c(n1)N[C@@H](C)CCO2] | (R)- or (S)-2-cyclopropyl-3,7-dimethyl-10- (trifluoromethyl)-2,5,6,7,8,14-hexahydro- 9,13-(azeno)pyrazolo[4,3- b][1]oxa[4,6,10]triazacyclotridecine |
| Ex-17.2 | 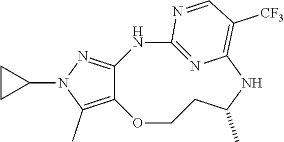 [SMILES: Cc1c2c(nn1C1CC1)Nc1ncc(C(F)(F)F)c(n1)N[C@H](C)CCO2] | (R)- or (S)-2-cyclopropyl-3,7-dimethyl-10- (trifluoromethyl)-2,5,6,7,8,14-hexahydro- 9,13-(azeno)pyrazolo[4,3- b][1]oxa[4,6,10]triazacyclotridecine |
| Ex-18.1 | 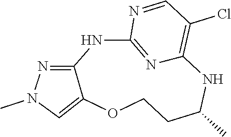 | (R)-10-chloro-2,7-dimethyl-2,5,6,7,8,14- hexahydro-9,13-(azeno)pyrazolo[4,3- b][1]oxa[4,6,10]triazacyclotridecine |
| Ex-19.1 | 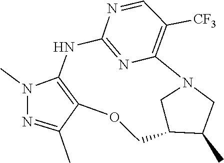 | tert-Butyl (3S,4S)-3-(((5-((4-chloro-5- (trifluoromethyl)pyrimidin-2-yl)amino)-1,3- dimethyl-1H-pyrazol-4-yl)oxy)methyl)-4- methylpyrrolidine-1-carboxylate |
| Ex-20.1 | 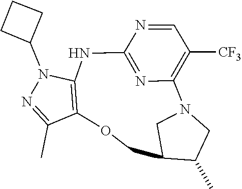 [SMILES: Cc1nn(C2CCC2)c2c1OC[C@H]1CN(C[C@@H]1C)c1nc(ncc1C(F)(F)F)N2] | (6S,7S)- or (6R,7R)-1-Cyclobutyl-3,7- dimethyl-11-(trifluoromethyl)-1,5,6,7,8,15- hexahydro-10,14-(azeno)-6,9- methanopyrazolo[4,3- b][1]oxa[4,6,10]triazacyclotridecine |
| Ex-20.2 | 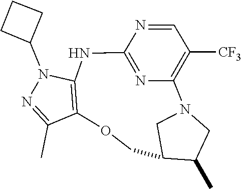 [SMILES: Cc1nn(C2CCC2)c2c1OC[C@@H]1CN(C[C@H]1C)c1nc(ncc1C(F)(F)F)N2] | (6S,7S)- or (6R,7R)-1-Cyclobutyl-3,7- dimethyl-11-(trifluoromethyl)-1,5,6,7,8,15- hexahydro-10,14-(azeno)-6,9- methanopyrazolo[4,3- b][1]oxa[4,6,10]triazacyclotridecine |
| Ex-21.1 | 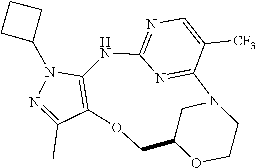 [SMILES: Cc1nn(C2CCC2)c2c1OC[C@H]1CN(CCO1)c1nc(ncc1C(F)(F)F)N2] | (6R)- or (6S)-1-cyclobutyl-3-methyl-12- (trifluoromethyl)-15,6,8,9,16-hexahydro- 11,15-(azeno)-6,10-methanopyrazolo[4,3- e][1,4]dioxa[7,9,13]triazacyclopentadecine |
| Ex-21.2 | 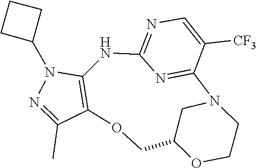 [SMILES: Cc1nn(C2CCC2)c2c1OC[C@@H]1CN(CCO1)c1nc(ncc1C(F)(F)F)N2] | (6R)- or (6S)-1-cyclobutyl-3-methyl-12- (trifluoromethyl)-1,5,6,8,9,16-hexahydro- 11,15-(azeno)-6,10-methanopyrazolo[4,3- e][1,4]dioxa[7,9,13]triazacyclopentadecine |
| Ex-21.3 | 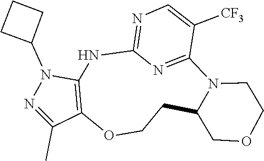 [SMILES: Cc1nn(C2CCC2)c2c1OCC[C@@H]1COCCN1c1nc(ncc1C(F)(F)F)N2] | (R) or (S)-3-cyclobutyl-1-methyl-8- (trifluoromethyl)-3,4,11,12,14,14a,15,16- octahydro-5,9-(azeno)[1,4]oxazino[4,3-j] pyrazolo[4,3- b][1]oxa[4,6,10]triazacyclotridecine |
| Ex-21.4 | 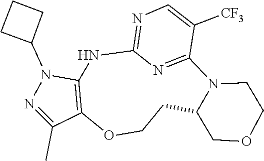 [SMILES: Cc1nn(C2CCC2)c2c1OCC[C@H]1COCCN1c1nc(ncc1C(F)(F)F)N2] | (R) or (S)-3-cyclobutyl-1-methyl-8- (trifluoromethyl)-3,4,11,12,14,14a,15,16- octahydro-5,9-(azeno)[1,4]oxazino[4,3-j] pyrazolo[4,3- b][1]oxa[4,6,10]triazacyclotridecine |

or a pharmaceutically acceptable salt thereof.

17. A pharmaceutical composition comprising a compound of claim 1 or a pharmaceutically acceptable salt thereof, and a pharmaceutically acceptable carrier.

18. (canceled)

19. A compound according to claim 1 for use in therapy.

20. A method of treating Parkinson's Disease comprising administering an effective amount of a compound of claim 1, or a pharmaceutically acceptable salt thereof, to a person in need thereof.

21. A method for the treatment or prophylaxis of an indication in which LRRK2 kinase is involved comprising administering to a subject in need thereof an effective amount of a compound according to claim 1, or a pharmaceutically acceptable salt thereof, said indication selected from:
abnormal motor symptoms associated with Parkinson's disease, non-motor symptoms associated with Parkinson's disease, Lewy body dementia, L-Dopa induced dyskinesias,
Alzheimer's disease, mild cognitive impairment, the transition from mild cognitive impairment to Alzheimer's disease, tauopathy disorders characterized by hyperphosphorylation of tau such as argyrophilic grain disease, Picks disease, corticobasal degeneration, progressive supranuclear palsy, inherited frontotemporal dementia, and Parkinson's disease linked to chromosome 17,
neuroinflammation associated with of microglial inflammatory responses associated with multiple sclerosis, HIV-induced dementia, ALS, ischemic stroke, traumatic brain injury and spinal cord injury,
lymphomas, leukemias, multiple sclerosis, rheumatoid arthritis, systemic lupus erythematosus, autoimmune hemolytic anemia, pure red cell aplasia, idiopathic thrombocytopenia pupura (ITP), Evans Syndrome, vasculitis, bullous skin disorder, type I diabetes mellitus, Sjorgen's syndrome, Delvic's disease, inflammatory myopathies, and ankylosing spondylitis,
renal cancer, breast cancer, lung cancer, prostate cancer, and acute myelogenous leukemia (AML) in subjects expressing the LRRK2 G2019S mutation,
papillary renal and thyroid carcinomas in a subject in whom LRRK2 is amplified or overexpressed, Crohn's disease and leprosy.
